(19) United States
(12) Patent Application Publication
(10) Pub. No.: US 2023/0165975 A1
(43) Pub. Date: Jun. 1, 2023

(54) ACTIVITY-DEPENDENT GENE THERAPY FOR NEUROLOGICAL DISORDERS

(71) Applicant: UCL BUSINESS LTD., London (GB)

(72) Inventors: Gabriele LIGNANI, London (GB); Dimitri Michael KULLMANN, London (GB); Stephanie SCHORGE, London (GB); Yichen QIU, London (GB); Matthew Charles WALKER, London (GB)

(21) Appl. No.: 17/915,043

(22) PCT Filed: Mar. 29, 2021
(86) PCT No.: PCT/EP2021/058210

§ 371 Date: Sep. 27, 2022

(30) Foreign Application Priority Data
Mar. 27, 2020 (GB) 2004498.8

(51) Int. Cl. A61K 48/00; C12N 7/00; A61P 25/08; C12N 15/86

(52) U.S. Cl. CPC A61K 48/0058; A61P 25/08; C12N 7/00; C12N 15/86; C12N 2750/14143; C12N 2830/002

(57) ABSTRACT

The invention provides expression vectors or vector systems comprising a polynucleotide sequence encoding a polypeptide, wherein the gene is operably linked to a neuronal activity-dependent promoter suitable to drive expression of the gene product in a subject’s neural cells. The features of the expression vectors combine to advantageously improve the treatment of a neurological disorder associated with neuronal hyperexcitability in a subject. The invention also provides the expression vectors or vector systems for use in related methods of treatment, as well as viral particles, cells, kits and methods using the expression vectors or vector systems.

This application claims priority from GB2004498.8 filed 27 Mar. 2020, the contents and elements of which are herein incorporated by reference for all purposes.

### TECHNICAL FIELD

The present invention relates generally to methods and materials involving gene products that are expressed in an activity-dependent manner, which can be used in treating neurological disorders, such as epilepsy.

### BACKGROUND ART

Neurological circuit disorders, characterized by abnormal firing of neurons, account for an enormous burden to society and are inadequately treated with drugs. For instance, epilepsy affects up to 1% of the population. Of these sufferers, 30% are refractory (“pharmacoresistant”) to pharmacological treatment, and surgical resection of the brain area where seizures arise (the epileptogenic zone) remains the best hope to achieve seizure freedom. However, such surgery is unsuitable for many due to risk of damage to eloquent regions of the cortex or white matter pathways involved in functions such as memory, language, vision or motor control (Kwan, P. et al (2011), N. Engl. J. Med. 365, 919-926; Picot, M.C. et al (2008), Epilepsia 49, 1230-1238).

New anti-epileptic drugs have had little impact on refractory epilepsy and people with uncontrolled seizures continue to experience co-morbidities, social exclusion, and a substantial risk of sudden unexpected death in epilepsy (SUDEP). Refractory epilepsy is mostly focal (that is, characterized by seizures arising from the epileptogenic zone) but primary generalized epilepsy can also be resistant to pharmacotherapy.

Although surgical resection of the epileptogenic zone can result in seizure freedom, it is unsuitable for 90% of people with refractory epilepsy. Furthermore, the extent of surgical resection is limited by risk of irreversible neurological deficit, meaning that many patients undergoing surgery continue to have seizures. Minimally invasive ablation procedures using lasers have a role in targeting inaccessible deep structures in the brain but are also limited by risk of damage to neighbouring structures. Deep brain stimulation and other neuromodulatory treatments are of limited effectiveness.

Gene therapy is a promising candidate as a rational replacement for surgical treatment of pharmacoresistant focal epilepsy. Examples include overexpression of neuropeptide Y and Y2 receptors (Woldbye et al, 2010), Kv1.1 overexpression (Wykes et al, 2012; Snowball et al. 2019; WO2018/229254); chemogenetics using designer receptors exclusively activated by designer drugs (DREADDs), e.g. hM4Di (Katzel, et al, 2014), and use of the enhanced glutamate-gated chloride channel eGluCI (Lieb et al, 2018).

However, current experimental gene therapies are based on either the permanent modification of neuronal excitability (neurotransmitter, ion channel or receptor overexpression) or the exogenous delivery of light or chemicals to achieve on-demand modulation of neuronal activity (optogenetics and chemogenetics). These approaches have limitations, due to off-target effects. They do not distinguish between neurons involved in seizures and intermingled ‘bystander’ neurons. By analogy, deep brain stimulation (DBS) is an example of a therapy (e.g., for Parkinson Disease, OCD and depression) targeted to specific brain sites, but it is not cell-type specific and can produce side effects. Furthermore, although optogenetics and chemogenetics can be used on demand, the decision when to activate the therapy by light or drug delivery requires an additional step, such as human intervention or a computer that detects seizures. A gene therapy that avoids some of these limitations is the use of eGluCl, which opens in response to accumulation of extracellular glutamate, as occurs in epilepsy, but the effectiveness of this approach in common forms of epilepsy is unknown, and it relies on permanent expression of a non-mammalian membrane protein, which may represent a risk of immunogenicity.

Thus, there is an urgent need to develop alternative therapies for refractory epilepsy, amongst other neurological disorders, with fewer off-target effects or side effects.

### DISCLOSURE OF THE INVENTION

The inventors have found that by using a neuronal activity-dependent promoter to drive or alter expression of genes that affect neuronal properties, they can achieve selective modulation of neurons driving seizures or contributing to propagation of seizures in the brain. In this way, neurological disorders, such as refractory epilepsy, can be treated with fewer off-target effects or side effects.

For instance, in one case, when the potassium channel gene KCNA1 is put under the control of the activity-dependent c-Fos promoter, up-regulation of KCNA1 expression is induced in response to intense neuronal activity (e.g. a seizure), and this leads to a decrease in neuronal excitability and neurotransmitter release, resulting in a decrease in susceptibility to seizure initiation or propagation. If the circuit activity returns to near-normal levels, promoter activity decreases, and expression of the potassium channel returns to baseline. This gene therapy is thus specific both for neurons that are over-active (as opposed to bystander neurons) and for the duration that the hyperactivity persists.

In another case, when a fusion protein, composed of dCas9 (also known as endonuclease deficient cas9) and transcriptional activators, is put under the control of the activity-dependent c-Fos promoter, up-regulation of this protein is induced in response to intense neuronal activity (e.g. a seizure), and, in the presence of an appropriate single guide RNA (sgRNA), this can lead to altered expression of an endogenous gene. Altered expression of the endogenous gene (for example, KCNA1) then leads to a decrease in neuronal excitability and neurotransmitter release, resulting in a decrease in susceptibility to seizure initiation or propagation. If the circuit activity returns to near-normal levels, promoter activity decreases, and expression of the fusion protein (and the endogenous gene) returns to baseline. This gene therapy is thus, again, specific both for neurons that are over-active (as opposed to bystander neurons) and for the duration that the hyperactivity persists.

The activity of the c-Fos promoter has been shown to increase in response to several forms of intense neuronal activation (e.g. Hunt et al., 1987 PMID: 3112583; Singewald et al., 2003 PMID: 12586446), and c-Fos activation has also been reported in astrocytes (Morishita et al., PMID: 21785243), oligodendrocytes (Muir & Compston, 1996 PMID: 8926624) and microglia (Eun et al., 2004 PMID: 15522236). Thus, it could not have been predicted that use of an activity-dependent promoter in the treatment of epilepsy would lead to fewer off-target effects. Further, it could not be predicted that activity-dependent promoters used in this way would provide sufficient expression to have a functional effect, or improved functional effect, in vivo.

Accordingly, in one aspect the invention provides an expression vector or vector system for use in a method of treatment of a neurological disorder associated with neuronal hyperexcitability in a subject, the vector or vector system being as defined in the claims. Where relevant, the term “vector” may refer to “vector system” in the detailed description,

In another aspect, the invention provides an expression vector or expression vector system as defined in the claims.

In another aspect, the invention provides an in vitro method of making viral particles as defined in the claims. In another aspect, the invention provides a viral particle as defined in the claims, and such viral particles for use in methods as defined in the claims.

In another aspect, the invention provides a kit as defined in the claims.

In another aspect the invention provides a method of treatment of a neurological disorder, as defined in the claims. In another aspect, the invention provides a method of confirming the presence of a gene product, the method being as defined in the claims.

In another aspect, the invention provides a cell as defined in the claims.

Some particular aspects of the invention will now be discussed in more detail.

### Activity-Dependent Promoters

The term “neuronal activity-dependent promoter” (or “activity-dependent promoter” as used interchangeably herein) refers to a promoter that alters or drives expression of a target gene in response to changes in neuronal activity in neural cells. Such changes in neuronal activity may result from a neural cell that becomes hyperexcited, for example during a seizure.

The neural cell may be a neuron or a glial cell. In particularly preferred embodiments, the neural cell is a neuron. In some embodiments, the neuron is a cortical neuron.

In some preferred embodiments, the neuronal activity-dependent promoter is an immediate early gene (IEG) promoter. As used herein, the term “immediate early gene” (IEG) is a gene whose expression is increased immediately following a stimulus to a cell comprising the IEG. For example, genes expressed by neurons that exhibit a rapid increase in expression immediately following neuronal stimulation are neuronal lEGs. Such neuronal lEGs have been found to encode a wide variety of polypeptides including transcription factors, cytoskeletal polypeptides, growth factors, and metabolic enzymes as well as polypeptides involved in signal transduction. The identification of neuronal lEGs and the polypeptides they encode provides important information about the function of neurons in, for example, learning, memory, synaptic transmission, tolerance, and neuronal plasticity.

A number of suitable IEG promoters can be used in accordance with the invention. In some preferred embodiments, the IEG promoter comprises c-Fos (or “cFos”). c-Fos is a nuclear proto-oncogene which has been implicated in a number of important cellular events, including cell proliferation (Holt et al. (1986) Proc. Natl. Acad. Set USA 831:4794-4798; Riabowol et al. (1988) J. Cell. Biol. 8: 1670-1676), differentiation (Distel et al. (1987) Cell 49: 835-844; Lord et al. (1993) Mol Cell. Biol. 13:841-851), and tumorigenesis (Cantor et al. (1993) Proc. Natl. Acad. Sci. USA90:10932-10936; Miller et al. (1984) Cell 36:51-60; Ruther et al. (1989) Oncogene 4:861-865.

c-Fos encodes a 62 kDa protein which forms heterodimers with c-Jun, forming an AP-1 transcription factor which binds to DNA at an AP-1 element and stimulates transcription. Fos gene products can also repress gene expression. Sassone et al. (1988) Nature 334:314-319 showed c-Fos inhibits its own promoter, and Gius et al. (1990) and Hay et al. (1989) showed c-Fos inhibits early response genes Egr-1 and c-myc. AP-1 factors have also been shown to inhibit expression of the MHC class l and PEPCK genes (see Gurney et al.(1992) J Biol. Chem. 267: 18133-18139).

c-Fos regulatory region activation can occur in multiple cell types. Where the target cell is a neuron, a stimulus sufficient for c-Fos regulatory region activation may include but is not limited to e.g., neuronal activation, including synaptic activation, electrophysiological activation and the like.

In some embodiments, the c-Fos promoter has a nucleotide sequence comprising or consisting of the nucleotide sequence of SEQ ID NO: 3. In some embodiments, the c-Fos promoter has a nucleotide sequence comprising or consisting of a nucleotide sequence having at least 70, 75, 80, 85, 90, 91, 92, 93, 94, 95, 96, 97, 98 or 99% identity to the nucleotide sequence of SEQ ID NO: 3.

In some cases, the c-Fos promoter comprises CREB, SRE, AP1 and SIF motifs. In some cases, the c-Fos promoter consists of CREB, SRE, AP1 and SIF motifs.

CREB-TF (CREB, cAMP response element-binding protein) is a cellular transcription factor. It binds to certain DNA sequences called cAMP response elements (CRE), thereby increasing or decreasing the transcription of the genes. Serum response factor, also known as SRF, is a transcription factor protein. This protein binds to the serum response element (SRE) in the promoter region of target genes. This protein regulates the activity of many immediate early genes, for example c-fos, and thereby participates in cell cycle regulation, apoptosis, cell growth, and cell differentiation. Activator protein 1 (AP1) is a transcription factor that regulates gene expression in response to a variety of stimuli, including cytokines, growth factors, stress, and bacterial and viral infections. Sis-inducible factor (SIF) binding element confers sis/PDGF inducibility to the c-fos promoter.

In other embodiments, the activity-dependent promoter is Egr1 (also known as Zif268), Arc, Homer1a, Bdnf, Creb, Srf, Mef2, Fosb, and Npas4 or synthetic activity-dependent promoters such as P_{RAM} (Sørensen et al., eLife 2016) and ESARE (Kawashima et al., Nature Methods 2013 PMID: 23852453), or part of them or combinations of the above, can be used instead of c-Fos.

In some embodiments, the Egr1 promoter has a nucleotide sequence comprising or consisting of the nucleotide sequence of SEQ ID NO: 18. In some embodiments, the Egr1 promoter has a nucleotide sequence comprising or consisting of a nucleotide sequence having at least 70, 75, 80, 85, 90, 91, 92, 93, 94, 95, 96, 97, 98 or 99% identity to the nucleotide sequence of SEQ ID NO: 18.

In some embodiments, the activity-dependent promoter is Arc or an Arc minimal sequence (mArc). Arc is an activity-regulated cytoskeleton-associated protein mostly expressed in glutamatergic neurons in hippocampus and neocortex, with little or no expression in glial cells. In some embodiments, the Arc or mArc promoter has a nucleotide sequence comprising or consisting of the nucleotide sequence of SEQ ID NO: 15. In some embodiments, the mArc promoter has a nucleotide sequence comprising or consisting of a nucleotide sequence having at least 70, 75, 80, 85, 90, 91, 92, 93, 94, 95, 96, 97, 98 or 99% identity to the nucleotide sequence of SEQ ID NO: 15. mArc promoter is a truncated version of the full-length Arc promoter.

In other embodiments, the activity-dependent promoter is PRAM (Promoter Robust Activity Marker) or parts of this synthetic promoter: NRAM (NPAS4 Robust Activity Marker) or FRAM (Fos Robust Activity Marker). PRAM consists of repeats of core NRE/AP-1 DNA motifs inserted into the central midline element (CME) to form a secondary structure favoured by transcriptional activation. They have a longer activation window, potentially able to drive more stable and less transient expression of the operatively linked gene. NRAM comprises the NPAS-4 Responsive Element (the consensus binding motif for NPAS4), with a minimal human c-fos promoter. FRAM consists of AP-1 modules (a consensus binding sequence for FOS/JUN family transcription factors) with a human c-fos minimal promoter (see e.g. Sun et al; Cell Volume 181, Issue 2, 16 Apr. 2020, Pages 410-423.e17). In some embodiments, the PRAM, FRAM and NRAM promoters comprise a nucleotide sequence comprising or consisting of the nucleotide sequence of SEQ ID NO: 17. In some embodiments, the promoter has a nucleotide sequence comprising or consisting of a nucleotide sequence having at least 70, 75, 80, 85, 90, 91, 92, 93, 94, 95, 96, 97, 98 or 99% identity to the nucleotide sequence of SEQ ID NO: 17.

In other embodiments, the activity-dependent promoter is E-SARE (Enhanced Synaptic Activity Responsive elements). This synthetic promoter contains five repeats of SARE motifs for CREB, MEF2 and SRF binding for transcription initiation, and a minimal Arc promoter (mArc). SARE is part of the Arc promoter. SARE motifs regulate the induction of the immediate-early gene Arc. Mef2 is a critical regulator in heart development and cardiac gene expression. In some embodiments, the E-SARE promoter has a nucleotide sequence comprising or consisting of the nucleotide sequence of SEQ ID NO: 16. In some embodiments, the E-SARE promoter has a nucleotide sequence comprising or consisting of a nucleotide sequence having at least 70, 75, 80, 85, 90, 91, 92, 93, 94, 95, 96, 97, 98 or 99% identity to the nucleotide sequence of SEQ ID NO: 16.

NRAM and E-SARE are both composed of sequences from natural promoters. NRAM comprises part of the Npas4 promoter. E-SARE is based on tandem repeats of sequences from the Arc promoter.

In some embodiments, the activity-dependent promoter suppresses the level of expression of a gene, for instance by driving transcription of a short hairpin RNA (shRNA), or another type of RNA that binds to the messenger RNA of an endogenous sodium channel, or other protein.

### Preferred Genes and Gene Products

In some preferred embodiments, the gene that is operably linked to the activity-dependent promoter defined in the claims is KCNA1. KCNA1 (Gene ID 3736, also known as the Potassium Voltage-Gated Channel Subfamily A Member 1, KV1.1, HBK1 and RBK1) is a human gene that encodes the human Kv1.1 potassium channel subunit (also known as Potassium voltage-gated channel subfamily A member 1). By “wild-type KCNA1 gene” it is meant the nucleic acid molecule that is found in human cells and encodes the human Kv1.1 potassium channel subunit. The KCNA1 gene may include regulatory sequences upstream or downstream of the coding sequence. A nucleotide sequence for the wild-type KCNA1 gene, including the non-coding 5′ and 3′ untranslated regions (5′ and 3′ UTRs) is provided in NCBI Reference Sequence NM_000217.2. The coding sequence for the wild-type KCNA1 gene has the nucleotide sequence of SEQ ID NO: 4, which corresponds to positions 1106 to 2593 of NCBI Reference Sequence NM_000217.2.

In some preferred embodiments, the gene product encoded by the gene defined in the claims is the Kv1.1 potassium channel subunit. Kv1 family channels are made up of four subunits. Although four Kv1.1 subunits on their own can make up a functional channel, Kv1.1-containing potassium channels that occur in the mammalian nervous system typically also contain other subunits from the Kv1 family, and so a complete tetrameric channel may contain Kv1.1 together with Kv1.2 or Kv1.4 in various stoichiometries. The term ‘Kv1.1 channel’ is used interchangeably either to indicate a Kv1.1 channel subunit or to indicate a homotetrameric or heterotetrameric channel that contains at least one Kv1.1 subunit.

The Kv1.1 potassium channel is a voltage-gated delayed-rectifier potassium channel that is phylogenetically related to the Drosophila Shaker channel. The amino acid sequence for the wild-type Kv1.1 potassium channel subunit has the amino acid sequence of SEQ ID NO: 5 which is identical to the NCBI Reference Sequence NP_000208.2. Voltage-dependent potassium channels modulate excitability by opening and closing a potassium-selective pore in response to voltage. In many cases, potassium ion flow can be interrupted when an intracellular particle occludes the pore, a process known as fast inactivation. Kv1 potassium channel subunits have six putative transmembrane segments, and the loop between the fifth and sixth segment of each of the four Kv1 subunits that make up a complete channel forms the pore.

During normal production in cells, some of the KCNA1 RNA in the cell is edited by an adenosine deaminase acting on RNA (ADAR) that causes an isoleucine/valine (I/V) recoding event at a single position I400 that lies within the sixth transmembrane domain and lines the inner vestibule of the ion-conducting pore (Hoopengardner et al., Science 301(5634):832-6, 2003). At negative membrane potentials, channels containing unedited 1400 recover from inactivation at a rate around twenty times slower than their edited (V400) counterparts (Bhalla et al., 2004).

In some preferred embodiments, the present invention involves activity-dependent expression of a gene product that is an edited Kv1.1 potassium channel. An “edited Kv1.1 potassium channel” is a functional Kv1.1 potassium channel but contains the isoleucine/valine mutation described above. It is believed that these edited Kv1.1 potassium channels are much quicker at recovering from inactivation than their unedited counterparts.

In some embodiments, an edited Kv1.1 potassium channel has an amino acid sequence having at least 70, 75, 80, 85, 90, 91, 92, 93, 94, 95, 96, 97, 98, 99 or 100% sequence identity to the amino acid sequence shown in SEQ ID NO: 2 provided it also contains a valine amino acid residue at a position corresponding to amino acid residue 400 shown in SEQ ID NO: 2(the ‘edited position’). In some preferred embodiments, the edited Kv1.1 potassium channel has an amino acid sequence comprising or consisting of the amino acid sequence shown in SEQ ID NO: 2.

An edited Kv1.1 potassium channel that contains a valine amino acid residue at a position corresponding to amino acid residue 400 shown in SEQ ID NO: 2 can be identified by methods known in the art. For example, the edited position can be identified by a sequence alignment between the amino acid sequence of SEQ ID NO: 2 and the amino acid sequence of the edited Kv1.1 potassium channel of interest. Such sequence alignments can then be used to identify the edited position in the edited Kv1.1 potassium channel of interest which, at least in the alignment, is near, or at the same position as, the edited position at amino acid residue 400 in the amino acid sequence shown in SEQ ID NO: 2.

A functional Kv1.1 potassium channel is a protein that retains the normal activity of a potassium channel, e.g. the channels are able to open and close in response to voltage. Methods of testing that the Kv1.1 potassium channels are functional are known in the art and some of which are described herein. Briefly, a suitable method for confirming that the Kv1.1 potassium channel is functional involves transfecting cells with an expression vector encoding a Kv1.1 potassium channel and using electrophysiological techniques such as patch clamping to record currents of the potassium channels.

The wild-type Kv1.1 potassium channel comprises a tyrosine amino acid at position 379 as shown in SEQ ID NO: 5. In some embodiments, an edited Kv1.1 potassium channel comprises a tyrosine amino acid residue at a position corresponding to amino acid residue 379 shown in SEQ ID NO: 2.

In other embodiments, an edited Kv1.1 potassium channel comprises a valine amino acid residue at a position corresponding to amino acid residue 379 shown in SEQ ID NO: 2. An example of an edited Kv1.1 potassium channel with this amino acid sequence is shown in SEQ ID NO: 12. Without wishing to be bound by any particular theory, it is believed that a Y379V mutation reduces the sensitivity of Kv1.1 channels to tetraethyl ammonium (TEA) without altering the functional properties of the potassium channel. For example, this change in sensitivity allows transgenic Kv1.1 channels to be pharmacologically isolated from their wild-type counterparts in patch clamp electrophysiology experiments (Heeroma et al. 2009).

In some embodiments, an “engineered KCNA1 gene” is used. An engineered KCNA1 gene differs from the nucleotide sequence of the wild-type KCNA1 gene as described herein but still encodes for a functional Kv1.1 potassium channel. As used herein, an engineered KCNA1 gene has a nucleotide sequence having at least 70, 75, 80, 85, 90, 91, 92, 93, 94, 95, 96, 97, 98, 99 or 100% sequence identity to the nucleotide sequence shown in SEQ ID NO: 1. In some preferred embodiments, the engineered KCNA1 gene has a nucleotide sequence comprising or consisting of the nucleotide sequence shown in SEQ ID NO: 7.

As described above, an embodiment of the invention includes an engineered KCNA1 gene encoding an edited potassium channel that comprises a valine amino acid residue at position 379, as shown in SEQ ID NO: 12. An example of an engineered KCNA1 gene that encodes the amino acid sequence shown in SEQ ID NO: 12 is the nucleotide sequence shown in SEQ ID NO: 11. In some embodiments, the engineered KCNA1 gene has a nucleotide sequence comprising or consisting of the nucleotide sequence shown in SEQ ID NO: 11, or has at least 75, 80, 85, 90, 91, 92, 93, 94, 95, 96, 97, 98, 99 or 100% sequence identity to the nucleotide sequence shown in SEQ ID NO: 11.

In other embodiments, the gene product is another protein that affects neuronal excitability or neurotransmitter release, including other potassium channels such as Kv1.2, or neurotransmitter receptors such as GABAa or GABAb receptors, adenosine A1 receptors, and NPY Y2 or Y5 receptors, or neuropeptides such as galanin, NPY or dynorphin.

In some preferred embodiments, the gene that is operably linked to the activity-dependent promoter is defined in the claims as KCNJ2. KCNJ2 encodes the inward-rectifying potassium chancel Kir2.1, which is normally expressed in skeletal muscle. Kir2.1 contributes to maintaining a negative resting membrane potential, thus reducing intrinsic excitability.

In some preferred embodiments, the gene product encoded by the gene defined in the claims is the inward-rectifying potassium channel Kir2.1, which is described above. The nucleotide sequence of KCNJ2 is provided herein.

In some embodiments, the KCNJ2 gene has a nucleotide sequence having at least 70, 75, 80, 85, 90, 91, 92, 93, 94, 95, 96, 97, 98, 99 or 100% sequence identity to the nucleotide sequence shown in SEQ ID NO: 13. In some embodiments, the Kir2.1 gene has an amino acid sequence having at least 70, 75, 80, 85, 90, 91, 92, 93, 94, 95, 96, 97, 98, 99 or 100% sequence identity to the amino acid sequence shown in SEQ ID NO: 14.

In another embodiment, the present invention involves activity-dependent expression of an intermediate gene product that indirectly affects neuronal excitability by altering (increasing or decreasing) the expression of a further gene or gene product, which may be an endogenous gene or gene product. The further/endogenous gene or gene product may be any gene or gene product described herein, such as KCNA1 or KCNJ2. Other further/endogenous genes or gene products include neurotransmitter receptors such as GABAa or GABAb receptors, adenosine A1 receptors, and NPY Y2 or Y5 receptors, or neuropeptides such as galanin, NPY or dynorphin.

Altering expression of the further gene or gene product by activity-dependent expression of the intermediate gene product can, in some cases, be achieved through activity-dependent expression of a fusion protein composed of dCas9 (also known as endonuclease deficient Cas9) and transcriptional activators. The fusion protein may also be composed of any suitable dcas protein, such spCas9 or saCas9. In the presence of an appropriate single guide RNA (sgRNA) this strategy, also known as CRISPR activation (CRISPRa) can lead to increased transcription of a further gene such as KCNA1 that reduces neuronal excitability. In some cases, the sgRNA targets a target sequence with 100% efficiency. The sgRNA may be constitutively expressed and operably linked to a separate promoter, such as RNA polymerase III (e.g. U6). The separate promoter may also be any promoter suitable to express sgRNA, such as an RNA polymerase, for example RNA polymerase II. The sgRNA and separate promoter may also be comprised by, or separate to, the expression vectors and vector systems disclosed herein. In some cases, the sgRNA may also be operably linked to the activity-dependent promoter, or to an intermediate inducible promoter such as Tet-On.

In some embodiments, the sgRNA comprises or consists of a nucleotide sequence having at least 70, 75, 80, 85, 90, 91, 92, 93, 94, 95, 96, 97, 98, 99 or 100% sequence identity to the nucleotide sequence shown in SEQ ID NO: 37.

Activity-dependent expression of an intermediate gene product to indirectly affect neuronal excitability may be achieved via an intermediate expression system, such as an intermediate inducible expression system. Such intermediate expression systems are, in a general sense, known in the art, and may be appropriately selected by the skilled person in order to optimise expression of the intermediate gene or further gene.

For example, the intermediate expression system may be an inducible expression system such as Tet-On. See e.g. Gaia Colasante et. al (Brain, Volume 143, Issue 3, March 2020, Pages 891-905, https://do|org/10.1093/brain/awaa045), the contents of which is incorporated herein by reference in its entirety. An exemplary embodiment of this aspect of the invention is shown in FIG. 25. In this embodiment, the intermediate gene is rtTA and/or dCas9, and may also encode further transcriptional activators.

Of the currently available inducible gene expression systems, Tet-On is the most widely characterised. In some embodiments, in order to improve brain penetration and reduce side-effects in human subjects, the intermediate inducible gene expression system may be a “GeneSwitch™” system. “GeneSwitch™”, uses a chimeric protein, consisting of a truncated human progesterone receptor that does not respond to endogenous steroids, along with a Gal4 DNA binding domain and a P65 activation domain. The receptor is activated by mifepristone, which frees the complex from co-repressors and allows it to initiate transcription of the desired gene in the nucleus by binding to an upstream activating sequence (UAS).

The intermediate expression system can also comprise expression of a modified ecdysone receptor that regulates an optimized ecdysone responsive promoter. The intermediate expression systems can also be based on cumate-induced binding of the cumate repressor to the cumate operator, rapamycin-induced interaction between FKBP12 and FRAP, FKCsA-induced interaction between FKBP and cyclophilin, ABA induced interaction between PYL1 and ABI1, and the “riboswitch” system. (Kallunki et al PMC6721553).

Constitutive CRISPRa to upregulate mouse Kcna1 expression has been reported to have an anti-epileptic effect (Colasante et al., 2020). However, it could not have been predicted that placing CRISPRa under the control of an activity-dependent promoter such as c-Fos, would lead to activity-dependent anti-epileptic activity with the advantageous properties disclosed herein, for example temporal reversibility and spatial specificity for neurons involved in seizures. The nucleotide sequences of dCas9 and the transcriptional activator VP64 are provided herein, as is the sequence of an sgRNA that recognises a promoter sequence of the mouse Kcna1 gene.

Alignment and calculation of percentage amino acid or nucleotide sequence identity can be achieved in various ways known to a person of skill in the art, for example, using publicly available computer software such as ClustalW 1.82, T-coffee or Megalign (DNASTAR) software. When using such software, the default parameters, e.g. for gap penalty and extension penalty, are preferably used. The default parameters of ClustalW 1.82 are: Protein Gap Open Penalty = 10.0, Protein Gap Extension Penalty = 0.2, Protein matrix = Gonnet, Protein/DNA ENDGAP = -1, Protein/DNA GAPDIST = 4.

The percentage identity can then be calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof.

Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid.

In some embodiments, the level of expression of the gene product increases when the neuron becomes more excited and decreases when the neuron becomes less excited.

### Disorders

One aspect of the invention provides expression vectors for use, as defined in the claims, in a method of treatment of a neurological disorder associated with neuronal hyperexcitability in a subject. Said methods of treatment may be prophylactic.

In certain aspects, the invention also provides the use of expression vectors and viral particles as described herein for the manufacture of a medicament for the treatment of said neurological disorder of a human or animal subject, expression vectors as described herein for use in the treatment of a said neurological disorder of a human or animal subject, and methods of treatment of said neurological disorder which comprises administering the expression vectors and viral particles as described herein to an individual in need thereof. The animal subject may be a mouse or a rat.

In some embodiments, the method of treatment is self-limiting after seizures end (“close loop” or “closed loop” therapy).

The neurological disorders as described herein are associated with neuronal hyperexcitability. As used herein, “hyperexcitability” is a characteristic feature of epilepsy in which the likelihood that neural networks become hypersynchronized, with excessive neuronal firing, is increased. The underlying mechanisms are incompletely understood and may include loss of inhibitory neurons, such as GABAergic interneurons, that would normally balance out the excitability of other neurons, or changes in the intrinsic properties of excitatory neurons that make them more likely to fire abnormally. Among other possible mechanisms are that the levels of GABA and the sensitivity of GABAA receptors to the neurotransmitter may decrease, resulting in less inhibition.

Non-limiting examples of neurological disorders associated with neuronal hyperexcitability include seizure disorders (such as epilepsy), Alzheimer’s disease, multiple sclerosis, Parkinson’s disease, tremor and other movement disorders, chronic pain, migraine, major depression, bipolar disorder, anxiety, and schizophrenia. In particularly preferred embodiments, the treatment is for epilepsy, for example idiopathic, symptomatic, and cryptogenic epilepsy. In particularly preferred embodiments, the epilepsy is neocortical epilepsy, temporal lobe epilepsy, especially if it is resistant to drugs used at therapeutic concentrations (pharmacoresistant or refractory epilepsy).

In some cases, seizures are accompanied by a profound depolarization and bursts of firing of pyramidal neurons in the cortex at frequencies greater than 50 Hz, which rarely if ever occur in physiological circumstances. Although activity-dependent promoters have been used to tag neurons that have been recruited by very strong sensory or other stimuli (peripheral nociceptor stimulation, fear-inducing electric shocks, cocaine), recordings from neurons imply that seizures induce much higher levels of activity than such stimuli. Furthermore, the CNS regions where such sensory stimuli have been shown to induce activity-dependent promoter function are different from those typically involved in seizures.

In some preferred embodiments, the neurological disorder is a disorder characterized by episodes of abnormal cellular activity, such as migraine, cluster headache, trigeminal neuralgia, post-herpetic neuralgia, paroxysmal movement disorders, uni- or bipolar affective disorders, anxiety and phobias. In some such disorders (migraine in particular), the abnormal activity may result in neuronal depolarization and electrical silence known as cortical spreading depolarization or cortical spreading depression, and this phenomenon has been implicated in sudden unexpected death in epilepsy (SUDEP).

The treatments described herein may be used to quench or block epileptic activity. The treatments may be used to reduce the frequency of seizures. The treatments may be used to temporally (for example, over 2, 6, 24, 48 or 72 hours) or permanently reduce abnormal neuronal excitability.

In some embodiments, the vector does not affect spontaneous locomotion or memory in a subject, optionally wherein spontaneous locomotion or memory is measured using an open field test, object localisation test, or T maze test.

In some embodiments, the expression vectors are only locally active in the seizure focus of the brain of a subject. In some cases, the expression vectors are only locally active in neurons capable of driving a seizure and/ generating sustained firing. In some cases, the expression vectors are only locally active in over-depolarised neurons.

In some embodiments, the vector or vector system can cause a reduction in the spike frequency of a neuron of the subject by more than 5%, or by more than 10%, or by more than 20%, or by more than 30%, or by more than 40%, or by more than 50%, or by more than 60%, or by more than 70%, or by more than 80%, or by more than 90%, or by more than 91%, or by more than 92%, or by more than 93%, or by more than 94%, or by more than 95%, or by more than 96%, or by more than 97%, or by more than 98%, or by more than 99%, or by 100%.

In some embodiments, the vector or vector system can cause a reduction in the spike frequency of a neuron of the subject by more than 75%. The reduction in the spike frequency of the neuron can be measured using multi-electrode arrays on or after 21 DIV (days in vitro). The reduction in the spike frequency may also be measured using calcium imaging or extracellular field potential recordings on or after 21 DIV. The reduction in the spike frequency of the neuron is measured relative to a vector comprising SEQ ID NO: 6. In some cases, the neuron is a primary cortical neuron.

In some embodiments, the vector or vector system can cause fewer than 10 action potentials per second, or fewer than 5 action potentials per second, or fewer than 4 action potentials per second, or fewer than 3 action potentials per second, or fewer than 2 action potentials per second, or no action potentials per second, in a neuron. In some embodiments, the vector or vector system can cause a greater than 50%, greater that 55%, greater that 60%, greater that 65%, greater that 70%, greater that 75%, greater that 80%, greater that 85%, greater that 90%, greater that 95%, or 100% reduction in action potentials per second. The number of action potentials may be measured using ex vivo acute hippocampal slice electrophysiology.

In some embodiments, the vector or vector system can cause a resting membrane potential in a neuron of less than -50 mV, or less than -60 mV, or less than -70 mV, or less than -80 mV, or less than -90 mV, or less than -100 mV. In some embodiments, the vector or vector system can increase the threshold for action potentials in a neuron to more than 50 pA, or more than 75 pA, or more than 100 pA, or more than 150 pA, or more than 200 pA, or more than 250 pA, or more than 300 pA, or more than 350 pA, or more than 400 pA, or more than 450 pA, or more than 500 pA, or more than 550 pA, or more than 600 pA, or more than 700 pA, or more than 800 pA, or more than 900 pA, or more than 1000 pA, wherein the threshold is the sum of current threshold and holding cu rre nt.

In some embodiments, the vector or vector system can cause less than 5 spikes/second in a primary neuronal culture grown on multi-electrode arrays (MEAs), as described in the examples. Spike is defined as aggregate neuronal activity. In some embodiments, the vector or vector system can cause less than 10, or less than 5 bursts /minute in a primary neuronal culture grown on MEAs, as described in the examples. In some embodiments, the vector or vector system can cause burst durations of less than 200 msec in a primary neuronal culture grown on MEAs, as described in the examples. In some embodiments, the vector or vector system can cause a mean number of spikes per burst of less than 20, or less than 15 in a primary neuronal culture grown on MEAs, as described in the examples.

In some embodiments, the number of action potentials, resting membrane potential, or threshold for action potentials is measured in an acute hippocampal slice from a subject. In some embodiments, the number of action potentials, resting membrane potential, or threshold for action potentials is measured using acute hippocampal slice electrophysiology and/or patch clamp electrophysiology.

In some embodiments, the vector or vector system can cause a greater anti-epileptic effect in a neuron driving a second seizure in a subject, than the anti-epileptic effect in the neuron driving the first seizure in the subject. In some embodiments, the anti-epileptic effect is measured using any of the appropriate methods described herein.

In some cases, the vector or vector system can prevent a second seizure in a subject, wherein the second seizure is subsequent to a first seizure in the subject.

### Administration and Dosage

The viral particles and expression vectors described herein can be delivered to the subject in a variety of ways, such as direct injection of the viral particles into the brain. For example, the treatment may involve direct injection of the viral particles into the cerebral cortex, in particular the neocortex or hippocampal formation. Another site of injection is an area of cortical malformation or hamartoma suspected of generating seizures, as occurs in focal cortical dysplasia or tuberous sclerosis. The treatment may involve direct injection of the viral particles into the location in the brain where it is believed to be functionally associated with the disorder. For example, where the treatment is for myoclonic epilepsy this may involve direct injection of the viral particles into the motor cortex; where the treatment is for chronic or episodic pain, this may involve direct injection of the viral particles into the dorsal root ganglia, trigeminal ganglia or sphenopalatine ganglia; and where the treatment is for Parkinson’s disease, this may involve direct injection of the viral particles into the substantia nigra, subthalamic nucleus, globus pallidus or putamen. The particular method and site of administration would be at the discretion of the physician who would also select administration techniques using his/her common general knowledge and those techniques known to a skilled practitioner.

The invention may also be used to treat multiple epileptic foci simultaneously by injection directly into the multiple identified loci.

The patient may be one who has been diagnosed as having drug-resistant or medically-refractory epilepsy, by which is meant that epileptic seizures continue despite adequate administration of antiepileptic drugs.

The subject may be one who has been diagnosed as having well defined focal epilepsy affecting a single area of the neocortex of the brain. Focal epilepsy can arise, for example, from developmental abnormalities or following strokes, tumours, penetrating brain injuries or infections.

Following administration of the viral particles, the recipient individual may exhibit reduction in symptoms of the disease or disorder being treated. For example, for an individual being treated who has a seizure disorder such as epilepsy, the recipient individual may exhibit a reduction in the frequency or severity of seizures. This may have a beneficial effect on the disease condition in the individual.

The term “treatment,” as used herein in the context of treating a condition, pertains generally to treatment and therapy of a human, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The viral particle can be delivered in a therapeutically-effective amount.

The term “therapeutically-effective amount” as used herein, pertains to that amount of the viral particle which is effective for producing some desired therapeutic effect, commensurate with a reasonable benefit/risk ratio, when administered in accordance with a desired treatment regimen.

Similarly, the term “prophylactically effective amount,” as used herein pertains to that amount of the viral particle which is effective for producing some desired prophylactic effect, commensurate with a reasonable benefit/risk ratio, when administered in accordance with a desired treatment regimen.

“Prophylaxis” in the context of the present specification should not be understood to describe complete success i.e. complete protection or complete prevention. Rather prophylaxis in the present context refers to a measure which is administered in advance of detection of a symptomatic condition with the aim of preserving health by helping to delay, mitigate or avoid that particular condition.

While it is possible for the viral particle to be used (e.g., administered) alone, it is often preferable to present it as a composition or formulation e.g. with a pharmaceutically acceptable carrier or diluent.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

In some embodiments, the composition is a pharmaceutical composition (e.g., formulation, preparation, medicament) comprising, or consisting essentially of, or consisting of as a sole active ingredient, viral particle as described herein, and a pharmaceutically acceptable carrier, diluent, or excipient.

As described in WO2008096268, in gene therapy embodiments employing delivery of the viral particle, the unit dose may be calculated in terms of the dose of viral particles being administered. Viral doses include a particular number of virus particles or plaque forming units (pfu). For embodiments involving adenovirus, particular unit doses include 10³, 10⁴, 10⁶, 10⁶, 10⁷, 10⁸, 10⁹, 10¹⁰, 10¹¹, 10¹², 10¹³ or 10¹⁴ pfu. Particle doses may be somewhat higher (10 to 100 fold) due to the presence of infection-defective particles.

In some embodiments the methods or treatments of the present invention may be combined with other therapies, whether symptomatic or disease modifying.

The term “treatment” includes combination treatments and therapies, in which two or more treatments or therapies are combined, for example, sequentially or simultaneously.

For example it may be beneficial to combine treatment with a compound as described herein with one or more other (e.g., 1, 2, 3, 4) agents or therapies.

Appropriate examples of co-therapeutics will be known to those skilled in the art on the basis of the disclosure herein. Typically the co-therapeutic may be any known in the art which it is believed may give therapeutic effect in treating the diseases described herein, subject to the diagnosis of the individual being treated. For example epilepsy can sometimes be ameliorated by directly treating the underlying etiology, but anticonvulsant drugs, such as phenytoin, gabapentin, lamotrigine, levetiracetam, carbamazepine, clobazam, topiramate, and others, which suppress the abnormal electrical discharges and seizures, are the mainstay of conventional treatment (Rho & Sankar, 1999, Epilepsia 40: 1471-1483).

The particular combination would be at the discretion of the physician who would also select dosages using his/her common general knowledge and dosing regimens known to a skilled practitioner.

The agents (i.e. viral particle, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

### Expression Vectors

An expression vector as used herein is a DNA molecule used to transfer and express foreign genetic material in a cell. Such vectors include a promoter sequence operably linked to the gene encoding the protein to be expressed. “Promoter” means a minimal DNA sequence sufficient to direct transcription of a DNA sequence to which it is operably linked. “Promoter” is also meant to encompass those promoter elements sufficient for promoter-dependent gene expression controllable for cell type specific expression; such elements may be located in the 5′ or 3′ regions of the native gene. Alternatively, an expression vector may be an RNA molecule that undergoes reverse transcription to DNA as a result of the reverse transcriptase enzyme.

An expression vector may also include a termination codon and expression enhancers. Any suitable vectors, enhancers and termination codons may be used to express the gene product, such as an edited Kv1.1 potassium channel, from an expression vector according to the invention. Suitable vectors include plasmids, binary vectors, phages, phagemids, viral vectors and artificial chromosomes (e.g. yeast artificial chromosomes or bacterial artificial chromosomes). As described in more detail below, preferred expression vectors include viral vectors such as AAV vectors.

An expression vector may additionally include a reporter gene encoding a reporter protein. An example of a reporter protein is a green fluorescent protein (“GFP”). A reporter gene may be operably linked to its own promoter or, more preferably, may be operably linked to the same promoter as the gene product as defined in the invention. As an example, the KCNA1 gene and reporter gene may be located either side of a sequence encoding a 2A peptide, such as a T2A peptide. 2A peptides are short (~20 amino acids) sequences that permit multicistronic gene expression from single promoters by impairing peptide bond formation during ribosome-mediated translation (Szymczak and Vignali, 2005). Having the reporter gene operably linked to the same promoter as the gene product, is thought to act as a reliable indicator of gene product expression. An expression vector including a reporter gene may be particularly useful in preclinical applications, for example for use in animal models where it can be used to help assess the localisation of gene expression. The gene encoding GFP may be GFP, dsGFP or dscGFP.

In other embodiments, the expression vector lacks a sequence encoding a reporter protein. This may be preferred for regulatory reasons, for example. In embodiments of the invention, reporting or detecting the gene product of the disclosure may be achieved in different ways - for example based on its engineered sequence. In some embodiments, the expression vector lacks a sequence encoding GFP and/or a sequence encoding a 2A peptide, such as a T2A peptide.

Generally speaking, those skilled in the art are well able to construct vectors and design protocols for recombinant gene expression. Suitable vectors can be chosen or constructed, containing, in addition to the elements of the invention described above, appropriate regulatory sequences, including promoter sequences, terminator fragments, polyadenylation sequences, marker genes and other sequences as appropriate. Molecular biology techniques suitable for the expression of polypeptides in cells are well known in the art. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, (1995, and periodic supplements).

The term “operably linked” used herein includes the situation where a selected gene and promoter are covalently linked in such a way as to place the expression of the gene (i.e. polypeptide coding) under the influence or control of the promoter. Thus, a promoter is operably linked to a gene if the promoter is capable of effecting transcription of the gene into RNA in a cell. Where appropriate, the resulting RNA transcript may then be translated into a desired protein or polypeptide. The promoter is suitable to effect expression of the operably linked gene in a mammalian cell. Preferably, the mammalian cell is a human cell.

The disclosed genes, such as an engineered KCNA1 gene, and gene products, such as an edited Kv1.1 potassium channel, can have the requisite features and sequence identity as described herein in relation to the expression vectors.

In some preferred embodiments, the expression vector comprises or consists of a nucleotide sequence having at least 70, 75, 80, 85, 90, 91, 92, 93, 94, 95, 96, 97, 98, 99 or 100% sequence identity to one of the following sequences:

mArc-dsGFP-KCNA1 (SEQ ID NO: 19); mArc-dsGFP-KCNJ2 (SEQ ID NO: 21); ESARE-dsGFP-KCNA1 (SEQ ID NO: 23); ESARE-dsGFP-KCNJ2 (SEQ ID NO: 25); NRAM-hCfos-dsGFP-KCNA1 (SEQ ID NO: 27); NRAM-hCfos -dsGFP-KCNJ2 (SEQ ID NO: 29); Egr1-dsGFP-KCNA1 (SEQ ID NO: 31); Egr1-dsGFP-KCNJ2 (SEQ ID NO: 33).

In some embodiments, the expression vector is as shown in any one of FIGS. 1-35.

### Viral Vectors

A preferred expression vector for use with the present invention is a viral vector, such as a lentiviral or AAV vector. A particularly preferred expression vector is an adeno associated viral vector (AAV vector).

In some instances, the vector is a recombinant AAV vector. AAV vectors are DNA viruses of relatively small size that can integrate, in a stable and site-specific manner, into the genome of the cells that they infect. They are able to infect a wide spectrum of cells without inducing significant effects on cellular growth, morphology or differentiation. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

AAV vectors may be prepared using standard methods in the art. Adeno-associated viruses of any serotype are suitable (see, e.g., Blacklow, pp. 165-174 of “Parvoviruses and Human Disease” J. R. Pattison, ed. (1988); Rose, Comprehensive Virology 3:1, 1974; P. Tattersall “The Evolution of Parvovirus Taxonomy” in Parvoviruses (J R Kerr, S F Cotmore. M E Bloom, R M Linden, C R Parrish, Eds.) p5-14, Hudder Arnold, London, UK (2006); and D E Bowles, J E Rabinowitz, R J Samulski “The Genus Dependovirus” (J R Kerr, S F Cotmore. M E Bloom, R M Linden, C R Parrish, Eds.) p15-23, Hudder Arnold, London, UK (2006), the disclosures of which are hereby incorporated by reference herein in their entireties). Methods for purifying for vectors may be found in, for example, U.S. Pat. Nos. 6,566,118, 6,989,264, and 6995006 and International Patent Application Publication No.: W0/1999/011764 titled “Methods for Generating High Titer Helper-free Preparation of Recombinant AAV Vectors”, the disclosures of which are herein incorporated by reference in their entirety.

Preparation of hybrid vectors is described in, for example, PCT Application No. PCT/US2005/027091, the disclosure of which is herein incorporated by reference in its entirety. The use of vectors derived from the AAVs for transferring genes in vitro and in vivo has been described (See e.g., International Patent Application Publication Nos: WO 1/18088 and WO 93/09239; U.S. Pat. Nos. 4,797,368, 6,596,535, and 5,139,941; and European Patent No: 0488528, all of which are herein incorporated by reference in their entirety). These publications describe various AAV-derived constructs in which the rep and/or cap genes are deleted and replaced by a gene of interest, and the use of these constructs for transferring the gene of interest in vitro (into cultured cells) or in viva (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In some instances, useful AAV vectors for the expression constructs as described herein include those encapsidated into a virus particle (e.g. AAV virus particle including, but not limited to, AAV1, AAV2, AAV3, AAV4, AAV5, AAV6, AAV7, AAV8, AAV9, AAV10, AAV11, AAV12, AAV13, AAV14, AAV15, AAV16 and AAVrh10). Accordingly, the instant disclosure includes a recombinant virus particle (recombinant because it contains a recombinant polynucleotide) comprising any of the vectors described herein.

In some embodiments, the viral vector contains a sequence encoding a reporter protein, such as a fluorescent protein. In other embodiments the viral vector lacks a sequence encoding a reporter protein, such as a fluorescent protein.

In some embodiments, the vector comprises a nucleotide sequence having at least 70, 75, 80, 85, 90, 91, 92, 93, 94, 95, 96, 97, 98 or 99% identity to the nucleotide sequence of SEQ ID NO: 8, SEQ ID NO: 9, or SEQ ID NO: 10. In some embodiments, the viral vector is the nucleotide sequence of SEQ ID NO: 8, SEQ ID NO: 9, or SEQ ID NO: 10.

In some preferred embodiments, the viral vector comprises or consists of a nucleotide sequence having at least 70, 75, 80, 85, 90, 91, 92, 93, 94, 95, 96, 97, 98, 99 or 100% sequence identity to one of the following sequences:

AAV- mArc-dsGFP-KCNA1 (SEQ ID NO: 20); AAV- mArc-dsGFP-KCNJ2 (SEQ ID NO: 22); AAV- ESARE-dsGFP-KCNA1 (SEQ ID NO: 24); AAV- ESARE-dsGFP-KCNJ2 (SEQ ID NO: 26); AAV- NRAM-hCfos -dsGFP-KCNA1 (SEQ ID NO: 28); AAV- NRAM-hCfos -dsGFP-KCNJ2 (SEQ ID NO: 30); AAV- Egr1-dsGFP-KCNA1 (SEQ ID NO: 32); Egr1-dsGFP-KCNJ2 (SEQ ID NO: 34).

In some embodiments, the viral vector additionally comprises genes encoding viral packaging and envelope proteins.

In some embodiments, the viral vector is a lentiviral vector. In some embodiments, the lentiviral vector is a non-integrating lentiviral vector (NILV). Vector particles produced from these vectors do not integrate their viral genome into the genome of the cells and therefore are useful in applications where transient expression is required or for sustained episomal expression such as in quiescent cells. NILVs can be developed by mutations in the integrase enzyme or by altering the 5′ LTR and/or the 3′ LTR to prevent integrase from attaching these sequences. These modifications eliminate integrase activity without affecting reverse transcription and transport of the pre-integration complex to the nucleus. Without wishing to be bound by any particular theory, when a NILV enters a cell the lentiviral DNA is expected to remain as remains in the nucleus as an episome, leading to sustained expression in non-dividing cells (post-mitotic cells) such as neurons.

In some embodiments, the vector further comprises an AmpR gene, and/or a hGh poly(A) signal gene, and/or one or more origin of replication genes.

### Viral Particles

The invention also includes in vitro methods of making viral particles, such as lentiviral particles or adeno-associated viral particles. In one embodiment, this method involves transducing mammalian cells with a viral vector as described herein and expressing viral packaging and envelope proteins necessary for particle formation in the cells and culturing the transduced cells in a culture medium, such that the cells produce viral particles that are released into the medium. An example of a suitable mammalian cell is a human embryonic kidney (HEK) 293 cell.

It is possible to use a single expression vector that encodes all the viral components required for viral particle formation and function. Most often, however, multiple plasmid expression vectors or individual expression cassettes integrated stably into a host cell are utilised to separate the various genetic components that generate the viral vector particles.

In some embodiments, expression cassettes encoding the one or more viral packaging and envelope proteins have been integrated stably into a mammalian cell. In these embodiments, transducing these cells with a viral vector described herein is sufficient to result in the production of viral particles without the addition of further expression vectors.

In other embodiments, the in vitro methods involve using multiple expression vectors. In some embodiments, the method comprises transducing the mammalian cells with one or more expression vectors encoding the viral packaging and envelope proteins that encode the viral packaging and envelope proteins necessary for particle formation.

Examples of suitable viral packaging and envelope proteins and expression vectors encoding these proteins are commercially available and well known in the art. In general, the viral packaging expression vector or expression cassette expresses the gag, pol, rev, and tat gene regions of HIV-1 which encode proteins required for vector particle formation and vector processing. In general, the viral envelope expression vector or expression cassette expresses an envelope protein such as VSV-G. In some cases, the packaging proteins are provided on two separate vectors - one encoding Rev and one encoding Gag and Pol. Examples of lentiviral vectors along with their associated packaging and envelope vectors include those of Dull, T. et al., “A Third-generation lentivirus vector with a conditional packaging system” J. Virol 72(11):8463-71 (1998), which is herein incorporated by reference.

The ssDNA AAV genome contains two open reading frames, Rep and Cap, flanked by two 145 base inverted terminal repeats (ITRs) fundamental for the synthesis of the complementary DNA strand. Rep and Cap produce multiple proteins (Rep78, Rep68, Rep52, Rep40, which are required for the AAV life cycle; and VP1, VP2, VP3, which are capsid proteins). The transgene will be inserted between the ITRs and Rep and Cap in trans. An AAV2 backbone is commonly used and is described in Srivastava et al., J. Virol., 45: 555-564 (1983). Cis-acting sequences directing viral DNA replication (ori), packaging (pkg) and host cell chromosome integration (int) are contained within the ITRs. AAVs also require a helper plasmid containing genes from adenovirus. These genes (E4, E2a and VA) mediate AAV replication. An example of a pAAV plasmid is available from Addgene (Cambridge, MA, USA) as plasmid number 112865 or 60958.

Following release of viral particles, the culture medium comprising the viral particles may be collected and, optionally the viral particles may be separated from the culture medium. Optionally, the viral particles may be concentrated.

Following production and optional concentration, the viral particles may be stored, for example by freezing at -80° C. ready for use by administering to a cell and/or use in therapy.

The invention also provides viral particles, for example those produced by the methods described herein. As used herein, a viral particle comprises a DNA or RNA genome packaged within the viral envelope that is capable of infecting a cell, e.g. a mammalian cell. A viral particle may be integrase deficient, e.g. it may contain a mutant integrase enzyme or contain alterations in the 5′ and/or 3′ LTRs as described herein.

### Cells

The invention also provides a cell comprising the nucleic acid or vector described above. In some embodiments, this cell is a mammalian cell such as a human cell. In some embodiments, the cell is a human embryonic kidney cell (HEK) 293. In some embodiments, the cell is derived from a neuroblastoma cell-line.

### Kits

The invention also provides kits that comprise an expression vector as described herein and one or more viral packaging and envelope expression vectors also described herein. In some embodiments the viral packaging expression vector is an integrase-deficient viral packaging expression vector.

### Methods of Confirming Presence of Gene Products

The invention also provides a method of confirming the presence of a gene product as described herein, such as engineered KCNA1, in a cell.

A limitation of clinical translation using certain gene sequences is that it is difficult to detect their expression against the background endogenous channels present in the brain.

The sequences of gene product as described herein may differ from endogenous wild-type gene products found in cells, such that when this gene is transcribed into RNA it incorporates a unique RNA sequence (an ‘RNA-fingerprint’). This RNA-fingerprint permits specific tracking of transgene expression with RNA-targeted techniques that would otherwise fail to distinguish between transgenic and endogenous gene products. This is particularly useful where it is important to determine the localisation of gene expression without having to include sequences encoding fluorescent tags or epitopes that could potentially result in immunogenicity.

For example, tissue removed from patients who have been treated with a gene product could be examined to determine where and in which cell types (excitatory neurons as expected, or inhibitory neurons or glial cells) the gene product was present. Such tissue could be obtained, for instance, from epilepsy surgery in the event of epilepsy gene therapy failure, or post-mortem. This data is expected to be useful for preclinical dosage calculation, biodistribution studies, regulatory approval and further clinical development on gene therapy.

Thus, in one embodiment the method comprises transducing a cell with an expression vector as described herein or administering a viral particle as described herein to a cell under conditions that permit expression of a gene product of interest and detecting the presence of the gene product RNA in the cell using a hybridisation assay. This method can be carried out in vitro or ex vivo, for example in cell culture or in cells explanted from a human or animal body. Alternatively, the method can be carried out in vivo, for example where the viral particles are administered to a cell in a human or animal subject before extracting the cells or tissues from the human or animal subject in order to detect the presence of gene product RNA in the cell using a hybridisation assay.

In some embodiments, cells or tissues are extracted from a subject who has been treated with viral particles of the invention in order to examine localisation of the expressed gene product. Such tissue could be obtained, for instance, from epilepsy surgery in the event of epilepsy gene therapy failure, or post-mortem.

The invention also provides an in vitro or ex vivo method of confirming the presence of gene product in a cell that has been obtained from a subject administered with a viral particle described herein, the method comprising detecting the presence of engineered gene product RNA in the cell using a hybridisation assay.

Hybridisation assays are known in the art and generally involve using complementary nucleic acid probes (such as in situ hybridization using labelled probe, Northern blot and related techniques). In some embodiments, the hybridisation assay is an in situ hybridisation assay using a labelled probe, such as a fluorescently labelled probe.

As used herein, the term “probe” refers to a nucleic acid used to detect a complementary nucleic acid. Typically the probe is an RNA probe.

Suitable selective hybridisation conditions for oligonucleotides of 17 to 30 bases include hybridization overnight at 42° C. in 6X SSC and washing in 6X SSC at a series of increasing temperatures from 42 oC to 65 oC. One common formula for calculating the stringency conditions required to achieve hybridization between nucleic acid molecules of a specified sequence homology is (Sambrook et al., 1989): Tm = 81.5 oC + 16.6 Log [Na+] + 0.41 (% G+C) - 0.63 (% formamide) - 600/#bp in duplex.

Any sub-titles herein are included for convenience only, and are not to be construed as limiting the disclosure in any way. The invention will now be further described with reference to the following non-limiting Figures and Examples. Other embodiments of the invention will occur to those skilled in the art in the light of these. The disclosure of all references cited herein, inasmuch as it may be used by those skilled in the art to carry out the invention, is hereby specifically incorporated herein by cross-reference.

### FIGURES

FIG. 1 is a schematic representation of certain aspects of the invention. FIG. 1A (upper) represents neurons with normal activity levels. FIG. 1A (lower) represents hyperexcited neurons with high activity (darker shading) driving a seizure. FIG. 1B represents current gene-therapy approaches, wherein all neurons are permanently modified in order to modulate neuron excitability and treat a seizure. FIG. 1C represents certain aspects of the present invention, wherein only hyperexcited neurons are modified in order to modulate neuron excitability and treat a seizure. FIG. 1D shows a hypothesized molecular mechanism of c-Fos-KCNA1 action, and an exemplary vector of the disclosure. Hyperactivity (strong increase in neuronal excitability) / epileptic activity or seizures will induce c-fos or other activity-dependent promoter activation that in turns will activate KCNA1 or other transgenes able to reduce neuronal excitability (Kv1.1 channel HL = 12 d). Activity-dependent promoter activation may lead to KCNA1 overexpression. The activation of the promoter is transient but the protein expressed will be expressed in the neuron for longer time (e.g. days) i.e. sustained anti-epileptic effect. Once the pathological state is corrected the tool is switched off (and will be reactivated if necessary) FIG. 1E shows an overview of activity-dependent genes suitable for use in the invention. FIG. 1F shows an example of c-fos activation induced by hyperactivity in rodents and human. FIG. 1G shows different combinations of activity-dependent promoters and transgenes suitable for use in this invention. Other transgenes as shown may also be suitable for use with the invention. The transgenes have different properties and functional effects on neuronal excitability. The promoters have different properties in terms of timing of activation, cell specificity and deactivation. FIG. 1 is described further in Example 1.

FIG. 2 shows the results of a c-Fos immunostaining experiment (FIG. 2A and FIG. 2B). Seizure-like activity (induced by 4-aminopyridine + Picrotoxin) leads to a rapid but transient increase in endogenous c-Fos expression. FIG. 2 is described further in Example 2.

FIG. 3 shows the results of a Lentivirus c-Fos-dsGFP (FIG. 3A) fluorescence imaging experiments (FIG. 3B and FIG. 3C). FIG. 3D shows the results of AAV9 cfos-dsGFP-KCNJ2 (middle) and mArc-dsGFP-KCNJ2 (right) fluorescence imaging experiments. These show that the promoters follow neuronal activity. FIG. 3 is described further in Example 3.

FIG. 4 shows that AAV c-Fos-dsGFP-KCNA1 reduced neuronal network excitability in cortical neurons, compared to AAV c-Fos-dsGFP, as measured by spikes/second, bursts/min, and mean number of spikes per burst (see lower panel). An example recording from the EEG experiment is shown in the upper panel (vertical scale bar corresponds to 20 µV; horizontal scale bar corresponds to 1 s). FIG. 4 is described further in Example 4.

FIG. 5 shows that AAV c-Fos-dsGFP-KCNA1 reduced neuronal network excitability in vitro over 48 hours, compared to AAV c-Fos-dsGFP, as measured by spikes/second (FIG. 4A), bursts/min (FIG. 4B), burst duration (msec) (FIG. 4C) and mean number of spikes per burst (FIG. 4D). PTX is a proconvulsant agent (picrotoxin). FIG. 5E shows that AAV c-Fos-dsGFP-KCNA1, cfos-dsGFP-KCNJ2, mArc-dsGFP-KCNA1, mArc-dsGFP-KCNJ2, and ESARE-dsGFP-KCNA1 reduced neuronal network excitability in cortical neurons, compared to AAV c-Fos-dsGFP, as measured by firing rate spikes/second. FIG. 5 is described further in Examples 4 and 5.

FIG. 6 shows the results of an in vivo fluorescence experiment demonstrating that, compared with cell-dependent gene-expression (FIG. 6A), activity-dependent gene expression (FIG. 6B) is specific for seizure focus. The scale bar for FIG. 6A is 500 µm; the scale bar for FIG. 6B is 50 µm. A schematic of the experimental procedure is shown in FIG. 6C. FIG. 6 is described further in Example 6.

FIG. 7 shows the results of an activity-dependent gene therapy preclinical trial performed in a rat epilepsy model. FIG. 7 is described further in Example 7. The horizontal scale car corresponds to 500 µm. “CA1” refers to the Cornu ammonis 1 sub-field of the hippocampus, and “DG” refers to dentate gyrus.

FIG. 8 shows a map of vector pX552-c-FosP-dscGFP-T2A-KCNA1co.1400V, which was used in Examples 4-11.

FIG. 9 shows a map of vector pX552-c-FosP-KCNA1co.1400V. FIG. 9 is described further in Example 7. FIG. 9 is also described further in Example 11.

FIG. 10 shows the experimental plan of an ex vivo hippocampal slice electrophysiology experiment to demonstrate the activation of activity-dependent promoters following a seizure and the effect on neuronal excitability when they drive either KCNA1 or KCNJ2. PTZ is an acute chemoconvulsant (pentylenetrazole). FIG. 10 is described further in Example 8.

FIGS. 11 and 12 show the results of an ex vivo electrophysiology experiment in acute hippocampal neurons demonstrating that activity-dependent KCNA1 expression activated by a seizure is enough to decrease neuronal excitability. FIG. 11 shows representative traces for neuronal firing. FIG. 12 is a graph showing number of action potential elicited with different current injections, demonstrating the efficiency of the activity-dependent gene therapy in selectively decreasing neuronal excitability. FIGS. 11 and 12 are described further in Example 8.

FIG. 13 shows the results of an ex vivo electrophysiology experiment demonstrating that either activity-dependent KCNA1or KCNJ2 expression activated by a seizure is enough to decrease neuronal excitability. On the left: KCNJ2 hyperpolarizes neurons (RMP: resting membrane potential). On the right: Activity-dependent promoter-driven KCNA1 or KCNJ2 expression increases the current required to elicit action potentials. FIG. 14 is described further in Example 8.

FIGS. 14 and 15 show the fluorescence of the slices after an ex vivo electrophysiology experiment demonstrating that activity-dependent promoters activated by a seizure selectively activated only some neurons. FIGS. 14 and 15 are described further in Example 8.

FIGS. 16 and 17 show the results of in vivo experiments showing the protective effect against repetitive seizures. Activity-dependent gene therapy is activated by a first seizures and when a second seizure is induced it showed an anti-epileptic effect. This experiment has been performed using c-Fos-dsGFP-KCNJ2 as an example. FIGS. 16 and 17 are described further in Example 9.

FIGS. 18, 19 and 20 show the results of an activity-dependent gene therapy preclinical trial performed in a mouse epilepsy model. These data show that activity-dependent gene therapy rescues the epileptic phenotype in a severe model of chronic intractable epilepsy. FIGS. 18, 19 and 20 are described further in Example 10.

FIG. 21 shows the results of an activity-dependent gene therapy preclinical trial performed in a mouse epilepsy model. These data show that activity-dependent gene therapy protect epileptic animals against a further severe insult that leads to death epileptic animals injected with a control virus. FIG. 21 is described further in Example 10.

FIG. 22 shows the results of an activity-dependent gene therapy preclinical trial performed in a mouse epilepsy model. These data show that activity-dependent gene therapy is self-regulated (closed-loop). Animals treated with the activity-dependent gene therapy do not exhibit seizures and do not show detectable fluorescence, meaning that the activity-dependent approach (and expression) is switched off because the animal was cured. FIG. 22 is described further in Example 10.

FIG. 23 summarizes the tests used to test the effect of activity-dependent gene therapy on behaviour. The data show that activity-dependent gene therapy has no effect on spontaneous locomotion, anxiety and memory. Open field, Object localisation Test and T-Maze were used to screen for effects of the activity-dependent gene therapy in healthy animals. FIG. 23 is described further in Example 11.

FIG. 24 shows further results of an activity-dependent gene therapy preclinical trial performed in a rat epilepsy model. The horizontal scale bar in B corresponds to 500 µm. FIG. 21 is described further in Example 7.

FIG. 25 shows that AAV c-Fos-dCas9-VP64-eGFP-Kcna1 (2 AAVs), reduced neuronal network excitability in cortical neurons exposed to PTX (proconvulsant agent), compared to AAV c-Fos-dCas9-VP64-eGFP (2 AWs), as measured by spikes/second over 48 hours. Doxycycline has been used to activate the inducible promoter driving the dCAS9-VP64. All the tool is controlled by the c-Fos promoter driving the transactivator of the inducible promoter. FIG. 25 is described further in Example 5.

FIG. 26 shows a map of vector pX552-c-FosP-dscGFP-T2A-KCNJ2. FIG. 26 is described further in Examples 4,8,and 9.

FIG. 27 shows a map of vector pX552-miniARC-dscGFP-T2A-KCNA1co.I400V. FIG. 27 is described further in Example 4 and 8.

FIG. 28 shows a map of vector pX552-miniARC-dscGFP-T2A-KCNJ2. FIG. 28 is described further in Example 4 and 8.

FIG. 29 shows a map of vector pX552-ESARE-dscGFP-T2A- KCNA1co.I400V. FIG. 29 is described further in Example 4 and 8.

FIG. 30 shows a map of vector pX552-ESARE-dscGFP-T2A-KCNJ2. FIG. 30 is described further in Example 8.

FIG. 31 shows a map of vector pX552-NRAM-hcfos-dscGFP-T2A- KCNA1co.I400V. FIG. 31 is described further in Example 8.

FIG. 32 shows a map of vector pX552-NRAM-hcfos-dscGFP-T2A-KCNJ2.

FIG. 32 shows a map of vector pX552-Egr1-dscGFP-T2A- KCNA1co.I400V.

FIG. 33 shows a map of vector pX552-Egr1-dscGFP-T2A-KCNJ2.

FIG. 34 shows maps of the CRISPRa vectors pAAV-TetO-dCAS9VP64 and pAAV-U6-sgRNA_Kcna1-cFos-rtTA-T2A-EGFP. FIG. 34 is described further in Example 5.

### EXAMPLES

### Example 1 - Illustration of Activity-Dependent Therapy and Hypothesized Molecular Mechanism of c-Fos-KCNA1 Action

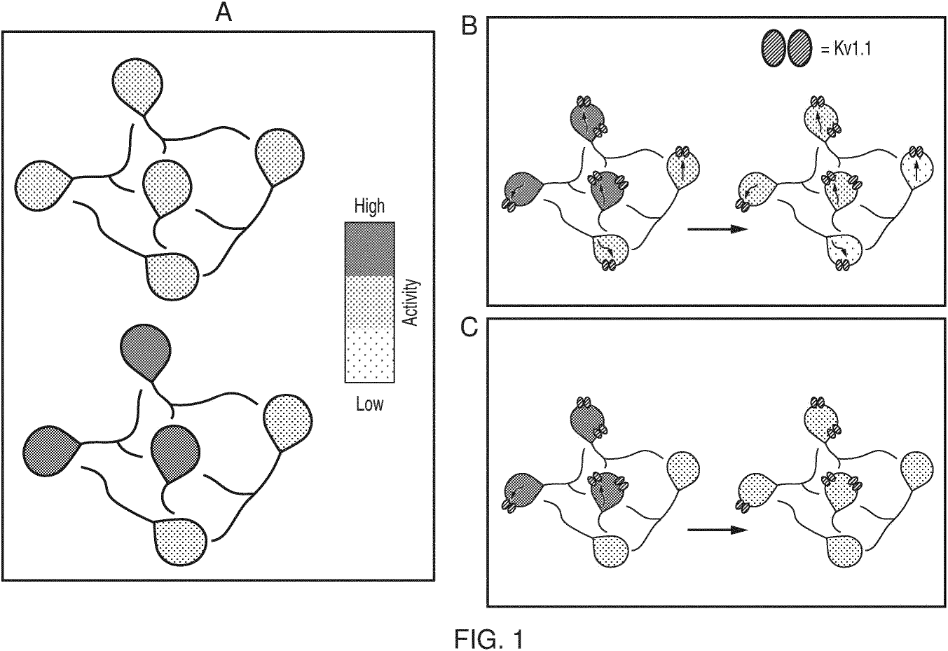

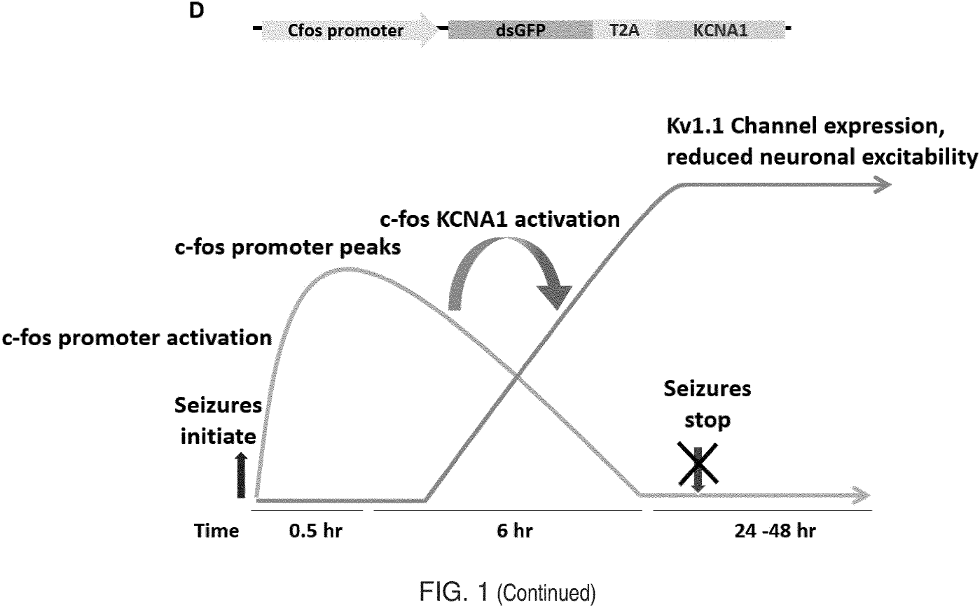

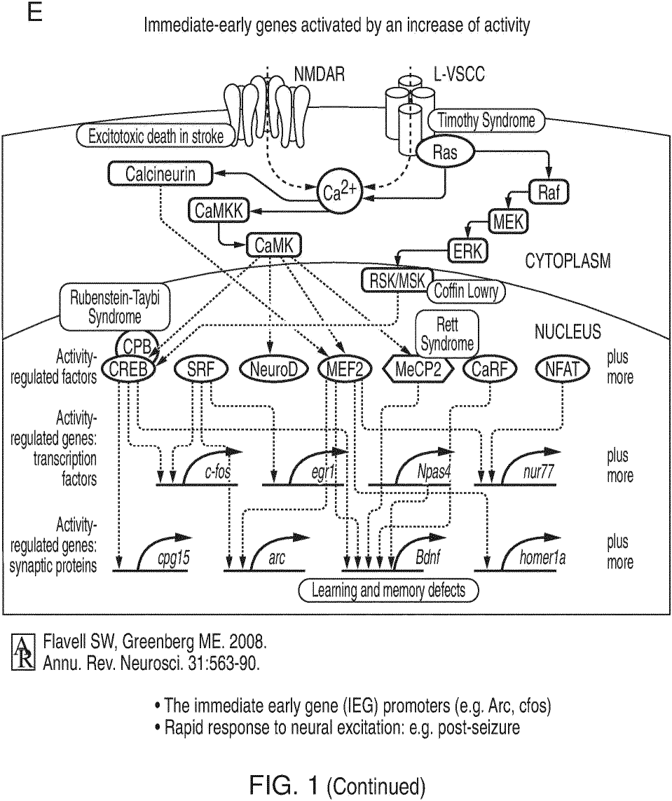

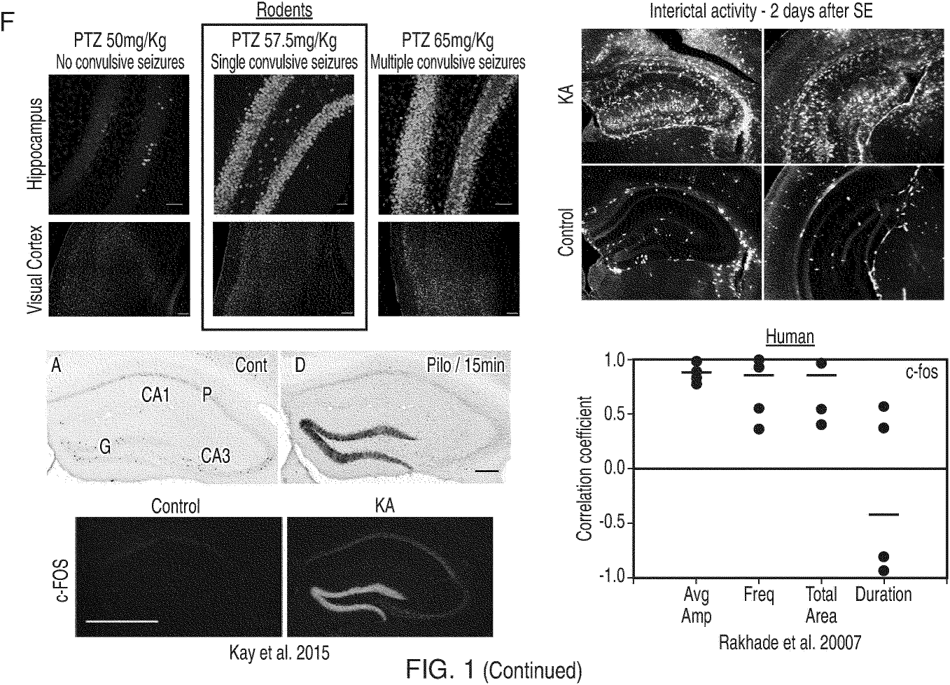

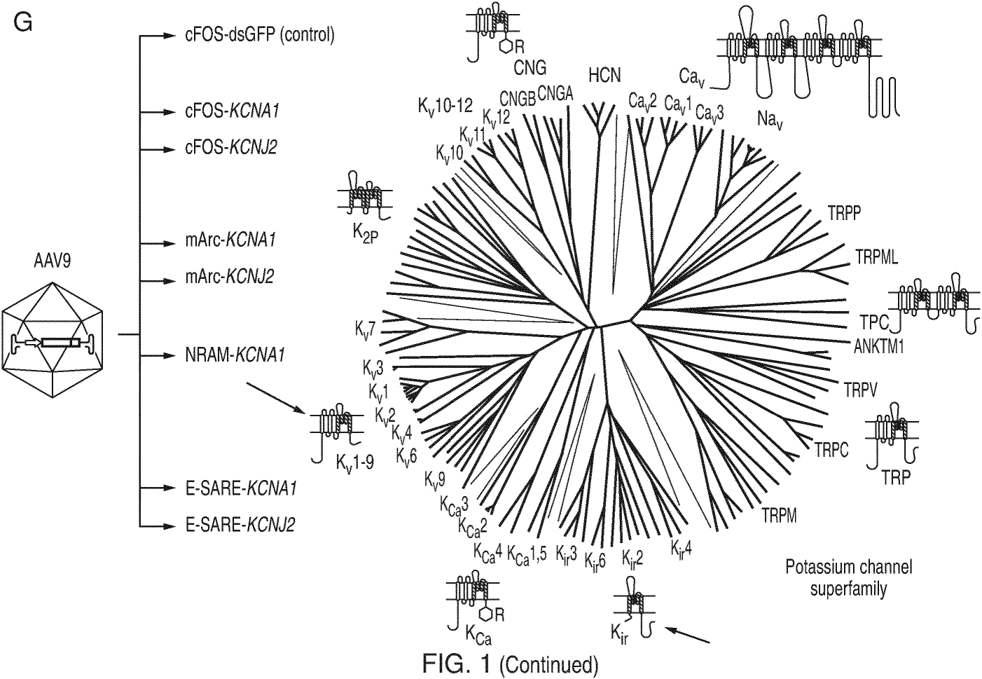

One aspect of the invention is a method to treat epilepsy using activity-dependent promoters in order to selectively target the neurons driving seizures, or contributing to propagating seizures, (darker shading in FIG. 1A) which in turn will alter the expression of genes that affect neuronal properties, compared to neurons that are not driving seizures (lighter shading in FIG. 1A).

Some current experimental gene therapies rely on permanent modification of neuronal excitability, for example using a Kv1.1 ion channel under the control of a cell-specific promoter, and which may not discriminate between neurons involved in seizure and healthy neurons (FIG. 1B).

Neuronal excitation elicits the rapid induction of a set of genes called immediate early genes (IEGs) such as c-Fos and Arc. c-Fos may discriminate between those neurons involved or not in the seizures, as increased expression of c-Fos in specific neurons after seizures has been observed in mouse models, and in human epileptic brains, where c-Fos has a transient expression.

Using a c-Fos promoter in an adeno-associated viral vector enables up-regulation of expression of the effector gene (KCN1A) encoding the potassium channel Kv1.1, which in turn reduces neuronal firing. The increased expression of KCNA1 is predicted to restore normal neuronal behaviour in the epileptic focus. After the circuit activity returns to near-normal levels, the promoter activity decreases and the expression of the potassium channel returns to baseline (FIG. 1D).

The c-Fos promoter will be activated by a seizure and then switch on immediately, staying on for 6-12 hours. In this lag of time the therapeutic gene will be express and protein transcribed. The protein will stay stable for longer time (KCNA1 is supposed to be stable in the membrane for >96 hrs).

In this case the patients are “protected” from seizures for days, and as many patients experience seizures in clusters, the treatment should reduce the number of seizures experienced within a cluster. Furthermore, a rescue of clustered seizures may lead to a restoration of a physiological state that can result in no more seizures at all.

If other seizures occur later, the system will be switched on again.

FIG. 1E shows an overview of activity-dependent genes suitable for use in the invention. FIG. 1F shows an example of c-fos activation induced by hyperactivity in rodents and human. FIG. 1G shows different combinations of activity-dependent promoters and transgenes suitable for use in this invention. Other transgenes such as other potassium channels (right) may also be suitable for use with the invention. The transgenes have different properties and functional effects on neuronal excitability. The promoters have different properties in terms of timing of activation, cell specificity and deactivation

### Example 2 - Seizure-Like Activity Increases IEG Expression

### Materials and Methods

Primary mature cortical neurons were stimulated with pro-convulsant drugs and c-fos expression was assessed by immunofluorescence at different time points (2, 6, 24 and 48) after fixation.

### Results and Discussion

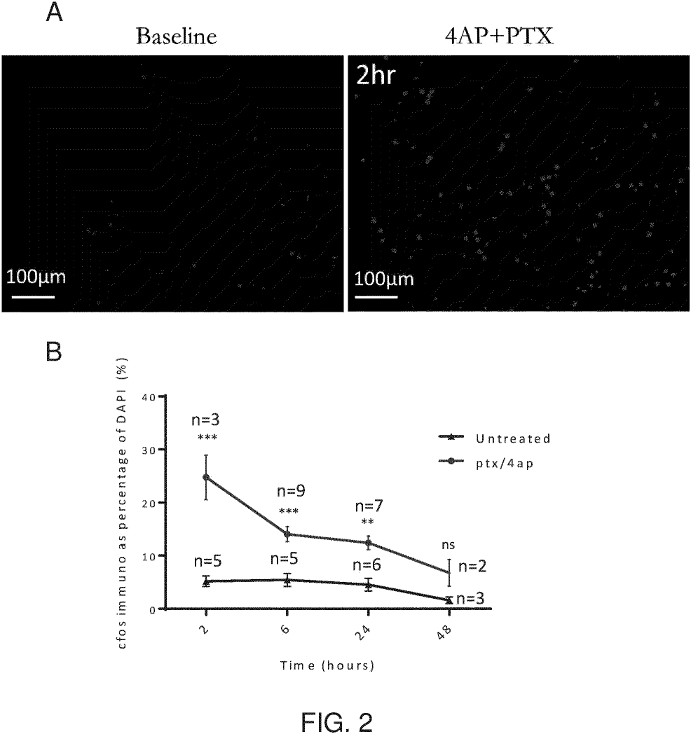

FIG. 2 shows that seizure-like activity (induced by 4-aminopyridine (“4AP”) + Picrotoxin (“PTX”)) leads to a rapid but transient increase in endogenous c-Fos expression.

### Example 3 - c-Fos Promoter Can Drive GFP Expression, and Arc Promoter Can Drive GFP Expression

### Materials and Methods

A minimal promoter of c-Fos with a part of the 5′UTR and a chimeric intron to boost the expression of the transgene was used. The promoter was then inserted into an AAV backbone with the dsGFP and KCNA1 codon optimised.

Also, a minimal promoter for Arc was used to boost the expression of the transgene. The promoter was inserted into an AAV backbone with KCNJ2.

### Results and Discussion

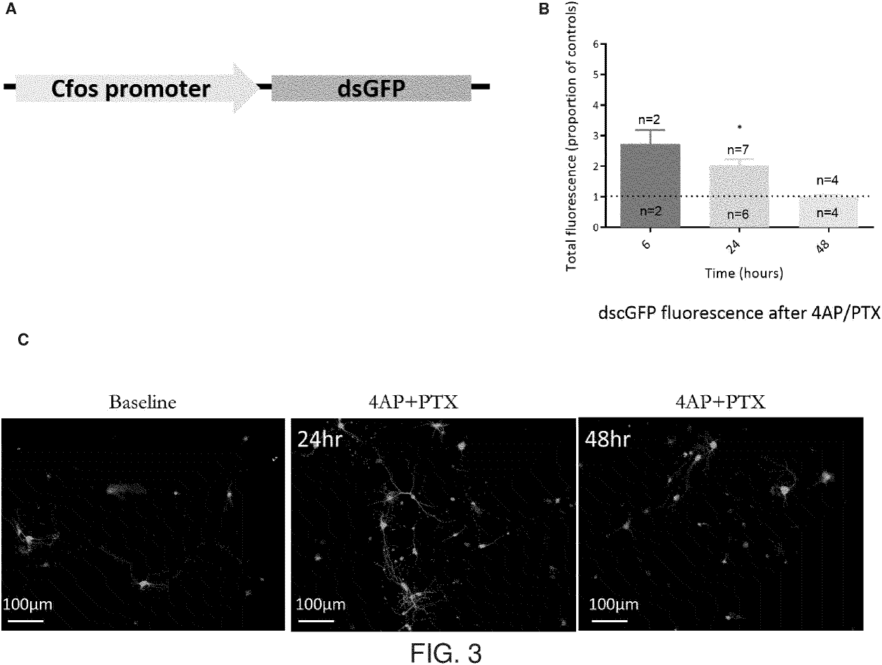

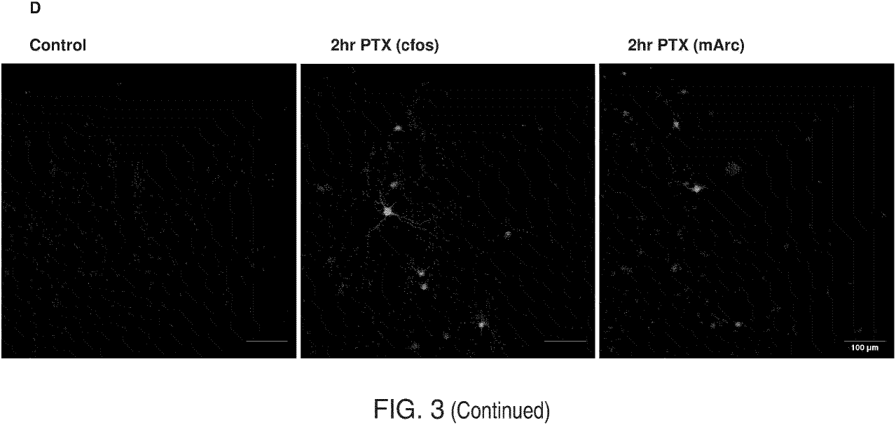

FIG. 3 shows that c-Fos promoter can drive GFP expression when seizure-like activity is induced in neural cells by 4AP and PTX.

Also, FIG. 3D shows that Arc can drive GFP expression when seizure-like activity is induced in neural cells with 4AP and PTX.

### Example 4 - Activity-Dependent Dampening of Excitability

### Materials and Methods

Primary cortical neurons were grown on multi-electrode arrays (MEAs) for 21 DIV and transduced at 7 DIV with either AAV c-Fos-dsGFP or AAV c-Fos-dsGFP-KCNA1. Network activity was assessed at 21 DIV. Repeats were n=6 (C-Fos-dsGFP) and n=7 (C-Fos-KCNA1).

Also, primary cortical neurons were grown on multi-electrode arrays (MEAs) for 21 DIV (days in vitro) and transduced at 7 DIV with either AAV c-Fos-dsGFP or AAV c-Fos-dsGFP-KCNA1 or c-Fos-dsGFP-KCNJ2 or mArc-dsGFP-KCNA1 or mArc-dsGFP-KCNJ2 or ESARE-dsGFP-KCNA1. Network activity was assessed at 21 DIV. Repeats were n=6 (C-Fos-dsGFP), n=7 (C-Fos-KCNA1), n=16 (c-Fos-dsGFP-KCNJ2), n=6 (mArc-dsGFP-KCNA1), n=5 (mArc-dsGFP-KCNJ2), and n= 5 (ESARE-dsGFP-KCNA1).

### Results and Discussion

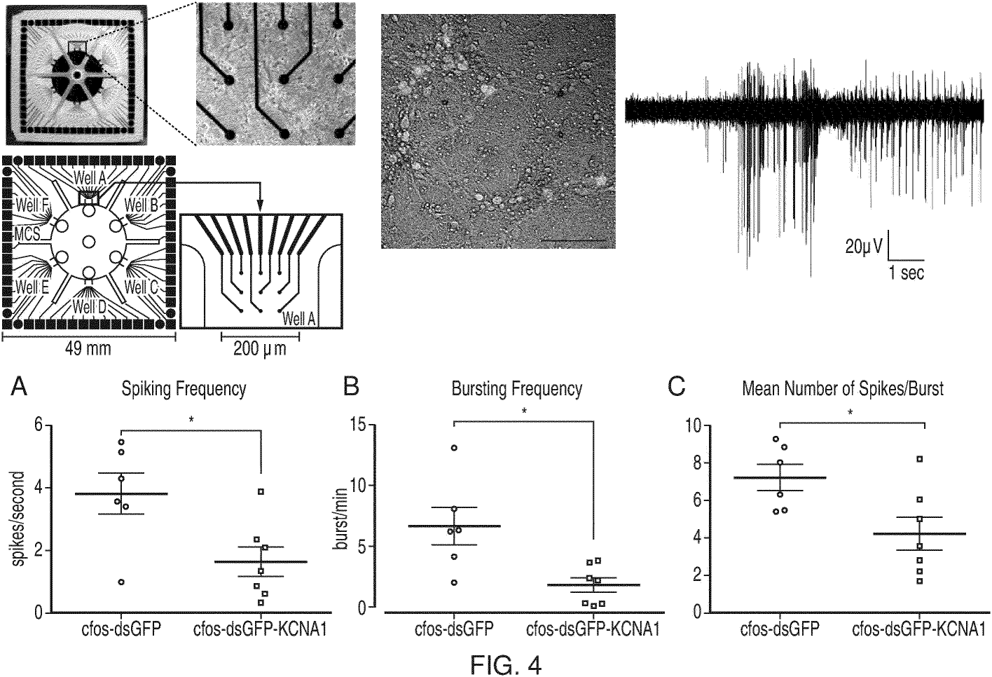

FIG. 4 shows that AAV c-Fos-dsGFP-KCNA1 reduced neuronal network excitability in cortical neurons, compared to AAV c-Fos-dsGFP, as measured by spikes/second (FIG. 4A), bursts/min (FIG. 4B), burst duration (msec) (FIG. 4C), and mean number of spikes per burst (FIG. 4C). An example recording from the MEA experiment is shown in in the upper panel.

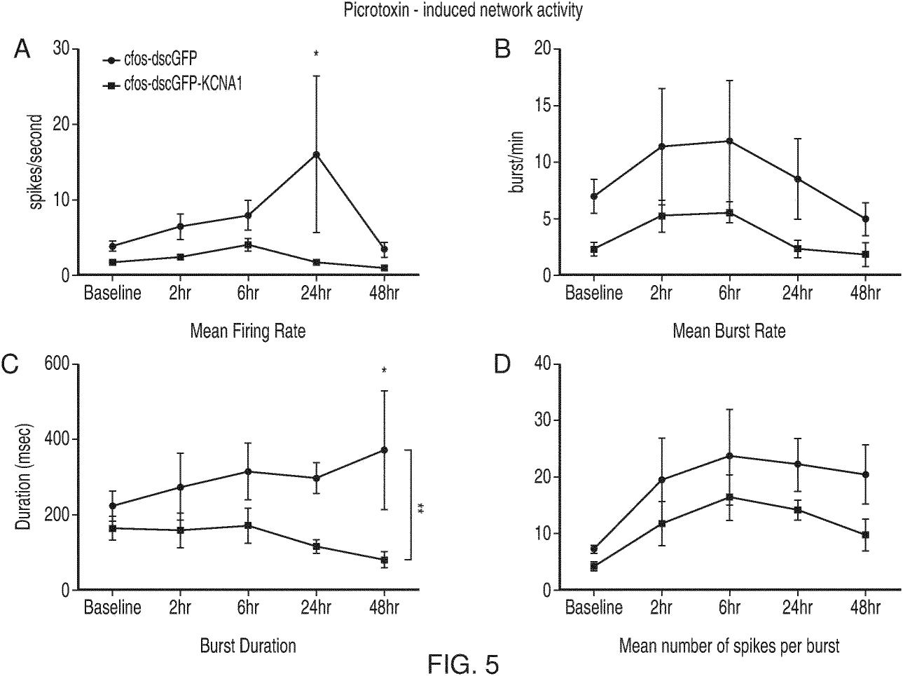

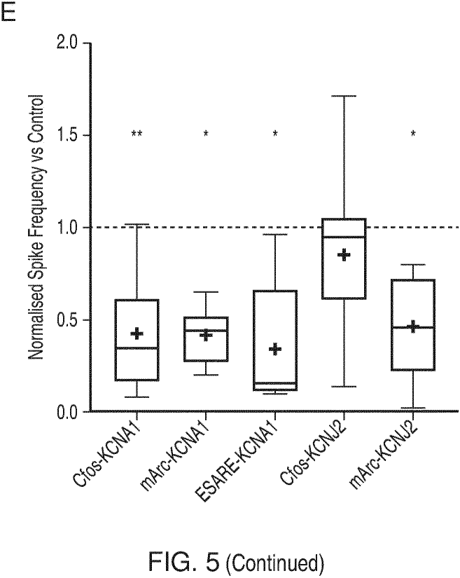

Also, FIG. 5E shows that AAV c-Fos-dsGFP-KCNA1, c-Fos-dsGFP-KCNJ2, mArc-dsGFP-KCNA1, mArc-dsGFP-KCNJ2 or ESARE-dsGFP-KCNA1 reduced neuronal network excitability in cortical neurons, compared to AAV c-Fos-dsGFP, as measured by spikes/second.

As discussed in example 5, FIG. 5 shows that AAV c-Fos-dsGFP-KCNA1 reduced neuronal network excitability in cortical neurons, compared to AAV c-Fos-dsGFP, as measured by spikes/second, bursts/min, burst duration (msec), and mean number of spikes per burst.

### Example 5 - Time-Course for Activity-Dependent Dampening of Excitability

### Materials and Methods

Primary cortical neurons were grown on multi-electrode arrays (MEAs) for 21 DIV and transduced at 7 DIV with either AAV c-Fos-dsGFP or AAV c-Fos-dsGFP-KCNA1. Network activity was assessed at 21 DIV, and at different time points (2, 6, 24, 48 hrs) after addition of 30 µM picrotoxin (baseline/ 0 hr). Repeats were n=6 (c-Fos-dsGFP) and n=7 (c-Fos-dsKCNA1).

Also, primary cortical neurons were grown on multi-electrode arrays (MEAs) for 21 DIV and transduced at 7 DIV with either c-Fos-dCAS9-VP64-GFP or c-Fos-dCAS9-VP64-GFP-KCNA1 (2 AAVs). Network activity was assessed at 21 DIV, and at different time points (2, 6, 24,48 hrs) after addition of 30 µM picrotoxin (baseline/ 0 hr). Repeats were n=16 (c-Fos-dCAS9-VP64-GFP) and n=10 (c-Fos-dCAS9-VP64-GFP-KCNA1).

### Results and Discussion

FIG. 5 shows that AAV c-Fos-dsGFP-KCNA1 slows down the increase neuronal network excitability induced by PTX, compared to AAV c-Fos-GFP, as clearly shown by burst duration (msec).

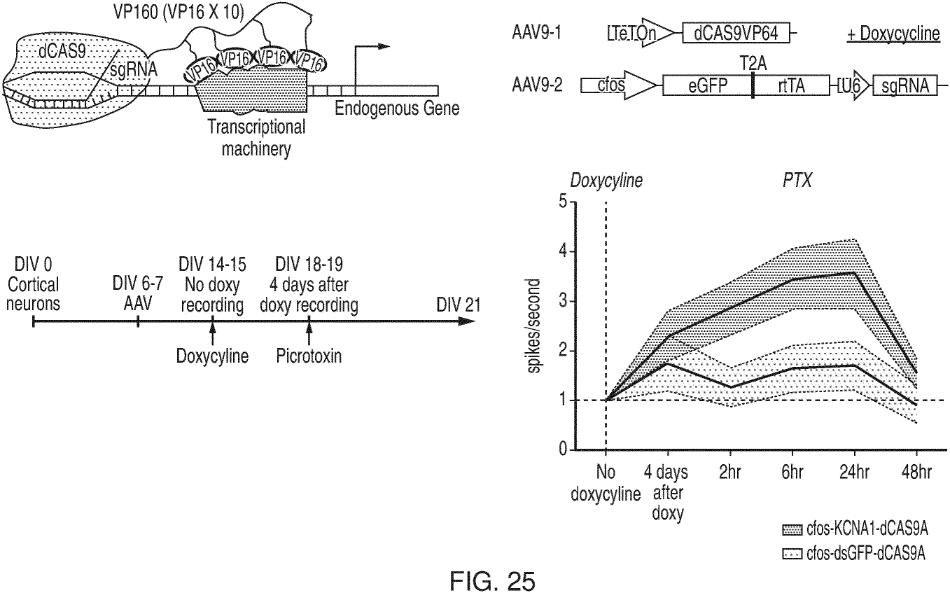

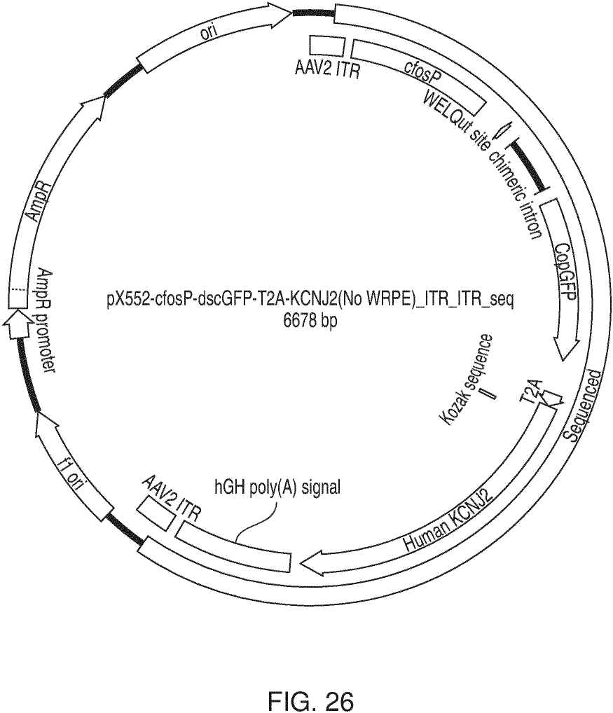

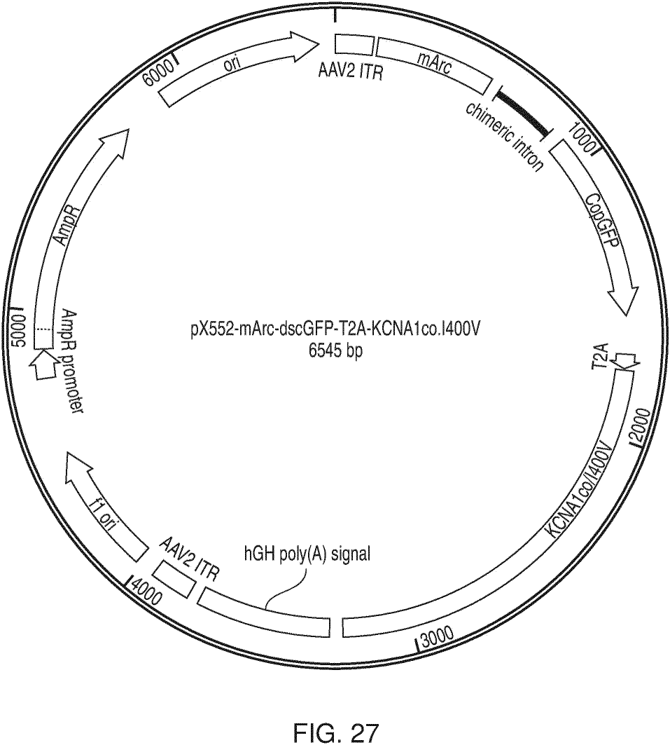

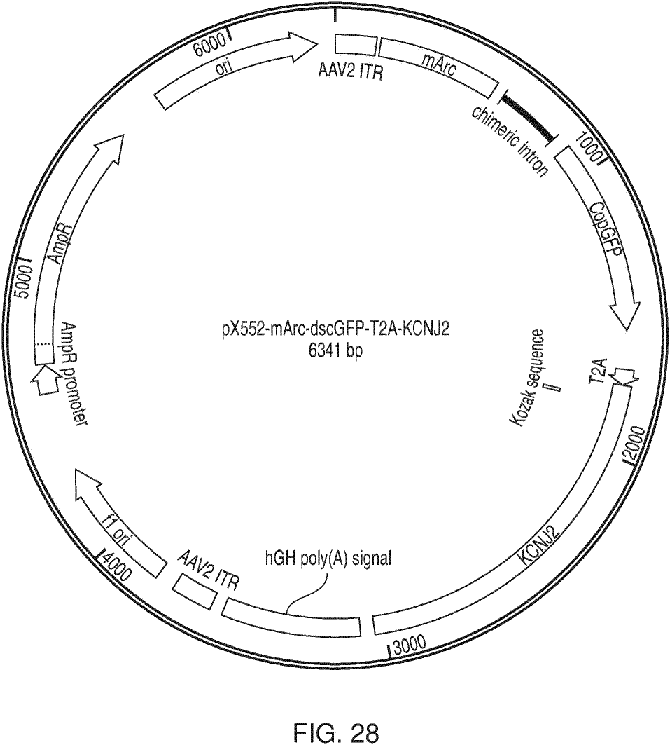

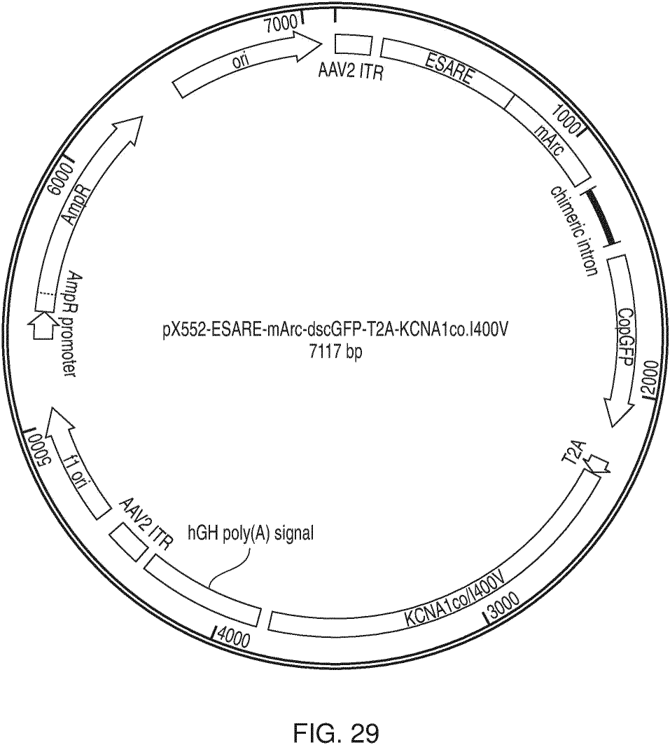

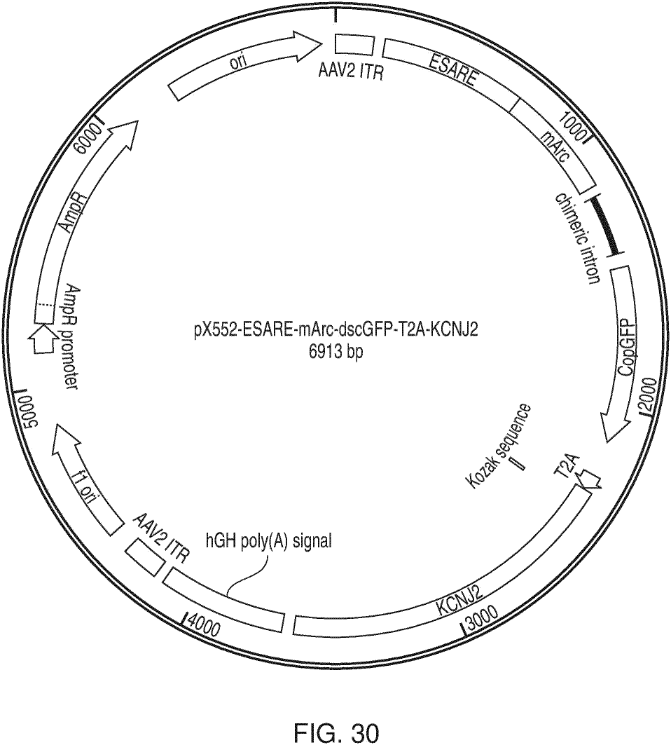

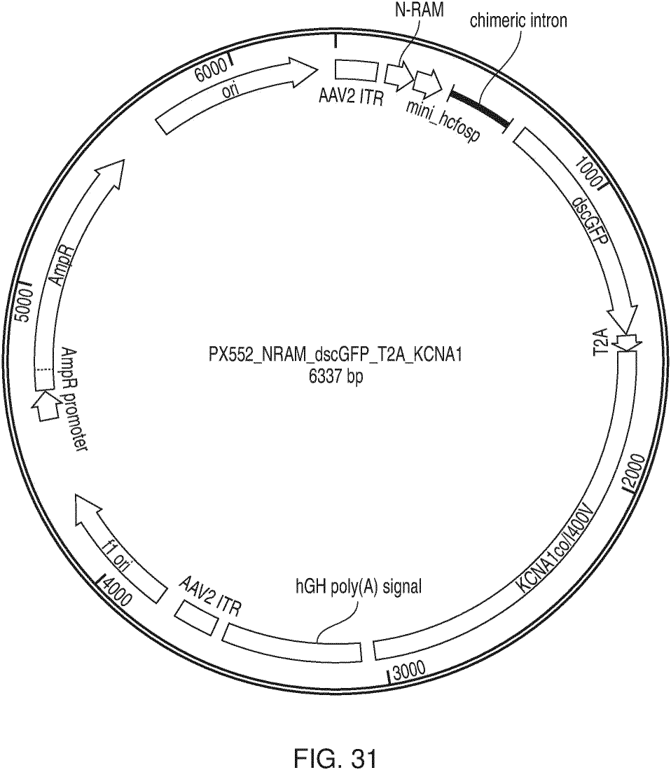

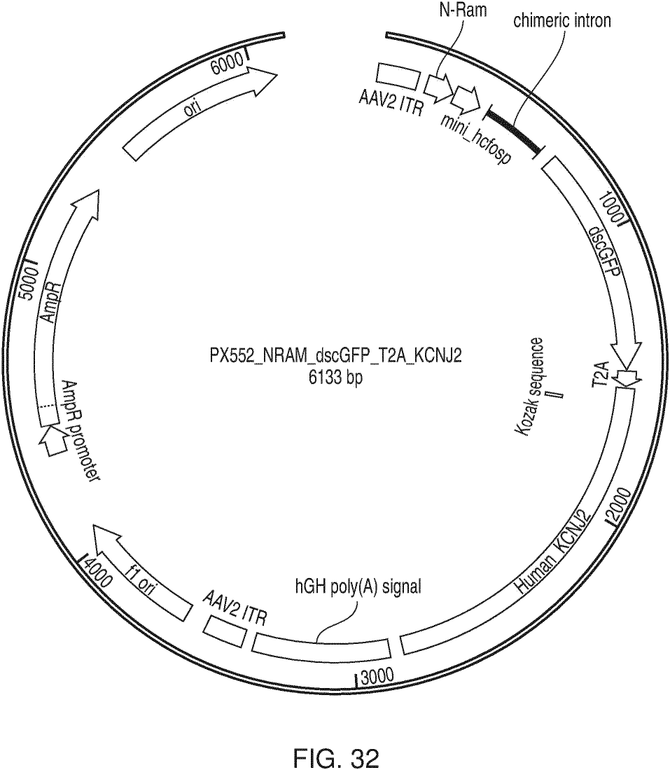

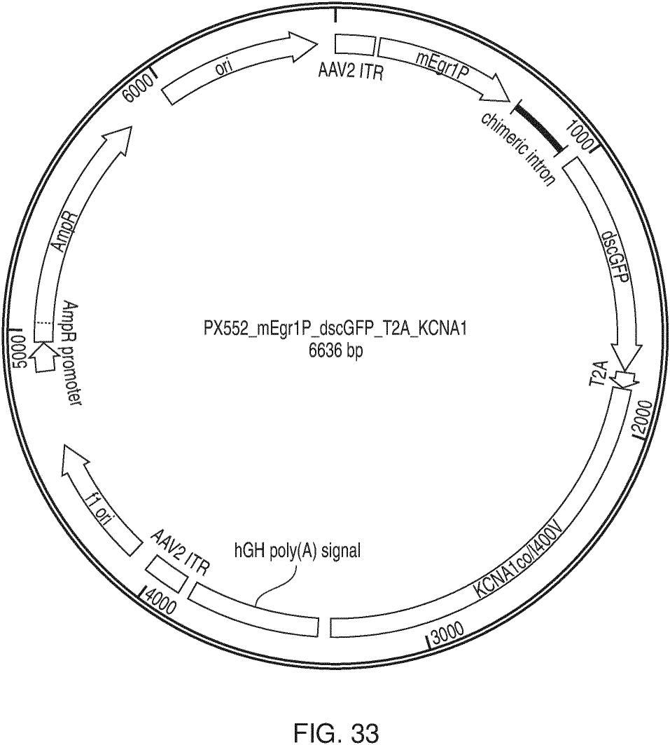

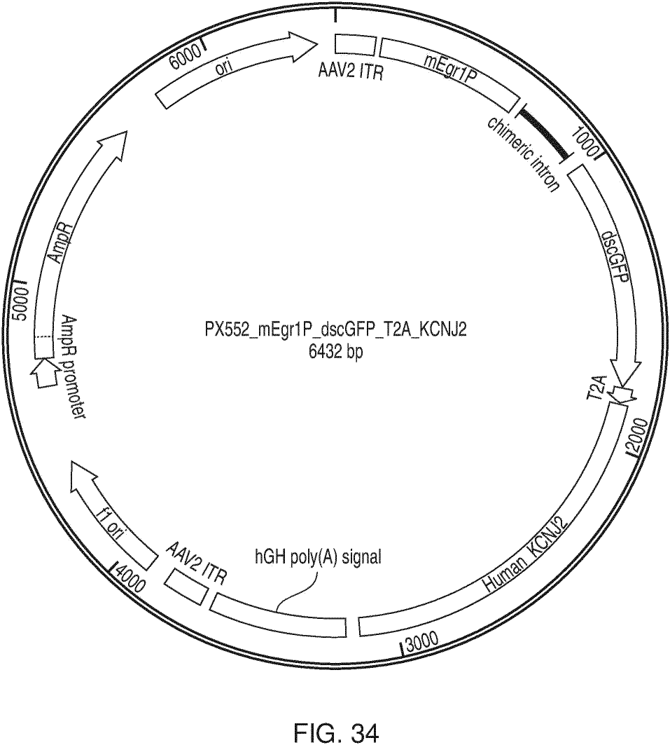

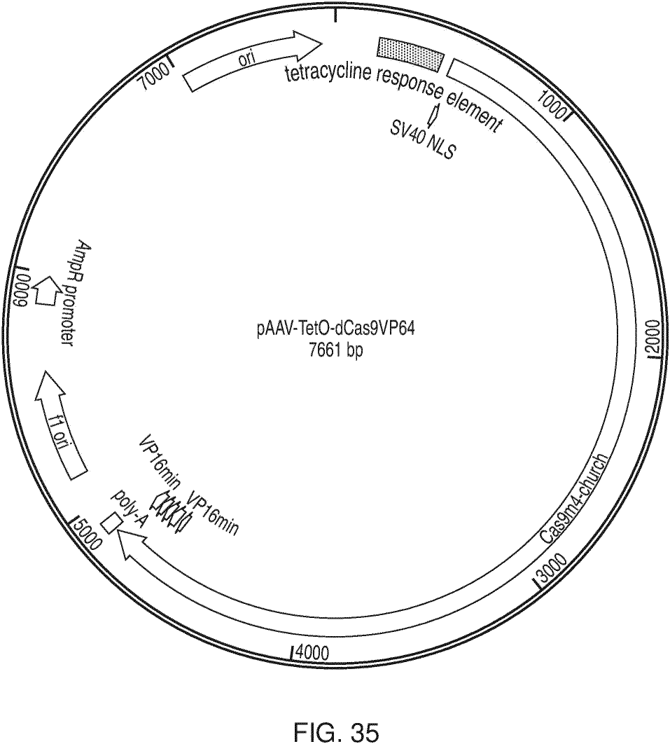

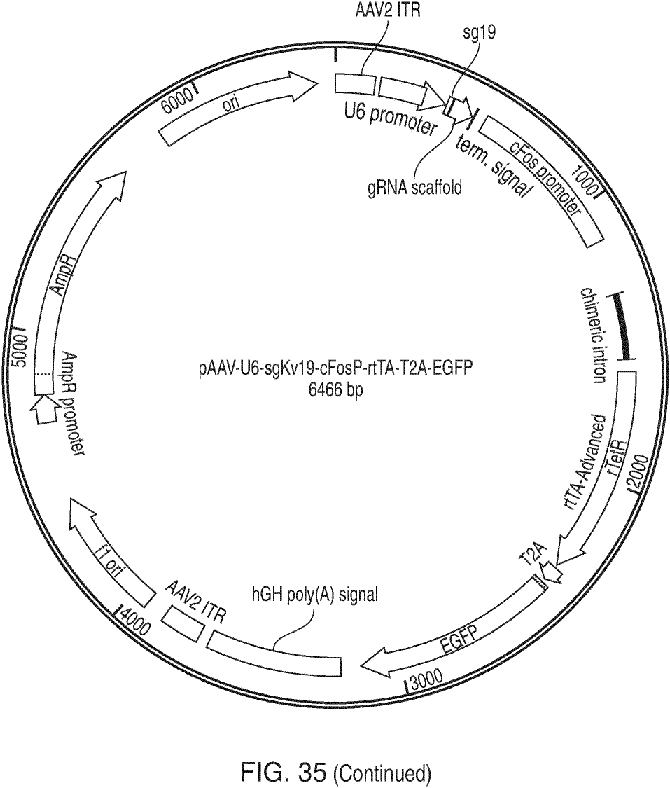

Also, FIG. 25 shows that c-Fos-dCAS9-VP64-GFP-KCNA1 slows down the increase neuronal network excitability induced by PTX, compared to c-Fos-dCAS9-VP64-GFP, as clearly shown by burst duration (msec) or spikes/seconds. Gene therapy delivered with two AAVs allows Doxycycline to switch it on using the TeT-On system.

### Example 6 - Activity-Dependent Gene Therapy Affects Fewer Neurons Than Conventional Over-Expression

### Materials and Methods

Acute pilocarpine injections in the visual cortex were performed after viral injection of either AAV Camk2a-GFP or AAV cfos-GFP. Acute pilocarpine injections lead to focal seizures. The spread of the virus and the number of neurons positive for GFP were evaluated.

### Results and Discussion

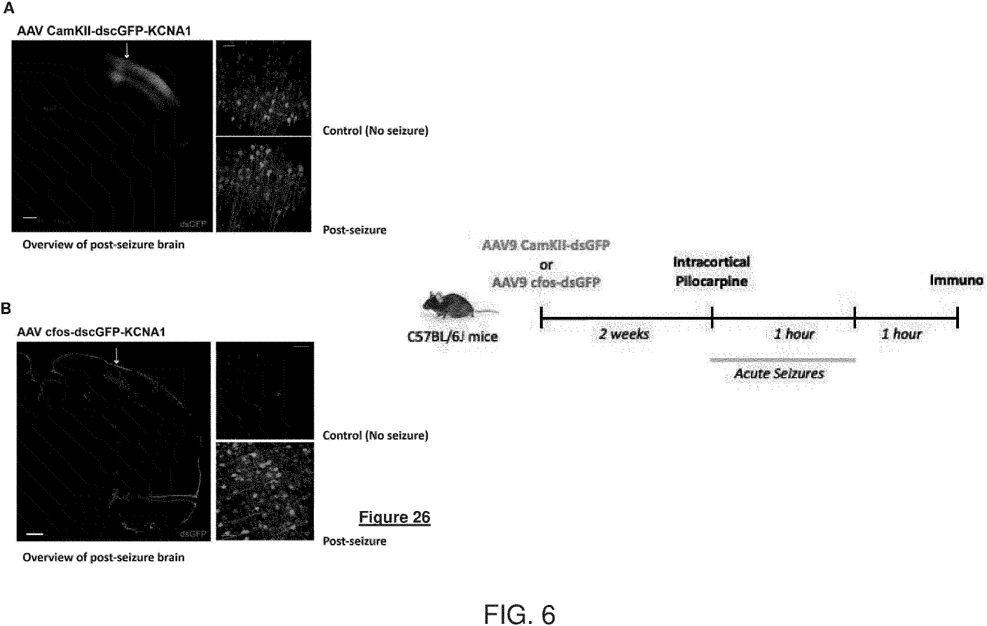

FIG. 6 shows that In vivo activity-dependent gene expression is specific for seizure focus, compared to constitutive gene expression. In contrast to conventional gene therapy (FIG. 6A), only a small number of neurons are targeted and the GFP reporter only lights up after a seizure (FIG. 6B) using activity-dependent gene expression.

Because the virus serotype used is the same (AAV9), the spread of transduction is comparable and this provides direct evidence that the treatment will not affect bystander neurons that do not participate in the seizure. Thus, the therapeutic effect is specifically targeted to neurons that become over-activated.

A schematic of the experimental procedure is shown in FIG. 6C.

### Example 7 - Preclinical Epilepsy Model

### Materials and Methods

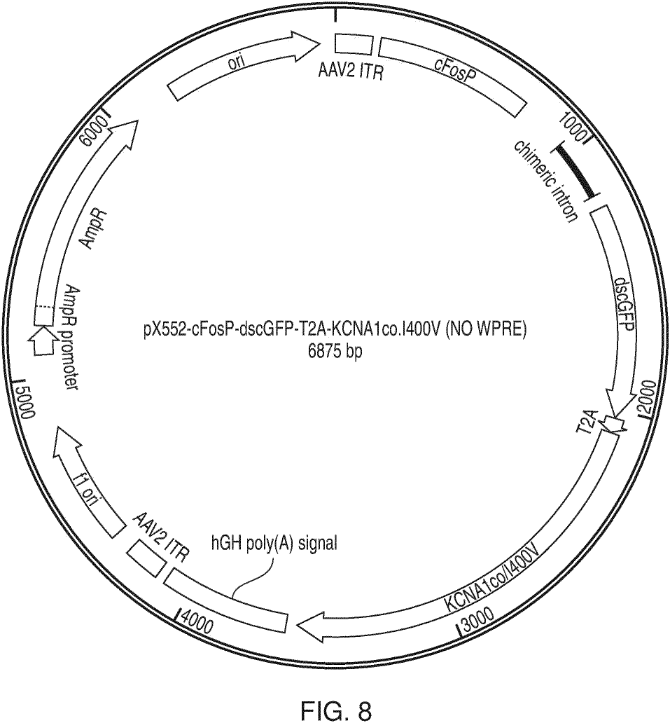

A chronic rat model of temporal lobe epilepsy (TLE) was created using intraperitoneal (IP) injection of kainic acid (KA). After 12 weeks EEG transmitters and cannulas were implanted and the rats were recorded continuously for 5 weeks (Baseline). Then, AAV-cfos-dsGFP or AAV-cfos-dsGFP-KCNA1 (as shown in FIG. 8) were injected through the cannulas and animals were recorded for a further 8 weeks.

### Results and Discussion

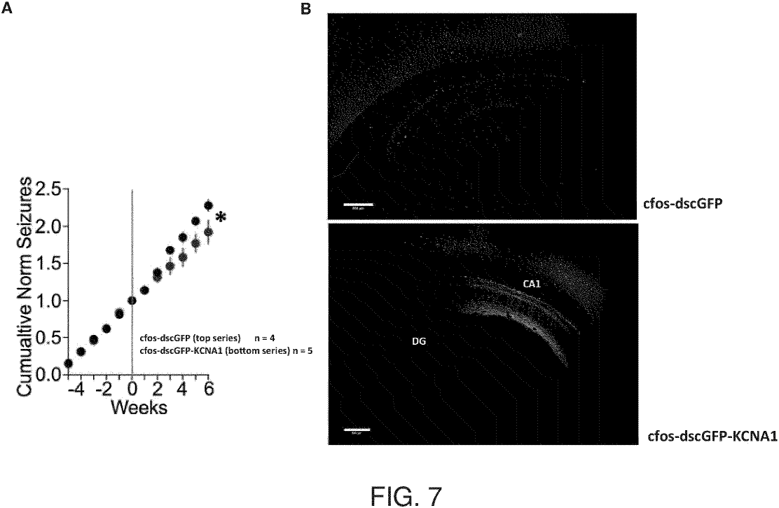

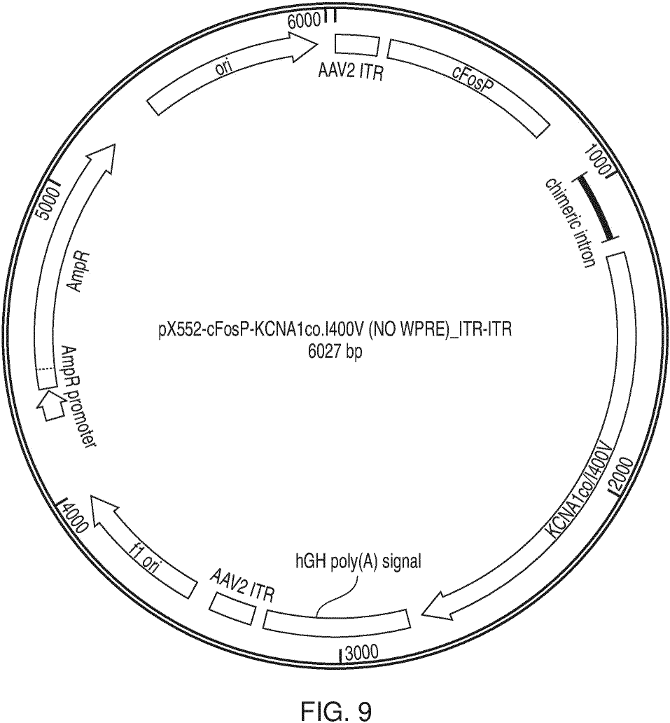

FIG. 7 demonstrates in vivo activity-dependent gene therapy in a rat epilepsy model, using the construct of FIG. 8. A decrease in number of seizures was observed in rats injected with AAV-cfos-dsGFP-KCNA1 compared to AAV-cfos-dsGFP (FIG. 7A). In some cases, the construct of FIG. 8 will lack a sequence encoding a reporter protein, as shown in FIG. 9, and in SEQ ID NO: 10. This may be preferred for regulatory reasons, for example.

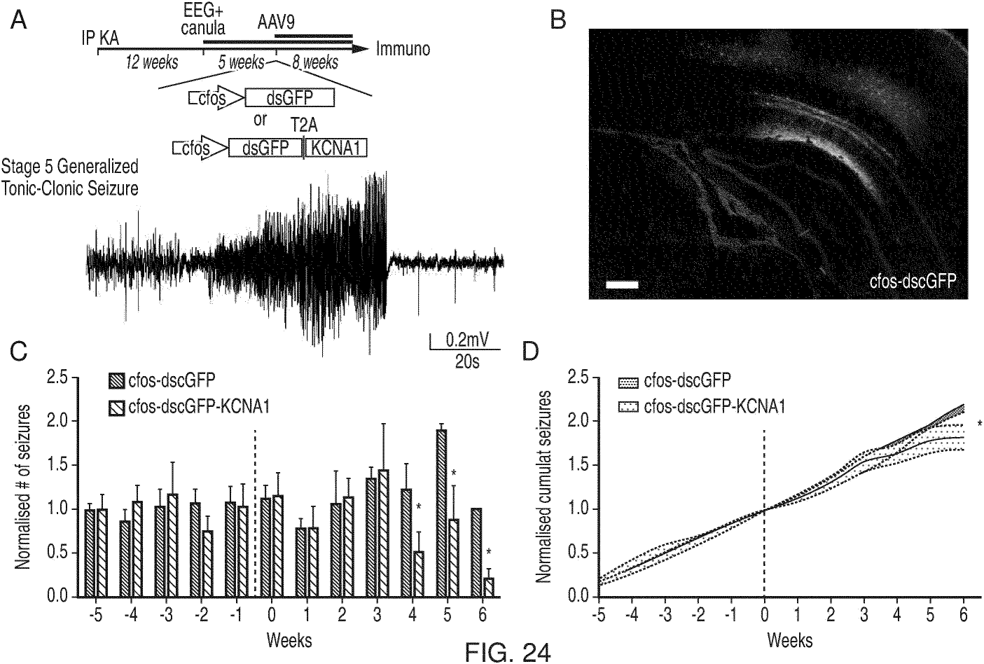

FIG. 24 provides further data to also demonstrate in vivo activity-dependent gene therapy in a rat epilepsy model, using the construct of FIG. 8. A decrease in number of seizures was observed in rats injected with AAV-cfos-dsGFP-KCNA1 compared to AAV-cfos-dsGFP (FIG. 24 C, D). In some cases, the construct of FIG. 8 will lack a sequence encoding a reporter protein, as shown in FIG. 9, and in SEQ ID NO: 10. This may be preferred for regulatory reasons, for example.

### Example 8 - Activity-Dependent Gene Therapy Is Activated by a Single Seizure and Selectively Damps Neuronal Excitability in Hyperactive Neurons

### Materials and Methods

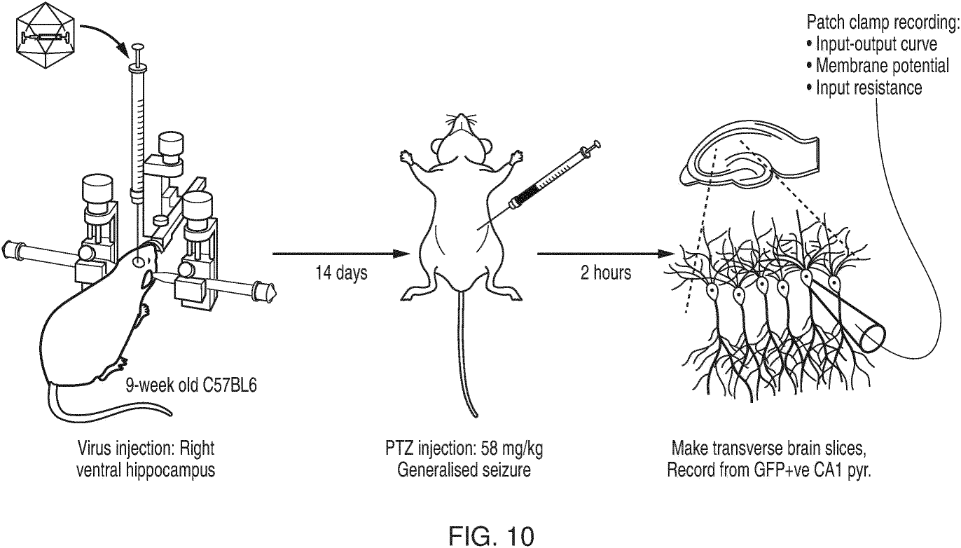

Acute intraperitoneal Pentylenetetrazole (PTZ) injections were performed after viral injection of either AAV cfos-GFP or c-Fos-dsGFP-KCNJ2 or mArc-dsGFP-KCNA1, mArc-dsGFP-KCNJ2 or ESARE-dsGFP-KCNA1, or ESARE-dsGFP-KCNA1 or NRAM-dsGFP-KCNA1. Acute PTZ injections lead to a single tonic-clonic generalised seizure. The effect on fluorescent cells (activated by the seizure) after >2 hours was evaluated with single cell patch clamp. The experimental setup is shown in FIG. 10.

### Results and Discussion

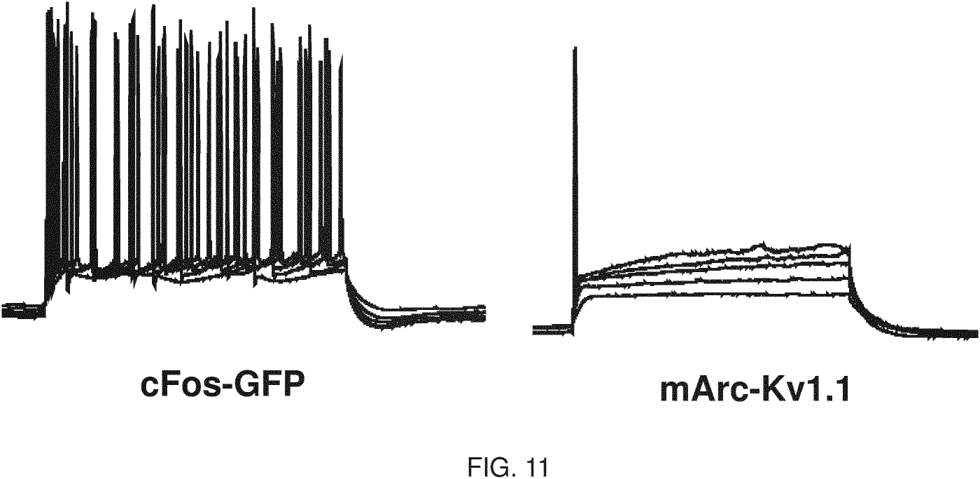

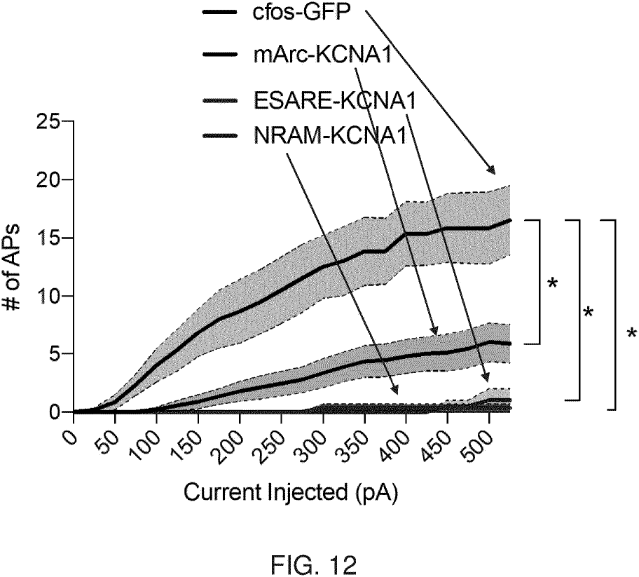

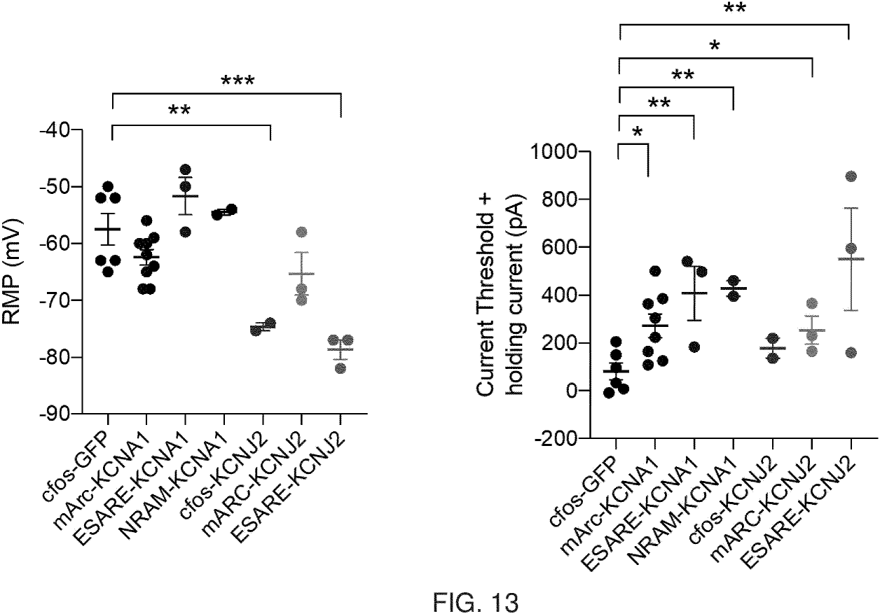

FIGS. 11 to 15 show that, in vivo, activity-dependent gene expression is specific for seizures, and is able to damp neuronal excitability with different promoters and transgenes. The strength of the promoters differed (FIGS. 12, 14 and 15). Expression was observed in Hippocampal CA3 dentate gyrus (granule cells and mossy cells), subiculum and deep hippocampal CA1 neurons. ESARE appears strongest, especially in CA1.The effect of either KCNA1 or KCNJ2 on neurons also differed (FIG. 13), but all permutations of promoter and transgene lead to a profound decrease in neuronal excitability. KCNA1 decreases the firing frequency while KCNJ2 hyperpolarizes the membrane resting potential to make neurons less excitable (FIGS. 11 to 13).

Because the fluorescence is selective to a small subset of neurons, this provides direct evidence that the treatment will not affect bystander neurons that do not participate in the seizure. Thus, the therapeutic effect is specifically targeted to neurons that become over-activated. The transient expression of either KCNA1 or KCNJ2 is enough to reduce neuronal excitability. This provides direct evidence that the treatment selectively decreases the activity of hyperexcitable neurons participating in the seizure.

### Example 9 - Activity-Dependent Gene Therapy Is Activated by a Single Seizure and Is Anti-Epileptic

### Materials and Methods

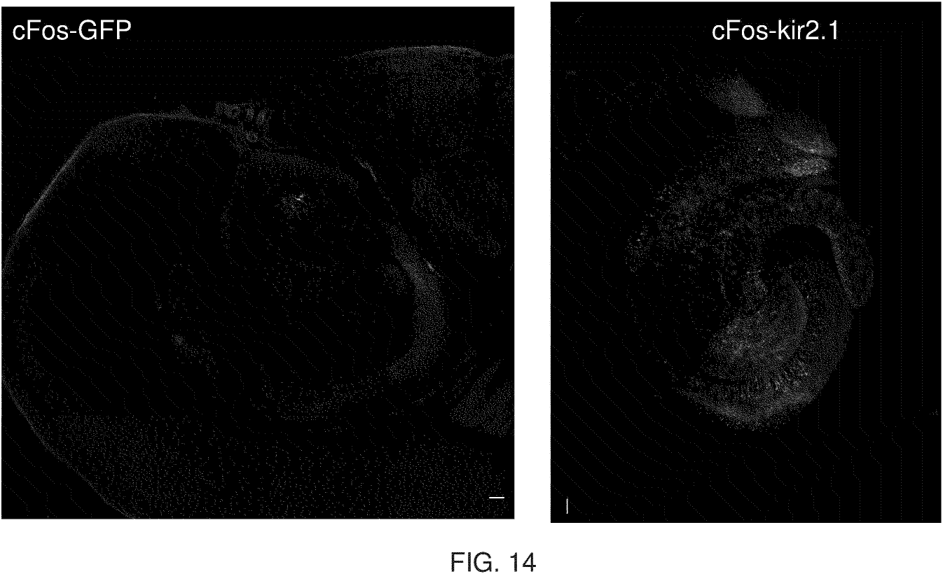

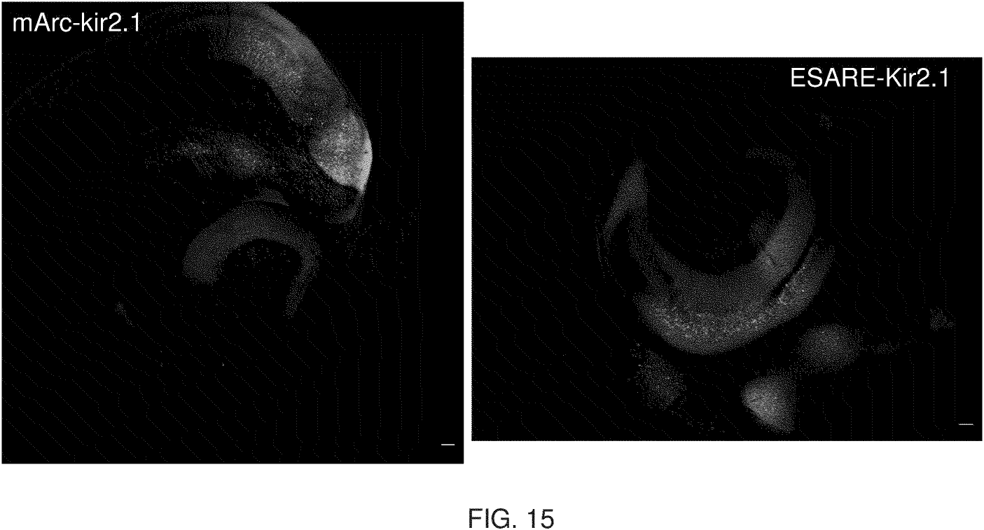

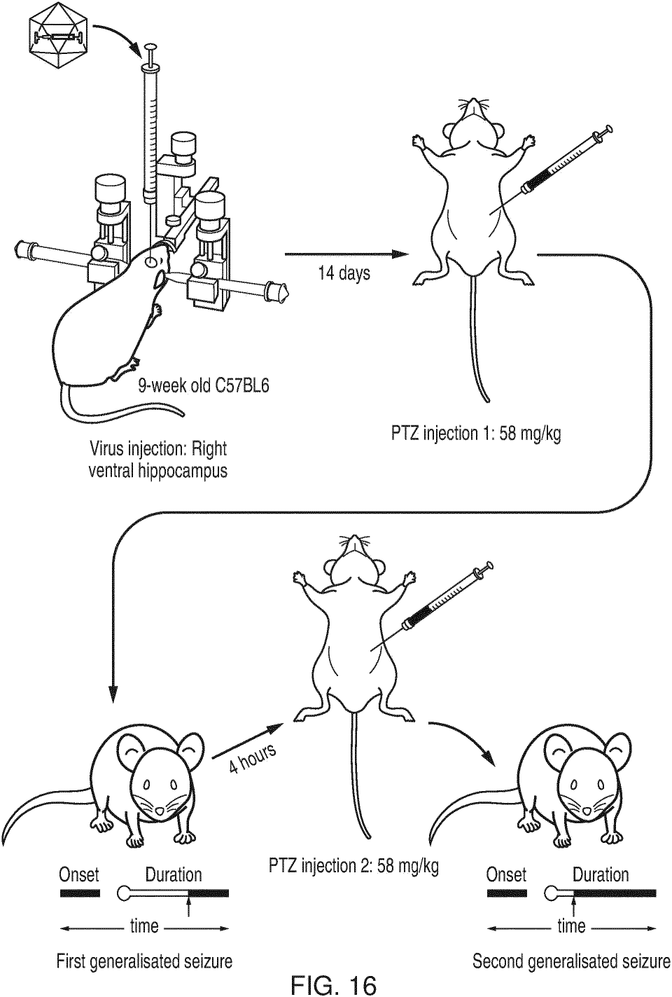

Two consecutive acute intraperitoneal Pentylenetetrazole (PTZ) injections were performed after viral injection of either AAV cfos-GFP or c-Fos-dsGFP-KCNJ2. Each PTZ injection normally leads to a single tonic-clonic generalised seizure allowing the protective effect of the activity-dependent therapy to be evaluated with the second injection. The experimental set up is shown in FIG. 16.

### Results and Discussion

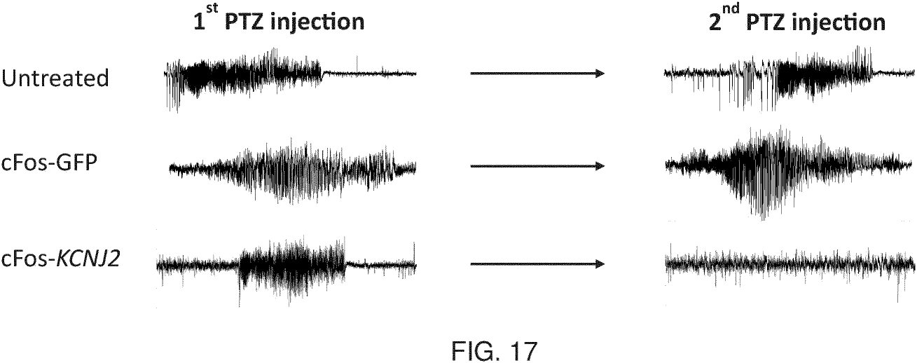

FIG. 17 shows a protective effect against the chemoconvulsant injection. Activity-dependent gene therapy is activated by the first seizure, and prevents the second chemoconvulsant injection from eliciting a seizure. This result provides direct evidence that the treatment will protect from repetitive seizures.

### Example 10 - Activity-Dependent Gene Therapy Suppresses Seizures in a Preclinical Epilepsy Model

### Materials and Methods

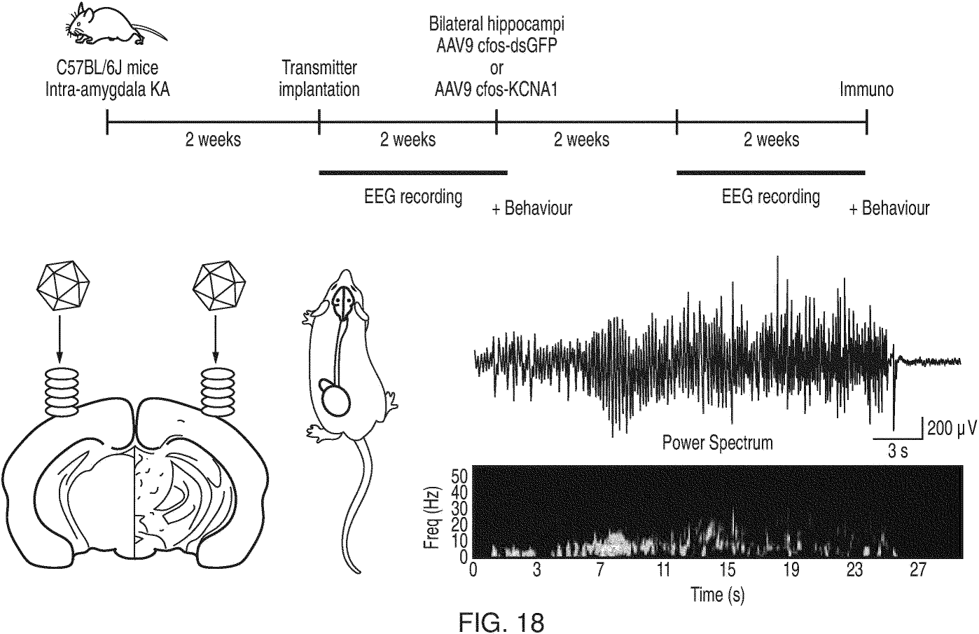

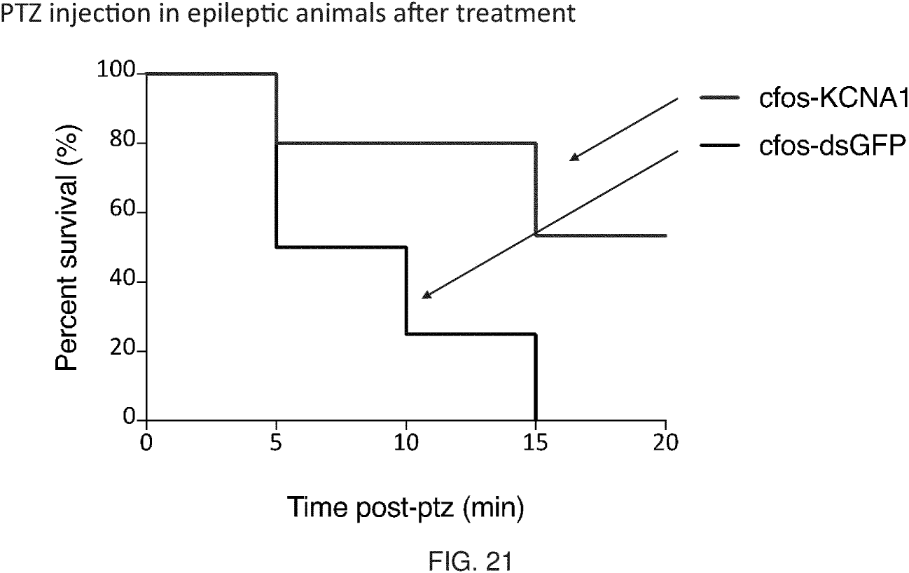

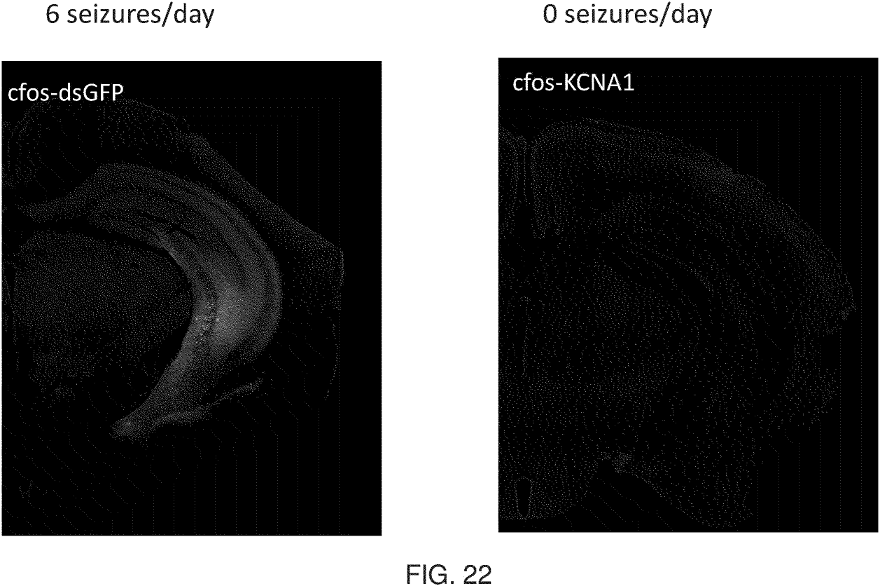

A chronic mouse model of temporal lobe epilepsy (TLE) was created using intra-amygdala injection of kainic acid (KA). After 2 weeks EEG transmitters and cannulas were implanted and the mice were recorded continuously for 2 weeks (Baseline). Then, AAV-cfos-dsGFP or AAV-cfos-dsGFP-KCNA1 (as shown in FIGS. 18-20) were injected through the cannulas and, after waiting 2 weeks for virus expression, animals were recorded for a further 2 weeks. After the recordings some animals were used to analyse fluorescence expression (FIG. 22) or to receive an acute PTZ injection (FIG. 21).

### Results and Discussion

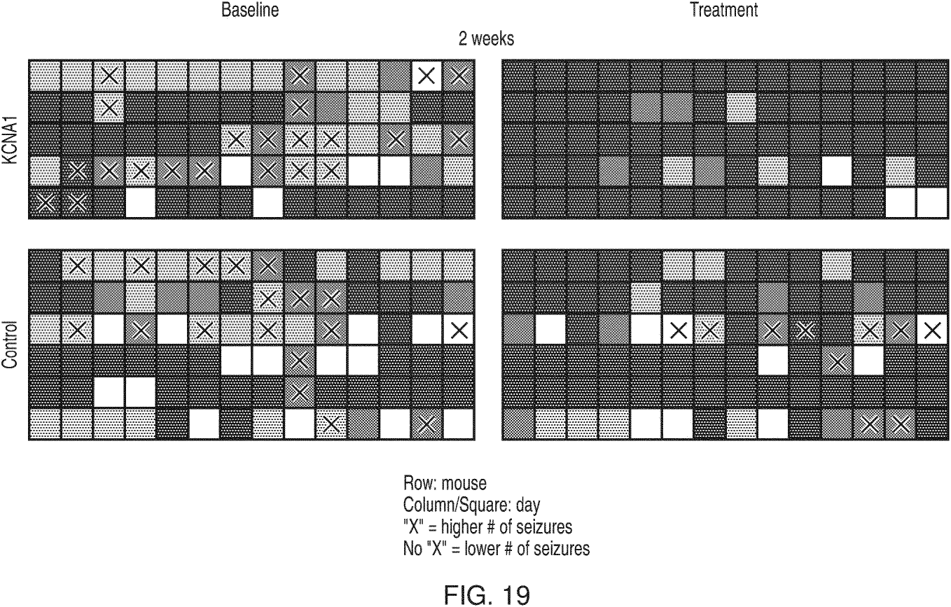

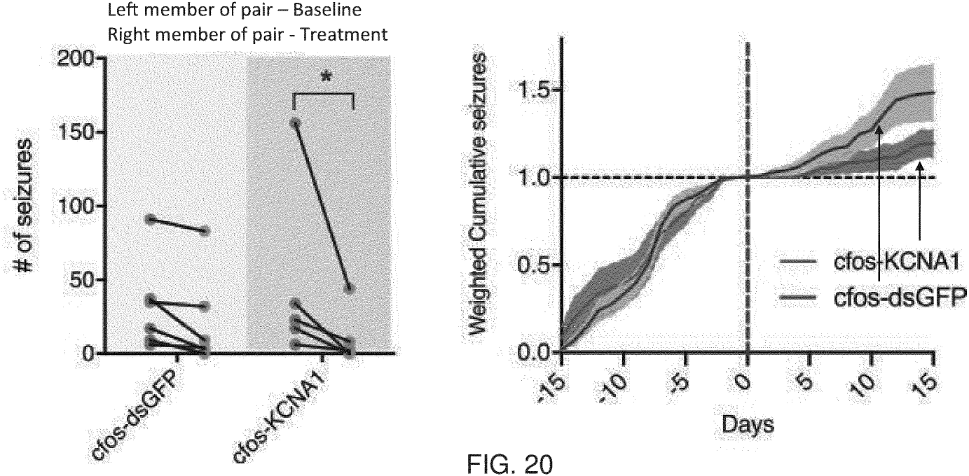

FIGS. 18-20 demonstrates in vivo activity-dependent gene therapy in a mouse epilepsy model. A strong decrease in number of seizures was observed in mice injected with AAV-cfos-dsGFP-KCNA1 compared to AAV-cfos-dsGFP (FIGS. 19 and 20). Animals injected with AAV-cfos-dsGFP-KCNA1 receiving a further PTZ injection showed a higher survival compared to the animals injected with AAV-cfos-dsGFP (FIG. 21). Furthermore, animals treated with AAV-cfos-dsGFP-KCNA1 in whom seizures were suppressed did not exhibit fluorescence, indicating that the therapy was switched off after successful treatment (FIG. 22). N=6 (AAV-cfos-dsGFP) and n=5 (AAV-cfos-dsGFP-KCNA1).

These data confirm the self-regulated anti-epileptic effect of the activity-dependent gene therapy.

In some cases, the construct of FIG. 8 will lack a sequence encoding a reporter protein, as shown in FIG. 9, and in SEQ ID NO: 10. This may be preferred for regulatory reasons, for example.

### Example 11 - Activity-Dependent Gene Therapy Has No Effect on Physiological Behaviour (Spontaneous Locomotion, Anxiety and Memory)

### Materials and Methods

Mice were tested for different behaviour using open field, Object Location Test and T-Maze Spontaneous Alternation before and after injection with either AAV-cfos-dsGFP or AAV-cfos-dsGFP-KCNA1.

### Results and Discussion

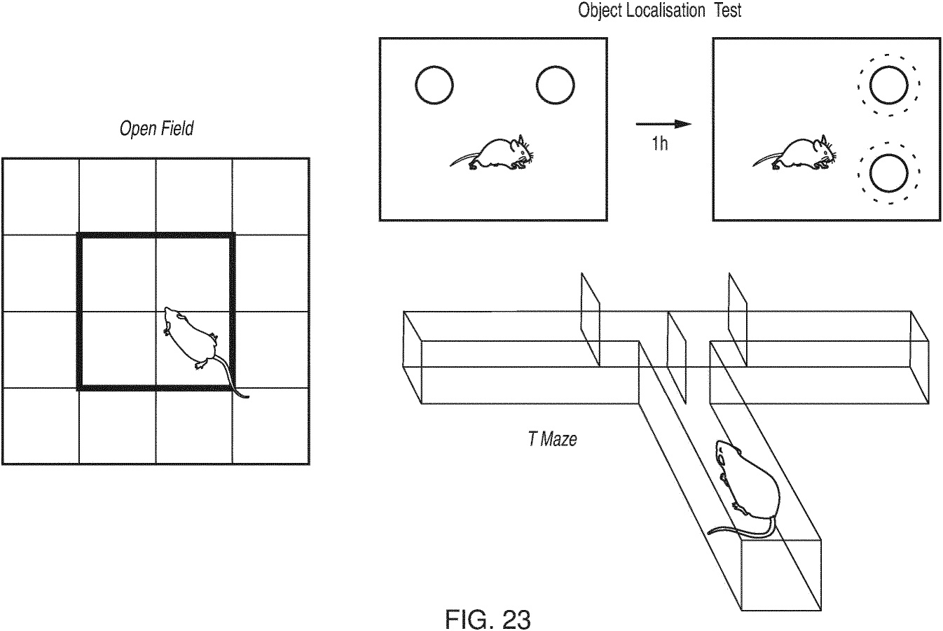

FIG. 23 summarizes the tests used to show that treatment with AAV-cfos-dsGFP-KCNA1 had no deleterious effects on physiological behaviour including spontaneous locomotion, and tests of anxiety and memory. N=5 (AAV-cfos-dsGFP) and n=3 (AAV-cfos-dsGFP-KCNA1).

These data confirm that activity-dependent gene therapy well tolerated.

### FURTHER EMBODIMENTS OF THE INVENTION

The following embodiments E1 to E33 also form part of the invention:

E1. An expression vector for use in a method of treatment of a neurological disorder associated with neuronal hyperexcitability in a subject, the vector comprising:
(i) a polynucleotide sequence (“gene”) encoding a polypeptide (“gene product”) which ameliorates said disorder when expressed in the subject’s neural cells, wherein the gene is operably linked to
(ii) a neuronal activity-dependent promoter suitable to drive expression of the gene product in the subject’s neural cells.

E2. The expression vector for use of E1, wherein the level of expression of the gene product increases when the neuron becomes more excited and decreases when the neuron becomes less excited.

E3. The expression vector for use according to any one of the above embodiments, wherein the promoter is a pyramidal neuronal activity-dependent promoter.

E4. The expression vector for use according to any one of the above embodiments, wherein the promoter is an immediate early gene (IEG) promoter.

E5. The expression vector for use according to any one of the above embodiments, wherein the promoter is c-Fos, Arc, or Egr1.

E6. The expression vector for use according to any one of the above embodiments, wherein the promoter has a nucleotide sequence comprising or consisting of the nucleotide sequence shown in SEQ ID NO: 3 or a nucleotide sequence having at least 80% identity to the nucleotide sequence shown in SEQ ID NO: 3.

E7. The expression vector for use according to any one of the above embodiments, wherein the gene is an ion channel gene, and the gene product is an ion channel.

E8. The expression vector for use according to any one of the above embodiments, wherein the gene is a potassium ion channel gene, and the gene product is a potassium ion channel.

E9. The expression vector for use according to any one of the above embodiments, wherein the gene is a KCNA1 gene, and the gene product is a Kv1.1 potassium channel.

E10. The expression vector for use according to any one of the above embodiments, wherein the gene is an engineered KCNA1 gene, and the gene product is an edited Kv1.1 potassium channel.

E11. The expression vector for use according to any one of the above embodiments, wherein the engineered KCNA1 gene has a nucleotide sequence having at least 90% sequence identity to the nucleotide sequence shown in SEQ ID NO: 1, and

wherein the edited Kv1.1 potassium channel has an amino acid sequence having at least 90% sequence identity to the amino acid sequence shown in SEQ ID NO: 2 and comprises a valine amino acid residue at a position corresponding to amino acid residue 400 shown in SEQ ID NO: 2.

E12. The expression vector for use of any of the above embodiments, wherein the method of treatment is close-loop therapy.

E13. The expression vector for use according to any one of the above embodiments, wherein the neurological disorder is a seizure disorder.

E14. The expression vector for use according to E13, wherein the seizure disorder is epilepsy, optionally neocortical epilepsy, temporal lobe epilepsy or refractory epilepsy.

E15. The expression vector for use according to any one of E1-12, wherein the neurological disorder is Parkinson’s disease, chronic pain, sudden unexpected death in epilepsy (SUDEP), migraine, cluster headache, trigeminal neuralgia, post-herpetic neuralgia, paroxysmal movement disorders, uni- or bipolar affective disorders, anxiety, or phobias.

E16. The expression vector for use according to any one of the above embodiments, wherein the vector is a viral vector.

E17. The expression vector for use according to E16, wherein the viral vector is a recombinant adeno-associated virus (AAV) vector, or a lentiviral vector, optionally wherein the lentiviral vector is a non-integrating lentiviral vector.

E18. The expression vector for use according to E16, wherein the vector comprises a nucleotide sequence having at least 95% identity to the nucleotide sequence of SEQ ID NO: 8, SEQ ID NO: 9 or SEQ ID NO: 10.

E19. An expression vector comprising:
(a) an engineered KCNA1 gene having a nucleotide sequence having at least 90% sequence identity to the nucleotide sequence shown in SEQ ID NO: 1, encoding an edited Kv1.1 potassium channel having an amino acid sequence having at least 90% sequence identity to the amino acid sequence shown in SEQ ID NO: 2 and comprises a valine amino acid residue at a position corresponding to amino acid residue 400 shown in SEQ ID NO: 2; and
(b) an activity-dependent promoter having a nucleotide sequence comprising or consisting of the nucleotide sequence shown in SEQ ID NO: 3 or a nucleotide sequence having at least 80% identity to the nucleotide sequence shown in SEQ ID NO: 3,
wherein the gene is operably linked to the promoter.

E20 An in vitro method of making viral particles comprising:
transducing mammalian cells with a vector according to any one of E1-19 and expressing viral packaging and envelope proteins necessary for particle formation in the cells; and
culturing the transduced cells in a culture medium, such that the cells produce viral particles that are released into the medium.

E21. An in vitro method of E20, wherein the method comprises transducing the mammalian cells with one or more viral packaging and envelope expression vectors that encode the viral packaging and envelope proteins necessary for particle formation.

E22. An in vitro method of E20 or E21, wherein the one or more packaging proteins includes a non-functional integrase enzyme such that the vector is unable to incorporate its viral genome into the genome of the cell.

E23. An in vitro method of any one of E20-22, further comprising separating the viral particles from the culture medium and optionally concentrating the viral particles.

E24. A viral particle produced by the method of any one of E20-23, the viral particle optionally comprising an RNA molecule or DNA molecule transcribed from the expression vector of any of E1-19.

E25. A viral particle comprising a single stranded RNA molecule or DNA molecule encoding a gene as described in any one of E1-19,
wherein the gene encodes a gene product as defined in any one of E1-19,
wherein the promoter is optionally as defined in any one of E1-19, and
wherein the viral particle is optionally an AAV.

E26. A kit comprising an expression vector of any one of E1-19 and one or more viral packaging and envelope expression vectors that encode viral packaging and envelope proteins necessary for particle formation when expressed in a cell.

E27. A kit of E26, wherein the viral packaging expression vector is an integrase-deficient viral packaging expression vector.

E28. A viral particle of E24 or E25 for use in a method of treatment, wherein the method of treatment is defined in any one of E12-15.

E29. A method of treatment of a neurological disorder as defined in any one of E1 and 12-15, comprising administering to an individual with the neurological disorder the expression vector as defined in any one of E1-19, or the viral particle of E24 or E25.

E30. A method of confirming the presence of a gene product as defined in any one of E1-19, the method comprising:
transducing a cell with an expression vector of any one of E1-19 or administering a viral particle of E24 or E25 to a cell under conditions that permit expression of the gene product; and
detecting the presence of the gene product in the cell using a hybridisation assay.

E31. An in vitro or ex vivo method of confirming the presence of a gene product as defined in any one of E1-19 that has been obtained from a subject administered with a viral particle of E24 or E25, the method comprising:

detecting the presence of the gene product in the cell using a hybridisation assay.

E32. A method of E29 or E30, wherein the hybridisation assay is an in situ hybridisation assay using a labelled RNA probe, optionally wherein the labelled RNA probe is fluorescently labelled.

E33. A cell comprising the expression vector of any one of E1-19.

### Sequence Annex

Nucleotide sequence of an exemplary engineered hum
an KCNA1 gene (SEQ ID NO: 1) ATGACCGTGATGAGCGGCGAG
AACGTGGACGAGGCCTCTGCCGCTCCTGGACACCCTCAGGATGGCAGCTA
TCCCAGACAGGCCGACCACGACGATCACGAGTGCTGCGAGCGGGTCGTGA
TCAACATCAGCGGCCTGAGATTCGAGACACAGCTGAAAACCCTGGCCCAG
TTCCCCAACACCCTGCTGGGCAACCCCAAGAAACGGATGCGGTACTTCGA
CCCCCTGCGGAACGAGTACTTCTTCGACCGGAACCGGCCCAGCTTCGACG
CCATCCTGTACTACTACCAGAGCGGCGGCAGACTGCGGAGGCCCGTGAAT
GTGCCCCTGGACATGTTCAGCGAGGAAATCAAGTTCTACGAGCTGGGCGA
GGAAGCCATGGAAAAGTTCAGAGAGGACGAGGGCTTCATCAAAGAGGAAG
AGAGGCCCCTGCCCGAGAAAGAATACCAGAGACAAGTGTGGCTGCTGTTC
GAGTACCCCGAGTCTAGCGGCCCTGCCAGAGTGATCGCCATCGTGTCCGT
GATGGTCATCCTGATCTCTATCGTGATCTTCTGCCTGGAAACCCTGCCTG
AGCTGAAGGACGACAAGGACTTCACCGGCACCGTGCACCGGATCGACAAC
ACCACCGTGATCTACAACAGCAATATCTTCACCGACCCATTCTTCATCGT
GGAAACACTGTGCATCATCTGGTTCAGCTTCGAGCTGGTCGTGCGGTTCT
TCGCCTGCCCCAGCAAGACCGACTTCTTCAAGAACATCATGAACTTCATT
GATATCGTGGCCATCATCCCCTACTTCATCACCCTGGGCACCGAGATCGC
CGAGCAGGAAGGCAATCAGAAGGGCGAGCAGGCCACCAGCCTGGCCATTC
TGAGAGTGATCAGACTCGTGCGGGTGTTCCGGATCTTCAAGCTGAGCCGG
CACAGCAAGGGCCTGCAGATCCTGGGCCAGACACTGAAGGCCAGCATGAG
AGAGCTGGGCCTGCTGATCTTCTTTCTGTTCATCGGCGTGATCCTGTTCA
GCAGCGCCGTGTACTTCGCCGAGGCCGAAGAAGCCGAGAGCCACTTCAGC
TCTATCCCCGACGCCTTTTGGTGGGCCGTGGTGTCCATGACCACAGTGGG
CTACGGCGACATGTAnCCCGTGACAATCGGCGGCAAGATCGTGGGCAGCC
TGTGTGCCATTGCCGGCGTGCTGACAGTCGCCCTGCCTGTGCCTGTGATC
GTGTCCAACTTCAACTACTTCTACCACCGGGAAACCGAGGGGGAGGAACA
GGCTCAGCTGCTGCACGTGTCCAGCCCCAATCTGGCCAGCGACAGCGACC
TGAGCAGACGGTCTAGCAGCACCATGAGCAAGAGCGAGTACATGGAAATC
GAAGAGGACATGAACAACTCTATCGCCCACTACCGCCAAGTGAACATCCG
GACCGCCAACTGCACCACCGCCAACCAGAACTGCGTGAACAAGAGCAAGC
TGCTGACCGATGTCTGA

wherein n is T or C

Amino acid sequence of an edited human Kv1.1 compr
ising a valine at position 400 (underlined) (SEQ I
D NO: 2) MTVMSGENVDEASAAPGHPQDGSYPRQADHDDHECCERWIN
ISGLRFETQLKTLAQFPNTLLGNPKKRMRYFDPLRNEYFFDRNRPSFDAI
LYYYQSGGRLRRPVNVPLDMFSEEIKFYELGEEAMEKFREDEGFIKEEER
PLPEKEYQRQVWLLFEYPESSGPARVIAIVSVMVILISIVIFCLETLPEL
KDDKDFTGTVHRIDNTTVIYNSNIFTDPFFIVETLCIIWFSFELWRFFAC
PSKTDFFKNIMNFIDIVAIIPYFITLGTEIAEQEGNQKGEQATSLAILRV
IRLVRVFRIFKLSRHSKGLQILGQTLKASMRELGLLIFFLFIGVILFSSA
VYFAEAEEAESHFSSIPDAFWWAWSMTTVGYGDMYPVTIGGKIVGSLCAI
AGVLTVALPVPVIVSNFNYFYHRETEGEEQAQLLHVSSPNLASDSDLSRR
SSSTMSKSEYMEIEEDMNNSIAHYRQVNIRTANCTTANQNCVNKSKLLTD
V

Nucleotide sequence of the cfos promoter (SEQ ID N
O: 3) GCGGCCGCAGCTTTCCTTTAGGAACAGAGGCTTCGAGCCTTTAA
GGCTGCGTACTTGCTTCTCCTAATACCAGAGACTCAAAAAAAAAAAAAAA
GTTCCAGATTGCTGGACAATGACCCGGGTCTCATCCCTTGACCCTGGGAA
CCGGGTCCACATTGAATCAGGTGCGAATGTTCGCTCGCCTTCTCTGCCTT
TCCCGCCTCCCCTCCCCCGGCCGCGGCCCCGGTTCCCCCCCTGCGCTGCA
CCCTCAGAGTTGGCTGCAGCCGGCGAGCTGTTCCCGTCAATCCCTCCCTC
CTTTACACAGGATGTCCATATTAGGACATCTGCGTCAGCAGGTTTCCACG
GCCGGTCCCTGTTGTTCTGGGGGGGGGACCATCTCCGAAATCCTACACGC
GGAAGGTCTAGGAGACCCCCTAAGATCCCAAATGTGAACACTCATAGGTG
AAAGATGTATGCCAAGACGGGGGTTGAAAGCCTGGGGCGTAGAGTTGACG
ACAGAGCGCCCGCAGAGGGCCTTGGGGCGCGCTTCCCCCCCCTTCCAGTT
CCGCCCAGTGACGTAGGAAGTCCATCCATTCACAGCGCT

Nucleotide sequence of wild-type KCNA1 coding sequ
ence, comprising an adenine at nucleotide position
1998(underlined) (SEQ ID NO: 4) ATGACGGTGATGTCTGGG
GAGAACGTGGACGAGGCTTCGGCCGCCCCGGGCCACCCCCAGGATGGCAG
CTACCCCCGGCAGGCCGACCACGACGACCACGAGTGCTGCGAGCGCGTGG
TGATCAACATCTCCGGGCTGCGCTTCGAGACGCAGCTCAAGACCCTGGCG
CAGTTCCCCAACACGCTGCTGGGCAACCCTAAGAAACGCATGCGCTACTT
CGACCCCCTGAGGAACGAGTACTTCTTCGACCGCAACCGGCCCAGCTTCG
ACGCCATCCTCTACTACTACCAGTCCGGCGGCCGCCTGCGGAGGCCGGTC
AACGTGCCCCTGGACATGTTCTCCGAGGAGATCAAGTTTTACGAGTTGGG
CGAGGAGGCCATGGAGAAGTTCCGGGAGGACGAGGGCTTCATCAAGGAGG
AGGAGCGCCCTCTGCCCGAGAAGGAGTACCAGCGCCAGGTGTGGCTGCTC
TTCGAGTACCCCGAGAGCTCGGGGCCCGCCAGGGTCATCGCCATCGTCTC
CGTCATGGTCATCCTCATCTCCATCGTCATCTTTTGCCTGGAGACGCTCC
CCGAGCTGAAGGATGACAAGGACTTCACGGGCACCGTCCACCGCATCGAC
AACACCACGGTCATCTACAATTCCAACATCTTCACAGACCCCTTCTTCAT
CGTGGAAACGCTGTGTATCATCTGGTTCTCCTTCGAGCTGGTGGTGCGCT
TCTTCGCCTGCCCCAGCAAGACGGACTTCTTCAAAAACATCATGAACTTC
ATAGACATTGTGGCCATCATTCCTTATTTCATCACGCTGGGCACCGAGAT
AGCTGAGCAGGAAGGAAACCAGAAGGGCGAGCAGGCCACCTCCCTGGCCA
TCCTCAGGGTCATCCGCTTGGTAAGGGTTTTTAGAATCTTCAAGCTCTCC
CGCCACTCTAAGGGCCTCCAGATCCTGGGCCAGACCCTCAAAGCTAGTAT
GAGAGAGCTAGGGCTGCTCATCTTTTTCCTCTTCATCGGGGTCATCCTGT
TTTCTAGTGCAGTGTACTTTGCCGAGGCGGAAGAAGCTGAGTCGCACTTC
TCCAGTATCCCCGATGCTTTCTGGTGGGCGGTGGTGTCCATGACCACTGT
AGGATACGGTGACATGTACCCTGTGACAATTGGAGGCAAGATCGTGGGCT
CCTTGTGTGCCATCGCTGGTGTGCTAACAATTGCCCTGCCCGTACCTGTC
ATTGTGTCCAATTTCAACTATTTCTACCACCGAGAAACTGAGGGGGAAGA
GCAGGCTCAGTTGCTCCACGTCAGTTCCCCTAACTTAGCCTCTGACAGTG
ACCTCAGTCGCCGCAGTTCCTCTACTATGAGCAAGTCTGAGTACATGGAG
ATCGAAGAGGATATGAATAATAGCATAGCCCATTATAGACAGGTCAATAT
CAGAACTGCCAATTGCACCACTGCTAACCAAAACTGCGTTAATAAGAGCA
AGCTACTGACCGATGTTTAA

Amino acid sequence of wild-type human Kv1.1, comp
rising a isoleucine at position 400 (underlined) (
SEQ ID NO: 5) MTVMSGENVDEASAAPGHPQDGSYPRQADHDDHECC
ERWINISGLRFETQLKTLAQFPNTLLGNPKKRMRYFDPLRNEYFFDRNRP
SFDAILYYYQSGGRLRRPVNVPLDMFSEEIKFYELGEEAMEKFREDEGFI
KEEERPLPEKEYQRQVWLLFEYPESSGPARVIAIVSVMVILISIVIFCLE
TLPELKDDKDFTGTVHRIDNTTVIYNSNIFTDPFFIVETLCIIWFSFELW
RFFACPSKTDFFKNIMNFIDIVAIIPYFITLGTEIAEQEGNQKGEQATSL
AILRVIRLVRVFRIFKLSRHSKGLQILGQTLKASMRELGLLIFFLFIGVI
LFSSAVYFAEAEEAESHFSSIPDAFWWAWSMTTVGYGDMYPVTIGGKIVG
SLCAIAGVLTIALPVPVIVSNFNYFYHRETEGEEQAQLLHVSSPNLASDS
DLSRRSSSTMSKSEYMEIEEDMNNSIAHYRQVNIRTANCTTANQNCVNKS
KLLTDV

Nucleotide sequence of cfos-GFP construct (SEQ ID
NO: 6) GCGGCCGCAGCTTTCCTTTAGGAACAGAGGCTTCGAGCCTTTA
AGGCTGCGTACTTGCTTCTCCTAATACCAGAGACTCAAAAAAAAAAAAAA
AGTTCCAGATTGCTGGACAATGACCCGGGTCTCATCCCTTGACCCTGGGA
ACCGGGTCCACATTGAATCAGGTGCGAATGTTCGCTCGCCTTCTCTGCCT
TTCCCGCCTCCCCTCCCCCGGCCGCGGCCCCGGTTCCCCCCCTGCGCTGC
ACCCTCAGAGTTGGCTGCAGCCGGCGAGCTGTTCCCGTCAATCCCTCCCT
CCTTTACACAGGATGTCCATATTAGGACATCTGCGTCAGCAGGTTTCCAC
GGCCGGTCCCTGTTGTTCTGGGGGGGGGACCATCTCCGAAATCCTACACG
CGGAAGGTCTAGGAGACCCCCTAAGATCCCAAATGTGAACACTCATAGGT
GAAAGATGTATGCCAAGACGGGGGTTGAAAGCCTGGGGCGTAGAGTTGAC
GACAGAGCGCCCGCAGAGGGCCTTGGGGCGCGCTTCCCCCCCCTTCCAGT
TCCGCCCAGTGACGTAGGAAGTCCATCCATTCACAGCGCTTCTATAAAGG
CGCCAGCTGAGGCGCCTACTACTCCAACCGCGACTGCAGCGAGCAACTGA
GAAGACTGGATAGAGCCGGCGGTTCCGCGAACGAGCAGTGACCGCGCTCC
CACCCAGCTCTGCTCTGCAGCTCCCACCAGTGTCTGGCCGCATCGATTCT
AGAATTCGCTGTCTGCGAGGGCCAGCTGTTGGGGTGAGTACTCCCTCTCA
AAAGCGGGCATGACTTCTGCGCTAAGATTGTCAGTTTCCAAAAACGAGGA
GGATTTGATATTCACCTGGCCCGCGGTGATGCCTTTGAGGGTGGCCGCGT
CCATCTGGTCAGAAAAGACAATCTTTTTGTTGTCAAGCTTGAGGTGTGGC
AGGCTTGAGATCTGGCCATACACTTGAGTGACAATGACATCCACTTTGCC
TTTCTCTCCACAGGTGTCCACTCCCAGGTCCAACTGCAGCCCAAGCGGAG
GATCCGCCACCatgcccgccatgaagatcgagtgccgcatcaccggcacc
ctgaacggcgtggagttcgagctggtgggcggcggagagggcacccccga
GCAGGGCCGCATGACCAACAAGATGAAGAGCACCAAAGGCGCCCTGACCT
TCAGCCCCTACCTGCTGAGCCACGTGATGGGCTACGGCTTCTACCACTTC
GGCACCTACCCCAGCGGCTACGAGAACCCCTTCCTGCACGCCATCAACAA
CGGCGGCTACACCAACACCCGCATCGAGAAGTACGAGGACGGCGGCGTGC
TGCACGTGAGCTTCAGCTACCGCTACGAGGCCGGCCGCGTGATCGGCGAC
TTCAAGGTGGTGGGCACCGGCTTCCCCGAGGACAGCGTGATCTTCACCGA
CAAGATCATCCGCAGCAACGCCACCGTGGAGCACCTGCACCCCATGGGCG
ATAACGTGCTGGTGGGCAGCTTCGCCCGCACCTTCAGCCTGCGCGACGGC
GGCTACTACAGCTTCGTGGTGGACAGCCACATGCACTTCAAGAGCGCCAT
CCACCCCAGCATCCTGCAGAACGGGGGCCCCATGTTCGCCTTCCGCCGCG
TGGAGGAGCTGCACAGCAACACCGAGCTGGGCATCGTGGAGTACCAGCAC
GCCTTCAAGACCCCCATCGCCTTCGCCAGATCTCGAGATATCAGCCATGG
CTTCCCGCCGGCGGTGGCGGCGCAGGATGATGGCACGCTGCCCATGTCTT
GTGCCCAGGAGAGCGGGATGGACCGTCACCCTGCAGCCTGTGCTTCTGCT
AGGATCAATGTGTGA

Nucleotide sequence of cfos-dsGFP-KCNA1 construct
(SEQ ID NO: 7) GCGGCCGCAGCTTTCCTTTAGGAACAGAGGCTTCG
AGCCTTTAAGGCTGCGTACTTGCTTCTCCTAATACCAGAGACTCAAAAAA
AAAAAAAAAGTTCCAGATTGCTGGACAATGACCCGGGTCTCATCCCTTGA
CCCTGGGAACCGGGTCCACATTGAATCAGGTGCGAATGTTCGCTCGCCTT
CTCTGCCTTTCCCGCCTCCCCTCCCCCGGCCGCGGCCCCGGTTCCCCCCC
TGCGCTGCACCCTCAGAGTTGGCTGCAGCCGGCGAGCTGTTCCCGTCAAT
CCCTCCCTCCTTTACACAGGATGTCCATATTAGGACATCTGCGTCAGCAG
GTTTCCACGGCCGGTCCCTGTTGTTCTGGGGGGGGGACCATCTCCGAAAT
CCTACACGCGGAAGGTCTAGGAGACCCCCTAAGATCCCAAATGTGAACAC
TCATAGGTGAAAGATGTATGCCAAGACGGGGGTTGAAAGCCTGGGGCGTA
GAGTTGACGACAGAGCGCCCGCAGAGGGCCTTGGGGCGCGCTTCCCCCCC
CTTCCAGTTCCGCCCAGTGACGTAGGAAGTCCATCCATTCACAGCGCTTC
TATAAAGGCGCCAGCTGAGGCGCCTACTACTCCAACCGCGACTGCAGCGA
GCAACTGAGAAGACTGGATAGAGCCGGCGGTTCCGCGAACGAGCAGTGAC
CGCGCTCCCACCCAGCTCTGCTCTGCAGCTCCCACCAGTGTCTGGCCGCA
TCGATTCTAGAATTCGCTGTCTGCGAGGGCCAGCTGTTGGGGTGAGTACT
CCCTCTCAAAAGCGGGCATGACTTCTGCGCTAAGATTGTCAGTTTCCAAA
AACGAGGAGGATTTGATATTCACCTGGCCCGCGGTGATGCCTTTGAGGGT
GGCCGCGTCCATCTGGTCAGAAAAGACAATCTTTTTGTTGTCAAGCTTGA
GGTGTGGCAGGCTTGAGATCTGGCCATACACTTGAGTGACAATGACATCC
ACTTTGCCTTTCTCTCCACAGGTGTCCACTCCCAGGTCCAACTGCAGCCC
AAGCGGAGGATCCGCCACCatgcccgccatgaagatcgagtgccgcatca
ccggcaccctgaacggcgtggagttcgagctggtgggcggcggagagggc
acccccgaGCAGGGCCGCATGACCAACAAGATGAAGAGCACCAAAGGCGC
CCTGACCTTCAGCCCCTACCTGCTGAGCCACGTGATGGGCTACGGCTTCT
ACCACTTCGGCACCTACCCCAGCGGCTACGAGAACCCCTTCCTGCACGCC
ATCAACAACGGCGGCTACACCAACACCCGCATCGAGAAGTACGAGGACGG
CGGCGTGCTGCACGTGAGCTTCAGCTACCGCTACGAGGCCGGCCGCGTGA
TCGGCGACTTCAAGGTGGTGGGCACCGGCTTCCCCGAGGACAGCGTGATC
TTCACCGACAAGATCATCCGCAGCAACGCCACCGTGGAGCACCTGCACCC
CATGGGCGATAACGTGCTGGTGGGCAGCTTCGCCCGCACCTTCAGCCTGC
GCGACGGCGGCTACTACAGCTTCGTGGTGGACAGCCACATGCACTTCAAG
AGCGCCATCCACCCCAGCATCCTGCAGAACGGGGGCCCCATGTTCGCCTT
CCGCCGCGTGGAGGAGCTGCACAGCAACACCGAGCTGGGCATCGTGGAGT
ACCAGCACGCCTTCAAGACCCCCATCGCCTTCGCCAGATCTCGAGATATC
AGCCATGGCTTCCCGCCGGCGGTGGCGGCGCAGGATGATGGCACGCTGCC
CATGTCTTGTGCCCAGGAGAGCGGGATGGACCGTCACCCTGCAGCCTGTG
CTTCTGCTAGGATCAATGTGACCGGTGAGGGCAGAGGAAGTCTTCTAACA
TGCGGTGACGTGGAGGAGAATCCCGGCCCTATGACCGTGATGAGCGGCGA
GAACGTGGACGAGGCCTCTGCCGCTCCTGGACACCCTCAGGATGGCAGCT
ATCCCAGACAGGCCGACCACGACGATCACGAGTGCTGCGAGCGGGTCGTG
ATCAACATCAGCGGCCTGAGATTCGAGACACAGCTGAAAACCCTGGCCCA
GTTCCCCAACACCCTGCTGGGCAACCCCAAGAAACGGATGCGGTACTTCG
ACCCCCTGCGGAACGAGTACTTCTTCGACCGGAACCGGCCCAGCTTCGAC
GCCATCCTGTACTACTACCAGAGCGGCGGCAGACTGCGGAGGCCCGTGAA
TGTGCCCCTGGACATGTTCAGCGAGGAAATCAAGTTCTACGAGCTGGGCG
AGGAAGCCATGGAAAAGTTCAGAGAGGACGAGGGCTTCATCAAAGAGGAA
GAGAGGCCCCTGCCCGAGAAAGAATACCAGAGACAAGTGTGGCTGCTGTT
CGAGTACCCCGAGTCTAGCGGCCCTGCCAGAGTGATCGCCATCGTGTCCG
TGATGGTCATCCTGATCTCTATCGTGATCTTCTGCCTGGAAACCCTGCCT
GAGCTGAAGGACGACAAGGACTTCACCGGCACCGTGCACCGGATCGACAA
CACCACCGTGATCTACAACAGCAATATCTTCACCGACCCATTCTTCATCG
TGGAAACACTGTGCATCATCTGGTTCAGCTTCGAGCTGGTCGTGCGGTTC
TTCGCCTGCCCCAGCAAGACCGACTTCTTCAAGAACATCATGAACTTCAT
TGATATCGTGGCCATCATCCCCTACTTCATCACCCTGGGCACCGAGATCG
CCGAGCAGGAAGGCAATCAGAAGGGCGAGCAGGCCACCAGCCTGGCCATT
CTGAGAGTGATCAGACTCGTGCGGGTGTTCCGGATCTTCAAGCTGAGCCG
GCACAGCAAGGGCCTGCAGATCCTGGGCCAGACACTGAAGGCCAGCATGA
GAGAGCTGGGCCTGCTGATCTTCTTTCTGTTCATCGGCGTGATCCTGTTC
AGCAGCGCCGTGTACTTCGCCGAGGCCGAAGAAGCCGAGAGCCACTTCAG
CTCTATCCCCGACGCCTTTTGGTGGGCCGTGGTGTCCATGACCACAGTGG
GCTACGGCGACATGGTGCCCGTGACAATCGGCGGCAAGATCGTGGGCAGC
CTGTGTGCCATTGCCGGCGTGCTGACAGTCGCCCTGCCTGTGCCTGTGAT
CGTGTCCAACTTCAACTACTTCTACCACCGGGAAACCGAGGGGGAGGAAC
AGGCTCAGCTGCTGCACGTGTCCAGCCCCAATCTGGCCAGCGACAGCGAC
CTGAGCAGACGGTCTAGCAGCACCATGAGCAAGAGCGAGTACATGGAAAT
CGAAGAGGACATGAACAACTCTATCGCCCACTACCGCCAAGTGAACATCC
GGACCGCCAACTGCACCACCGCCAACCAGAACTGCGTGAACAAGAGCAAG
CTGCTGACCGATGTCTGA

Nucleotide sequence of optimised AAV-cfos-dsGFP-KC
NA1 vector (SEQ ID NO: 8) cctgcaggcagctgcgcgctcgct
cgctcactgaggccgcccgggcaaagcccgggcgtcgggcgacctttggt
cgcccggcctcagtgagcgagcgagcgcgcagagagggagtggccaactc
catcactaggggttcctgcggccgcacgcgtTTCGCTATTACGCCAGTTT
TATTGCGGCCGCAGCTTTCCTTTAGGAACAGAGGCTTCGAGCCTTTAAGG
CTGCGTACTTGCTTCTCCTAATACCAGAGACTCAAAAAAAAAAAAAAAGT
TCCAGATTGCTGGACAATGACCCGGGTCTCATCCCTTGACCCTGGGAACC
GGGTCCACATTGAATCAGGTGCGAATGTTCGCTCGCCTTCTCTGCCTTTC
CCGCCTCCCCTCCCCCGGCCGCGGCCCCGGTTCCCCCCCTGCGCTGCACC
CTCAGAGTTGGCTGCAGCCGGCGAGCTGTTCCCGTCAATCCCTCCCTCCT
TTACACAGGATGTCCATATTAGGACATCTGCGTCAGCAGGTTTCCACGGC
CGGTCCCTGTTGTTCTGGGGGGGGGACCATCTCCGAAATCCTACACGCGG
AAGGTCTAGGAGACCCCCTAAGATCCCAAATGTGAACACTCATAGGTGAA
AGATGTATGCCAAGACGGGGGTTGAAAGCCTGGGGCGTAGAGTTGACGAC
AGAGCGCCCGCAGAGGGCCTTGGGGCGCGCTTCCCCCCCCTTCCAGTTCC
GCCCAGTGACGTAGGAAGTCCATCCATTCACAGCGCTTCTATAAAGGCGC
CAGCTGAGGCGCCTACTACTCCAACCGCGACTGCAGCGAGCAACTGAGAA
GACTGGATAGAGCCGGCGGTTCCGCGAACGAGCAGTGACCGCGCTCCCAC
CCAGCTCTGCTCTGCAGCTCCCACCAGTGTCTGGCCGCATCGATTCTAGA
ATTCGCTGTCTGCGAGGGCCAGCTGTTGGGGTGAGTACTCCCTCTCAAAA
GCGGGCATGACTTCTGCGCTAAGATTGTCAGTTTCCAAAAACGAGGAGGA
TTTGATATTCACCTGGCCCGCGGTGATGCCTTTGAGGGTGGCCGCGTCCA
TCTGGTCAGAAAAGACAATCTTTTTGTTGTCAAGCTTGAGGTGTGGCAGG
CTTGAGATCTGGCCATACACTTGAGTGACAATGACATCCACTTTGCCTTT
CTCTCCACAGGTGTCCACTCCCAGGTCCAACTGCAGCCCAAGCGGAGGAT
CCGCCACCatgcccgccatgaagatcgagtgccgcatcaccggcaccctg
aacggcgtggagttcgagctggtgggcggcggagagggcacccccgaGCA
GGGCCGCATGACCAACAAGATGAAGAGCACCAAAGGCGCCCTGACCTTCA
GCCCCTACCTGCTGAGCCACGTGATGGGCTACGGCTTCTACCACTTCGGC
ACCTACCCCAGCGGCTACGAGAACCCCTTCCTGCACGCCATCAACAACGG
CGGCTACACCAACACCCGCATCGAGAAGTACGAGGACGGCGGCGTGCTGC
ACGTGAGCTTCAGCTACCGCTACGAGGCCGGCCGCGTGATCGGCGACTTC
AAGGTGGTGGGCACCGGCTTCCCCGAGGACAGCGTGATCTTCACCGACAA
GATCATCCGCAGCAACGCCACCGTGGAGCACCTGCACCCCATGGGCGATA
ACGTGCTGGTGGGCAGCTTCGCCCGCACCTTCAGCCTGCGCGACGGCGGC
TACTACAGCTTCGTGGTGGACAGCCACATGCACTTCAAGAGCGCCATCCA
CCCCAGCATCCTGCAGAACGGGGGCCCCATGTTCGCCTTCCGCCGCGTGG
AGGAGCTGCACAGCAACACCGAGCTGGGCATCGTGGAGTACCAGCACGCC
TTCAAGACCCCCATCGCCTTCGCCAGATCTCGAGATATCAGCCATGGCTT
CCCGCCGGCGGTGGCGGCGCAGGATGATGGCACGCTGCCCATGTCTTGTG
CCCAGGAGAGCGGGATGGACCGTCACCCTGCAGCCTGTGCTTCTGCTAGG
ATCAATGTGACCGGTGAGGGCAGAGGAAGTCTTCTAACATGCGGTGACGT
GGAGGAGAATCCCGGCCCTATGACCGTGATGAGCGGCGAGAACGTGGACG
AGGCCTCTGCCGCTCCTGGACACCCTCAGGATGGCAGCTATCCCAGACAG
GCCGACCACGACGATCACGAGTGCTGCGAGCGGGTCGTGATCAACATCAG
CGGCCTGAGATTCGAGACACAGCTGAAAACCCTGGCCCAGTTCCCCAACA
CCCTGCTGGGCAACCCCAAGAAACGGATGCGGTACTTCGACCCCCTGCGG
AACGAGTACTTCTTCGACCGGAACCGGCCCAGCTTCGACGCCATCCTGTA
CTACTACCAGAGCGGCGGCAGACTGCGGAGGCCCGTGAATGTGCCCCTGG
ACATGTTCAGCGAGGAAATCAAGTTCTACGAGCTGGGCGAGGAAGCCATG
GAAAAGTTCAGAGAGGACGAGGGCTTCATCAAAGAGGAAGAGAGGCCCCT
GCCCGAGAAAGAATACCAGAGACAAGTGTGGCTGCTGTTCGAGTACCCCG
AGTCTAGCGGCCCTGCCAGAGTGATCGCCATCGTGTCCGTGATGGTCATC
CTGATCTCTATCGTGATCTTCTGCCTGGAAACCCTGCCTGAGCTGAAGGA
CGACAAGGACTTCACCGGCACCGTGCACCGGATCGACAACACCACCGTGA
TCTACAACAGCAATATCTTCACCGACCCATTCTTCATCGTGGAAACACTG
TGCATCATCTGGTTCAGCTTCGAGCTGGTCGTGCGGTTCTTCGCCTGCCC
CAGCAAGACCGACTTCTTCAAGAACATCATGAACTTCATTGATATCGTGG
CCATCATCCCCTACTTCATCACCCTGGGCACCGAGATCGCCGAGCAGGAA
GGCAATCAGAAGGGCGAGCAGGCCACCAGCCTGGCCATTCTGAGAGTGAT
CAGACTCGTGCGGGTGTTCCGGATCTTCAAGCTGAGCCGGCACAGCAAGG
GCCTGCAGATCCTGGGCCAGACACTGAAGGCCAGCATGAGAGAGCTGGGC
CTGCTGATCTTCTTTCTGTTCATCGGCGTGATCCTGTTCAGCAGCGCCGT
GTACTTCGCCGAGGCCGAAGAAGCCGAGAGCCACTTCAGCTCTATCCCCG
ACGCCTTTTGGTGGGCCGTGGTGTCCATGACCACAGTGGGCTACGGCGAC
ATGGTGCCCGTGACAATCGGCGGCAAGATCGTGGGCAGCCTGTGTGCCAT
TGCCGGCGTGCTGACAGTCGCCCTGCCTGTGCCTGTGATCGTGTCCAACT
TCAACTACTTCTACCACCGGGAAACCGAGGGGGAGGAACAGGCTCAGCTG
CTGCACGTGTCCAGCCCCAATCTGGCCAGCGACAGCGACCTGAGCAGACG
GTCTAGCAGCACCATGAGCAAGAGCGAGTACATGGAAATCGAAGAGGACA
TGAACAACTCTATCGCCCACTACCGCCAAGTGAACATCCGGACCGCCAAC
TGCACCACCGCCAACCAGAACTGCGTGAACAAGAGCAAGCTGCTGACCGA
TGTCTGAgTCGACAATCAACCTCATcgataccgagcgctgctcgagagat
ctacgggtggcatccctgtgacccctccccagtgcctctcctggccctgg
aagttgccactccagtgcccaccagccttgtcctaataaaattaagttgc
atcattttgtctgactaggtgtccttctataatattatggggtggagggg
ggtggtatggagcaaggggcaagttgggaagacaacctgtagggcctgcg
gggtctattgggaaccaagctggagtgcagtggcacaatcttggctcact
gcaatctccgcctcctgggttcaagcgattctcctgcctcagcctcccga
gttgttgggattccaggcatgcatgaccaggctcagctaatttttgtttt
tttggtagagacggggtttcaccatattggccaggctggtctccaactcc
taatctcaggtgatctacccaccttggcctcccaaattgctgggattaca
ggcgtgaaccactgctcccttccctgtccttctgattttgtaggtaacca
cgtgcggaccgagcggccgcaggaacccctagtgatggagttggccactc
cctctctgcgcgctcgctcgctcactgaggccgggcgaccaaaggtcgcc
cgacgcccgggctttgcccgggcggcctcagtgagcgagcgagcgcgcag
ctgcctgcaggggcgcctgatgcggtattttctccttacgcatctgtgcg
gtatttcacaccgcatacgtcaaagcaaccatagtacgcgccctgtagcg
gcgcattaagcgcggcgggtgtggtggttacgcgcagcgtgaccgctaca
cttgccagcgccctagcgcccgctcctttcgctttcttcccttcctttct
cgccacgttcgccggctttccccgtcaagctctaaatcgggggctccctt
tagggttccgatttagtgctttacggcacctcgaccccaaaaaacttgat
ttgggtgatggttcacgtagtgggccatcgccctgatagacggtttttcg
ccctttgacgttggagtccacgttctttaatagtggactcttgttccaaa
ctggaacaacactcaaccctatctcgggctattcttttgatttataaggg
attttgccgatttcggcctattggttaaaaaatgagctgatttaacaaaa
atttaacgcgaattttaacaaaatattaacgtttacaattttatggtgca
ctctcagtacaatctgctctgatgccgcatagttaagccagccccgacac
ccgccaacacccgctgacgcgccctgacgggcttgtctgctcccggcatc
cgcttacagacaagctgtgaccgtctccgggagctgcatgtgtcagaggt
tttcaccgtcatcaccgaaacgcgcgagacgaaagggcctcgtgatacgc
ctatttttataggttaatgtcatgataataatggtttcttagacgtcagg
tggcacttttcggggaaatgtgcgcggaacccctatttgtttatttttct
aaatacattcaaatatgtatccgctcatgagacaataaccctgataaatg
cttcaataatattgaaaaaggaagagtatgagtattcaacatttccgtgt
cgcccttattcccttttttgcggcattttgccttcctgtttttgctcacc
cagaaacgctggtgaaagtaaaagatgctgaagatcagttgggtgcacga
gtgggttacatcgaactggatctcaacagcggtaagatccttgagagttt
tcgccccgaagaacgttttccaatgatgagcacttttaaagttctgctat
gtggcgcggtattatcccgtattgacgccgggcaagagcaactcggtcgc
cgcatacactattctcagaatgacttggttgagtactcaccagtcacaga
aaagcatcttacggatggcatgacagtaagagaattatgcagtgctgcca
taaccatgagtgataacactgcggccaacttacttctgacaacgatcgga
ggaccgaaggagctaaccgcttttttgcacaacatgggggatcatgtaac
tcgccttgatcgttgggaaccggagctgaatgaagccataccaaacgacg
agcgtgacaccacgatgcctgtagcaatggcaacaacgttgcgcaaacta
ttaactggcgaactacttactctagcttcccggcaacaattaatagactg
gatggaggcggataaagttgcaggaccacttctgcgctcggcccttccgg
ctggctggtttattgctgataaatctggagccggtgagcgtgggtctcgc
ggtatcattgcagcactggggccagatggtaagccctcccgtatcgtagt
tatctacacgacggggagtcaggcaactatggatgaacgaaatagacaga
tcgctgagataggtgcctcactgattaagcattggtaactgtcagaccaa
gtttactcatatatactttagattgatttaaaacttcatttttaatttaa
aaggatctaggtgaagatcctttttgataatctcatgaccaaaatccctt
aacgtgagttttcgttccactgagcgtcagaccccgtagaaaagatcaaa
ggatcttcttgagatcctttttttctgcgcgtaatctgctgcttgcaaac
aaaaaaaccaccgctaccagcggtggtttgtttgccggatcaagagctac
caactctttttccgaaggtaactggcttcagcagagcgcagataccaaat
actgtccttctagtgtagccgtagttaggccaccacttcaagaactctgt
agcaccgcctacatacctcgctctgctaatcctgttaccagtggctgctg
ccagtggcgataagtcgtgtcttaccgggttggactcaagacgatagtta
ccggataaggcgcagcggtcgggctgaacggggggttcgtgcacacagcc
cagcttggagcgaacgacctacaccgaactgagatacctacagcgtgagc
tatgagaaagcgccacgcttcccgaagggagaaaggcggacaggtatccg
gtaagcggcagggtcggaacaggagagcgcacgagggagcttccaggggg
aaacgcctggtatctttatagtcctgtcgggtttcgccacctctgacttg
agcgtcgatttttgtgatgctcgtcaggggggcggagcctatggaaaaac
gccagcaacgcggcctttttacggttcctggccttttgctggccttttgc
tcacatgt

Nucleotide sequence of cfos-KCNA1 construct (SEQ I
D NO: 9) GCGGCCGCAGCTTTCCTTTAGGAACAGAGGCTTCGAGCCTT
TAAGGCTGCGTACTTGCTTCTCCTAATACCAGAGACTCAAAAAAAAAAAA
AAAGTTCCAGATTGCTGGACAATGACCCGGGTCTCATCCCTTGACCCTGG
GAACCGGGTCCACATTGAATCAGGTGCGAATGTTCGCTCGCCTTCTCTGC
CTTTCCCGCCTCCCCTCCCCCGGCCGCGGCCCCGGTTCCCCCCCTGCGCT
GCACCCTCAGAGTTGGCTGCAGCCGGCGAGCTGTTCCCGTCAATCCCTCC
CTCCTTTACACAGGATGTCCATATTAGGACATCTGCGTCAGCAGGTTTCC
ACGGCCGGTCCCTGTTGTTCTGGGGGGGGGACCATCTCCGAAATCCTACA
CGCGGAAGGTCTAGGAGACCCCCTAAGATCCCAAATGTGAACACTCATAG
GTGAAAGATGTATGCCAAGACGGGGGTTGAAAGCCTGGGGCGTAGAGTTG
ACGACAGAGCGCCCGCAGAGGGCCTTGGGGCGCGCTTCCCCCCCCTTCCA
GTTCCGCCCAGTGACGTAGGAAGTCCATCCATTCACAGCGCTTCTATAAA
GGCGCCAGCTGAGGCGCCTACTACTCCAACCGCGACTGCAGCGAGCAACT
GAGAAGACTGGATAGAGCCGGCGGTTCCGCGAACGAGCAGTGACCGCGCT
CCCACCCAGCTCTGCTCTGCAGCTCCCACCAGTGTCTGGCCGCATCGATT
CTAGAATTCGCTGTCTGCGAGGGCCAGCTGTTGGGGTGAGTACTCCCTCT
CAAAAGCGGGCATGACTTCTGCGCTAAGATTGTCAGTTTCCAAAAACGAG
GAGGATTTGATATTCACCTGGCCCGCGGTGATGCCTTTGAGGGTGGCCGC
GTCCATCTGGTCAGAAAAGACAATCTTTTTGTTGTCAAGCTTGAGGTGTG
GCAGGCTTGAGATCTGGCCATACACTTGAGTGACAATGACATCCACTTTG
AGGATCCGCCACCATGACCGTGATGAGCGGCGAGAACGTGGACGAGGCCT
CTGCCGCTCCTGGACACCCTCAGGATGGCAGCTATCCCAGACAGGCCGAC
CACGACGATCACGAGTGCTGCGAGCGGGTCGTGATCAACATCAGCGGCCT
GAGATTCGAGACACAGCTGAAAACCCTGGCCCAGTTCCCCAACACCCTGC
TGGGCAACCCCAAGAAACGGATGCGGTACTTCGACCCCCTGCGGAACGAG
TACTTCTTCGACCGGAACCGGCCCAGCTTCGACGCCATCCTGTACTACTA
CCAGAGCGGCGGCAGACTGCGGAGGCCCGTGAATGTGCCCCTGGACATGT
TCAGCGAGGAAATCAAGTTCTACGAGCTGGGCGAGGAAGCCATGGAAAAG
TTCAGAGAGGACGAGGGCTTCATCAAAGAGGAAGAGAGGCCCCTGCCCGA
GAAAGAATACCAGAGACAAGTGTGGCTGCTGTTCGAGTACCCCGAGTCTA
GCGGCCCTGCCAGAGTGATCGCCATCGTGTCCGTGATGGTCATCCTGATC
TCTATCGTGATCTTCTGCCTGGAAACCCTGCCTGAGCTGAAGGACGACAA
GGACTTCACCGGCACCGTGCACCGGATCGACAACACCACCGTGATCTACA
ACAGCAATATCTTCACCGACCCATTCTTCATCGTGGAAACACTGTGCATC
ATCTGGTTCAGCTTCGAGCTGGTCGTGCGGTTCTTCGCCTGCCCCAGCAA
GACCGACTTCTTCAAGAACATCATGAACTTCATTGATATCGTGGCCATCA
TCCCCTACTTCATCACCCTGGGCACCGAGATCGCCGAGCAGGAAGGCAAT
CAGAAGGGCGAGCAGGCCACCAGCCTGGCCATTCTGAGAGTGATCAGACT
CGTGCGGGTGTTCCGGATCTTCAAGCTGAGCCGGCACAGCAAGGGCCTGC
AGATCCTGGGCCAGACACTGAAGGCCAGCATGAGAGAGCTGGGCCTGCTG
ATCTTCTTTCTGTTCATCGGCGTGATCCTGTTCAGCAGCGCCGTGTACTT
CGCCGAGGCCGAAGAAGCCGAGAGCCACTTCAGCTCTATCCCCGACGCCT
TTTGGTGGGCCGTGGTGTCCATGACCACAGTGGGCTACGGCGACATGGTG
CCCGTGACAATCGGCGGCAAGATCGTGGGCAGCCTGTGTGCCATTGCCGG
CGTGCTGACAGTCGCCCTGCCTGTGCCTGTGATCGTGTCCAACTTCAACT
ACTTCTACCACCGGGAAACCGAGGGGGAGGAACAGGCTCAGCTGCTGCAC
GTGTCCAGCCCCAATCTGGCCAGCGACAGCGACCTGAGCAGACGGTCTAG
CAGCACCATGAGCAAGAGCGAGTACATGGAAATCGAAGAGGACATGAACA
ACTCTATCGCCCACTACCGCCAAGTGAACATCCGGACCGCCAACTGCACC
ACCGCCAACCAGAACTGCGTGAACAAGAGCAAGCTGCTGACCGATGTCTG
A

Nucleotide sequence of optimised AAV-cfos-KCNA1 ve
ctor (SEQ ID NO: 10) cctgcaggcagctgcgcgctcgctcgctc
actgaggccgcccgggcaaagcccgggcgtcgggcgacctttggtcgccc
ggcctcagtgagcgagcgagcgcgcagagagggagtggccaactccatca
ctaggggttcctgcggccgcacgcgtTTCGCTATTACGCCAGTTTTATTG
CGGCCGCAGCTTTCCTTTAGGAACAGAGGCTTCGAGCCTTTAAGGCTGCG
TACTTGCTTCTCCTAATACCAGAGACTCAAAAAAAAAAAAAAAGTTCCAG
ATTGCTGGACAATGACCCGGGTCTCATCCCTTGACCCTGGGAACCGGGTC
CACATTGAATCAGGTGCGAATGTTCGCTCGCCTTCTCTGCCTTTCCCGCC
TCCCCTCCCCCGGCCGCGGCCCCGGTTCCCCCCCTGCGCTGCACCCTCAG
AGTTGGCTGCAGCCGGCGAGCTGTTCCCGTCAATCCCTCCCTCCTTTACA
CAGGATGTCCATATTAGGACATCTGCGTCAGCAGGTTTCCACGGCCGGTC
CCTGTTGTTCTGGGGGGGGGACCATCTCCGAAATCCTACACGCGGAAGGT
CTAGGAGACCCCCTAAGATCCCAAATGTGAACACTCATAGGTGAAAGATG
TATGCCAAGACGGGGGTTGAAAGCCTGGGGCGTAGAGTTGACGACAGAGC
GCCCGCAGAGGGCCTTGGGGCGCGCTTCCCCCCCCTTCCAGTTCCGCCCA
GTGACGTAGGAAGTCCATCCATTCACAGCGCTTCTATAAAGGCGCCAGCT
GAGGCGCCTACTACTCCAACCGCGACTGCAGCGAGCAACTGAGAAGACTG
GATAGAGCCGGCGGTTCCGCGAACGAGCAGTGACCGCGCTCCCACCCAGC
TCTGCTCTGCAGCTCCCACCAGTGTCTGGCCGCATCGATTCTAGAATTCG
CTGTCTGCGAGGGCCAGCTGTTGGGGTGAGTACTCCCTCTCAAAAGCGGG
CATGACTTCTGCGCTAAGATTGTCAGTTTCCAAAAACGAGGAGGATTTGA
TATTCACCTGGCCCGCGGTGATGCCTTTGAGGGTGGCCGCGTCCATCTGG
TCAGAAAAGACAATCTTTTTGTTGTCAAGCTTGAGGTGTGGCAGGCTTGA
CACAGGTGTCCACTCCCAGGTCCAACTGCAGCCCAAGCGGAGGATCCGCC
ACCACCGGTATGACCGTGATGAGCGGCGAGAACGTGGACGAGGCCTCTGC
CGCTCCTGGACACCCTCAGGATGGCAGCTATCCCAGACAGGCCGACCACG
ACGATCACGAGTGCTGCGAGCGGGTCGTGATCAACATCAGCGGCCTGAGA
TTCGAGACACAGCTGAAAACCCTGGCCCAGTTCCCCAACACCCTGCTGGG
CAACCCCAAGAAACGGATGCGGTACTTCGACCCCCTGCGGAACGAGTACT
TCTTCGACCGGAACCGGCCCAGCTTCGACGCCATCCTGTACTACTACCAG
AGCGGCGGCAGACTGCGGAGGCCCGTGAATGTGCCCCTGGACATGTTCAG
CGAGGAAATCAAGTTCTACGAGCTGGGCGAGGAAGCCATGGAAAAGTTCA
GAGAGGACGAGGGCTTCATCAAAGAGGAAGAGAGGCCCCTGCCCGAGAAA
GAATACCAGAGACAAGTGTGGCTGCTGTTCGAGTACCCCGAGTCTAGCGG
CCCTGCCAGAGTGATCGCCATCGTGTCCGTGATGGTCATCCTGATCTCTA
TCGTGATCTTCTGCCTGGAAACCCTGCCTGAGCTGAAGGACGACAAGGAC
TTCACCGGCACCGTGCACCGGATCGACAACACCACCGTGATCTACAACAG
CAATATCTTCACCGACCCATTCTTCATCGTGGAAACACTGTGCATCATCT
GGTTCAGCTTCGAGCTGGTCGTGCGGTTCTTCGCCTGCCCCAGCAAGACC
GACTTCTTCAAGAACATCATGAACTTCATTGATATCGTGGCCATCATCCC
CTACTTCATCACCCTGGGCACCGAGATCGCCGAGCAGGAAGGCAATCAGA
AGGGCGAGCAGGCCACCAGCCTGGCCATTCTGAGAGTGATCAGACTCGTG
CGGGTGTTCCGGATCTTCAAGCTGAGCCGGCACAGCAAGGGCCTGCAGAT
CCTGGGCCAGACACTGAAGGCCAGCATGAGAGAGCTGGGCCTGCTGATCT
TCTTTCTGTTCATCGGCGTGATCCTGTTCAGCAGCGCCGTGTACTTCGCC
GAGGCCGAAGAAGCCGAGAGCCACTTCAGCTCTATCCCCGACGCCTTTTG
GTGGGCCGTGGTGTCCATGACCACAGTGGGCTACGGCGACATGGTGCCCG
TGACAATCGGCGGCAAGATCGTGGGCAGCCTGTGTGCCATTGCCGGCGTG
CTGACAGTCGCCCTGCCTGTGCCTGTGATCGTGTCCAACTTCAACTACTT
CTACCACCGGGAAACCGAGGGGGAGGAACAGGCTCAGCTGCTGCACGTGT
CCAGCCCCAATCTGGCCAGCGACAGCGACCTGAGCAGACGGTCTAGCAGC
ACCATGAGCAAGAGCGAGTACATGGAAATCGAAGAGGACATGAACAACTC
TATCGCCCACTACCGCCAAGTGAACATCCGGACCGCCAACTGCACCACCG
CCAACCAGAACTGCGTGAACAAGAGCAAGCTGCTGACCGATGTCTGAgTC
GACAATCAACCTCATcgataccgagcgctgctcgagagatctacgggtgg
catccctgtgacccctccccagtgcctctcctggccctggaagttgccac
tccagtgcccaccagccttgtcctaataaaattaagttgcatcattttgt
ctgactaggtgtccttctataatattatggggtggaggggggtggtatgg
agcaaggggcaagttgggaagacaacctgtagggcctgcggggtctattg
ggaaccaagctggagtgcagtggcacaatcttggctcactgcaatctccg
cctcctgggttcaagcgattctcctgcctcagcctcccgagttgttggga
ttccaggcatgcatgaccaggctcagctaatttttgtttttttggtagag
acggggtttcaccatattggccaggctggtctccaactcctaatctcagg
tgatctacccaccttggcctcccaaattgctgggattacaggcgtgaacc
actgctcccttccctgtccttctgattttgtaggtaaccacgtgcggacc
gagcggccgcaggaacccctagtgatggagttggccactccctctctgcg
cgctcgctcgctcactgaggccgggcgaccaaaggtcgcccgacgcccgg
gctttgcccgggcggcctcagtgagcgagcgagcgcgcagctgcctgcag
gggcgcctgatgcggtattttctccttacgcatctgtgcggtatttcaca
ccgcatacgtcaaagcaaccatagtacgcgccctgtagcggcgcattaag
cgcggcgggtgtggtggttacgcgcagcgtgaccgctacacttgccagcg
ccctagcgcccgctcctttcgctttcttcccttcctttctcgccacgttc
gccggctttccccgtcaagctctaaatcgggggctccctttagggttccg
atttagtgctttacggcacctcgaccccaaaaaacttgatttgggtgatg
gttcacgtagtgggccatcgccctgatagacggtttttcgccctttgacg
ttggagtccacgttctttaatagtggactcttgttccaaactggaacaac
actcaaccctatctcgggctattcttttgatttataagggattttgccga
tttcggcctattggttaaaaaatgagctgatttaacaaaaatttaacgcg
aattttaacaaaatattaacgtttacaattttatggtgcactctcagtac
aatctgctctgatgccgcatagttaagccagccccgacacccgccaacac
ccgctgacgcgccctgacgggcttgtctgctcccggcatccgcttacaga
caagctgtgaccgtctccgggagctgcatgtgtcagaggttttcaccgtc
atcaccgaaacgcgcgagacgaaagggcctcgtgatacgcctatttttat
aggttaatgtcatgataataatggtttcttagacgtcaggtggcactttt
cggggaaatgtgcgcggaacccctatttgtttatttttctaaatacattc
aaatatgtatccgctcatgagacaataaccctgataaatgcttcaataat
attgaaaaaggaagagtatgagtattcaacatttccgtgtcgcccttatt
cccttttttgcggcattttgccttcctgtttttgctcacccagaaacgct
ggtgaaagtaaaagatgctgaagatcagttgggtgcacgagtgggttaca
tcgaactggatctcaacagcggtaagatccttgagagttttcgccccgaa
gaacgttttccaatgatgagcacttttaaagttctgctatgtggcgcggt
attatcccgtattgacgccgggcaagagcaactcggtcgccgcatacact
attctcagaatgacttggttgagtactcaccagtcacagaaaagcatctt
acggatggcatgacagtaagagaattatgcagtgctgccataaccatgag
tgataacactgcggccaacttacttctgacaacgatcggaggaccgaagg
agctaaccgcttttttgcacaacatgggggatcatgtaactcgccttgat
cgttgggaaccggagctgaatgaagccataccaaacgacgagcgtgacac
cacgatgcctgtagcaatggcaacaacgttgcgcaaactattaactggcg
aactacttactctagcttcccggcaacaattaatagactggatggaggcg
gataaagttgcaggaccacttctgcgctcggcccttccggctggctggtt
tattgctgataaatctggagccggtgagcgtgggtctcgcggtatcattg
cagcactggggccagatggtaagccctcccgtatcgtagttatctacacg
acggggagtcaggcaactatggatgaacgaaatagacagatcgctgagat
aggtgcctcactgattaagcattggtaactgtcagaccaagtttactcat
atatactttagattgatttaaaacttcatttttaatttaaaaggatctag
gtgaagatcctttttgataatctcatgaccaaaatcccttaacgtgagtt
ttcgttccactgagcgtcagaccccgtagaaaagatcaaaggatcttctt
gagatcctttttttctgcgcgtaatctgctgcttgcaaacaaaaaaacca
ccgctaccagcggtggtttgtttgccggatcaagagctaccaactctttt
tccgaaggtaactggcttcagcagagcgcagataccaaatactgtccttc
tagtgtagccgtagttaggccaccacttcaagaactctgtagcaccgcct
acatacctcgctctgctaatcctgttaccagtggctgctgccagtggcga
taagtcgtgtcttaccgggttggactcaagacgatagttaccggataagg
cgcagcggtcgggctgaacggggggttcgtgcacacagcccagcttggag
cgaacgacctacaccgaactgagatacctacagcgtgagctatgagaaag
cgccacgcttcccgaagggagaaaggcggacaggtatccggtaagcggca
gggtcggaacaggagagcgcacgagggagcttccagggggaaacgcctgg
tatctttatagtcctgtcgggtttcgccacctctgacttgagcgtcgatt
tttgtgatgctcgtcaggggggcggagcctatggaaaaacgccagcaacg
cggcctttttacggttcctggccttttgctggccttttgctcacatgt

Engineered human KCNA1 gene encoding an edited Kv1
.1 with a Y379V substitution (SEQ ID NO: 11) atgac
cgtgatgagcggcgagaacgtggacgaggcctctgccgctcctggacacc
ctcaggatggcagctatcccagacaggccgaccacgacgatcacgagtgc
tgcgagcgggtcgtgatcaacatcagcggcctgagattcgagacacagct
gaaaaccctggcccagttccccaacaccctgctgggcaaccccaagaaac
ggatgcggtacttcgaccccctgcggaacgagtacttcttcgaccggaac
cggcccagcttcgacgccatcctgtactactaccagagcggcggcagact
gcggaggcccgtgaatgtgcccctggacatgttcagcgaggaaatcaagt
tctacgagctgggcgaggaagccatggaaaagttcagagaggacgagggc
ttcatcaaagaggaagagaggcccctgcccgagaaagaataccagagaca
agtgtggctgctgttcgagtaccccgagtctagcggccctgccagagtga
tcgccatcgtgtccgtgatggtcatcctgatctctatcgtgatcttctgc
ctggaaaccctgcctgagctgaaggacgacaaggacttcaccggcaccgt
gcaccggatcgacaacaccaccgtgatctacaacagcaatatcttcaccg
acccattcttcatcgtggaaacactgtgcatcatctggttcagcttcgag
ctggtcgtgcggttcttcgcctgccccagcaagaccgacttcttcaagaa
catcatgaacttcattgatatcgtggccatcatcccctacttcatcaccc
tgggcaccgagatcgccgagcaggaaggcaatcagaagggcgagcaggcc
accagcctggccattctgagagtgatcagactcgtgcgggtgttccggat
cttcaagctgagccggcacagcaagggcctgcagatcctgggccagacac
tgaaggccagcatgagagagctgggcctgctgatcttctttctgttcatc
ggcgtgatcctgttcagcagcgccgtgtacttcgccgaggccgaagaagc
cgagagccacttcagctctatccccgacgccttttggtgggccgtggtgt
ccatgaccacagtgggctacggcgacatggtgcccgtgacaatcggcggc
aagatcgtgggcagcctgtgtgccattgccggcgtgctgacagtcgccct
gcctgtgcctgtgatcgtgtccaacttcaactacttctaccaccgggaaa
ccgagggggaggaacaggctcagctgctgcacgtgtccagccccaatctg
gccagcgacagcgacctgagcagacggtctagcagcaccatgagcaagag
cgagtacatggaaatcgaagaggacatgaacaactctatcgcccactacc
gccaagtgaacatccggaccgccaactgcaccaccgccaaccagaactgc
gtgaacaagagcaagctgctgaccgatgtctga

Amino acid sequence of an edited human Kv1.1 compr
ising a valine at position 400 (underlined) and a
valine at position 379 substitution (bolded) (SEQ
ID NO: 12)MTVMSGENVDEASAAPGHPQDGSYPRQADHDDHECCERWI
NISGLRFETQLKTLAQFPNTLLGNPKKRMRYFDPLRNEYFFDRNRPSFDA
ILYYYQSGGRLRRPVNVPLDMFSEEIKFYELGEEAMEKFREDEGFIKEEE
RPLPEKEYQRQVWLLFEYPESSGPARVIAIVSVMVILISIVIFCLETLPE
LKDDKDFTGTVHRIDNTTVIYNSNIFTDPFFIVETLCIIWFSFELWRFFA
CPSKTDFFKNIMNFIDIVAIIPYFITLGTEIAEQEGNQKGEQATSLAILR
VIRLVRVFRIFKLSRHSKGLQILGQTLKASMRELGLLIFFLFIGVILFSS
AVYFAEAEEAESHFSSIPDAFWWAWSMTTVGYGDMVPVTIGGKIVGSLCA
IAGVLTVALPVPVIVSNFNYFYHRETEGEEQAQLLHVSSPNLASDSDLSR
RSSSTMSKSEYMEIEEDMNNSIAHYRQVNIRTANCTTANQNCVNKSKLLT
DV

Nucleotide sequence of an exemplary KCNJ2 gene (SE
Q ID NO: 13) ATGGGCAGTGTGAGAACCAACCGCTACAGCATCGTCT
CTTCAGAAGAAGACGGTATGAAGTTGGCCACCATGGCAGTTGCAAATGGC
TTTGGGAACGGGAAGAGTAAAGTCCACACCCGACAACAGTGCAGGAGCCG
CTTTGTGAAGAAAGATGGCCACTGTAATGTTCACCACGTGTGTGGACATT
CGCTGGCGGTGGATGCTGGTTATCTTCTGCCTGGCTTTCGTCCTGTCATG
GCTGTTTTTTGGCTGTGTGTTTTGGTTGATAGCTCTGCTCCATGGGGACC
TGGATGCATCCAAAGAGGGCAAAGCTTGTGTGTCCGAGGTCAACAGCTTC
ACGGCTGCCTTCCTCTTCTCCATTGAGACCCAGACAACCATAGGCTATGG
TTTCAGATGTGTCACGGATGAATGCCCAATTGCTGTTTTCATGGTGGTGT
TCCAGTCAATCGTGGGCTGCATCATCGATGCTTTCATCATTGGCGCAGTC
ATGGCCAAGATGGCAAAGCCAAAGAAGAGAAACGAGACTCTTGTCTTCAG
TCACAATGCCGTGATTGCCATGAGAGACGGCAAGCTGTGTTTGATGTGGC
GAGTGGGCAATCTTCGGAAAAGCCACTTGGTGGAAGCTCATGTTCGAGCA
CAGCTCCTCAAATCCAGAATTACTTCTGAAGGGGAGTATATCCCTCTGGA
TCAAATAGACATCAATGTTGGGTTTGACAGTGGAATCGATCGTATATTTC
TGGTGTCCCCAATCACTATAGTCCATGAAATAGATGAAGACAGTCCTTTA
TATGATTTGAGTAAACAGGACATTGACAACGCAGACTTTGAAATCGTGGT
CATACTGGAAGGCATGGTGGAAGCCACTGCCATGACGACACAGTGCCGTA
GCTCTTATCTAGCAAATGAAATCCTGTGGGGCCACCGCTATGAGCCTGTG
CTCTTTGAAGAGAAGCACTACTACAAAGTGGACTACTCCAGGTTCCACAA
AACTTACGAAGTCCCCAACACTCCCCTTTGTAGTGCCAGAGACTTAGCAG
AAAAGAAATATATCCTCTCAAATGCAAATTCATTTTGCTATGAAAATGAA
GTTGCCCTCACAAGCAAAGAGGAAGACGACAGTGAAAATGGAGTTCCAGA
AAGCACTAGTACGGACACGCCCCCTGACATAGACCTTCACAACCAGGCAA
GTGTACCTCTAGAGCCCAGGCCCTTACGGCGAGAGTCGGAGATATGA

Amino acid sequence of Kir2.1 (SEQ ID NO: 14) MGSV
RTNRYSIVSSEEDGMKLATMAVANGFGNGKSKVHTRQQCRSRFVKKDGHC
NVQFINVGEKGQRYLADIFTTCVDIRWRWMLVIFCLAFVLSWLFFGCVFW
LIALLHGDLDASKEGKACVSEVNSFTAAFLFSIETQTTIGYGFRCVTDEC
PIAVFMVVFQSIVGCIIDAFIIGAVMAKMAKPKKRNETLVFSHNAVIAMR
DGKLCLMWRVGNLRKSHLVEAHVRAQLLKSRITSEGEYIPLDQIDINVGF
DSGIDRIFLVSPITIVHEIDEDSPLYDLSKQDIDNADFEIWILEGMVEAT
AMTTQCRSSYLANEILWGHRYEPVLFEEKHYYKVDYSRFHKTYEVPNTPL
CSARDLAEKKYILSNANSFCYENEVALTSKEEDDSENGVPESTSTDTPPD
IDLHNQASVPLEPRPLRRESEI

Nucleotide sequence of the mArc promoter (SEQ ID NO
: 15) CGCGCAGCAGAGCACATTAGTCACTCGGGGCTGTGAAGGGGCGGG
TCCTTGAGGGCACCCACGGGAGGGGAGCGAGTAGGCGCGGAAGGCGGGGCC
TGCGGCAGGAGAGGGCGCGGGCGGGCTCTGGCGCGGAGCCTGGGCGCCGCC
AATGGGAGCCAGGGCTCCACGAGCTGCCGCCCACGGGCCCCGCGCAGCATA
AATAGCCGCTGGTGGCGGTTTCGGTGCAGAGCTCAAGCGAGTTCTCCCGCA
GCCGCAGTCTCTGGGCCTCTCTAGCTTCAGCGGCGACGAGCCTGCCACACT
CGCTAAGCTCCTCCGGCACCGCACACCTGCCACTGCCGCTGCAGCCGCCGG
CTCTGCTCCCTTCCGGCTTCTGCCTCAGAGGAGTTCTTAGCCTGTTCGGAG
CCGCAGCACCGACGACCAG

Nucleotide sequence of the ESARE promoter (SEQ ID
NO: 16) AGCGCACAGAGCCTTCCTGCGTGGGGAAGCTCCTTGCTGCGT
CATGGCTCAGCTATTCTCAGCCTCTCTCCTTTTATGGTGCCGGAAGCAGG
CAGGCTGCTGCTAGATCCAGCGCACAGAGCCTTCCTGCGTGGGGAAGCTC
CTTGCTGCGTCATGGCTCAGCTATTCTCAGCCTCTCTCCTTTTATGGTGC
CGGAAGCAGGCAGGCTGCTGCTAGATCCAGCGCACAGAGCCTTCCTGCGT
GGGGAAGCTCCTTGCTGCGTCATGGCTCAGCTATTCTCAGCCTCTCTCCT
TTTATGGTGCCGGAAGCAGGCAGGCTGCTGCTAGATCCAGCGCACAGAGC
CTTCCTGCGTGGGGAAGCTCCTTGCTGCGTCATGGCTCAGCTATTCTCAG
CCTCTCTCCTTTTATGGTGCCGGAAGCAGGCAGGCTGCTGCTAGATCCAG
CGCACAGAGCCTTCCTGCGTGGGGAAGCTCCTTGCTGCGTCATGGCTCAG
CTATTCTCAGCCTCTCTCCTTTTATGGTGCCGGAAGCAGGCAGGCTGCTG
CTCGCGCAGCAGAGCACATTAGTCACTCGGGGCTGTGAAGGGGCGGGTCC
TTGAGGGCACCCACGGGAGGGGAGCGAGTAGGCGCGGAAGGCGGGGCCTG
CGGCAGGAGAGGGCGCGGGCGGGCTCTGGCGCGGAGCCTGGGCGCCGCCA
ATGGGAGCCAGGGCTCCACGAGCTGCCGCCCACGGGCCCCGCGCAGCATA
AATAGCCGCTGGTGGCGGTTTCGGTGCAGAGCTCAAGCGAGTTCTCCCGC
AGCCGCAGTCTCTGGGCCTCTCTAGCTTCAGCGGCGACGAGCCTGCCACA
CTCGCTAAGCTCCTCCGGCACCGCACACCTGCCACTGCCGCTGCAGCCGC
CGGCTCTGCTCCCTTCCGGCTTCTGCCTCAGAGGAGTTCTTAGCCTGTTC
GGAGCCGCAGCACCGACGACCAG

Nucleotide sequence of the NRAM-human cFos promote
r (SEQ ID NO: 17) CTAGAAGTTTGTTCGTGACTGTGACTAGAAGT
TTGTTCGTGACTGTGACTAGAAGTTTGTTCGTGACTGTGACTAGAAGTTT
GTTCGTGACTGTGAACTCATTCATAAAACGCTTGTTATAAAAGCAGTGGC
TGCGGCGCCTCGTACTCCAACCGCATCTGCAGCGAGCAACTGAGAAGCCA
AGACTGAGCCGGCGGCC

Nucleotide sequence of the Eqr1 promoter (SEQ ID N
O: 18) GCTGGCCCTCCCCACGCGGGCGTCCCCGACTCCCGCGCGCGCT
CAGGCTCCCAGTTGGGAACCAAGGAGGGGGAGGATGGGGGGGGGGGTGTG
CGCCGACCCGGAAACGCCATATAAGGAGCAGGAAGGATCCCCCGCCGGAA
CAGACCTTATTTGGGCAGCGCCTTATATGGAGTGGCCCAATATGGCCCTG
CCGCTTCCGGCTCTGGGAGGAGGGGCGAGCGGGGGTTGGGGCGGGGGCAA
GCTGGGAACTCCAGGCGCCTGGCCCGGGAGGCCACTGCTGCTGTTCCAAT
ACTAGGCTTTCCAGGAGCCTGAGCGCTCGCGATGCCGGAGCGGGTCGCAG
GGTGGAGGTGCCCACCACTCTTGGATGGGAGGGCTTCACGTCACTCCGGG
TCCTCCCGGCCGGTCCTTCCATATTAGGGCTTCCTGCTTCCCATATATGG
CCATGTACGTCACGGCGGAGGCGGGCCCGTGCTGTTCCAGACCCTTGAAA
TAGAGGCCGATTCGGGGAGTCGC

Nucleotide sequence of mArc-dsGFP-KCNA1 construct
(SEQ ID NO: 19) CAGAGCACATTAGTCACTCGGGGCTGTGAAGGGG
CGGGTCCTTGAGGGCACCCACGGGAGGGGAGCGAGTAGGCGCGGAAGGCG
GGGCCTGCGGCAGGAGAGGGCGCGGGCGGGCTCTGGCGCGGAGCCTGGGC
GCCGCCAATGGGAGCCAGGGCTCCACGAGCTGCCGCCCACGGGCCCCGCG
CAGCATAAATAGCCGCTGGTGGCGGTTTCGGTGCAGAGCTCAAGCGAGTT
CTCCCGCAGCCGCAGTCTCTGGGCCTCTCTAGCTTCAGCGGCGACGAGCC
TGCCACACTCGCTAAGCTCCTCCGGCACCGCACACCTGCCACTGCCGCTG
CAGCCGCCGGCTCTGCTCCCTTCCGGCTTCTGCCTCAGAGGAGTTCTTAG
CCTGTTCGGAGCCGCAGCACCGACGACCAGGCTAGCAGagaattcGCTGT
CTGCGAGGGCCAGCTGTTGGGGTGAGTACTCCCTCTCAAAAGCGGGCATG
ACTTCTGCGCTAAGATTGTCAGTTTCCAAAAACGAGGAGGATTTGATATT
CACCTGGCCCGCGGTGATGCCTTTGAGGGTGGCCGCGTCCATCTGGTCAG
AAAAGACAATCTTTTTGTTGTCAAGCTTGAGGTGTGGCAGGCTTGAGATC
TGGCCATACACTTGAGTGACAATGACATCCACTTTGCCTTTCTCTCCACA
GGTGTCCACTCCCAGGTCCAACTGCAGCCCAAGCGGAGGATCCGCCACCa
tgcccgccatgaagatcgagtgccgcatcaccggcaccctgaacggcgtg
gagttcgagctggtgggcggcggagagggcacccccgaGCAGGGCCGCAT
GACCAACAAGATGAAGAGCACCAAAGGCGCCCTGACCTTCAGCCCCTACC
TGCTGAGCCACGTGATGGGCTACGGCTTCTACCACTTCGGCACCTACCCC
AGCGGCTACGAGAACCCCTTCCTGCACGCCATCAACAACGGCGGCTACAC
CAACACCCGCATCGAGAAGTACGAGGACGGCGGCGTGCTGCACGTGAGCT
TCAGCTACCGCTACGAGGCCGGCCGCGTGATCGGCGACTTCAAGGTGGTG
GGCACCGGCTTCCCCGAGGACAGCGTGATCTTCACCGACAAGATCATCCG
CAGCAACGCCACCGTGGAGCACCTGCACCCCATGGGCGATAACGTGCTGG
TGGGCAGCTTCGCCCGCACCTTCAGCCTGCGCGACGGCGGCTACTACAGC
TTCGTGGTGGACAGCCACATGCACTTCAAGAGCGCCATCCACCCCAGCAT
CCTGCAGAACGGGGGCCCCATGTTCGCCTTCCGCCGCGTGGAGGAGCTGC
ACAGCAACACCGAGCTGGGCATCGTGGAGTACCAGCACGCCTTCAAGACC
CCCATCGCCTTCGCCAGATCTCGAGATATCAGCCATGGCTTCCCGCCGGC
GGTGGCGGCGCAGGATGATGGCACGCTGCCCATGTCTTGTGCCCAGGAGA
GCGGGATGGACCGTCACCCTGCAGCCTGTGCTTCTGCTAGGATCAATGTG
ACCGGTGAGGGCAGAGGAAGTCTTCTAACATGCGGTGACGTGGAGGAGAA
TCCCGGCCCTATGACCGTGATGAGCGGCGAGAACGTGGACGAGGCCTCTG
CCGCTCCTGGACACCCTCAGGATGGCAGCTATCCCAGACAGGCCGACCAC
GACGATCACGAGTGCTGCGAGCGGGTCGTGATCAACATCAGCGGCCTGAG
ATTCGAGACACAGCTGAAAACCCTGGCCCAGTTCCCCAACACCCTGCTGG
GCAACCCCAAGAAACGGATGCGGTACTTCGACCCCCTGCGGAACGAGTAC
TTCTTCGACCGGAACCGGCCCAGCTTCGACGCCATCCTGTACTACTACCA
GAGCGGCGGCAGACTGCGGAGGCCCGTGAATGTGCCCCTGGACATGTTCA
GCGAGGAAATCAAGTTCTACGAGCTGGGCGAGGAAGCCATGGAAAAGTTC
AGAGAGGACGAGGGCTTCATCAAAGAGGAAGAGAGGCCCCTGCCCGAGAA
AGAATACCAGAGACAAGTGTGGCTGCTGTTCGAGTACCCCGAGTCTAGCG
GCCCTGCCAGAGTGATCGCCATCGTGTCCGTGATGGTCATCCTGATCTCT
ATCGTGATCTTCTGCCTGGAAACCCTGCCTGAGCTGAAGGACGACAAGGA
CTTCACCGGCACCGTGCACCGGATCGACAACACCACCGTGATCTACAACA
GCAATATCTTCACCGACCCATTCTTCATCGTGGAAACACTGTGCATCATC
TGGTTCAGCTTCGAGCTGGTCGTGCGGTTCTTCGCCTGCCCCAGCAAGAC
CGACTTCTTCAAGAACATCATGAACTTCATTGATATCGTGGCCATCATCC
CCTACTTCATCACCCTGGGCACCGAGATCGCCGAGCAGGAAGGCAATCAG
AAGGGCGAGCAGGCCACCAGCCTGGCCATTCTGAGAGTGATCAGACTCGT
GCGGGTGTTCCGGATCTTCAAGCTGAGCCGGCACAGCAAGGGCCTGCAGA
TCCTGGGCCAGACACTGAAGGCCAGCATGAGAGAGCTGGGCCTGCTGATC
TTCTTTCTGTTCATCGGCGTGATCCTGTTCAGCAGCGCCGTGTACTTCGC
CGAGGCCGAAGAAGCCGAGAGCCACTTCAGCTCTATCCCCGACGCCTTTT
GGTGGGCCGTGGTGTCCATGACCACAGTGGGCTACGGCGACATGGTGCCC
GTGACAATCGGCGGCAAGATCGTGGGCAGCCTGTGTGCCATTGCCGGCGT
GCTGACAGTCGCCCTGCCTGTGCCTGTGATCGTGTCCAACTTCAACTACT
TCTACCACCGGGAAACCGAGGGGGAGGAACAGGCTCAGCTGCTGCACGTG
TCCAGCCCCAATCTGGCCAGCGACAGCGACCTGAGCAGACGGTCTAGCAG
CACCATGAGCAAGAGCGAGTACATGGAAATCGAAGAGGACATGAACAACT
CTATCGCCCACTACCGCCAAGTGAACATCCGGACCGCCAACTGCACCACC
GCCAACCAGAACTGCGTGAACAAGAGCAAGCTGCTGACCGATGTC

Nucleotide sequence of optimised AAV- mArc-dsGFP-K
CNA1 vector (SEQ ID NO: 20) gcaggcagctgcgcgctcgctc
gctcactgaggccgcccgggcaaagcccgggcgtcgggcgacctttggtc
gcccggcctcagtgagcgagcgagcgcgcagagagggagtggccaactcc
atcactaggggttcctgcggccgcacgcgtCGCGCAGCAGAGCACATTAG
TCACTCGGGGCTGTGAAGGGGCGGGTCCTTGAGGGCACCCACGGGAGGGG
AGCGAGTAGGCGCGGAAGGCGGGGCCTGCGGCAGGAGAGGGCGCGGGCGG
GCTCTGGCGCGGAGCCTGGGCGCCGCCAATGGGAGCCAGGGCTCCACGAG
CTGCCGCCCACGGGCCCCGCGCAGCATAAATAGCCGCTGGTGGCGGTTTC
GGTGCAGAGCTCAAGCGAGTTCTCCCGCAGCCGCAGTCTCTGGGCCTCTC
TAGCTTCAGCGGCGACGAGCCTGCCACACTCGCTAAGCTCCTCCGGCACC
GCACACCTGCCACTGCCGCTGCAGCCGCCGGCTCTGCTCCCTTCCGGCTT
CTGCCTCAGAGGAGTTCTTAGCCTGTTCGGAGCCGCAGCACCGACGACCA
GGCTAGCAGagaattcGCTGTCTGCGAGGGCCAGCTGTTGGGGTGAGTAC
TCCCTCTCAAAAGCGGGCATGACTTCTGCGCTAAGATTGTCAGTTTCCAA
AAACGAGGAGGATTTGATATTCACCTGGCCCGCGGTGATGCCTTTGAGGG
TGGCCGCGTCCATCTGGTCAGAAAAGACAATCTTTTTGTTGTCAAGCTTG
AGGTGTGGCAGGCTTGAGATCTGGCCATACACTTGAGTGACAATGACATC
CACTTTGCCTTTCTCTCCACAGGTGTCCACTCCCAGGTCCAACTGCAGCC
CAAGCGGAGGATCCGCCACCatgcccgccatgaagatcgagtgccgcatc
accggcaccctgaacggcgtggagttcgagctggtgggcggcggagaggg
cacccccgaGCAGGGCCGCATGACCAACAAGATGAAGAGCACCAAAGGCG
CCCTGACCTTCAGCCCCTACCTGCTGAGCCACGTGATGGGCTACGGCTTC
TACCACTTCGGCACCTACCCCAGCGGCTACGAGAACCCCTTCCTGCACGC
CATCAACAACGGCGGCTACACCAACACCCGCATCGAGAAGTACGAGGACG
GCGGCGTGCTGCACGTGAGCTTCAGCTACCGCTACGAGGCCGGCCGCGTG
ATCGGCGACTTCAAGGTGGTGGGCACCGGCTTCCCCGAGGACAGCGTGAT
CTTCACCGACAAGATCATCCGCAGCAACGCCACCGTGGAGCACCTGCACC
CCATGGGCGATAACGTGCTGGTGGGCAGCTTCGCCCGCACCTTCAGCCTG
CGCGACGGCGGCTACTACAGCTTCGTGGTGGACAGCCACATGCACTTCAA
GAGCGCCATCCACCCCAGCATCCTGCAGAACGGGGGCCCCATGTTCGCCT
TCCGCCGCGTGGAGGAGCTGCACAGCAACACCGAGCTGGGCATCGTGGAG
TACCAGCACGCCTTCAAGACCCCCATCGCCTTCGCCAGATCTCGAGATAT
CAGCCATGGCTTCCCGCCGGCGGTGGCGGCGCAGGATGATGGCACGCTGC
CCATGTCTTGTGCCCAGGAGAGCGGGATGGACCGTCACCCTGCAGCCTGT
GCTTCTGCTAGGATCAATGTGACCGGTGAGGGCAGAGGAAGTCTTCTAAC
ATGCGGTGACGTGGAGGAGAATCCCGGCCCTATGACCGTGATGAGCGGCG
AGAACGTGGACGAGGCCTCTGCCGCTCCTGGACACCCTCAGGATGGCAGC
TATCCCAGACAGGCCGACCACGACGATCACGAGTGCTGCGAGCGGGTCGT
GATCAACATCAGCGGCCTGAGATTCGAGACACAGCTGAAAACCCTGGCCC
AGTTCCCCAACACCCTGCTGGGCAACCCCAAGAAACGGATGCGGTACTTC
GACCCCCTGCGGAACGAGTACTTCTTCGACCGGAACCGGCCCAGCTTCGA
CGCCATCCTGTACTACTACCAGAGCGGCGGCAGACTGCGGAGGCCCGTGA
ATGTGCCCCTGGACATGTTCAGCGAGGAAATCAAGTTCTACGAGCTGGGC
GAGGAAGCCATGGAAAAGTTCAGAGAGGACGAGGGCTTCATCAAAGAGGA
AGAGAGGCCCCTGCCCGAGAAAGAATACCAGAGACAAGTGTGGCTGCTGT
TCGAGTACCCCGAGTCTAGCGGCCCTGCCAGAGTGATCGCCATCGTGTCC
GTGATGGTCATCCTGATCTCTATCGTGATCTTCTGCCTGGAAACCCTGCC
TGAGCTGAAGGACGACAAGGACTTCACCGGCACCGTGCACCGGATCGACA
ACACCACCGTGATCTACAACAGCAATATCTTCACCGACCCATTCTTCATC
GTGGAAACACTGTGCATCATCTGGTTCAGCTTCGAGCTGGTCGTGCGGTT
CTTCGCCTGCCCCAGCAAGACCGACTTCTTCAAGAACATCATGAACTTCA
TTGATATCGTGGCCATCATCCCCTACTTCATCACCCTGGGCACCGAGATC
GCCGAGCAGGAAGGCAATCAGAAGGGCGAGCAGGCCACCAGCCTGGCCAT
TCTGAGAGTGATCAGACTCGTGCGGGTGTTCCGGATCTTCAAGCTGAGCC
GGCACAGCAAGGGCCTGCAGATCCTGGGCCAGACACTGAAGGCCAGCATG
AGAGAGCTGGGCCTGCTGATCTTCTTTCTGTTCATCGGCGTGATCCTGTT
CAGCAGCGCCGTGTACTTCGCCGAGGCCGAAGAAGCCGAGAGCCACTTCA
GCTCTATCCCCGACGCCTTTTGGTGGGCCGTGGTGTCCATGACCACAGTG
GGCTACGGCGACATGGTGCCCGTGACAATCGGCGGCAAGATCGTGGGCAG
CCTGTGTGCCATTGCCGGCGTGCTGACAGTCGCCCTGCCTGTGCCTGTGA
TCGTGTCCAACTTCAACTACTTCTACCACCGGGAAACCGAGGGGGAGGAA
CAGGCTCAGCTGCTGCACGTGTCCAGCCCCAATCTGGCCAGCGACAGCGA
CCTGAGCAGACGGTCTAGCAGCACCATGAGCAAGAGCGAGTACATGGAAA
TCGAAGAGGACATGAACAACTCTATCGCCCACTACCGCCAAGTGAACATC
CGGACCGCCAACTGCACCACCGCCAACCAGAACTGCGTGAACAAGAGCAA
GCTGCTGACCGATGTCTGAgTCGACAATCAACCTCATcgataccgagcgc
tgctcgagagatctacgggtggcatccctgtgacccctccccagtgcctc
tcctggccctggaagttgccactccagtgcccaccagccttgtcctaata
aaattaagttgcatcattttgtctgactaggtgtccttctataatattat
ggggtggaggggggtggtatggagcaaggggcaagttgggaagacaacct
gtagggcctgcggggtctattgggaaccaagctggagtgcagtggcacaa
tcttggctcactgcaatctccgcctcctgggttcaagcgattctcctgcc
tcagcctcccgagttgttgggattccaggcatgcatgaccaggctcagct
aatttttgtttttttggtagagacggggtttcaccatattggccaggctg
gtctccaactcctaatctcaggtgatctacccaccttggcctcccaaatt
gctgggattacaggcgtgaaccactgctcccttccctgtccttctgattt
tgtaggtaaccacgtgcggaccgagcggccgcaggaacccctagtgatgg
agttggccactccctctctgcgcgctcgctcgctcactgaggccgggcga
ccaaaggtcgcccgacgcccgggctttgcccgggcggcctcagtgagcga
gcgagcgcgcagctgcctgcaggggcgcctgatgcggtattttctcctta
cgcatctgtgcggtatttcacaccgcatacgtcaaagcaaccatagtacg
cgccctgtagcggcgcattaagcgcggcgggtgtggtggttacgcgcagc
gtgaccgctacacttgccagcgccctagcgcccgctcctttcgctttctt
cccttcctttctcgccacgttcgccggctttccccgtcaagctctaaatc
gggggctccctttagggttccgatttagtgctttacggcacctcgacccc
aaaaaacttgatttgggtgatggttcacgtagtgggccatcgccctgata
gacggtttttcgccctttgacgttggagtccacgttctttaatagtggac
tcttgttccaaactggaacaacactcaaccctatctcgggctattctttt
gatttataagggattttgccgatttcggcctattggttaaaaaatgagct
gatttaacaaaaatttaacgcgaattttaacaaaatattaacgtttacaa
ttttatggtgcactctcagtacaatctgctctgatgccgcatagttaagc
cagccccgacacccgccaacacccgctgacgcgccctgacgggcttgtct
gctcccggcatccgcttacagacaagctgtgaccgtctccgggagctgca
tgtgtcagaggttttcaccgtcatcaccgaaacgcgcgagacgaaagggc
ctcgtgatacgcctatttttataggttaatgtcatgataataatggtttc
ttagacgtcaggtggcacttttcggggaaatgtgcgcggaacccctattt
gtttatttttctaaatacattcaaatatgtatccgctcatgagacaataa
ccctgataaatgcttcaataatattgaaaaaggaagagtatgagtattca
acatttccgtgtcgcccttattcccttttttgcggcattttgccttcctg
tttttgctcacccagaaacgctggtgaaagtaaaagatgctgaagatcag
ttgggtgcacgagtgggttacatcgaactggatctcaacagcggtaagat
ccttgagagttttcgccccgaagaacgttttccaatgatgagcactttta
aagttctgctatgtggcgcggtattatcccgtattgacgccgggcaagag
caactcggtcgccgcatacactattctcagaatgacttggttgagtactc
accagtcacagaaaagcatcttacggatggcatgacagtaagagaattat
gcagtgctgccataaccatgagtgataacactgcggccaacttacttctg
acaacgatcggaggaccgaaggagctaaccgcttttttgcacaacatggg
ggatcatgtaactcgccttgatcgttgggaaccggagctgaatgaagcca
taccaaacgacgagcgtgacaccacgatgcctgtagcaatggcaacaacg
ttgcgcaaactattaactggcgaactacttactctagcttcccggcaaca
attaatagactggatggaggcggataaagttgcaggaccacttctgcgct
cggcccttccggctggctggtttattgctgataaatctggagccggtgag
cgtgggtctcgcggtatcattgcagcactggggccagatggtaagccctc
ccgtatcgtagttatctacacgacggggagtcaggcaactatggatgaac
gaaatagacagatcgctgagataggtgcctcactgattaagcattggtaa
ctgtcagaccaagtttactcatatatactttagattgatttaaaacttca
tttttaatttaaaaggatctaggtgaagatcctttttgataatctcatga
ccaaaatcccttaacgtgagttttcgttccactgagcgtcagaccccgta
gaaaagatcaaaggatcttcttgagatcctttttttctgcgcgtaatctg
ctgcttgcaaacaaaaaaaccaccgctaccagcggtggtttgtttgccgg
atcaagagctaccaactctttttccgaaggtaactggcttcagcagagcg
cagataccaaatactgtccttctagtgtagccgtagttaggccaccactt
caagaactctgtagcaccgcctacatacctcgctctgctaatcctgttac
cagtggctgctgccagtggcgataagtcgtgtcttaccgggttggactca
agacgatagttaccggataaggcgcagcggtcgggctgaacggggggttc
gtgcacacagcccagcttggagcgaacgacctacaccgaactgagatacc
tacagcgtgagctatgagaaagcgccacgcttcccgaagggagaaaggcg
gacaggtatccggtaagcggcagggtcggaacaggagagcgcacgaggga
gcttccagggggaaacgcctggtatctttatagtcctgtcgggtttcgcc
acctctgacttgagcgtcgatttttgtgatgctcgtcaggggggcggagc
ctatggaaaaacgccagcaacgcggcctttttacggttcctggccttttg
ctggccttttgctcac

Nucleotide sequence of mArc-dsGFP-KCNJ2 construct
(SEQ ID NO: 21) GCAGCAGAGCACATTAGTCACTCGGGGCTGTGAA
GGGGCGGGTCCTTGAGGGCACCCACGGGAGGGGAGCGAGTAGGCGCGGAA
GGCGGGGCCTGCGGCAGGAGAGGGCGCGGGCGGGCTCTGGCGCGGAGCCT
GGGCGCCGCCAATGGGAGCCAGGGCTCCACGAGCTGCCGCCCACGGGCCC
CGCGCAGCATAAATAGCCGCTGGTGGCGGTTTCGGTGCAGAGCTCAAGCG
AGTTCTCCCGCAGCCGCAGTCTCTGGGCCTCTCTAGCTTCAGCGGCGACG
AGCCTGCCACACTCGCTAAGCTCCTCCGGCACCGCACACCTGCCACTGCC
GCTGCAGCCGCCGGCTCTGCTCCCTTCCGGCTTCTGCCTCAGAGGAGTTC
TTAGCCTGTTCGGAGCCGCAGCACCGACGACCAGGCTAGCAGagaattcG
CTGTCTGCGAGGGCCAGCTGTTGGGGTGAGTACTCCCTCTCAAAAGCGGG
CATGACTTCTGCGCTAAGATTGTCAGTTTCCAAAAACGAGGAGGATTTGA
TATTCACCTGGCCCGCGGTGATGCCTTTGAGGGTGGCCGCGTCCATCTGG
TCAGAAAAGACAATCTTTTTGTTGTCAAGCTTGAGGTGTGGCAGGCTTGA
GATCTGGCCATACACTTGAGTGACAATGACATCCACTTTGCCTTTCTCTC
CACAGGTGTCCACTCCCAGGTCCAACTGCAGCCCAAGCGGAGGATCCGCC
ACCatgcccgccatgaagatcgagtgccgcatcaccggcaccctgaacgg
cgtggagttcgagctggtgggcggcggagagggcacccccgaGCAGGGCC
GCATGACCAACAAGATGAAGAGCACCAAAGGCGCCCTGACCTTCAGCCCC
TACCTGCTGAGCCACGTGATGGGCTACGGCTTCTACCACTTCGGCACCTA
CCCCAGCGGCTACGAGAACCCCTTCCTGCACGCCATCAACAACGGCGGCT
ACACCAACACCCGCATCGAGAAGTACGAGGACGGCGGCGTGCTGCACGTG
AGCTTCAGCTACCGCTACGAGGCCGGCCGCGTGATCGGCGACTTCAAGGT
GGTGGGCACCGGCTTCCCCGAGGACAGCGTGATCTTCACCGACAAGATCA
TCCGCAGCAACGCCACCGTGGAGCACCTGCACCCCATGGGCGATAACGTG
CTGGTGGGCAGCTTCGCCCGCACCTTCAGCCTGCGCGACGGCGGCTACTA
CAGCTTCGTGGTGGACAGCCACATGCACTTCAAGAGCGCCATCCACCCCA
GCATCCTGCAGAACGGGGGCCCCATGTTCGCCTTCCGCCGCGTGGAGGAG
CTGCACAGCAACACCGAGCTGGGCATCGTGGAGTACCAGCACGCCTTCAA
GACCCCCATCGCCTTCGCCAGATCTCGAGATATCAGCCATGGCTTCCCGC
CGGCGGTGGCGGCGCAGGATGATGGCACGCTGCCCATGTCTTGTGCCCAG
GAGAGCGGGATGGACCGTCACCCTGCAGCCTGTGCTTCTGCTAGGATCAA
TGTGACCGGTGAGGGCAGAGGAAGTCTTCTAACATGCGGTGACGTGGAGG
AGAATCCCGGCCCTatgggcagtgtgagaaccaaccgctacagcatcgtc
tcttcagaagaagacggtatgaagttggccaccatggcagttgcaaatgg
ctttgggaacgggaagagtaaagtccacacccgacaacagtgcaggagcc
gctttgtgaagaaagatggccactgtaatgttcagttcatcaatgtgggt
gagaaggggcaacggtacctcgcagacatcttcaccacgtgtgtggacat
tcgctggcggtggatgctggttatcttctgcctggctttcgtcctgtcat
ggctgttttttggctgtgtgttttggttgatagctctgctccatggggac
ctggatgcatccaaagagggcaaagcttgtgtgtccgaggtcaacagctt
cacggctgccttcctcttctccattgagacccagacaaccataggctatg
gtttcagatgtgtcacggatgaatgcccaattgctgttttcatggtggtg
ttccagtcaatcgtgggctgcatcatcgatgctttcatcattggcgcagt
catggccaagatggcaaagccaaagaagagaaacgagactcttgtcttca
gtcacaatgccgtgattgccatgagagacggcaagctgtgtttgatgtgg
cgagtgggcaatcttcggaaaagccacttggtggaagctcatgttcgagc
acagctcctcaaatccagaattacttctgaaggggagtatatccctctgg
atcaaatagacatcaatgttgggtttgacagtggaatcgatcgtatattt
ctggtgtccccaatcactatagtccatgaaatagatgaagacagtccttt
atatgatttgagtaaacaggacattgacaacgcagactttgaaatcgtgg
tcatactggaaggcatggtggaagccactgccatgacgacacagtgccgt
agctcttatctagcaaatgaaatcctgtggggccaccgctatgagcctgt
gctctttgaagagaagcactactacaaagtggactactccaggttccaca
aaacttacgaagtccccaacactcccctttgtagtgccagagacttagca
gaaaagaaatatatcctctcaaatgcaaattcattttgctatgaaaatga
agttgccctcacaagcaaagaggaagacgacagtgaaaatggagttccag
aaagcactagtacggacacgccccctgacatagaccttcacaaccaggca
agtgtacctctagagcccaggcccttacggcgagagtcggaga

Nucleotide sequence of optimised AAV- mArc-dsGFP-K
CNJ2 vector (SEQ ID NO: 22) ggcagctgcgcgctcgctcgct
cactgaggccgcccgggcaaagcccgggcgtcgggcgacctttggtcgcc
cggcctcagtgagcgagcgagcgcgcagagagggagtggccaactccatc
actaggggttcctgcggccgcacgcgtCGCGCAGCAGAGCACATTAGTCA
CTCGGGGCTGTGAAGGGGCGGGTCCTTGAGGGCACCCACGGGAGGGGAGC
GAGTAGGCGCGGAAGGCGGGGCCTGCGGCAGGAGAGGGCGCGGGCGGGCT
CTGGCGCGGAGCCTGGGCGCCGCCAATGGGAGCCAGGGCTCCACGAGCTG
CCGCCCACGGGCCCCGCGCAGCATAAATAGCCGCTGGTGGCGGTTTCGGT
GCAGAGCTCAAGCGAGTTCTCCCGCAGCCGCAGTCTCTGGGCCTCTCTAG
CTTCAGCGGCGACGAGCCTGCCACACTCGCTAAGCTCCTCCGGCACCGCA
CACCTGCCACTGCCGCTGCAGCCGCCGGCTCTGCTCCCTTCCGGCTTCTG
CCTCAGAGGAGTTCTTAGCCTGTTCGGAGCCGCAGCACCGACGACCAGGC
TAGCAGagaattcGCTGTCTGCGAGGGCCAGCTGTTGGGGTGAGTACTCC
CTCTCAAAAGCGGGCATGACTTCTGCGCTAAGATTGTCAGTTTCCAAAAA
CGAGGAGGATTTGATATTCACCTGGCCCGCGGTGATGCCTTTGAGGGTGG
CCGCGTCCATCTGGTCAGAAAAGACAATCTTTTTGTTGTCAAGCTTGAGG
TGTGGCAGGCTTGAGATCTGGCCATACACTTGAGTGACAATGACATCCAC
TTTGCCTTTCTCTCCACAGGTGTCCACTCCCAGGTCCAACTGCAGCCCAA
GCGGAGGATCCGCCACCatgcccgccatgaagatcgagtgccgcatcacc
ggcaccctgaacggcgtggagttcgagctggtgggcggcggagagggcac
ccccgaGCAGGGCCGCATGACCAACAAGATGAAGAGCACCAAAGGCGCCC
TGACCTTCAGCCCCTACCTGCTGAGCCACGTGATGGGCTACGGCTTCTAC
CACTTCGGCACCTACCCCAGCGGCTACGAGAACCCCTTCCTGCACGCCAT
CAACAACGGCGGCTACACCAACACCCGCATCGAGAAGTACGAGGACGGCG
GCGTGCTGCACGTGAGCTTCAGCTACCGCTACGAGGCCGGCCGCGTGATC
GGCGACTTCAAGGTGGTGGGCACCGGCTTCCCCGAGGACAGCGTGATCTT
CACCGACAAGATCATCCGCAGCAACGCCACCGTGGAGCACCTGCACCCCA
TGGGCGATAACGTGCTGGTGGGCAGCTTCGCCCGCACCTTCAGCCTGCGC
GACGGCGGCTACTACAGCTTCGTGGTGGACAGCCACATGCACTTCAAGAG
CGCCATCCACCCCAGCATCCTGCAGAACGGGGGCCCCATGTTCGCCTTCC
GCCGCGTGGAGGAGCTGCACAGCAACACCGAGCTGGGCATCGTGGAGTAC
CAGCACGCCTTCAAGACCCCCATCGCCTTCGCCAGATCTCGAGATATCAG
CCATGGCTTCCCGCCGGCGGTGGCGGCGCAGGATGATGGCACGCTGCCCA
TGTCTTGTGCCCAGGAGAGCGGGATGGACCGTCACCCTGCAGCCTGTGCT
TCTGCTAGGATCAATGTGACCGGTGAGGGCAGAGGAAGTCTTCTAACATG
CGGTGACGTGGAGGAGAATCCCGGCCCTatgggcagtgtgagaaccaacc
gctacagcatcgtctcttcagaagaagacggtatgaagttggccaccatg
gcagttgcaaatggctttgggaacgggaagagtaaagtccacacccgaca
acagtgcaggagccgctttgtgaagaaagatggccactgtaatgttcagt
tcatcaatgtgggtgagaaggggcaacggtacctcgcagacatcttcacc
acgtgtgtggacattcgctggcggtggatgctggttatcttctgcctggc
tttcgtcctgtcatggctgttttttggctgtgtgttttggttgatagctc
tgctccatggggacctggatgcatccaaagagggcaaagcttgtgtgtcc
gaggtcaacagcttcacggctgccttcctcttctccattgagacccagac
aaccataggctatggtttcagatgtgtcacggatgaatgcccaattgctg
ttttcatggtggtgttccagtcaatcgtgggctgcatcatcgatgctttc
atcattggcgcagtcatggccaagatggcaaagccaaagaagagaaacga
gactcttgtcttcagtcacaatgccgtgattgccatgagagacggcaagc
tgtgtttgatgtggcgagtgggcaatcttcggaaaagccacttggtggaa
gctcatgttcgagcacagctcctcaaatccagaattacttctgaagggga
gtatatccctctggatcaaatagacatcaatgttgggtttgacagtggaa
tcgatcgtatatttctggtgtccccaatcactatagtccatgaaatagat
gaagacagtcctttatatgatttgagtaaacaggacattgacaacgcaga
ctttgaaatcgtggtcatactggaaggcatggtggaagccactgccatga
cgacacagtgccgtagctcttatctagcaaatgaaatcctgtggggccac
cgctatgagcctgtgctctttgaagagaagcactactacaaagtggacta
ctccaggttccacaaaacttacgaagtccccaacactcccctttgtagtg
ccagagacttagcagaaaagaaatatatcctctcaaatgcaaattcattt
tgctatgaaaatgaagttgccctcacaagcaaagaggaagacgacagtga
aaatggagttccagaaagcactagtacggacacgccccctgacatagacc
ttcacaaccaggcaagtgtacctctagagcccaggcccttacggcgagag
tcggagatatgagTCGACAATCAACCTCATcgataccgagcgctgctcga
gagatctacgggtggcatccctgtgacccctccccagtgcctctcctggc
cctggaagttgccactccagtgcccaccagccttgtcctaataaaattaa
gttgcatcattttgtctgactaggtgtccttctataatattatggggtgg
aggggggtggtatggagcaaggggcaagttgggaagacaacctgtagggc
ctgcggggtctattgggaaccaagctggagtgcagtggcacaatcttggc
tcactgcaatctccgcctcctgggttcaagcgattctcctgcctcagcct
cccgagttgttgggattccaggcatgcatgaccaggctcagctaattttt
gtttttttggtagagacggggtttcaccatattggccaggctggtctcca
actcctaatctcaggtgatctacccaccttggcctcccaaattgctggga
ttacaggcgtgaaccactgctcccttccctgtccttctgattttgtaggt
aaccacgtgcggaccgagcggccgcaggaacccctagtgatggagttggc
cactccctctctgcgcgctcgctcgctcactgaggccgggcgaccaaagg
tcgcccgacgcccgggctttgcccgggcggcctcagtgagcgagcgagcg
cgcagctgcctgcaggggcgcctgatgcggtattttctccttacgcatct
gtgcggtatttcacaccgcatacgtcaaagcaaccatagtacgcgccctg
tagcggcgcattaagcgcggcgggtgtggtggttacgcgcagcgtgaccg
ctacacttgccagcgccctagcgcccgctcctttcgctttcttcccttcc
tttctcgccacgttcgccggctttccccgtcaagctctaaatcgggggct
ccctttagggttccgatttagtgctttacggcacctcgaccccaaaaaac
ttgatttgggtgatggttcacgtagtgggccatcgccctgatagacggtt
tttcgccctttgacgttggagtccacgttctttaatagtggactcttgtt
ccaaactggaacaacactcaaccctatctcgggctattcttttgatttat
aagggattttgccgatttcggcctattggttaaaaaatgagctgatttaa
caaaaatttaacgcgaattttaacaaaatattaacgtttacaattttatg
gtgcactctcagtacaatctgctctgatgccgcatagttaagccagcccc
gacacccgccaacacccgctgacgcgccctgacgggcttgtctgctcccg
gcatccgcttacagacaagctgtgaccgtctccgggagctgcatgtgtca
gaggttttcaccgtcatcaccgaaacgcgcgagacgaaagggcctcgtga
tacgcctatttttataggttaatgtcatgataataatggtttcttagacg
tcaggtggcacttttcggggaaatgtgcgcggaacccctatttgtttatt
tttctaaatacattcaaatatgtatccgctcatgagacaataaccctgat
aaatgcttcaataatattgaaaaaggaagagtatgagtattcaacatttc
cgtgtcgcccttattcccttttttgcggcattttgccttcctgtttttgc
tcacccagaaacgctggtgaaagtaaaagatgctgaagatcagttgggtg
cacgagtgggttacatcgaactggatctcaacagcggtaagatccttgag
agttttcgccccgaagaacgttttccaatgatgagcacttttaaagttct
gctatgtggcgcggtattatcccgtattgacgccgggcaagagcaactcg
gtcgccgcatacactattctcagaatgacttggttgagtactcaccagtc
acagaaaagcatcttacggatggcatgacagtaagagaattatgcagtgc
tgccataaccatgagtgataacactgcggccaacttacttctgacaacga
tcggaggaccgaaggagctaaccgcttttttgcacaacatgggggatcat
gtaactcgccttgatcgttgggaaccggagctgaatgaagccataccaaa
cgacgagcgtgacaccacgatgcctgtagcaatggcaacaacgttgcgca
aactattaactggcgaactacttactctagcttcccggcaacaattaata
gactggatggaggcggataaagttgcaggaccacttctgcgctcggccct
tccggctggctggtttattgctgataaatctggagccggtgagcgtgggt
ctcgcggtatcattgcagcactggggccagatggtaagccctcccgtatc
gtagttatctacacgacggggagtcaggcaactatggatgaacgaaatag
acagatcgctgagataggtgcctcactgattaagcattggtaactgtcag
accaagtttactcatatatactttagattgatttaaaacttcatttttaa
tttaaaaggatctaggtgaagatcctttttgataatctcatgaccaaaat
cccttaacgtgagttttcgttccactgagcgtcagaccccgtagaaaaga
tcaaaggatcttcttgagatcctttttttctgcgcgtaatctgctgcttg
caaacaaaaaaaccaccgctaccagcggtggtttgtttgccggatcaaga
gctaccaactctttttccgaaggtaactggcttcagcagagcgcagatac
caaatactgtccttctagtgtagccgtagttaggccaccacttcaagaac
tctgtagcaccgcctacatacctcgctctgctaatcctgttaccagtggc
tgctgccagtggcgataagtcgtgtcttaccgggttggactcaagacgat
agttaccggataaggcgcagcggtcgggctgaacggggggttcgtgcaca
cagcccagcttggagcgaacgacctacaccgaactgagatacctacagcg
tgagctatgagaaagcgccacgcttcccgaagggagaaaggcggacaggt
atccggtaagcggcagggtcggaacaggagagcgcacgagggagcttcca
gggggaaacgcctggtatctttatagtcctgtcgggtttcgccacctctg
acttgagcgtcgatttttgtgatgctcgtcaggggggcggagcctatgga
aaaacgccagcaacgcggcctttttacggttcctggccttttgctggcct
tttgctcaca

Nucleotide sequence of ESARE-dsGFP-KCNA1 construct
(SEQ ID NO: 23) TTCCTGCGTGGGGAAGCTCCTTGCTGCGTCATG
GCTCAGCTATTCTCAGCCTCTCTCCTTTTATGGTGCCGGAAGCAGGCAGG
CTGCTGCTAGATCCAGCGCACAGAGCCTTCCTGCGTGGGGAAGCTCCTTG
CTGCGTCATGGCTCAGCTATTCTCAGCCTCTCTCCTTTTATGGTGCCGGA
AGCAGGCAGGCTGCTGCTAGATCCAGCGCACAGAGCCTTCCTGCGTGGGG
AAGCTCCTTGCTGCGTCATGGCTCAGCTATTCTCAGCCTCTCTCCTTTTA
TGGTGCCGGAAGCAGGCAGGCTGCTGCTAGATCCAGCGCACAGAGCCTTC
CTGCGTGGGGAAGCTCCTTGCTGCGTCATGGCTCAGCTATTCTCAGCCTC
TCTCCTTTTATGGTGCCGGAAGCAGGCAGGCTGCTGCTAGATCCAGCGCA
CAGAGCCTTCCTGCGTGGGGAAGCTCCTTGCTGCGTCATGGCTCAGCTAT
TCTCAGCCTCTCTCCTTTTATGGTGCCGGAAGCAGGCAGGCTGCTGCTCG
CGCAGCAGAGCACATTAGTCACTCGGGGCTGTGAAGGGGCGGGTCCTTGA
AGGAGAGGGCGCGGGCGGGCTCTGGCGCGGAGCCTGGGCGCCGCCAATGG
GAGCCAGGGCTCCACGAGCTGCCGCCCACGGGCCCCGCGCAGCATAAATA
GCCGCTGGTGGCGGTTTCGGTGCAGAGCTCAAGCGAGTTCTCCCGCAGCC
GCAGTCTCTGGGCCTCTCTAGCTTCAGCGGCGACGAGCCTGCCACACTCG
CTAAGCTCCTCCGGCACCGCACACCTGCCACTGCCGCTGCAGCCGCCGGC
TCTGCTCCCTTCCGGCTTCTGCCTCAGAGGAGTTCTTAGCCTGTTCGGAG
CCGCAGCACCGACGACCAGGCTAGCAGagaattcGCTGTCTGCGAGGGCC
AGCTGTTGGGGTGAGTACTCCCTCTCAAAAGCGGGCATGACTTCTGCGCT
AAGATTGTCAGTTTCCAAAAACGAGGAGGATTTGATATTCACCTGGCCCG
CGGTGATGCCTTTGAGGGTGGCCGCGTCCATCTGGTCAGAAAAGACAATC
TTTTTGTTGTCAAGCTTGAGGTGTGGCAGGCTTGAGATCTGGCCATACAC
TTGAGTGACAATGACATCCACTTTGCCTTTCTCTCCACAGGTGTCCACTC
CCAGGTCCAACTGCAGCCCAAGCGGAGGATCCGCCACCatgcccgccatg
aagatcgagtgccgcatcaccggcaccctgaacggcgtggagttcgagct
ggtgggcggcggagagggcacccccgaGCAGGGCCGCATGACCAACAAGA
TGAAGAGCACCAAAGGCGCCCTGACCTTCAGCCCCTACCTGCTGAGCCAC
GTGATGGGCTACGGCTTCTACCACTTCGGCACCTACCCCAGCGGCTACGA
GAACCCCTTCCTGCACGCCATCAACAACGGCGGCTACACCAACACCCGCA
TCGAGAAGTACGAGGACGGCGGCGTGCTGCACGTGAGCTTCAGCTACCGC
TACGAGGCCGGCCGCGTGATCGGCGACTTCAAGGTGGTGGGCACCGGCTT
CCCCGAGGACAGCGTGATCTTCACCGACAAGATCATCCGCAGCAACGCCA
CCGTGGAGCACCTGCACCCCATGGGCGATAACGTGCTGGTGGGCAGCTTC
GCCCGCACCTTCAGCCTGCGCGACGGCGGCTACTACAGCTTCGTGGTGGA
CAGCCACATGCACTTCAAGAGCGCCATCCACCCCAGCATCCTGCAGAACG
GGGGCCCCATGTTCGCCTTCCGCCGCGTGGAGGAGCTGCACAGCAACACC
GAGCTGGGCATCGTGGAGTACCAGCACGCCTTCAAGACCCCCATCGCCTT
AGGATGATGGCACGCTGCCCATGTCTTGTGCCCAGGAGAGCGGGATGGAC
CGTCACCCTGCAGCCTGTGCTTCTGCTAGGATCAATGTGACCGGTGAGGG
CAGAGGAAGTCTTCTAACATGCGGTGACGTGGAGGAGAATCCCGGCCCTA
TGACCGTGATGAGCGGCGAGAACGTGGACGAGGCCTCTGCCGCTCCTGGA
CACCCTCAGGATGGCAGCTATCCCAGACAGGCCGACCACGACGATCACGA
GTGCTGCGAGCGGGTCGTGATCAACATCAGCGGCCTGAGATTCGAGACAC
AGCTGAAAACCCTGGCCCAGTTCCCCAACACCCTGCTGGGCAACCCCAAG
AAACGGATGCGGTACTTCGACCCCCTGCGGAACGAGTACTTCTTCGACCG
GAACCGGCCCAGCTTCGACGCCATCCTGTACTACTACCAGAGCGGCGGCA
GACTGCGGAGGCCCGTGAATGTGCCCCTGGACATGTTCAGCGAGGAAATC
AAGTTCTACGAGCTGGGCGAGGAAGCCATGGAAAAGTTCAGAGAGGACGA
GGGCTTCATCAAAGAGGAAGAGAGGCCCCTGCCCGAGAAAGAATACCAGA
GACAAGTGTGGCTGCTGTTCGAGTACCCCGAGTCTAGCGGCCCTGCCAGA
GTGATCGCCATCGTGTCCGTGATGGTCATCCTGATCTCTATCGTGATCTT
CTGCCTGGAAACCCTGCCTGAGCTGAAGGACGACAAGGACTTCACCGGCA
CCGTGCACCGGATCGACAACACCACCGTGATCTACAACAGCAATATCTTC
ACCGACCCATTCTTCATCGTGGAAACACTGTGCATCATCTGGTTCAGCTT
CGAGCTGGTCGTGCGGTTCTTCGCCTGCCCCAGCAAGACCGACTTCTTCA
AGAACATCATGAACTTCATTGATATCGTGGCCATCATCCCCTACTTCATC
ACCCTGGGCACCGAGATCGCCGAGCAGGAAGGCAATCAGAAGGGCGAGCA
GGCCACCAGCCTGGCCATTCTGAGAGTGATCAGACTCGTGCGGGTGTTCC
GGATCTTCAAGCTGAGCCGGCACAGCAAGGGCCTGCAGATCCTGGGCCAG
ACACTGAAGGCCAGCATGAGAGAGCTGGGCCTGCTGATCTTCTTTCTGTT
CATCGGCGTGATCCTGTTCAGCAGCGCCGTGTACTTCGCCGAGGCCGAAG
AAGCCGAGAGCCACTTCAGCTCTATCCCCGACGCCTTTTGGTGGGCCGTG
GTGTCCATGACCACAGTGGGCTACGGCGACATGGTGCCCGTGACAATCGG
CGGCAAGATCGTGGGCAGCCTGTGTGCCATTGCCGGCGTGCTGACAGTCG
CCCTGCCTGTGCCTGTGATCGTGTCCAACTTCAACTACTTCTACCACCGG
GAAACCGAGGGGGAGGAACAGGCTCAGCTGCTGCACGTGTCCAGCCCCAA
TCTGGCCAGCGACAGCGACCTGAGCAGACGGTCTAGCAGCACCATGAGCA
AGAGCGAGTACATGGAAATCGAAGAGGACATGAACAACTCTATCGCCCAC
TACCGCCAAGTGAACATCCGGACCGCCAACTGCACCACCGCCAACCAGAA
CTGCGTGAACAAGAGCAAGCTGCTGACCGATGTCTGA

Nucleotide sequence of optimised AAV- ESARE-dsGFP-
KCNA1 vector (SEQ ID No: 24) gcgctcgctcactgaggccgc
ccgggcaaagcccgggcgtcgggcgacctttggtcgcccggcctcagtga
gcgagcgagcgcgcagagagggagtggccaactccatcactaggggttcc
tgcggccgcACGCGTGTGTCTAGACTGCAGACCATGGGGATCCAGCGCAC
AGAGCCTTCCTGCGTGGGGAAGCTCCTTGCTGCGTCATGGCTCAGCTATT
CTCAGCCTCTCTCCTTTTATGGTGCCGGAAGCAGGCAGGCTGCTGCTAGA
TCCAGCGCACAGAGCCTTCCTGCGTGGGGAAGCTCCTTGCTGCGTCATGG
CTCAGCTATTCTCAGCCTCTCTCCTTTTATGGTGCCGGAAGCAGGCAGGC
GCTGCTAGATCCAGCGCACAGAGCCTTCCTGCGTGGGGAAGCTCCTTGCT
GCGTCATGGCTCAGCTATTCTCAGCCTCTCTCCTTTTATGGTGCCGGAAG
CAGGCAGGCTGCTGCTAGATCCAGCGCACAGAGCCTTCCTGCGTGGGGAA
GCTCCTTGCTGCGTCATGGCTCAGCTATTCTCAGCCTCTCTCCTTTTATG
GTGCCGGAAGCAGGCAGGCTGCTGCTAGATCCAGCGCACAGAGCCTTCCT
GCGTGGGGAAGCTCCTTGCTGCGTCATGGCTCAGCTATTCTCAGCCTCTC
TCCTTTTATGGTGCCGGAAGCAGGCAGGCTGCTGCTCGCGCAGCAGAGCA
CATTAGTCACTCGGGGCTGTGAAGGGGCGGGTCCTTGAGGGCACCCACGG
GAGGGGAGCGAGTAGGCGCGGAAGGCGGGGCCTGCGGCAGGAGAGGGCGC
GGGCGGGCTCTGGCGCGGAGCCTGGGCGCCGCCAATGGGAGCCAGGGCTC
CACGAGCTGCCGCCCACGGGCCCCGCGCAGCATAAATAGCCGCTGGTGGC
GGTTTCGGTGCAGAGCTCAAGCGAGTTCTCCCGCAGCCGCAGTCTCTGGG
CCTCTCTAGCTTCAGCGGCGACGAGCCTGCCACACTCGCTAAGCTCCTCC
GGCACCGCACACCTGCCACTGCCGCTGCAGCCGCCGGCTCTGCTCCCTTC
CGGCTTCTGCCTCAGAGGAGTTCTTAGCCTGTTCGGAGCCGCAGCACCGA
CGACCAGGCTAGCAGagaattcGCTGTCTGCGAGGGCCAGCTGTTGGGGT
GAGTACTCCCTCTCAAAAGCGGGCATGACTTCTGCGCTAAGATTGTCAGT
TTCCAAAAACGAGGAGGATTTGATATTCACCTGGCCCGCGGTGATGCCTT
TGAGGGTGGCCGCGTCCATCTGGTCAGAAAAGACAATCTTTTTGTTGTCA
AGCTTGAGGTGTGGCAGGCTTGAGATCTGGCCATACACTTGAGTGACAAT
GACATCCACTTTGCCTTTCTCTCCACAGGTGTCCACTCCCAGGTCCAACT
GCAGCCCAAGCGGAGGATCCGCCACCatgcccgccatgaagatcgagtgc
cgcatcaccggcaccctgaacggcgtggagttcgagctggtgggcggcgg
agagggcacccccgaGCAGGGCCGCATGACCAACAAGATGAAGAGCACCA
AAGGCGCCCTGACCTTCAGCCCCTACCTGCTGAGCCACGTGATGGGCTAC
GGCTTCTACCACTTCGGCACCTACCCCAGCGGCTACGAGAACCCCTTCCT
GCACGCCATCAACAACGGCGGCTACACCAACACCCGCATCGAGAAGTACG
AGGACGGCGGCGTGCTGCACGTGAGCTTCAGCTACCGCTACGAGGCCGGC
CGCGTGATCGGCGACTTCAAGGTGGTGGGCACCGGCTTCCCCGAGGACAG
CGTGATCTTCACCGACAAGATCATCCGCAGCAACGCCACCGTGGAGCACC
TGCACCCCATGGGCGATAACGTGCTGGTGGGCAGCTTCGCCCGCACCTTC
AGCCTGCGCGACGGCGGCTACTACAGCTTCGTGGTGGACAGCCACATGCA
CTTCAAGAGCGCCATCCACCCCAGCATCCTGCAGAACGGGGGCCCCATGT
TCGCCTTCCGCCGCGTGGAGGAGCTGCACAGCAACACCGAGCTGGGCATC
GTGGAGTACCAGCACGCCTTCAAGACCCCCATCGCCTTCGCCAGATCTCG
AGATATCAGCCATGGCTTCCCGCCGGCGGTGGCGGCGCAGGATGATGGCA
CGCTGCCCATGTCTTGTGCCCAGGAGAGCGGGATGGACCGTCACCCTGCA
GCCTGTGCTTCTGCTAGGATCAATGTGACCGGTGAGGGCAGAGGAAGTCT
TCTAACATGCGGTGACGTGGAGGAGAATCCCGGCCCTATGACCGTGATGA
GCGGCGAGAACGTGGACGAGGCCTCTGCCGCTCCTGGACACCCTCAGGAT
GGCAGCTATCCCAGACAGGCCGACCACGACGATCACGAGTGCTGCGAGCG
GGTCGTGATCAACATCAGCGGCCTGAGATTCGAGACACAGCTGAAAACCC
TGGCCCAGTTCCCCAACACCCTGCTGGGCAACCCCAAGAAACGGATGCGG
TACTTCGACCCCCTGCGGAACGAGTACTTCTTCGACCGGAACCGGCCCAG
CTTCGACGCCATCCTGTACTACTACCAGAGCGGCGGCAGACTGCGGAGGC
CCGTGAATGTGCCCCTGGACATGTTCAGCGAGGAAATCAAGTTCTACGAG
CTGGGCGAGGAAGCCATGGAAAAGTTCAGAGAGGACGAGGGCTTCATCAA
AGAGGAAGAGAGGCCCCTGCCCGAGAAAGAATACCAGAGACAAGTGTGGC
TGCTGTTCGAGTACCCCGAGTCTAGCGGCCCTGCCAGAGTGATCGCCATC
GTGTCCGTGATGGTCATCCTGATCTCTATCGTGATCTTCTGCCTGGAAAC
CCTGCCTGAGCTGAAGGACGACAAGGACTTCACCGGCACCGTGCACCGGA
TCGACAACACCACCGTGATCTACAACAGCAATATCTTCACCGACCCATTC
TTCATCGTGGAAACACTGTGCATCATCTGGTTCAGCTTCGAGCTGGTCGT
GCGGTTCTTCGCCTGCCCCAGCAAGACCGACTTCTTCAAGAACATCATGA
ACTTCATTGATATCGTGGCCATCATCCCCTACTTCATCACCCTGGGCACC
GAGATCGCCGAGCAGGAAGGCAATCAGAAGGGCGAGCAGGCCACCAGCCT
GGCCATTCTGAGAGTGATCAGACTCGTGCGGGTGTTCCGGATCTTCAAGC
TGAGCCGGCACAGCAAGGGCCTGCAGATCCTGGGCCAGACACTGAAGGCC
AGCATGAGAGAGCTGGGCCTGCTGATCTTCTTTCTGTTCATCGGCGTGAT
CCTGTTCAGCAGCGCCGTGTACTTCGCCGAGGCCGAAGAAGCCGAGAGCC
ACTTCAGCTCTATCCCCGACGCCTTTTGGTGGGCCGTGGTGTCCATGACC
ACAGTGGGCTACGGCGACATGGTGCCCGTGACAATCGGCGGCAAGATCGT
GGGCAGCCTGTGTGCCATTGCCGGCGTGCTGACAGTCGCCCTGCCTGTGC
CTGTGATCGTGTCCAACTTCAACTACTTCTACCACCGGGAAACCGAGGGG
GAGGAACAGGCTCAGCTGCTGCACGTGTCCAGCCCCAATCTGGCCAGCGA
CAGCGACCTGAGCAGACGGTCTAGCAGCACCATGAGCAAGAGCGAGTACA
TGGAAATCGAAGAGGACATGAACAACTCTATCGCCCACTACCGCCAAGTG
AACATCCGGACCGCCAACTGCACCACCGCCAACCAGAACTGCGTGAACAA
GAGCAAGCTGCTGACCGATGTCTGAgTCGACAATCAACCTCATcgatacc
gagcgctgctcgagagatctacgggtggcatccctgtgacccctccccag
tgcctctcctggccctggaagttgccactccagtgcccaccagccttgtc
ctaataaaattaagttgcatcattttgtctgactaggtgtccttctataa
tattatggggtggaggggggtggtatggagcaaggggcaagttgggaaga
caacctgtagggcctgcggggtctattgggaaccaagctggagtgcagtg
gcacaatcttggctcactgcaatctccgcctcctgggttcaagcgattct
cctgcctcagcctcccgagttgttgggattccaggcatgcatgaccaggc
tcagctaatttttgtttttttggtagagacggggtttcaccatattggcc
aggctggtctccaactcctaatctcaggtgatctacccaccttggcctcc
caaattgctgggattacaggcgtgaaccactgctcccttccctgtccttc
tgattttgtaggtaaccacgtgcggaccgagcggccgcaggaacccctag
tgatggagttggccactccctctctgcgcgctcgctcgctcactgaggcc
gggcgaccaaaggtcgcccgacgcccgggctttgcccgggcggcctcagt
gagcgagcgagcgcgcagctgcctgcaggggcgcctgatgcggtattttc
tccttacgcatctgtgcggtatttcacaccgcatacgtcaaagcaaccat
agtacgcgccctgtagcggcgcattaagcgcggcgggtgtggtggttacg
cgcagcgtgaccgctacacttgccagcgccctagcgcccgctcctttcgc
tttcttcccttcctttctcgccacgttcgccggctttccccgtcaagctc
taaatcgggggctccctttagggttccgatttagtgctttacggcacctc
gaccccaaaaaacttgatttgggtgatggttcacgtagtgggccatcgcc
ctgatagacggtttttcgccctttgacgttggagtccacgttctttaata
gtggactcttgttccaaactggaacaacactcaaccctatctcgggctat
tcttttgatttataagggattttgccgatttcggcctattggttaaaaaa
tgagctgatttaacaaaaatttaacgcgaattttaacaaaatattaacgt
ttacaattttatggtgcactctcagtacaatctgctctgatgccgcatag
ttaagccagccccgacacccgccaacacccgctgacgcgccctgacgggc
ttgtctgctcccggcatccgcttacagacaagctgtgaccgtctccggga
gctgcatgtgtcagaggttttcaccgtcatcaccgaaacgcgcagacgaa
agggcctcgtgatacgcctatttttataggttaatgtcatgataataatg
gtttcttagacgtcaggtggcacttttcggggaaatgtgcgcggaacccc
tatttgtttatttttctaaatacattcaaatatgtatccgctcatgagac
aataaccctgataaatgcttcaataatattgaaaaaggaagagtatgagt
attcaacatttccgtgtcgcccttattcccttttttgcggcattttgcct
tcctgtttttgctcacccagaaacgctggtgaaagtaaaagatgctgaag
atcagttgggtgcacgagtgggttacatcgaactggatctcaacagcggt
aagatccttgagagttttcgccccgaagaacgttttccaatgatgagcac
ttttaaagttctgctatgtggcgcggtattatcccgtattgacgccgggc
aagagcaactcggtcgccgcatacactattctcagaatgacttggttgag
tactcaccagtcacagaaaagcatcttacggatggcatgacagtaagaga
attatgcagtgctgccataaccatgagtgataacactgcggccaacttac
ttctgacaacgatcggaggaccgaaggagctaaccgcttttttgcacaac
atgggggatcatgtaactcgccttgatcgttgggaaccggagctgaatga
agccataccaaacgacgagcgtgacaccacgatgcctgtagcaatggcaa
caacgttgcgcaaactattaactggcgaactacttactctagcttcccgg
caacaattaatagactggatggaggcggataaagttgcaggaccacttct
gcgctcggcccttccggctggctggtttattgctgataaatctggagccg
gtgagcgtgggtctcgcggtatcattgcagcactggggccagatggtaag
ccctcccgtatcgtagttatctacacgacggggagtcaggcaactatgga
tgaacgaaatagacagatcgctgagataggtgcctcactgattaagcatt
ggtaactgtcagaccaagtttactcatatatactttagattgatttaaaa
cttcatttttaatttaaaaggatctaggtgaagatcctttttgataatct
catgaccaaaatcccttaacgtgagttttcgttccactgagcgtcagacc
ccgtagaaaagatcaaaggatcttcttgagatcctttttttctgcgcgta
atctgctgcttgcaaacaaaaaaaccaccgctaccagcggtggtttgttt
gccggatcaagagctaccaactctttttccgaaggtaactggcttcagca
gagcgcagataccaaatactgtccttctagtgtagccgtagttaggccac
cacttcaagaactctgtagcaccgcctacatacctcgctctgctaatcct
gttaccagtggctgctgccagtggcgataagtcgtgtcttaccgggttgg
actcaagacgatagttaccggataaggcgcagcggtcgggctgaacgggg
ggttcgtgcacacagcccagcttggagcgaacgacctacaccgaactgag
atacctacagcgtgagctatgagaaagcgccacgcttcccgaagggagaa
aggcggacaggtatccggtaagcggcagggtcggaacaggagagcgcacg
agggagcttccagggggaaacgcctggtatctttatagtcctgtcgggtt
tcgccacctctgacttgagcgtcgatttttgtgatgctcgtcaggggggc
ggagcctatggaaaaacgccagcaacgcggcctttttacggttcctggcc
ttttgctggccttttgctcacatgtcctgcag

Nucleotide sequence of ESARE-dsGFP-KCNJ2 construct
(SEQ ID NO: 25) AGAGCCTTCCTGCGTGGGGAAGCTCCTTGCTGC
GTCATGGCTCAGCTATTCTCAGCCTCTCTCCTTTTATGGTGCCGGAAGCA
GGCAGGCTGCTGCTAGATCCAGCGCACAGAGCCTTCCTGCGTGGGGAAGC
TCCTTGCTGCGTCATGGCTCAGCTATTCTCAGCCTCTCTCCTTTTATGGT
GCCGGAAGCAGGCAGGCTGCTGCTAGATCCAGCGCACAGAGCCTTCCTGC
GTGGGGAAGCTCCTTGCTGCGTCATGGCTCAGCTATTCTCAGCCTCTCTC
CTTTTATGGTGCCGGAAGCAGGCAGGCTGCTGCTAGATCCAGCGCACAGA
GCCTTCCTGCGTGGGGAAGCTCCTTGCTGCGTCATGGCTCAGCTATTCTC
AGCCTCTCTCCTTTTATGGTGCCGGAAGCAGGCAGGCTGCTGCTAGATCC
AGCGCACAGAGCCTTCCTGCGTGGGGAAGCTCCTTGCTGCGTCATGGCTC
AGCTATTCTCAGCCTCTCTCCTTTTATGGTGCCGGAAGCAGGCAGGCTGC
TGCTCGCGCAGCAGAGCACATTAGTCACTCGGGGCTGTGAAGGGGCGGGT
CCTTGAGGGCACCCACGGGAGGGGAGCGAGTAGGCGCGGAAGGCGGGGCC
TGCGGCAGGAGAGGGCGCGGGCGGGCTCTGGCGCGGAGCCTGGGCGCCGC
CAATGGGAGCCAGGGCTCCACGAGCTGCCGCCCACGGGCCCCGCGCAGCA
TAAATAGCCGCTGGTGGCGGTTTCGGTGCAGAGCTCAAGCGAGTTCTCCC
GCAGCCGCAGTCTCTGGGCCTCTCTAGCTTCAGCGGCGACGAGCCTGCCA
CACTCGCTAAGCTCCTCCGGCACCGCACACCTGCCACTGCCGCTGCAGCC
GCCGGCTCTGCTCCCTTCCGGCTTCTGCCTCAGAGGAGTTCTTAGCCTGT
TCGGAGCCGCAGCACCGACGACCAGGCTAGCAGagaattcGCTGTCTGCG
AGGGCCAGCTGTTGGGGTGAGTACTCCCTCTCAAAAGCGGGCATGACTTC
TGCGCTAAGATTGTCAGTTTCCAAAAACGAGGAGGATTTGATATTCACCT
GGCCCGCGGTGATGCCTTTGAGGGTGGCCGCGTCCATCTGGTCAGAAAAG
ACAATCTTTTTGTTGTCAAGCTTGAGGTGTGGCAGGCTTGAGATCTGGCC
ATACACTTGAGTGACAATGACATCCACTTTGCCTTTCTCTCCACAGGTGT
CCACTCCCAGGTCCAACTGCAGCCCAAGCGGAGGATCCGCCACCatgccc
gccatgaagatcgagtgccgcatcaccggcaccctgaacggcgtggagtt
cgagctggtgggcggcggagagggcacccccgaGCAGGGCCGCATGACCA
ACAAGATGAAGAGCACCAAAGGCGCCCTGACCTTCAGCCCCTACCTGCTG
AGCCACGTGATGGGCTACGGCTTCTACCACTTCGGCACCTACCCCAGCGG
CTACGAGAACCCCTTCCTGCACGCCATCAACAACGGCGGCTACACCAACA
CCCGCATCGAGAAGTACGAGGACGGCGGCGTGCTGCACGTGAGCTTCAGC
TACCGCTACGAGGCCGGCCGCGTGATCGGCGACTTCAAGGTGGTGGGCAC
CGGCTTCCCCGAGGACAGCGTGATCTTCACCGACAAGATCATCCGCAGCA
ACGCCACCGTGGAGCACCTGCACCCCATGGGCGATAACGTGCTGGTGGGC
AGCTTCGCCCGCACCTTCAGCCTGCGCGACGGCGGCTACTACAGCTTCGT
GGTGGACAGCCACATGCACTTCAAGAGCGCCATCCACCCCAGCATCCTGC
AGAACGGGGGCCCCATGTTCGCCTTCCGCCGCGTGGAGGAGCTGCACAGC
AACACCGAGCTGGGCATCGTGGAGTACCAGCACGCCTTCAAGACCCCCAT
CGCCTTCGCCAGATCTCGAGATATCAGCCATGGCTTCCCGCCGGCGGTGG
CGGCGCAGGATGATGGCACGCTGCCCATGTCTTGTGCCCAGGAGAGCGGG
ATGGACCGTCACCCTGCAGCCTGTGCTTCTGCTAGGATCAATGTGACCGG
TGAGGGCAGAGGAAGTCTTCTAACATGCGGTGACGTGGAGGAGAATCCCG
GCCCTatgggcagtgtgagaaccaaccgctacagcatcgtctcttcagaa
gaagacggtatgaagttggccaccatggcagttgcaaatggctttgggaa
cgggaagagtaaagtccacacccgacaacagtgcaggagccgctttgtga
agaaagatggccactgtaatgttcagttcatcaatgtgggtgagaagggg
caacggtacctcgcagacatcttcaccacgtgtgtggacattcgctggcg
gtggatgctggttatcttctgcctggctttcgtcctgtcatggctgtttt
ttggctgtgtgttttggttgatagctctgctccatggggacctggatgca
tccaaagagggcaaagcttgtgtgtccgaggtcaacagcttcacggctgc
cttcctcttctccattgagacccagacaaccataggctatggtttcagat
gtgtcacggatgaatgcccaattgctgttttcatggtggtgttccagtca
atcgtgggctgcatcatcgatgctttcatcattggcgcagtcatggccaa
gatggcaaagccaaagaagagaaacgagactcttgtcttcagtcacaatg
ccgtgattgccatgagagacggcaagctgtgtttgatgtggcgagtgggc
aatcttcggaaaagccacttggtggaagctcatgttcgagcacagctcct
caaatccagaattacttctgaaggggagtatatccctctggatcaaatag
acatcaatgttgggtttgacagtggaatcgatcgtatatttctggtgtcc
ccaatcactatagtccatgaaatagatgaagacagtcctttatatgattt
gagtaaacaggacattgacaacgcagactttgaaatcgtggtcatactgg
aaggcatggtggaagccactgccatgacgacacagtgccgtagctcttat
ctagcaaatgaaatcctgtggggccaccgctatgagcctgtgctctttga
agagaagcactactacaaagtggactactccaggttccacaaaacttacg
aagtccccaacactcccctttgtagtgccagagacttagcagaaaagaaa
tatatcctctcaaatgcaaattcattttgctatgaaaatgaagttgccct
cacaagcaaagaggaagacgacagtgaaaatggagttccagaaagcacta
gtacggacacgccccctgacatagaccttcacaaccaggcaagtgtacct
ctagagcccaggcccttacggcgagagtcggagatatga

Nucleotide sequence of optimised AAV- ESARE-dsGFP-
KCNJ2 vector (SEQ ID NO: 26) cctgcaggcagctgcgcgctc
gctcgctcactgaggccgcccgggcaaagcccgggcgtcgggcgaccttt
ggtcgcccggcctcagtgagcgagcgagcgcgcagagagggagtggccaa
ctccatcactaggggttcctgcggccgcACGCGTGTGTCTAGACTGCAGA
CCATGGGGATCCAGCGCACAGAGCCTTCCTGCGTGGGGAAGCTCCTTGCT
GCGTCATGGCTCAGCTATTCTCAGCCTCTCTCCTTTTATGGTGCCGGAAG
CAGGCAGGCTGCTGCTAGATCCAGCGCACAGAGCCTTCCTGCGTGGGGAA
GCTCCTTGCTGCGTCATGGCTCAGCTATTCTCAGCCTCTCTCCTTTTATG
GTGCCGGAAGCAGGCAGGCTGCTGCTAGATCCAGCGCACAGAGCCTTCCT
GCGTGGGGAAGCTCCTTGCTGCGTCATGGCTCAGCTATTCTCAGCCTCTC
TCCTTTTATGGTGCCGGAAGCAGGCAGGCTGCTGCTAGATCCAGCGCACA
GAGCCTTCCTGCGTGGGGAAGCTCCTTGCTGCGTCATGGCTCAGCTATTC
TCAGCCTCTCTCCTTTTATGGTGCCGGAAGCAGGCAGGCTGCTGCTAGAT
CCAGCGCACAGAGCCTTCCTGCGTGGGGAAGCTCCTTGCTGCGTCATGGC
TCAGCTATTCTCAGCCTCTCTCCTTTTATGGTGCCGGAAGCAGGCAGGCT
GCTGCTCGCGCAGCAGAGCACATTAGTCACTCGGGGCTGTGAAGGGGCGG
GTCCTTGAGGGCACCCACGGGAGGGGAGCGAGTAGGCGCGGAAGGCGGGG
CCTGCGGCAGGAGAGGGCGCGGGCGGGCTCTGGCGCGGAGCCTGGGCGCC
GCCAATGGGAGCCAGGGCTCCACGAGCTGCCGCCCACGGGCCCCGCGCAG
CATAAATAGCCGCTGGTGGCGGTTTCGGTGCAGAGCTCAAGCGAGTTCTC
CCGCAGCCGCAGTCTCTGGGCCTCTCTAGCTTCAGCGGCGACGAGCCTGC
CACACTCGCTAAGCTCCTCCGGCACCGCACACCTGCCACTGCCGCTGCAG
CCGCCGGCTCTGCTCCCTTCCGGCTTCTGCCTCAGAGGAGTTCTTAGCCT
GTTCGGAGCCGCAGCACCGACGACCAGGCTAGCAGagaattcGCTGTCTG
CGAGGGCCAGCTGTTGGGGTGAGTACTCCCTCTCAAAAGCGGGCATGACT
TCTGCGCTAAGATTGTCAGTTTCCAAAAACGAGGAGGATTTGATATTCAC
CTGGCCCGCGGTGATGCCTTTGAGGGTGGCCGCGTCCATCTGGTCAGAAA
AGACAATCTTTTTGTTGTCAAGCTTGAGGTGTGGCAGGCTTGAGATCTGG
CCATACACTTGAGTGACAATGACATCCACTTTGCCTTTCTCTCCACAGGT
GTCCACTCCCAGGTCCAACTGCAGCCCAAGCGGAGGATCCGCCACCatgc
ccgccatgaagatcgagtgccgcatcaccggcaccctgaacggcgtggag
ttcgagctggtgggcggcggagagggcacccccgaGCAGGGCCGCATGAC
CAACAAGATGAAGAGCACCAAAGGCGCCCTGACCTTCAGCCCCTACCTGC
TGAGCCACGTGATGGGCTACGGCTTCTACCACTTCGGCACCTACCCCAGC
GGCTACGAGAACCCCTTCCTGCACGCCATCAACAACGGCGGCTACACCAA
CACCCGCATCGAGAAGTACGAGGACGGCGGCGTGCTGCACGTGAGCTTCA
GCTACCGCTACGAGGCCGGCCGCGTGATCGGCGACTTCAAGGTGGTGGGC
ACCGGCTTCCCCGAGGACAGCGTGATCTTCACCGACAAGATCATCCGCAG
CAACGCCACCGTGGAGCACCTGCACCCCATGGGCGATAACGTGCTGGTGG
GCAGCTTCGCCCGCACCTTCAGCCTGCGCGACGGCGGCTACTACAGCTTC
GTGGTGGACAGCCACATGCACTTCAAGAGCGCCATCCACCCCAGCATCCT
GCAGAACGGGGGCCCCATGTTCGCCTTCCGCCGCGTGGAGGAGCTGCACA
GCAACACCGAGCTGGGCATCGTGGAGTACCAGCACGCCTTCAAGACCCCC
ATCGCCTTCGCCAGATCTCGAGATATCAGCCATGGCTTCCCGCCGGCGGT
GGCGGCGCAGGATGATGGCACGCTGCCCATGTCTTGTGCCCAGGAGAGCG
GGATGGACCGTCACCCTGCAGCCTGTGCTTCTGCTAGGATCAATGTGACC
GGTGAGGGCAGAGGAAGTCTTCTAACATGCGGTGACGTGGAGGAGAATCC
CGGCCCTatgggcagtgtgagaaccaaccgctacagcatcgtctcttcag
aagaagacggtatgaagttggccaccatggcagttgcaaatggctttggg
aacgggaagagtaaagtccacacccgacaacagtgcaggagccgctttgt
gaagaaagatggccactgtaatgttcagttcatcaatgtgggtgagaagg
ggcaacggtacctcgcagacatcttcaccacgtgtgtggacattcgctgg
cggtggatgctggttatcttctgcctggctttcgtcctgtcatggctgtt
ttttggctgtgtgttttggttgatagctctgctccatggggacctggatg
catccaaagagggcaaagcttgtgtgtccgaggtcaacagcttcacggct
gccttcctcttctccattgagacccagacaaccataggctatggtttcag
atgtgtcacggatgaatgcccaattgctgttttcatggtggtgttccagt
caatcgtgggctgcatcatcgatgctttcatcattggcgcagtcatggcc
aagatggcaaagccaaagaagagaaacgagactcttgtcttcagtcacaa
tgccgtgattgccatgagagacggcaagctgtgtttgatgtggcgagtgg
gcaatcttcggaaaagccacttggtggaagctcatgttcgagcacagctc
ctcaaatccagaattacttctgaaggggagtatatccctctggatcaaat
agacatcaatgttgggtttgacagtggaatcgatcgtatatttctggtgt
ccccaatcactatagtccatgaaatagatgaagacagtcctttatatgat
ttgagtaaacaggacattgacaacgcagactttgaaatcgtggtcatact
ggaaggcatggtggaagccactgccatgacgacacagtgccgtagctctt
atctagcaaatgaaatcctgtggggccaccgctatgagcctgtgctcttt
gaagagaagcactactacaaagtggactactccaggttccacaaaactta
cgaagtccccaacactcccctttgtagtgccagagacttagcagaaaaga
aatatatcctctcaaatgcaaattcattttgctatgaaaatgaagttgcc
ctcacaagcaaagaggaagacgacagtgaaaatggagttccagaaagcac
tagtacggacacgccccctgacatagaccttcacaaccaggcaagtgtac
ctctagagcccaggcccttacggcgagagtcggagatatgagTCGACAAT
CAACCTCATcgataccgagcgctgctcgagagatctacgggtggcatccc
tgtgacccctccccagtgcctctcctggccctggaagttgccactccagt
gcccaccagccttgtcctaataaaattaagttgcatcattttgtctgact
aggtgtccttctataatattatggggtggaggggggtggtatggagcaag
gggcaagttgggaagacaacctgtagggcctgcggggtctattgggaacc
aagctggagtgcagtggcacaatcttggctcactgcaatctccgcctcct
gggttcaagcgattctcctgcctcagcctcccgagttgttgggattccag
gcatgcatgaccaggctcagctaatttttgtttttttggtagagacgggg
tttcaccatattggccaggctggtctccaactcctaatctcaggtgatct
acccaccttggcctcccaaattgctgggattacaggcgtgaaccactgct
cccttccctgtccttctgattttgtaggtaaccacgtgcggaccgagcgg
ccgcaggaacccctagtgatggagttggccactccctctctgcgcgctcg
ctcgctcactgaggccgggcgaccaaaggtcgcccgacgcccgggctttg
cccgggcggcctcagtgagcgagcgagcgcgcagctgcctgcaggggcgc
ctgatgcggtattttctccttacgcatctgtgcggtatttcacaccgcat
acgtcaaagcaaccatagtacgcgccctgtagcggcgcattaagcgcggc
gggtgtggtggttacgcgcagcgtgaccgctacacttgccagcgccctag
cgcccgctcctttcgctttcttcccttcctttctcgccacgttcgccggc
tttccccgtcaagctctaaatcgggggctccctttagggttccgatttag
tgctttacggcacctcgaccccaaaaaacttgatttgggtgatggttcac
gtagtgggccatcgccctgatagacggtttttcgccctttgacgttggag
tccacgttctttaatagtggactcttgttccaaactggaacaacactcaa
ccctatctcgggctattcttttgatttataagggattttgccgatttcgg
cctattggttaaaaaatgagctgatttaacaaaaatttaacgcgaatttt
aacaaaatattaacgtttacaattttatggtgcactctcagtacaatctg
ctctgatgccgcatagttaagccagccccgacacccgccaacacccgctg
acgcgccctgacgggcttgtctgctcccggcatccgcttacagacaagct
gtgaccgtctccgggagctgcatgtgtcagaggttttcaccgtcatcacc
gaaacgcgcgagacgaaagggcctcgtgatacgcctatttttataggtta
atgtcatgataataatggtttcttagacgtcaggtggcacttttcgggga
aatgtgcgcggaacccctatttgtttatttttctaaatacattcaaatat
gtatccgctcatgagacaataaccctgataaatgcttcaataatattgaa
aaaggaagagtatgagtattcaacatttccgtgtcgcccttattcccttt
tttgcggcattttgccttcctgtttttgctcacccagaaacgctggtgaa
agtaaaagatgctgaagatcagttgggtgcacgagtgggttacatcgaac
tggatctcaacagcggtaagatccttgagagttttcgccccgaagaacgt
tttccaatgatgagcacttttaaagttctgctatgtggcgcggtattatc
ccgtattgacgccgggcaagagcaactcggtcgccgcatacactattctc
agaatgacttggttgagtactcaccagtcacagaaaagcatcttacggat
ggcatgacagtaagagaattatgcagtgctgccataaccatgagtgataa
cactgcggccaacttacttctgacaacgatcggaggaccgaaggagctaa
ccgcttttttgcacaacatgggggatcatgtaactcgccttgatcgttgg
gaaccggagctgaatgaagccataccaaacgacgagcgtgacaccacgat
gcctgtagcaatggcaacaacgttgcgcaaactattaactggcgaactac
ttactctagcttcccggcaacaattaatagactggatggaggcggataaa
gttgcaggaccacttctgcgctcggcccttccggctggctggtttattgc
tgataaatctggagccggtgagcgtgggtctcgcggtatcattgcagcac
tggggccagatggtaagccctcccgtatcgtagttatctacacgacgggg
agtcaggcaactatggatgaacgaaatagacagatcgctgagataggtgc
ctcactgattaagcattggtaactgtcagaccaagtttactcatatatac
tttagattgatttaaaacttcatttttaatttaaaaggatctaggtgaag
atcctttttgataatctcatgaccaaaatcccttaacgtgagttttcgtt
ccactgagcgtcagaccccgtagaaaagatcaaaggatcttcttgagatc
ctttttttctgcgcgtaatctgctgcttgcaaacaaaaaaaccaccgcta
ccagcggtggtttgtttgccggatcaagagctaccaactctttttccgaa
ggtaactggcttcagcagagcgcagataccaaatactgtccttctagtgt
agccgtagttaggccaccacttcaagaactctgtagcaccgcctacatac
ctcgctctgctaatcctgttaccagtggctgctgccagtggcgataagtc
gtgtcttaccgggttggactcaagacgatagttaccggataaggcgcagc
ggtcgggctgaacggggggttcgtgcacacagcccagcttggagcgaacg
acctacaccgaactgagatacctacagcgtgagctatgagaaagcgccac
gcttcccgaagggagaaaggcggacaggtatccggtaagcggcagggtcg
gaacaggagagcgcacgagggagcttccagggggaaacgcctggtatctt
tatagtcctgtcgggtttcgccacctctgacttgagcgtcgatttttgtg
atgctcgtcaggggggcggagcctatggaaaaacgccagcaacgcggcct
ttttacggttcctggccttttgctggccttttgctcacatgt

Nucleotide sequence of NRAM-hCfos-dsGFP-KCNA1 cons
truct (SEQ ID NO: 27) TGTTCGTGACTGTGACTAGAAGTTTGTT
CGTGACTGTGACTAGAAGTTTGTTCGTGACTGTGACTAGAAGTTTGTTCG
TGACTGTGAactcattcataaaacgcttgttataaaagcagtggctgcgg
cgcctcgtactccaaccgcatctgcagcgagcaactgagaagccaagact
gagccggcggccGAATTCGCTGTCTGCGAGGGCCAGCTGTTGGGGTGAGT
ACTCCCTCTCAAAAGCGGGCATGACTTCTGCGCTAAGATTGTCAGTTTCC
AAAAACGAGGAGGATTTGATATTCACCTGGCCCGCGGTGATGCCTTTGAG
GGTGGCCGCGTCCATCTGGTCAGAAAAGACAATCTTTTTGTTGTCAAGCT
TGAGGTGTGGCAGGCTTGAGATCTGGCCATACACTTGAGTGACAATGACA
TCCACTTTGCCTTTCTCTCCACAGGTGTCCACTCCCAGGTCCAACTGCAG
CCCAAGCGGAGGATCCGCCACCatgcccgccatgaagatcgagtgccgca
tcaccggcaccctgaacggcgtggagttcgagctggtgggcggcggagag
ggcacccccgaGCAGGGCCGCATGACCAACAAGATGAAGAGCACCAAAGG
CGCCCTGACCTTCAGCCCCTACCTGCTGAGCCACGTGATGGGCTACGGCT
TCTACCACTTCGGCACCTACCCCAGCGGCTACGAGAACCCCTTCCTGCAC
GCCATCAACAACGGCGGCTACACCAACACCCGCATCGAGAAGTACGAGGA
CGGCGGCGTGCTGCACGTGAGCTTCAGCTACCGCTACGAGGCCGGCCGCG
TGATCGGCGACTTCAAGGTGGTGGGCACCGGCTTCCCCGAGGACAGCGTG
ATCTTCACCGACAAGATCATCCGCAGCAACGCCACCGTGGAGCACCTGCA
CCCCATGGGCGATAACGTGCTGGTGGGCAGCTTCGCCCGCACCTTCAGCC
TGCGCGACGGCGGCTACTACAGCTTCGTGGTGGACAGCCACATGCACTTC
AAGAGCGCCATCCACCCCAGCATCCTGCAGAACGGGGGCCCCATGTTCGC
CTTCCGCCGCGTGGAGGAGCTGCACAGCAACACCGAGCTGGGCATCGTGG
AGTACCAGCACGCCTTCAAGACCCCCATCGCCTTCGCCAGATCTCGAGAT
ATCAGCCATGGCTTCCCGCCGGCGGTGGCGGCGCAGGATGATGGCACGCT
GCCCATGTCTTGTGCCCAGGAGAGCGGGATGGACCGTCACCCTGCAGCCT
GTGCTTCTGCTAGGATCAATGTGACCGGTGAGGGCAGAGGAAGTCTTCTA
ACATGCGGTGACGTGGAGGAGAATCCCGGCCCTATGACCGTGATGAGCGG
CGAGAACGTGGACGAGGCCTCTGCCGCTCCTGGACACCCTCAGGATGGCA
GCTATCCCAGACAGGCCGACCACGACGATCACGAGTGCTGCGAGCGGGTC
GTGATCAACATCAGCGGCCTGAGATTCGAGACACAGCTGAAAACCCTGGC
CCAGTTCCCCAACACCCTGCTGGGCAACCCCAAGAAACGGATGCGGTACT
TCGACCCCCTGCGGAACGAGTACTTCTTCGACCGGAACCGGCCCAGCTTC
GACGCCATCCTGTACTACTACCAGAGCGGCGGCAGACTGCGGAGGCCCGT
GAATGTGCCCCTGGACATGTTCAGCGAGGAAATCAAGTTCTACGAGCTGG
GCGAGGAAGCCATGGAAAAGTTCAGAGAGGACGAGGGCTTCATCAAAGAG
GAAGAGAGGCCCCTGCCCGAGAAAGAATACCAGAGACAAGTGTGGCTGCT
GTTCGAGTACCCCGAGTCTAGCGGCCCTGCCAGAGTGATCGCCATCGTGT
CCGTGATGGTCATCCTGATCTCTATCGTGATCTTCTGCCTGGAAACCCTG
CCTGAGCTGAAGGACGACAAGGACTTCACCGGCACCGTGCACCGGATCGA
CAACACCACCGTGATCTACAACAGCAATATCTTCACCGACCCATTCTTCA
TCGTGGAAACACTGTGCATCATCTGGTTCAGCTTCGAGCTGGTCGTGCGG
TTCTTCGCCTGCCCCAGCAAGACCGACTTCTTCAAGAACATCATGAACTT
CATTGATATCGTGGCCATCATCCCCTACTTCATCACCCTGGGCACCGAGA
TCGCCGAGCAGGAAGGCAATCAGAAGGGCGAGCAGGCCACCAGCCTGGCC
ATTCTGAGAGTGATCAGACTCGTGCGGGTGTTCCGGATCTTCAAGCTGAG
CCGGCACAGCAAGGGCCTGCAGATCCTGGGCCAGACACTGAAGGCCAGCA
TGAGAGAGCTGGGCCTGCTGATCTTCTTTCTGTTCATCGGCGTGATCCTG
TTCAGCAGCGCCGTGTACTTCGCCGAGGCCGAAGAAGCCGAGAGCCACTT
CAGCTCTATCCCCGACGCCTTTTGGTGGGCCGTGGTGTCCATGACCACAG
TGGGCTACGGCGACATGGTGCCCGTGACAATCGGCGGCAAGATCGTGGGC
AGCCTGTGTGCCATTGCCGGCGTGCTGACAGTCGCCCTGCCTGTGCCTGT
GATCGTGTCCAACTTCAACTACTTCTACCACCGGGAAACCGAGGGGGAGG
AACAGGCTCAGCTGCTGCACGTGTCCAGCCCCAATCTGGCCAGCGACAGC
GACCTGAGCAGACGGTCTAGCAGCACCATGAGCAAGAGCGAGTACATGGA
AATCGAAGAGGACATGAACAACTCTATCGCCCACTACCGCCAAGTGAACA
TCCGGACCGCCAACTGCACCACCGCCAACCAGAACTGCGTGAACAAGAGC
AAGCTGCTGACCGATGTCTGAgTCGACAATCAACCTCA

Nucleotide sequence of optimised AAV- NRAM-hCfos -
dsGFP-KCNA1 vector (SEQ ID NO: 28) cctgcaggcagctgc
gcgctcgctcgctcactgaggccgcccgggcaaagcccgggcgtcgggcg
acctttggtcgcccggcctcagtgagcgagcgagcgcgcagagagggagt
ggccaactccatcactaggggttcctgcggccgcacgcgtTTCGCTATTA
CGCCAGTTTTATTCTAGAAGTTTGTTCGTGACTGTGACTAGAAGTTTGTT
CGTGACTGTGACTAGAAGTTTGTTCGTGACTGTGACTAGAAGTTTGTTCG
TGACTGTGAactcattcataaaacgcttgttataaaagcagtggctgcgg
cgcctcgtactccaaccgcatctgcagcgagcaactgagaagccaagact
gagccggcggccGAATTCGCTGTCTGCGAGGGCCAGCTGTTGGGGTGAGT
ACTCCCTCTCAAAAGCGGGCATGACTTCTGCGCTAAGATTGTCAGTTTCC
AAAAACGAGGAGGATTTGATATTCACCTGGCCCGCGGTGATGCCTTTGAG
GGTGGCCGCGTCCATCTGGTCAGAAAAGACAATCTTTTTGTTGTCAAGCT
TGAGGTGTGGCAGGCTTGAGATCTGGCCATACACTTGAGTGACAATGACA
TCCACTTTGCCTTTCTCTCCACAGGTGTCCACTCCCAGGTCCAACTGCAG
CCCAAGCGGAGGATCCGCCACCatgcccgccatgaagatcgagtgccgca
tcaccggcaccctgaacggcgtggagttcgagctggtgggcggcggagag
ggcacccccgaGCAGGGCCGCATGACCAACAAGATGAAGAGCACCAAAGG
CGCCCTGACCTTCAGCCCCTACCTGCTGAGCCACGTGATGGGCTACGGCT
TCTACCACTTCGGCACCTACCCCAGCGGCTACGAGAACCCCTTCCTGCAC
GCCATCAACAACGGCGGCTACACCAACACCCGCATCGAGAAGTACGAGGA
CGGCGGCGTGCTGCACGTGAGCTTCAGCTACCGCTACGAGGCCGGCCGCG
TGATCGGCGACTTCAAGGTGGTGGGCACCGGCTTCCCCGAGGACAGCGTG
ATCTTCACCGACAAGATCATCCGCAGCAACGCCACCGTGGAGCACCTGCA
CCCCATGGGCGATAACGTGCTGGTGGGCAGCTTCGCCCGCACCTTCAGCC
TGCGCGACGGCGGCTACTACAGCTTCGTGGTGGACAGCCACATGCACTTC
AAGAGCGCCATCCACCCCAGCATCCTGCAGAACGGGGGCCCCATGTTCGC
CTTCCGCCGCGTGGAGGAGCTGCACAGCAACACCGAGCTGGGCATCGTGG
AGTACCAGCACGCCTTCAAGACCCCCATCGCCTTCGCCAGATCTCGAGAT
ATCAGCCATGGCTTCCCGCCGGCGGTGGCGGCGCAGGATGATGGCACGCT
GCCCATGTCTTGTGCCCAGGAGAGCGGGATGGACCGTCACCCTGCAGCCT
GTGCTTCTGCTAGGATCAATGTGACCGGTGAGGGCAGAGGAAGTCTTCTA
ACATGCGGTGACGTGGAGGAGAATCCCGGCCCTATGACCGTGATGAGCGG
CGAGAACGTGGACGAGGCCTCTGCCGCTCCTGGACACCCTCAGGATGGCA
GCTATCCCAGACAGGCCGACCACGACGATCACGAGTGCTGCGAGCGGGTC
GTGATCAACATCAGCGGCCTGAGATTCGAGACACAGCTGAAAACCCTGGC
CCAGTTCCCCAACACCCTGCTGGGCAACCCCAAGAAACGGATGCGGTACT
TCGACCCCCTGCGGAACGAGTACTTCTTCGACCGGAACCGGCCCAGCTTC
GACGCCATCCTGTACTACTACCAGAGCGGCGGCAGACTGCGGAGGCCCGT
GAATGTGCCCCTGGACATGTTCAGCGAGGAAATCAAGTTCTACGAGCTGG
GCGAGGAAGCCATGGAAAAGTTCAGAGAGGACGAGGGCTTCATCAAAGAG
GAAGAGAGGCCCCTGCCCGAGAAAGAATACCAGAGACAAGTGTGGCTGCT
GTTCGAGTACCCCGAGTCTAGCGGCCCTGCCAGAGTGATCGCCATCGTGT
CCGTGATGGTCATCCTGATCTCTATCGTGATCTTCTGCCTGGAAACCCTG
CCTGAGCTGAAGGACGACAAGGACTTCACCGGCACCGTGCACCGGATCGA
CAACACCACCGTGATCTACAACAGCAATATCTTCACCGACCCATTCTTCA
TCGTGGAAACACTGTGCATCATCTGGTTCAGCTTCGAGCTGGTCGTGCGG
TTCTTCGCCTGCCCCAGCAAGACCGACTTCTTCAAGAACATCATGAACTT
CATTGATATCGTGGCCATCATCCCCTACTTCATCACCCTGGGCACCGAGA
TCGCCGAGCAGGAAGGCAATCAGAAGGGCGAGCAGGCCACCAGCCTGGCC
ATTCTGAGAGTGATCAGACTCGTGCGGGTGTTCCGGATCTTCAAGCTGAG
CCGGCACAGCAAGGGCCTGCAGATCCTGGGCCAGACACTGAAGGCCAGCA
TGAGAGAGCTGGGCCTGCTGATCTTCTTTCTGTTCATCGGCGTGATCCTG
TTCAGCAGCGCCGTGTACTTCGCCGAGGCCGAAGAAGCCGAGAGCCACTT
CAGCTCTATCCCCGACGCCTTTTGGTGGGCCGTGGTGTCCATGACCACAG
TGGGCTACGGCGACATGGTGCCCGTGACAATCGGCGGCAAGATCGTGGGC
AGCCTGTGTGCCATTGCCGGCGTGCTGACAGTCGCCCTGCCTGTGCCTGT
GATCGTGTCCAACTTCAACTACTTCTACCACCGGGAAACCGAGGGGGAGG
AACAGGCTCAGCTGCTGCACGTGTCCAGCCCCAATCTGGCCAGCGACAGC
GACCTGAGCAGACGGTCTAGCAGCACCATGAGCAAGAGCGAGTACATGGA
AATCGAAGAGGACATGAACAACTCTATCGCCCACTACCGCCAAGTGAACA
TCCGGACCGCCAACTGCACCACCGCCAACCAGAACTGCGTGAACAAGAGC
AAGCTGCTGACCGATGTCTGAgTCGACAATCAACCTCATcgataccgagc
gctgctcgagagatctacgggtggcatccctgtgacccctccccagtgcc
tctcctggccctggaagttgccactccagtgcccaccagccttgtcctaa
taaaattaagttgcatcattttgtctgactaggtgtccttctataatatt
atggggtggaggggggtggtatggagcaaggggcaagttgggaagacaac
ctgtagggcctgcggggtctattgggaaccaagctggagtgcagtggcac
aatcttggctcactgcaatctccgcctcctgggttcaagcgattctcctg
cctcagcctcccgagttgttgggattccaggcatgcatgaccaggctcag
ctaatttttgtttttttggtagagacggggtttcaccatattggccaggc
tggtctccaactcctaatctcaggtgatctacccaccttggcctcccaaa
ttgctgggattacaggcgtgaaccactgctcccttccctgtccttctgat
tttgtaggtaaccacgtgcggaccgagcggccgcaggaacccctagtgat
ggagttggccactccctctctgcgcgctcgctcgctcactgaggccgggc
gaccaaaggtcgcccgacgcccgggctttgcccgggcggcctcagtgagc
gagcgagcgcgcagctgcctgcaggggcgcctgatgcggtattttctcct
tacgcatctgtgcggtatttcacaccgcatacgtcaaagcaaccatagta
cgcgccctgtagcggcgcattaagcgcggcgggtgtggtggttacgcgca
gcgtgaccgctacacttgccagcgccctagcgcccgctcctttcgctttc
ttcccttcctttctcgccacgttcgccggctttccccgtcaagctctaaa
tcgggggctccctttagggttccgatttagtgctttacggcacctcgacc
ccaaaaaacttgatttgggtgatggttcacgtagtgggccatcgccctga
tagacggtttttcgccctttgacgttggagtccacgttctttaatagtgg
actcttgttccaaactggaacaacactcaaccctatctcgggctattctt
ttgatttataagggattttgccgatttcggcctattggttaaaaaatgag
ctgatttaacaaaaatttaacgcgaattttaacaaaatattaacgtttac
aattttatggtgcactctcagtacaatctgctctgatgccgcatagttaa
gccagccccgacacccgccaacacccgctgacgcgccctgacgggcttgt
ctgctcccggcatccgcttacagacaagctgtgaccgtctccgggagctg
catgtgtcagaggttttcaccgtcatcaccgaaacgcgcgagacgaaagg
gcctcgtgatacgcctatttttataggttaatgtcatgataataatggtt
tcttagacgtcaggtggcacttttcggggaaatgtgcgcggaacccctat
ttgtttatttttctaaatacattcaaatatgtatccgctcatgagacaat
aaccctgataaatgcttcaataatattgaaaaaggaagagtatgagtatt
caacatttccgtgtcgcccttattcccttttttgcggcattttgccttcc
tgtttttgctcacccagaaacgctggtgaaagtaaaagatgctgaagatc
agttgggtgcacgagtgggttacatcgaactggatctcaacagcggtaag
atccttgagagttttcgccccgaagaacgttttccaatgatgagcacttt
taaagttctgctatgtggcgcggtattatcccgtattgacgccgggcaag
agcaactcggtcgccgcatacactattctcagaatgacttggttgagtac
tcaccagtcacagaaaagcatcttacggatggcatgacagtaagagaatt
atgcagtgctgccataaccatgagtgataacactgcggccaacttacttc
tgacaacgatcggaggaccgaaggagctaaccgcttttttgcacaacatg
ggggatcatgtaactcgccttgatcgttgggaaccggagctgaatgaagc
cataccaaacgacgagcgtgacaccacgatgcctgtagcaatggcaacaa
cgttgcgcaaactattaactggcgaactacttactctagcttcccggcaa
caattaatagactggatggaggcggataaagttgcaggaccacttctgcg
ctcggcccttccggctggctggtttattgctgataaatctggagccggtg
agcgtgggtctcgcggtatcattgcagcactggggccagatggtaagccc
tcccgtatcgtagttatctacacgacggggagtcaggcaactatggatga
acgaaatagacagatcgctgagataggtgcctcactgattaagcattggt
aactgtcagaccaagtttactcatatatactttagattgatttaaaactt
catttttaatttaaaaggatctaggtgaagatcctttttgataatctcat
gaccaaaatcccttaacgtgagttttcgttccactgagcgtcagaccccg
tagaaaagatcaaaggatcttcttgagatcctttttttctgcgcgtaatc
tgctgcttgcaaacaaaaaaaccaccgctaccagcggtggtttgtttgcc
ggatcaagagctaccaactctttttccgaaggtaactggcttcagcagag
cgcagataccaaatactgtccttctagtgtagccgtagttaggccaccac
ttcaagaactctgtagcaccgcctacatacctcgctctgctaatcctgtt
accagtggctgctgccagtggcgataagtcgtgtcttaccgggttggact
caagacgatagttaccggataaggcgcagcggtcgggctgaacggggggt
tcgtgcacacagcccagcttggagcgaacgacctacaccgaactgagata
cctacagcgtgagctatgagaaagcgccacgcttcccgaagggagaaagg
cggacaggtatccggtaagcggcagggtcggaacaggagagcgcacgagg
gagcttccagggggaaacgcctggtatctttatagtcctgtcgggtttcg
ccacctctgacttgagcgtcgatttttgtgatgctcgtcaggggggcgga
gcctatggaaaaacgccagcaacgcggcctttttacggttcctggccttt
tgctggccttttgctcacatgt

Nucleotide sequence of NRAM-hCfos -dsGFP-KCNJ2 con
struct (SEQ ID NO: 29) GAAGTTTGTTCGTGACTGTGACTAGAA
GTTTGTTCGTGACTGTGACTAGAAGTTTGTTCGTGACTGTGACTAGAAGT
TTGTTCGTGACTGTGAACTCATTCATAAAACGCTTGTTATAAAAGCAGTG
GCTGCGGCGCCTCGTACTCCAACCGCATCTGCAGCGAGCAACTGAGAAGC
CAAGACTGAGCCGGCGGCCGAATTCGCTGTCTGCGAGGGCCAGCTGTTGG
GGTGAGTACTCCCTCTCAAAAGCGGGCATGACTTCTGCGCTAAGATTGTC
AGTTTCCAAAAACGAGGAGGATTTGATATTCACCTGGCCCGCGGTGATGC
CTTTGAGGGTGGCCGCGTCCATCTGGTCAGAAAAGACAATCTTTTTGTTG
TCAAGCTTGAGGTGTGGCAGGCTTGAGATCTGGCCATACACTTGAGTGAC
AATGACATCCACTTTGCCTTTCTCTCCACAGGTGTCCACTCCCAGGTCCA
ACTGCAGCCCAAGCGGAGGATCCGCCACCATGCCCGCCATGAAGATCGAG
TGCCGCATCACCGGCACCCTGAACGGCGTGGAGTTCGAGCTGGTGGGCGG
CGGAGAGGGCACCCCCGAGCAGGGCCGCATGACCAACAAGATGAAGAGCA
CCAAAGGCGCCCTGACCTTCAGCCCCTACCTGCTGAGCCACGTGATGGGC
TACGGCTTCTACCACTTCGGCACCTACCCCAGCGGCTACGAGAACCCCTT
CCTGCACGCCATCAACAACGGCGGCTACACCAACACCCGCATCGAGAAGT
ACGAGGACGGCGGCGTGCTGCACGTGAGCTTCAGCTACCGCTACGAGGCC
GGCCGCGTGATCGGCGACTTCAAGGTGGTGGGCACCGGCTTCCCCGAGGA
CAGCGTGATCTTCACCGACAAGATCATCCGCAGCAACGCCACCGTGGAGC
ACCTGCACCCCATGGGCGATAACGTGCTGGTGGGCAGCTTCGCCCGCACC
TTCAGCCTGCGCGACGGCGGCTACTACAGCTTCGTGGTGGACAGCCACAT
GCACTTCAAGAGCGCCATCCACCCCAGCATCCTGCAGAACGGGGGCCCCA
TGTTCGCCTTCCGCCGCGTGGAGGAGCTGCACAGCAACACCGAGCTGGGC
ATCGTGGAGTACCAGCACGCCTTCAAGACCCCCATCGCCTTCGCCAGATC
TCGAGATATCAGCCATGGCTTCCCGCCGGCGGTGGCGGCGCAGGATGATG
GCACGCTGCCCATGTCTTGTGCCCAGGAGAGCGGGATGGACCGTCACCCT
GCAGCCTGTGCTTCTGCTAGGATCAATGTGACCGGTGAGGGCAGAGGAAG
TCTTCTAACATGCGGTGACGTGGAGGAGAATCCCGGCCCTATGGGCAGTG
TGAGAACCAACCGCTACAGCATCGTCTCTTCAGAAGAAGACGGTATGAAG
TTGGCCACCATGGCAGTTGCAAATGGCTTTGGGAACGGGAAGAGTAAAGT
CCACACCCGACAACAGTGCAGGAGCCGCTTTGTGAAGAAAGATGGCCACT
GTAATGTTCAGTTCATCAATGTGGGTGAGAAGGGGCAACGGTACCTCGCA
GACATCTTCACCACGTGTGTGGACATTCGCTGGCGGTGGATGCTGGTTAT
CTTCTGCCTGGCTTTCGTCCTGTCATGGCTGTTTTTTGGCTGTGTGTTTT
GGTTGATAGCTCTGCTCCATGGGGACCTGGATGCATCCAAAGAGGGCAAA
GCTTGTGTGTCCGAGGTCAACAGCTTCACGGCTGCCTTCCTCTTCTCCAT
TGAGACCCAGACAACCATAGGCTATGGTTTCAGATGTGTCACGGATGAAT
GCCCAATTGCTGTTTTCATGGTGGTGTTCCAGTCAATCGTGGGCTGCATC
ATCGATGCTTTCATCATTGGCGCAGTCATGGCCAAGATGGCAAAGCCAAA
GAAGAGAAACGAGACTCTTGTCTTCAGTCACAATGCCGTGATTGCCATGA
GAGACGGCAAGCTGTGTTTGATGTGGCGAGTGGGCAATCTTCGGAAAAGC
CACTTGGTGGAAGCTCATGTTCGAGCACAGCTCCTCAAATCCAGAATTAC
TTCTGAAGGGGAGTATATCCCTCTGGATCAAATAGACATCAATGTTGGGT
TTGACAGTGGAATCGATCGTATATTTCTGGTGTCCCCAATCACTATAGTC
CATGAAATAGATGAAGACAGTCCTTTATATGATTTGAGTAAACAGGACAT
TGACAACGCAGACTTTGAAATCGTGGTCATACTGGAAGGCATGGTGGAAG
CCACTGCCATGACGACACAGTGCCGTAGCTCTTATCTAGCAAATGAAATC
CTGTGGGGCCACCGCTATGAGCCTGTGCTCTTTGAAGAGAAGCACTACTA
CAAAGTGGACTACTCCAGGTTCCACAAAACTTACGAAGTCCCCAACACTC
CCCTTTGTAGTGCCAGAGACTTAGCAGAAAAGAAATATATCCTCTCAAAT
GCAAATTCATTTTGCTATGAAAATGAAGTTGCCCTCACAAGCAAAGAGGA
AGACGACAGTGAAAATGGAGTTCCAGAAAGCACTAGTACGGACACGCCCC
CTGACATAGACCTTCACAACCAGGCAAGTGTACCTCTAGAGCCCAGGCCC
TTACGGCGAGAGTCGGAGATATGA

Nucleotide sequence of optimised AAV- NRAM-hCfos -
dsGFP-KCNJ2 vector (SEQ ID NO: 30) CAGGCAGCTGCGCGC
TCGCTCGCTCACTGAGGCCGCCCGGGCAAAGCCCGGGCGTCGGGCGACCT
TTGGTCGCCCGGCCTCAGTGAGCGAGCGAGCGCGCAGAGAGGGAGTGGCC
AACTCCATCACTAGGGGTTCCTGCGGCCGCACGCGTTTCGCTATTACGCC
AGTTTTATTCTAGAAGTTTGTTCGTGACTGTGACTAGAAGTTTGTTCGTG
ACTGTGACTAGAAGTTTGTTCGTGACTGTGACTAGAAGTTTGTTCGTGAC
TGTGAACTCATTCATAAAACGCTTGTTATAAAAGCAGTGGCTGCGGCGCC
TCGTACTCCAACCGCATCTGCAGCGAGCAACTGAGAAGCCAAGACTGAGC
CGGCGGCCGAATTCGCTGTCTGCGAGGGCCAGCTGTTGGGGTGAGTACTC
CCTCTCAAAAGCGGGCATGACTTCTGCGCTAAGATTGTCAGTTTCCAAAA
ACGAGGAGGATTTGATATTCACCTGGCCCGCGGTGATGCCTTTGAGGGTG
GCCGCGTCCATCTGGTCAGAAAAGACAATCTTTTTGTTGTCAAGCTTGAG
GTGTGGCAGGCTTGAGATCTGGCCATACACTTGAGTGACAATGACATCCA
CTTTGCCTTTCTCTCCACAGGTGTCCACTCCCAGGTCCAACTGCAGCCCA
AGCGGAGGATCCGCCACCATGCCCGCCATGAAGATCGAGTGCCGCATCAC
CGGCACCCTGAACGGCGTGGAGTTCGAGCTGGTGGGCGGCGGAGAGGGCA
CCCCCGAGCAGGGCCGCATGACCAACAAGATGAAGAGCACCAAAGGCGCC
CTGACCTTCAGCCCCTACCTGCTGAGCCACGTGATGGGCTACGGCTTCTA
CCACTTCGGCACCTACCCCAGCGGCTACGAGAACCCCTTCCTGCACGCCA
TCAACAACGGCGGCTACACCAACACCCGCATCGAGAAGTACGAGGACGGC
GGCGTGCTGCACGTGAGCTTCAGCTACCGCTACGAGGCCGGCCGCGTGAT
CGGCGACTTCAAGGTGGTGGGCACCGGCTTCCCCGAGGACAGCGTGATCT
TCACCGACAAGATCATCCGCAGCAACGCCACCGTGGAGCACCTGCACCCC
ATGGGCGATAACGTGCTGGTGGGCAGCTTCGCCCGCACCTTCAGCCTGCG
CGACGGCGGCTACTACAGCTTCGTGGTGGACAGCCACATGCACTTCAAGA
GCGCCATCCACCCCAGCATCCTGCAGAACGGGGGCCCCATGTTCGCCTTC
CGCCGCGTGGAGGAGCTGCACAGCAACACCGAGCTGGGCATCGTGGAGTA
CCAGCACGCCTTCAAGACCCCCATCGCCTTCGCCAGATCTCGAGATATCA
GCCATGGCTTCCCGCCGGCGGTGGCGGCGCAGGATGATGGCACGCTGCCC
ATGTCTTGTGCCCAGGAGAGCGGGATGGACCGTCACCCTGCAGCCTGTGC
TTCTGCTAGGATCAATGTGACCGGTGAGGGCAGAGGAAGTCTTCTAACAT
GCGGTGACGTGGAGGAGAATCCCGGCCCTATGGGCAGTGTGAGAACCAAC
CGCTACAGCATCGTCTCTTCAGAAGAAGACGGTATGAAGTTGGCCACCAT
GGCAGTTCAAATGGCTTTGGGAACGGGAAGAGTAAAGTCCACACCCGACA
ACAGTGCAGGAGCCGCTTTGTGAAGAAAGATGGCCACTGTAATGTTCAGT
TCATCAATGTGGGTGAGAAGGGGCAACGGTACCTCGCAGACATCTTCACC
ACGTGTGTGGACATTCGCTGGCGGTGGATGCTGGTTATCTTCTGCCTGGC
TTTCGTCCTGTCATGGCTGTTTTTTGGCTGTGTGTTTTGGTTGATAGCTC
TGCTCCATGGGGACCTGGATGCATCCAAAGAGGGCAAAGCTTGTGTGTCC
GAGGTCAACAGCTTCACGGCTGCCTTCCTCTTCTCCATTGAGACCCAGAC
AACCATAGGCTATGGTTTCAGATGTGTCACGGATGAATGCCCAATTGCTG
TTTTCATGGTGGTGTTCCAGTCAATCGTGGGCTGCATCATCGATGCTTTC
ATCATTGGCGCAGTCATGGCCAAGATGGCAAAGCCAAAGAAGAGAAACGA
GACTCTTGTCTTCAGTCACAATGCCGTGATTGCCATGAGAGACGGCAAGC
TGTGTTTGATGTGGCGAGTGGGCAATCTTCGGAAAAGCCACTTGGTGGAA
GCTCATGTTCGAGCACAGCTCCTCAAATCCAGAATTACTTCTGAAGGGGA
GTATATCCCTCTGGATCAAATAGACATCAATGTTGGGTTTGACAGTGGAA
TCGATCGTATATTTCTGGTGTCCCCAATCACTATAGTCCATGAAATAGAT
GAAGACAGTCCTTTATATGATTTGAGTAAACAGGACATTGACAACGCAGA
CTTTGAAATCGTGGTCATACTGGAAGGCATGGTGGAAGCCACTGCCATGA
CGACACAGTGCCGTAGCTCTTATCTAGCAAATGAAATCCTGTGGGGCCAC
CGCTATGAGCCTGTGCTCTTTGAAGAGAAGCACTACTACAAAGTGGACTA
CTCCAGGTTCCACAAAACTTACGAAGTCCCCAACACTCCCCTTTGTAGTG
CCAGAGACTTAGCAGAAAAGAAATATATCCTCTCAAATGCAAATTCATTT
TGCTATGAAAATGAAGTTGCCCTCACAAGCAAAGAGGAAGACGACAGTGA
AAATGGAGTTCCAGAAAGCACTAGTACGGACACGCCCCCTGACATAGACC
TTCACAACCAGGCAAGTGTACCTCTAGAGCCCAGGCCCTTACGGCGAGAG
TCGGAGATATGAGTCGACAATCAACCTCATCGATACCGAGCGCTGCTCGA
GAGATCTACGGGTGGCATCCCTGTGACCCCTCCCCAGTGCCTCTCCTGGC
CCTGGAAGTTGCCACTCCAGTGCCCACCAGCCTTGTCCTAATAAAATTAA
GTTGCATCATTTTGTCTGACTAGGTGTCCTTCTATAATATTATGGGGTGG
AGGGGGGTGGTATGGAGCAAGGGGCAAGTTGGGAAGACAACCTGTAGGGC
CTGCGGGGTCTATTGGGAACCAAGCTGGAGTGCAGTGGCACAATCTTGGC
TCACTGCAATCTCCGCCTCCTGGGTTCAAGCGATTCTCCTGCCTCAGCCT
CCCGAGTTGTTGGGATTCCAGGCATGCATGACCAGGCTCAGCTAATTTTT
GTTTTTTTGGTAGAGACGGGGTTTCACCATATTGGCCAGGCTGGTCTCCA
ACTCCTAATCTCAGGTGATCTACCCACCTTGGCCTCCCAAATTGCTGGGA
TTACAGGCGTGAACCACTGCTCCCTTCCCTGTCCTTCTGATTTTGTAGGT
AACCACGTGCGGACCGAGCGGCCGCAGGAACCCCTAGTGATGGAGTTGGC
CACTCCCTCTCTGCGCGCTCGCTCGCTCACTGAGGCCGGGCGACCAAAGG
TCGCCCGACGCCCGGGCTTTGCCCGGGCGGCCTCAGTGAGCGAGCGAGCG
CGCAGCTGCCTGCAGGGGCGCCTGATGCGGTATTTTCTCCTTACGCATCT
GTGCGGTATTTCACACCGCATACGTCAAAGCAACCATAGTACGCGCCCTG
TAGCGGCGCATTAAGCGCGGCGGGTGTGGTGGTTACGCGCAGCGTGACCG
CTACACTTGCCAGCGCCCTAGCGCCCGCTCCTTTCGCTTTCTTCCCTTCC
TTTCTCGCCACGTTCGCCGGCTTTCCCCGTCAAGCTCTAAATCGGGGGCT
CCCTTTAGGGTTCCGATTTAGTGCTTTACGGCACCTCGACCCCAAAAAAC
TTGATTTGGGTGATGGTTCACGTAGTGGGCCATCGCCCTGATAGACGGTT
TTTCGCCCTTTGACGTTGGAGTCCACGTTCTTTAATAGTGGACTCTTGTT
CCAAACTGGAACAACACTCAACCCTATCTCGGGCTATTCTTTTGATTTAT
AAGGGATTTTGCCGATTTCGGCCTATTGGTTAAAAAATGAGCTGATTTAA
CAAAAATTTAACGCGAATTTTAACAAAATATTAACGTTTACAATTTTATG
GTGCACTCTCAGTACAATCTGCTCTGATGCCGCATAGTTAAGCCAGCCCC
GACACCCGCCAACACCCGCTGACGCGCCCTGACGGGCTTGTCTGCTCCCG
GCATCCGCTTACAGACAAGCTGTGACCGTCTCCGGGAGCTGCATGTGTCA
GAGGTTTTCACCGTCATCACCGAAACGCGCGAGACGAAAGGGCCTCGTGA
TACGCCTATTTTTATAGGTTAATGTCATGATAATAATGGTTTCTTAGACG
TCAGGTGGCACTTTTCGGGGAAATGTGCGCGGAACCCCTATTTGTTTATT
TTTCTAAATACATTCAAATATGTATCCGCTCATGAGACAATAACCCTGAT
AAATGCTTCAATAATATTGAAAAAGGAAGAGTATGAGTATTCAACATTTC
CGTGTCGCCCTTATTCCCTTTTTTGCGGCATTTTGCCTTCCTGTTTTTGC
TCACCCAGAAACGCTGGTGAAAGTAAAAGATGCTGAAGATCAGTTGGGTG
CACGAGTGGGTTACATCGAACTGGATCTCAACAGCGGTAAGATCCTTGAG
AGTTTTCGCCCCGAAGAACGTTTTCCAATGATGAGCACTTTTAAAGTTCT
GCTATGTGGCGCGGTATTATCCCGTATTGACGCCGGGCAAGAGCAACTCG
GTCGCCGCATACACTATTCTCAGAATGACTTGGTTGAGTACTCACCAGTC
ACAGAAAAGCATCTTACGGATGGCATGACAGTAAGAGAATTATGCAGTGC
TGCCATAACCATGAGTGATAACACTGCGGCCAACTTACTTCTGACAACGA
TCGGAGGACCGAAGGAGCTAACCGCTTTTTTGCACAACATGGGGGATCAT
GTAACTCGCCTTGATCGTTGGGAACCGGAGCTGAATGAAGCCATACCAAA
CGACGAGCGTGACACCACGATGCCTGTAGCAATGGCAACAACGTTGCGCA
AACTATTAACTGGCGAACTACTTACTCTAGCTTCCCGGCAACAATTAATA
GACTGGATGGAGGCGGATAAAGTTGCAGGACCACTTCTGCGCTCGGCCCT
TCCGGCTGGCTGGTTTATTGCTGATAAATCTGGAGCCGGTGAGCGTGGGT
CTCGCGGTATCATTGCAGCACTGGGGCCAGATGGTAAGCCCTCCCGTATC
GTAGTTATCTACACGACGGGGAGTCAGGCAACTATGGATGAACGAAATAG
ACAGATCGCTGAGATAGGTGCCTCACTGATTAAGCATTGGTAACTGTCAG
ACCAAGTTTACTCATATATACTTTAGATTGATTTAAAACTTCATTTTTAA
TTTAAAAGGATCTAGGTGAAGATCCTTTTTGATAATCTCATGACCAAAAT
CCCTTAACGTGAGTTTTCGTTCCACTGAGCGTCAGACCCCGTAGAAAAGA
TCAAAGGATCTTCTTGAGATCCTTTTTTTCTGCGCGTAATCTGCTGCTTG
CAAACAAAAAAACCACCGCTACCAGCGGTGGTTTGTTTGCCGGATCAAGA
GCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAAC
TCTGTAGCACCGCCTACATACCTCGCTCTGCTAATCCTGTTACCAGTGGC
TGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACA
CAGCCCAGCTTGGAGCGAACGACCTACACCGAACTGAGATACCTACAGCG
TGAGCTATGAGAAAGCGCCACGCTTCCCGAAGGGAGAAAGGCGGACAGGT
ATCCGGTAAGCGGCAGGGTCGGAACAGGAGAGCGCACGAGGGAGCTTCCA
GGGGGAAACGCCTGGTATCTTTATAGTCCTGTCGGGTTTCGCCACCTCTG
ACTTGAGCGTCGATTTTTGTGATGCTCGTCAGGGGGGCGGAGCCTATGGA
AAAACGCCAGCAACGCGGCCTTTTTACGGTTCCTGGCCTTTTGCTGGCCT
TTTGCTCACATGT

Nucleotide sequence of Eqr1-dsGFP-KCNA1 construct
(SEQ ID NO: 31) cgtctcgagctggccctccccacgcgggcgtccc
cgactcccgcgcgcgctcaggctcccagttgggaaccaaggagggggagg
atgggggggggggtgtgcgccgacccggaaacgccatataaggagcagga
aggatcccccgccggaacagaccttatttgggcagcgccttatatggagt
ggcccaatatggccctgccgcttccggctctgggaggaggggcgagcggg
ggttggggcgggggcaagctgggaactccaggcgcctggcccgggaggcc
actgctgctgttccaatactaggctttccaggagcctgagcgctcgcgat
gccggagcgggtcgcagggtggaggtgcccaccactcttggatgggaggg
cttcacgtcactccgggtcctcccggccggtccttccatattagggcttc
ctgcttcccatatatggccatgtacgtcacggcggaggcgggcccgtgct
gttccagacccttgaaatagaggccgattcggggagtcgcGAATTCGCTG
TCTGCGAGGGCCAGCTGTTGGGGTGAGTACTCCCTCTCAAAAGCGGGCAT
GACTTCTGCGCTAAGATTGTCAGTTTCCAAAAACGAGGAGGATTTGATAT
TCACCTGGCCCGCGGTGATGCCTTTGAGGGTGGCCGCGTCCATCTGGTCA
GAAAAGACAATCTTTTTGTTGTCAAGCTTGAGGTGTGGCAGGCTTGAGAT
CTGGCCATACACTTGAGTGACAATGACATCCACTTTGCCTTTCTCTCCAC
AGGTGTCCACTCCCAGGTCCAACTGCAGCCCAAGCGGAGGATCCGCCACC
atgcccgccatgaagatcgagtgccgcatcaccggcaccctgaacggcgt
ggagttcgagctggtgggcggcggagagggcacccccgaGCAGGGCCGCA
TGACCAACAAGATGAAGAGCACCAAAGGCGCCCTGACCTTCAGCCCCTAC
CTGCTGAGCCACGTGATGGGCTACGGCTTCTACCACTTCGGCACCTACCC
CAGCGGCTACGAGAACCCCTTCCTGCACGCCATCAACAACGGCGGCTACA
CCAACACCCGCATCGAGAAGTACGAGGACGGCGGCGTGCTGCACGTGAGC
TTCAGCTACCGCTACGAGGCCGGCCGCGTGATCGGCGACTTCAAGGTGGT
GGGCACCGGCTTCCCCGAGGACAGCGTGATCTTCACCGACAAGATCATCC
GCAGCAACGCCACCGTGGAGCACCTGCACCCCATGGGCGATAACGTGCTG
GTGGGCAGCTTCGCCCGCACCTTCAGCCTGCGCGACGGCGGCTACTACAG
CTTCGTGGTGGACAGCCACATGCACTTCAAGAGCGCCATCCACCCCAGCA
TCCTGCAGAACGGGGGCCCCATGTTCGCCTTCCGCCGCGTGGAGGAGCTG
CACAGCAACACCGAGCTGGGCATCGTGGAGTACCAGCACGCCTTCAAGAC
CCCCATCGCCTTCGCCAGATCTCGAGATATCAGCCATGGCTTCCCGCCGG
CGGTGGCGGCGCAGGATGATGGCACGCTGCCCATGTCTTGTGCCCAGGAG
AGCGGGATGGACCGTCACCCTGCAGCCTGTGCTTCTGCTAGGATCAATGT
GACCGGTGAGGGCAGAGGAAGTCTTCTAACATGCGGTGACGTGGAGGAGA
ATCCCGGCCCTATGACCGTGATGAGCGGCGAGAACGTGGACGAGGCCTCT
GCCGCTCCTGGACACCCTCAGGATGGCAGCTATCCCAGACAGGCCGACCA
CGACGATCACGAGTGCTGCGAGCGGGTCGTGATCAACATCAGCGGCCTGA
GATTCGAGACACAGCTGAAAACCCTGGCCCAGTTCCCCAACACCCTGCTG
GGCAACCCCAAGAAACGGATGCGGTACTTCGACCCCCTGCGGAACGAGTA
CTTCTTCGACCGGAACCGGCCCAGCTTCGACGCCATCCTGTACTACTACC
AGAGCGGCGGCAGACTGCGGAGGCCCGTGAATGTGCCCCTGGACATGTTC
AGCGAGGAAATCAAGTTCTACGAGCTGGGCGAGGAAGCCATGGAAAAGTT
CAGAGAGGACGAGGGCTTCATCAAAGAGGAAGAGAGGCCCCTGCCCGAGA
AAGAATACCAGAGACAAGTGTGGCTGCTGTTCGAGTACCCCGAGTCTAGC
GGCCCTGCCAGAGTGATCGCCATCGTGTCCGTGATGGTCATCCTGATCTC
TATCGTGATCTTCTGCCTGGAAACCCTGCCTGAGCTGAAGGACGACAAGG
ACTTCACCGGCACCGTGCACCGGATCGACAACACCACCGTGATCTACAAC
AGCAATATCTTCACCGACCCATTCTTCATCGTGGAAACACTGTGCATCAT
CTGGTTCAGCTTCGAGCTGGTCGTGCGGTTCTTCGCCTGCCCCAGCAAGA
CCGACTTCTTCAAGAACATCATGAACTTCATTGATATCGTGGCCATCATC
CCCTACTTCATCACCCTGGGCACCGAGATCGCCGAGCAGGAAGGCAATCA
GAAGGGCGAGCAGGCCACCAGCCTGGCCATTCTGAGAGTGATCAGACTCG
TGCGGGTGTTCCGGATCTTCAAGCTGAGCCGGCACAGCAAGGGCCTGCAG
ATCCTGGGCCAGACACTGAAGGCCAGCATGAGAGAGCTGGGCCTGCTGAT
CTTCTTTCTGTTCATCGGCGTGATCCTGTTCAGCAGCGCCGTGTACTTCG
CCGAGGCCGAAGAAGCCGAGAGCCACTTCAGCTCTATCCCCGACGCCTTT
TGGTGGGCCGTGGTGTCCATGACCACAGTGGGCTACGGCGACATGGTGCC
CGTGACAATCGGCGGCAAGATCGTGGGCAGCCTGTGTGCCATTGCCGGCG
TGCTGACAGTCGCCCTGCCTGTGCCTGTGATCGTGTCCAACTTCAACTAC
TTCTACCACCGGGAAACCGAGGGGGAGGAACAGGCTCAGCTGCTGCACGT
GTCCAGCCCCAATCTGGCCAGCGACAGCGACCTGAGCAGACGGTCTAGCA
GCACCATGAGCAAGAGCGAGTACATGGAAATCGAAGAGGACATGAACAAC
TCTATCGCCCACTACCGCCAAGTGAACATCCGGACCGCCAACTGCACCAC
CGCCAACCAGAACTGCGTGAACAAGAGCAAGCTGCTGACCGATGTCTGAg
TC

Nucleotide sequence of optimised AAV- Eqr1-dsGFP-K
CNA1 vector (SEQ ID NO: 32) cctgcaggcagctgcgcgctcg
ctcgctcactgaggccgcccgggcaaagcccgggcgtcgggcgacctttg
gtcgcccggcctcagtgagcgagcgagcgcgcagagagggagtggccaac
tccatcactaggggttcctgcggccgcacgcgtctcgagctggccctccc
cacgcgggcgtccccgactcccgcgcgcgctcaggctcccagttgggaac
caaggagggggaggatgggggggggggtgtgcgccgacccggaaacgcca
tataaggagcaggaaggatcccccgccggaacagaccttatttgggcagc
gccttatatggagtggcccaatatggccctgccgcttccggctctgggag
gaggggcgagcgggggttggggcgggggcaagctgggaactccaggcgcc
tggcccgggaggccactgctgctgttccaatactaggctttccaggagcc
tgagcgctcgcgatgccggagcgggtcgcagggtggaggtgcccaccact
cttggatgggagggcttcacgtcactccgggtcctcccggccggtccttc
catattagggcttcctgcttcccatatatggccatgtacgtcacggcgga
ggcgggcccgtgctgttccagacccttgaaatagaggccgattcggggag
tcgcGAATTCGCTGTCTGCGAGGGCCAGCTGTTGGGGTGAGTACTCCCTC
TCAAAAGCGGGCATGACTTCTGCGCTAAGATTGTCAGTTTCCAAAAACGA
GGAGGATTTGATATTCACCTGGCCCGCGGTGATGCCTTTGAGGGTGGCCG
CGTCCATCTGGTCAGAAAAGACAATCTTTTTGTTGTCAAGCTTGAGGTGT
GGCAGGCTTGAGATCTGGCCATACACTTGAGTGACAATGACATCCACTTT
GCCTTTCTCTCCACAGGTGTCCACTCCCAGGTCCAACTGCAGCCCAAGCG
GAGGATCCGCCACCatgcccgccatgaagatcgagtgccgcatcaccggc
accctgaacggcgtggagttcgagctggtgggcggcggagagggcacccc
cgaGCAGGGCCGCATGACCAACAAGATGAAGAGCACCAAAGGCGCCCTGA
CCTTCAGCCCCTACCTGCTGAGCCACGTGATGGGCTACGGCTTCTACCAC
TTCGGCACCTACCCCAGCGGCTACGAGAACCCCTTCCTGCACGCCATCAA
CAACGGCGGCTACACCAACACCCGCATCGAGAAGTACGAGGACGGCGGCG
TGCTGCACGTGAGCTTCAGCTACCGCTACGAGGCCGGCCGCGTGATCGGC
GACTTCAAGGTGGTGGGCACCGGCTTCCCCGAGGACAGCGTGATCTTCAC
CGACAAGATCATCCGCAGCAACGCCACCGTGGAGCACCTGCACCCCATGG
GCGATAACGTGCTGGTGGGCAGCTTCGCCCGCACCTTCAGCCTGCGCGAC
GGCGGCTACTACAGCTTCGTGGTGGACAGCCACATGCACTTCAAGAGCGC
CATCCACCCCAGCATCCTGCAGAACGGGGGCCCCATGTTCGCCTTCCGCC
GCGTGGAGGAGCTGCACAGCAACACCGAGCTGGGCATCGTGGAGTACCAG
CACGCCTTCAAGACCCCCATCGCCTTCGCCAGATCTCGAGATATCAGCCA
TGGCTTCCCGCCGGCGGTGGCGGCGCAGGATGATGGCACGCTGCCCATGT
CTTGTGCCCAGGAGAGCGGGATGGACCGTCACCCTGCAGCCTGTGCTTCT
GCTAGGATCAATGTGACCGGTGAGGGCAGAGGAAGTCTTCTAACATGCGG
TGACGTGGAGGAGAATCCCGGCCCTATGACCGTGATGAGCGGCGAGAACG
TGGACGAGGCCTCTGCCGCTCCTGGACACCCTCAGGATGGCAGCTATCCC
AGACAGGCCGACCACGACGATCACGAGTGCTGCGAGCGGGTCGTGATCAA
CATCAGCGGCCTGAGATTCGAGACACAGCTGAAAACCCTGGCCCAGTTCC
CCAACACCCTGCTGGGCAACCCCAAGAAACGGATGCGGTACTTCGACCCC
CTGCGGAACGAGTACTTCTTCGACCGGAACCGGCCCAGCTTCGACGCCAT
CCTGTACTACTACCAGAGCGGCGGCAGACTGCGGAGGCCCGTGAATGTGC
CCCTGGACATGTTCAGCGAGGAAATCAAGTTCTACGAGCTGGGCGAGGAA
GCCATGGAAAAGTTCAGAGAGGACGAGGGCTTCATCAAAGAGGAAGAGAG
GCCCCTGCCCGAGAAAGAATACCAGAGACAAGTGTGGCTGCTGTTCGAGT
ACCCCGAGTCTAGCGGCCCTGCCAGAGTGATCGCCATCGTGTCCGTGATG
GTCATCCTGATCTCTATCGTGATCTTCTGCCTGGAAACCCTGCCTGAGCT
GAAGGACGACAAGGACTTCACCGGCACCGTGCACCGGATCGACAACACCA
CCGTGATCTACAACAGCAATATCTTCACCGACCCATTCTTCATCGTGGAA
ACACTGTGCATCATCTGGTTCAGCTTCGAGCTGGTCGTGCGGTTCTTCGC
CTGCCCCAGCAAGACCGACTTCTTCAAGAACATCATGAACTTCATTGATA
TCGTGGCCATCATCCCCTACTTCATCACCCTGGGACCGAGATCGCCGAGC
AGGAAGGCAATCAGAAGGGCGAGCAGGCCACCAGCCTGGCCATTCTGAGA
GTGATCAGACTCGTGCGGGTGTTCCGGATCTTCAAGCTGAGCCGGCACAG
CAAGGGCCTGCAGATCCTGGGCCAGACACTGAAGGCCAGCATGAGAGAGC
TGGGCCTGCTGATCTTCTTTCTGTTCATCGGCGTGATCCTGTTCAGCAGC
GCCGTGTACTTCGCCGAGGCCGAAGAAGCCGAGAGCCACTTCAGCTCTAT
CCCCGACGCCTTTTGGTGGGCCGTGGTGTCCATGACCACAGTGGGCTACG
GCGACATGGTGCCCGTGACAATCGGCGGCAAGATCGTGGGCAGCCTGTGT
GCCATTGCCGGCGTGCTGACAGTCGCCCTGCCTGTGCCTGTGATCGTGTC
CAACTTCAACTACTTCTACCACCGGGAAACCGAGGGGGAGGAACAGGCTC
AGCTGCTGCACGTGTCCAGCCCCAATCTGGCCAGCGACAGCGACCTGAGC
AGACGGTCTAGCAGCACCATGAGCAAGAGCGAGTACATGGAAATCGAAGA
GGACATGAACAACTCTATCGCCCACTACCGCCAAGTGAACATCCGGACCG
CCAACTGCACCACCGCCAACCAGAACTGCGTGAACAAGAGCAAGCTGCTG
ACCGATGTCTGAgTCGACAATCAACCTCATcgataccgagcgctgctcga
gagatctacgggtggcatccctgtgacccctccccagtgcctctcctggc
cctggaagttgccactccagtgcccaccagccttgtcctaataaaattaa
gttgcatcattttgtctgactaggtgtccttctataatattatggggtgg
aggggggtggtatggagcaaggggcaagttgggaagacaacctgtagggc
ctgcggggtctattgggaaccaagctggagtgcagtggcacaatcttggc
tcactgcaatctccgcctcctgggttcaagcgattctcctgcctcagcct
cccgagttgttgggattccaggcatgcatgaccaggctcagctaattttt
gtttttttggtagagacggggtttcaccatattggccaggctggtctcca
actcctaatctcaggtgatctacccaccttggcctcccaaattgctggga
ttacaggcgtgaaccactgctcccttccctgtccttctgattttgtaggt
aaccacgtgcggaccgagcggccgcaggaacccctagtgatggagttggc
cactccctctctgcgcgctcgctcgctcactgaggccgggcgaccaaagg
tcgcccgacgcccgggctttgcccgggcggcctcagtgagcgagcgagcg
cgcagctgcctgcaggggcgcctgatgcggtattttctccttacgcatct
gtgcggtatttcacaccgcatacgtcaaagcaaccatagtacgcgccctg
tagcggcgcattaagcgcggcgggtgtggtggttacgcgcagcgtgaccg
ctacacttgccagcgccctagcgcccgctcctttcgctttcttcccttcc
tttctcgccacgttcgccggctttccccgtcaagctctaaatcgggggct
ccctttagggttccgatttagtgctttacggcacctcgaccccaaaaaac
ttgatttgggtgatggttcacgtagtgggccatcgccctgatagacggtt
tttcgccctttgacgttggagtccacgttctttaatagtggactcttgtt
ccaaactggaacaacactcaaccctatctcgggctattcttttgatttat
aagggattttgccgatttcggcctattggttaaaaaatgagctgatttaa
caaaaatttaacgcgaattttaacaaaatattaacgtttacaattttatg
gtgcactctcagtacaatctgctctgatgccgcatagttaagccagcccc
gacacccgccaacacccgctgacgcgccctgacgggcttgtctgctcccg
gcatccgcttacagacaagctgtgaccgtctccgggagctgcatgtgtca
gaggttttcaccgtcatcaccgaaacgcgcgagacgaaagggcctcgtga
tacgcctatttttataggttaatgtcatgataataatggtttcttagacg
tcaggtggcacttttcggggaaatgtgcgcggaacccctatttgtttatt
tttctaaatacattcaaatatgtatccgctcatgagacaataaccctgat
aaatgcttcaataatattgaaaaaggaagagtatgagtattcaacatttc
cgtgtcgcccttattcccttttttgcggcattttgccttcctgtttttgc
tcacccagaaacgctggtgaaagtaaaagatgctgaagatcagttgggtg
cacgagtgggttacatcgaactggatctcaacagcggtaagatccttgag
agttttcgccccgaagaacgttttccaatgatgagcacttttaaagttct
gctatgtggcgcggtattatcccgtattgacgccgggcaagagcaactcg
gtcgccgcatacactattctcagaatgacttggttgagtactcaccagtc
acagaaaagcatcttacggatggcatgacagtaagagaattatgcagtgc
tgccataaccatgagtgataacactgcggccaacttacttctgacaacga
tcggaggaccgaaggagctaaccgcttttttgcacaacatgggggatcat
gtaactcgccttgatcgttgggaaccggagctgaatgaagccataccaaa
cgacgagcgtgacaccacgatgcctgtagcaatggcaacaacgttgcgca
aactattaactggcgaactacttactctagcttcccggcaacaattaata
gactggatggaggcggataaagttgcaggaccacttctgcgctcggccct
tccggctggctggtttattgctgataaatctggagccggtgagcgtgggt
ctcgcggtatcattgcagcactggggccagatggtaagccctcccgtatc
gtagttatctacacgacggggagtcaggcaactatggatgaacgaaatag
acagatcgctgagataggtgcctcactgattaagcattggtaactgtcag
accaagtttactcatatatactttagattgatttaaaacttcatttttaa
tttaaaaggatctaggtgaagatcctttttgataatctcatgaccaaaat
cccttaacgtgagttttcgttccactgagcgtcagaccccgtagaaaaga
tcaaaggatcttcttgagatcctttttttctgcgcgtaatctgctgcttg
caaacaaaaaaaccaccgctaccagcggtggtttgtttgccggatcaaga
gctaccaactctttttccgaaggtaactggcttcagcagagcgcagatac
caaatactgtccttctagtgtagccgtagttaggccaccacttcaagaac
tctgtagcaccgcctacatacctcgctctgctaatcctgttaccagtggc
tgctgccagtggcgataagtcgtgtcttaccgggttggactcaagacgat
agttaccggataaggcgcagcggtcgggctgaacggggggttcgtgcaca
cagcccagcttggagcgaacgacctacaccgaactgagatacctacagcg
tgagctatgagaaagcgccacgcttcccgaagggagaaaggcggacaggt
atccggtaagcggcagggtcggaacaggagagcgcacgagggagcttcca
gggggaaacgcctggtatctttatagtcctgtcgggtttcgccacctctg
acttgagcgtcgatttttgtgatgctcgtcaggggggcggagcctatgga
aaaacgccagcaacgcggcctttttacggttcctggccttttgctggcct
tttgctcacatgt

Nucleotide sequence of Egr1-dsGFP-KCNJ2 construct
(SEQ ID NO: 33) ctggccctccccacgcgggcgtccccgactcccg
cgcgcgctcaggctcccagttgggaaccaaggagggggaggatggggggg
ggggtgtgcgccgacccggaaacgccatataaggagcaggaaggatcccc
cgccggaacagaccttatttgggcagcgccttatatggagtggcccaata
tggccctgccgcttccggctctgggaggaggggcgagcgggggttggggc
gggggcaagctgggaactccaggcgcctggcccgggaggccactgctgct
gttccaatactaggctttccaggagcctgagcgctcgcgatgccggagcg
ggtcgcagggtggaggtgcccaccactcttggatgggagggcttcacgtc
actccgggtcctcccggccggtccttccatattagggcttcctgcttccc
atatatggccatgtacgtcacggcggaggcgggcccgtgctgttccagac
ccttgaaatagaggccgattcggggagtcgcGAATTCGCTGTCTGCGAGG
GCCAGCTGTTGGGGTGAGTACTCCCTCTCAAAAGCGGGCATGACTTCTGC
GCTAAGATTGTCAGTTTCCAAAAACGAGGAGGATTTGATATTCACCTGGC
CCGCGGTGATGCCTTTGAGGGTGGCCGCGTCCATCTGGTCAGAAAAGACA
ATCTTTTTGTTGTCAAGCTTGAGGTGTGGCAGGCTTGAGATCTGGCCATA
CACTTGAGTGACAATGACATCCACTTTGCCTTTCTCTCCACAGGTGTCCA
CTCCCAGGTCCAACTGCAGCCCAAGCGGAGGATCCGCCACCatgcccgcc
atgaagatcgagtgccgcatcaccggcaccctgaacggcgtggagttcga
gctggtgggcggcggagagggcacccccgaGCAGGGCCGCATGACCAACA
AGATGAAGAGCACCAAAGGCGCCCTGACCTTCAGCCCCTACCTGCTGAGC
CACGTGATGGGCTACGGCTTCTACCACTTCGGCACCTACCCCAGCGGCTA
CGAGAACCCCTTCCTGCACGCCATCAACAACGGCGGCTACACCAACACCC
GCATCGAGAAGTACGAGGACGGCGGCGTGCTGCACGTGAGCTTCAGCTAC
CGCTACGAGGCCGGCCGCGTGATCGGCGACTTCAAGGTGGTGGGCACCGG
CTTCCCCGAGGACAGCGTGATCTTCACCGACAAGATCATCCGCAGCAACG
CCACCGTGGAGCACCTGCACCCCATGGGCGATAACGTGCTGGTGGGCAGC
TTCGCCCGCACCTTCAGCCTGCGCGACGGCGGCTACTACAGCTTCGTGGT
GGACAGCCACATGCACTTCAAGAGCGCCATCCACCCCAGCATCCTGCAGA
ACGGGGGCCCCATGTTCGCCTTCCGCCGCGTGGAGGAGCTGCACAGCAAC
ACCGAGCTGGGCATCGTGGAGTACCAGCACGCCTTCAAGACCCCCATCGC
CTTCGCCAGATCTCGAGATATCAGCCATGGCTTCCCGCCGGCGGTGGCGG
CGCAGGATGATGGCACGCTGCCCATGTCTTGTGCCCAGGAGAGCGGGATG
GACCGTCACCCTGCAGCCTGTGCTTCTGCTAGGATCAATGTGACCGGTGA
GGGCAGAGGAAGTCTTCTAACATGCGGTGACGTGGAGGAGAATCCCGGCC
CTatgggcagtgtgagaaccaaccgctacagcatcgtctcttcagaagaa
gacggtatgaagttggccaccatggcagttgcaaatggctttgggaacgg
gaagagtaaagtccacacccgacaacagtgcaggagccgctttgtgaaga
aagatggccactgtaatgttcagttcatcaatgtgggtgagaaggggcaa
cggtacctcgcagacatcttcaccacgtgtgtggacattcgctggcggtg
gatgctggttatcttctgcctggctttcgtcctgtcatggctgttttttg
gctgtgtgttttggttgatagctctgctccatggggacctggatgcatcc
aaagagggcaaagcttgtgtgtccgaggtcaacagcttcacggctgcctt
cctcttctccattgagacccagacaaccataggctatggtttcagatgtg
tcacggatgaatgcccaattgctgttttcatggtggtgttccagtcaatc
gtgggctgcatcatcgatgctttcatcattggcgcagtcatggccaagat
ggcaaagccaaagaagagaaacgagactcttgtcttcagtcacaatgccg
tgattgccatgagagacggcaagctgtgtttgatgtggcgagtgggcaat
cttcggaaaagccacttggtggaagctcatgttcgagcacagctcctcaa
atccagaattacttctgaaggggagtatatccctctggatcaaatagaca
tcaatgttgggtttgacagtggaatcgatcgtatatttctggtgtcccca
atcactatagtccatgaaatagatgaagacagtcctttatatgatttgag
taaacaggacattgacaacgcagactttgaaatcgtggtcatactggaag
gcatggtggaagccactgccatgacgacacagtgccgtagctcttatcta
gcaaatgaaatcctgtggggccaccgctatgagcctgtgctctttgaaga
gaagcactactacaaagtggactactccaggttccacaaaacttacgaag
tccccaacactcccctttgtagtgccagagacttagcagaaaagaaatat
atcctctcaaatgcaaattcattttgctatgaaaatgaagttgccctcac
aagcaaagaggaagacgacagtgaaaatggagttccagaaagcactagta
cggacacgccccctgacatagaccttcacaaccaggcaagtgtacctcta
gagcccaggcccttacggcgagagtcggagatatgagTCGACAATC

Nucleotide sequence of optimised AAV- Egr1-dsGFP-K
CNJ2 vector (SEQ ID NO: 34) cctgcaggcagctgcgcgctcg
ctcgctcactgaggccgcccgggcaaagcccgggcgtcgggcgacctttg
gtcgcccggcctcagtgagcgagcgagcgcgcagagagggagtggccaac
tccatcactaggggttcctgcggccgcacgcgtctcgagctggccctccc
cacgcgggcgtccccgactcccgcgcgcgctcaggctcccagttgggaac
caaggagggggaggatgggggggggggtgtgcgccgacccggaaacgcca
tataaggagcaggaaggatcccccgccggaacagaccttatttgggcagc
gccttatatggagtggcccaatatggccctgccgcttccggctctgggag
gaggggcgagcgggggttggggcgggggcaagctgggaactccaggcgcc
tggcccgggaggccactgctgctgttccaatactaggctttccaggagcc
tgagcgctcgcgatgccggagcgggtcgcagggtggaggtgcccaccact
cttggatgggagggcttcacgtcactccgggtcctcccggccggtccttc
catattagggcttcctgcttcccatatatggccatgtacgtcacggcgga
ggcgggcccgtgctgttccagacccttgaaatagaggccgattcggggag
tcgcGAATTCGCTGTCTGCGAGGGCCAGCTGTTGGGGTGAGTACTCCCTC
TCAAAAGCGGGCATGACTTCTGCGCTAAGATTGTCAGTTTCCAAAAACGA
GGAGGATTTGATATTCACCTGGCCCGCGGTGATGCCTTTGAGGGTGGCCG
CGTCCATCTGGTCAGAAAAGACAATCTTTTTGTTGTCAAGCTTGAGGTGT
GGCAGGCTTGAGATCTGGCCATACACTTGAGTGACAATGACATCCACTTT
GCCTTTCTCTCCACAGGTGTCCACTCCCAGGTCCAACTGCAGCCCAAGCG
GAGGATCCGCCACCatgcccgccatgaagatcgagtgccgcatcaccggc
accctgaacggcgtggagttcgagctggtgggcggcggagagggcacccc
cgaGCAGGGCCGCATGACCAACAAGATGAAGAGCACCAAAGGCGCCCTGA
CCTTCAGCCCCTACCTGCTGAGCCACGTGAGGGCTACGGCTTCTACCACT
TCGGCACCTACCCCAGCGGCTACGAGAACCCCTTCCTGCACGCCATCAAC
AACGGCGGCTACACCAACACCCGCATCGAGAAGTACGAGGACGGCGGCGT
GCTGCACGTGAGCTTCAGCTACCGCTACGAGGCCGGCCGCGTGATCGGCG
ACTTCAAGGTGGTGGGCACCGGCTTCCCCGAGGACAGCGTGATCTTCACC
GACAAGATCATCCGCAGCAACGCCACCGTGGAGCACCTGCACCCCATGGG
CGATAACGTGCTGGTGGGCAGCTTCGCCCGCACCTTCAGCCTGCGCGACG
GCGGCTACTACAGCTTCGTGGTGGACAGCCACATGCACTTCAAGAGCGCC
ATCCACCCCAGCATCCTGCAGAACGGGGGCCCCATGTTCGCCTTCCGCCG
CGTGGAGGAGCTGCACAGCAACACCGAGCTGGGCATCGTGGAGTACCAGC
ACGCCTTCAAGACCCCCATCGCCTTCGCCAGATCTCGAGATATCAGCCAT
GGCTTCCCGCCGGCGGTGGCGGCGCAGGATGATGGCACGCTGCCCATGTC
TTGTGCCCAGGAGAGCGGGATGGACCGTCACCCTGCAGCCTGTGCTTCTG
CTAGGATCAATGTGACCGGTGAGGGCAGAGGAAGTCTTCTAACATGCGGT
GACGTGGAGGAGAATCCCGGCCCTatgggcagtgtgagaaccaaccgcta
cagcatcgtctcttcagaagaagacggtatgaagttggccaccatggcag
ttgcaaatggctttgggaacgggaagagtaaagtccacacccgacaacag
tgcaggagccgctttgtgaagaaagatggccactgtaatgttcagttcat
caatgtgggtgagaaggggcaacggtacctcgcagacatcttcaccacgt
gtgtggacattcgctggcggtggatgctggttatcttctgcctggctttc
gtcctgtcatggctgttttttggctgtgtgttttggttgatagctctgct
ccatggggacctggatgcatccaaagagggcaaagcttgtgtgtccgagg
tcaacagcttcacggctgccttcctcttctccattgagacccagacaacc
ataggctatggtttcagatgtgtcacggatgaatgcccaattgctgtttt
catggtggtgttccagtcaatcgtgggctgcatcatcgatgctttcatca
ttggcgcagtcatggccaagatggcaaagccaaagaagagaaacgagact
cttgtcttcagtcacaatgccgtgattgccatgagagacggcaagctgtg
tttgatgtggcgagtgggcaatcttcggaaaagccacttggtggaagctc
atgttcgagcacagctcctcaaatccagaattacttctgaaggggagtat
atccctctggatcaaatagacatcaatgttgggtttgacagtggaatcga
tcgtatatttctggtgtccccaatcactatagtccatgaaatagatgaag
acagtcctttatatgatttgagtaaacaggacattgacaacgcagacttt
gaaatcgtggtcatactggaaggcatggtggaagccactgccatgacgac
acagtgccgtagctcttatctagcaaatgaaatcctgtggggccaccgct
atgagcctgtgctctttgaagagaagcactactacaaagtggactactcc
aggttccacaaaacttacgaagtccccaacactcccctttgtagtgccag
agacttagcagaaaagaaatatatcctctcaaatgcaaattcattttgct
atgaaaatgaagttgccctcacaagcaaagaggaagacgacagtgaaaat
ggagttccagaaagcactagtacggacacgccccctgacatagaccttca
caaccaggcaagtgtacctctagagcccaggcccttacggcgagagtcgg
agatatgagTCGACAATCAACCTCATcgataccgagcgctgctcgagaga
tctacgggtggcatccctgtgacccctccccagtgcctctcctggccctg
gaagttgccactccagtgcccaccagccttgtcctaataaaattaagttg
catcattttgtctgactaggtgtccttctataatattatggggtggaggg
gggtggtatggagcaaggggcaagttgggaagacaacctgtagggcctgc
ggggtctattgggaaccaagctggagtgcagtggcacaatcttggctcac
tgcaatctccgcctcctgggttcaagcgattctcctgcctcagcctcccg
agttgttgggattccaggcatgcatgaccaggctcagctaatttttgttt
ttttggtagagacggggtttcaccatattggccaggctggtctccaactc
ctaatctcaggtgatctacccaccttggcctcccaaattgctgggattac
aggcgtgaaccactgctcccttccctgtccttctgattttgtaggtaacc
acgtgcggaccgagcggccgcaggaacccctagtgatggagttggccact
ccctctctgcgcgctcgctcgctcactgaggccgggcgaccaaaggtcgc
ccgacgcccgggctttgcccgggcggcctcagtgagcgagcgagcgcgca
gctgcctgcaggggcgcctgatgcggtattttctccttacgcatctgtgc
ggtatttcacaccgcatacgtcaaagcaaccatagtacgcgccctgtagc
ggcgcattaagcgcggcgggtgtggtggttacgcgcagcgtgaccgctac
acttgccagcgccctagcgcccgctcctttcgctttcttcccttcctttc
tcgccacgttcgccggctttccccgtcaagctctaaatcgggggctccct
ttagggttccgatttagtgctttacggcacctcgaccccaaaaaacttga
tttgggtgatggttcacgtagtgggccatcgccctgatagacggtttttc
gccctttgacgttggagtccacgttctttaatagtggactcttgttccaa
actggaacaacactcaaccctatctcgggctattcttttgatttataagg
gattttgccgatttcggcctattggttaaaaaatgagctgatttaacaaa
aatttaacgcgaattttaacaaaatattaacgtttacaattttatggtgc
actctcagtacaatctgctctgatgccgcatagttaagccagccccgaca
cccgccaacacccgctgacgcgccctgacgggcttgtctgctcccggcat
ccgcttacagacaagctgtgaccgtctccgggagctgcatgtgtcagagg
ttttcaccgtcatcaccgaaacgcgcgagacgaaagggcctcgtgatacg
cctatttttataggttaatgtcatgataataatggtttcttagacgtcag
gtggcacttttcggggaaatgtgcgcggaacccctatttgtttatttttc
taaatacattcaaatatgtatccgctcatgagacaataaccctgataaat
gcttcaataatattgaaaaaggaagagtatgagtattcaacatttccgtg
tcgcccttattcccttttttgcggcattttgccttcctgtttttgctcac
ccagaaacgctggtgaaagtaaaagatgctgaagatcagttgggtgcacg
agtgggttacatcgaactggatctcaacagcggtaagatccttgagagtt
ttcgccccgaagaacgttttccaatgatgagcacttttaaagttctgcta
tgtggcgcggtattatcccgtattgacgccgggcaagagcaactcggtcg
ccgcatacactattctcagaatgacttggttgagtactcaccagtcacag
aaaagcatcttacggatggcatgacagtaagagaattatgcagtgctgcc
ataaccatgagtgataacactgcggccaacttacttctgacaacgatcgg
aggaccgaaggagctaaccgcttttttgcacaacatgggggatcatgtaa
ctcgccttgatcgttgggaaccggagctgaatgaagccataccaaacgac
gagcgtgacaccacgatgcctgtagcaatggcaacaacgttgcgcaaact
attaactggcgaactacttactctagcttcccggcaacaattaatagact
ggatggaggcggataaagttgcaggaccacttctgcgctcggcccttccg
gctggctggtttattgctgataaatctggagccggtgagcgtgggtctcg
cggtatcattgcagcactggggccagatggtaagccctcccgtatcgtag
ttatctacacgacggggagtcaggcaactatggatgaacgaaatagacag
atcgctgagataggtgcctcactgattaagcattggtaactgtcagacca
agtttactcatatatactttagattgatttaaaacttcatttttaattta
aaaggatctaggtgaagatcctttttgataatctcatgaccaaaatccct
taacgtgagttttcgttccactgagcgtcagaccccgtagaaaagatcaa
aggatcttcttgagatcctttttttctgcgcgtaatctgctgcttgcaaa
caaaaaaaccaccgctaccagcggtggtttgtttgccggatcaagagcta
ccaactctttttccgaaggtaactggcttcagcagagcgcagataccaaa
tactgtccttctagtgtagccgtagttaggccaccacttcaagaactctg
tagcaccgcctacatacctcgctctgctaatcctgttaccagtggctgct
gccagtggcgataagtcgtgtcttaccgggttggactcaagacgatagtt
accggataaggcgcagcggtcgggctgaacggggggttcgtgcacacagc
ccagcttggagcgaacgacctacaccgaactgagatacctacagcgtgag
ctatgagaaagcgccacgcttcccgaagggagaaaggcggacaggtatcc
ggtaagcggcagggtcggaacaggagagcgcacgagggagcttccagggg
gaaacgcctggtatctttatagtcctgtcgggtttcgccacctctgactt
gagcgtcgatttttgtgatgctcgtcaggggggcggagcctatggaaaaa
cgccagcaacgcggcctttttacggttcctggccttttgctggccttttg
ctcacatgt

Nucleotide sequence of Tet-On-dCAS9VP64 construct
(SEQ ID NO: 35) ATCAGTGATAGAGAAAAGTGAAAGTCGAGTTTAC
CACTCCCTATCAGTGATAGAGAAAAGTGAAAGTCGAGTTTACCACTCCCT
ATCAGTGATAGAGAAAAGTGAAAGTCGAGTTTACCACTCCCTATCAGTGA
TAGAGAAAAGTGAAAGTCGAGTTTACCACTCCCTATCAGTGATAGAGAAA
AGTGAAAGTCGAGTTTACCACTCCCTATCAGTGATAGAGAAAAGTGAAAG
TCGAGTTTACCACTCCCTATCAGTGATAGAGAAAAGTGAAAGTCGAGCTC
GGTACCCACCGGTGTCGACTCTAGAgccaccATGCCCAAGAAGAAGAGGA
AGGTGGGAAGGGGGATGGACAAGAAGTACTCCATTGGGCTCGCTATCGGC
ACAAACAGCGTCGGCTGGGCCGTCATTACGGACGAGTACAAGGTGCCGAG
CAAAAAATTCAAAGTTCTGGGCAATACCGATCGCCACAGCATAAAGAAGA
ACCTCATTGGCGCCCTCCTGTTCGACTCCGGGGAGACGGCCGAAGCCACG
CGGCTCAAAAGAACAGCACGGCGCAGATATACCCGCAGAAAGAATCGGAT
CTGCTACCTGCAGGAGATCTTTAGTAATGAGATGGCTAAGGTGGATGACT
CTTTCTTCCATAGGCTGGAGGAGTCCTTTTTGGTGGAGGAGGATAAAAAG
CACGAGCGCCACCCAATCTTTGGCAATATCGTGGACGAGGTGGCGTACCA
TGAAAAGTACCCAACCATATATCATCTGAGGAAGAAGCTTGTAGACAGTA
CTGATAAGGCTGACTTGCGGTTGATCTATCTCGCGCTGGCGCATATGATC
AAATTTCGGGGACACTTCCTCATCGAGGGGGACCTGAACCCAGACAACAG
CGATGTCGACAAACTCTTTATCCAACTGGTTCAGACTTACAATCAGCTTT
TCGAAGAGAACCCGATCAACGCATCCGGAGTTGACGCCAAAGCAATCCTG
AGCGCTAGGCTGTCCAAATCCCGGCGGCTCGAAAACCTCATCGCACAGCT
CCCTGGGGAGAAGAAGAACGGCCTGTTTGGTAATCTTATCGCCCTGTCAC
TCGGGCTGACCCCCAACTTTAAATCTAACTTCGACCTGGCCGAAGATGCC
AAGCTTCAACTGAGCAAAGACACCTACGATGATGATCTCGACAATCTGCT
GGCCCAGATCGGCGACCAGTACGCAGACCTTTTTTTGGCGGCAAAGAACC
TGTCAGACGCCATTCTGCTGAGTGATATTCTGCGAGTGAACACGGAGATC
ACCAAAGCTCCGCTGAGCGCTAGTATGATCAAGCGCTATGATGAGCACCA
CCAAGACTTGACTTTGCTGAAGGCCCTTGTCAGACAGCAACTGCCTGAGA
AGTACAAGGAAATTTTCTTCGATCAGTCTAAAAATGGCTACGCCGGATAC
ATTGACGGCGGAGCAAGCCAGGAGGAATTTTACAAATTTATTAAGCCCAT
CTTGGAAAAAATGGACGGCACCGAGGAGCTGCTGGTAAAGCTTAACAGAG
AAGATCTGTTGCGCAAACAGCGCACTTTCGACAATGGAAGCATCCCCCAC
CAGATTCACCTGGGCGAACTGCACGCTATCCTCAGGCGGCAAGAGGATTT
CTACCCCTTTTTGAAAGATAACAGGGAAAAGATTGAGAAAATCCTCACAT
TTCGGATACCCTACTATGTAGGCCCCCTCGCCCGGGGAAATTCCAGATTC
GCGTGGATGACTCGCAAATCAGAAGAGACCATCACTCCCTGGAACTTCGA
GGAAGTCGTGGATAAGGGGGCCTCTGCCCAGTCCTTCATCGAAAGGATGA
CTAACTTTGATAAAAATCTGCCTAACGAAAAGGTGCTTCCTAAACACTCT
CTGCTGTACGAGTACTTCACAGTTTATAACGAGCTCACCAAGGTCAAATA
CGTCACAGAAGGGATGAGAAAGCCAGCATTCCTGTCTGGAGAGCAGAAGA
AAGCTATCGTGGACCTCCTCTTCAAGACGAACCGGAAAGTTACCGTGAAA
CAGCTCAAAGAAGACTATTTCAAAAAGATTGAATGTTTCGACTCTGTTGA
AATCAGCGGAGTGGAGGATCGCTTCAACGCATCCCTGGGAACGTATCACG
ATCTCCTGAAAATCATTAAAGACAAGGACTTCCTGGACAATGAGGAGAAC
GAGGACATTCTTGAGGACATTGTCCTCACCCTTACGTTGTTTGAAGATAG
GGAGATGATTGAAGAACGCTTGAAAACTTACGCTCATCTCTTCGACGACA
AAGTCATGAAACAGCTCAAGAGGCGCCGATATACAGGATGGGGGCGGCTG
TCAAGAAAACTGATCAATGGGATCCGAGACAAGCAGAGTGGAAAGACAAT
CCTGGATTTTCTTAAGTCCGATGGATTTGCCAACCGGAACTTCATGCAGT
TGATCCATGATGACTCTCTCACCTTTAAGGAGGACATCCAGAAAGCACAA
GTTTCTGGCCAGGGGGACAGTCTTCACGAGCACATCGCTAATCTTGCAGG
TAGCCCAGCTATCAAAAAGGGAATACTGCAGACCGTTAAGGTCGTGGATG
AACTCGTCAAAGTAATGGGAAGGCATAAGCCCGAGAATATCGTTATCGAG
ATGGCCCGAGAGAACCAAACTACCCAGAAGGGACAGAAGAACAGTAGGGA
AAGGATGAAGAGGATTGAAGAGGGTATAAAAGAACTGGGGTCCCAAATCC
TTAAGGAACACCCAGTTGAAAACACCCAGCTTCAGAATGAGAAGCTCTAC
CTGTACTACCTGCAGAACGGCAGGGACATGTACGTGGATCAGGAACTGGA
CATCAATCGGCTCTCCGACTACGACGTGGCTGCTATCGTGCCCCAGTCTT
TTCTCAAAGATGATTCTATTGATAATAAAGTGTTGACAAGATCCGATAAA
gcTAGAGGGAAGAGTGATAACGTCCCCTCAGAAGAAGTTGTCAAGAAAAT
GAAAAATTATTGGCGGCAGCTGCTGAACGCCAAACTGATCACACAACGGA
AGTTCGATAATCTGACTAAGGCTGAACGAGGTGGCCTGTCTGAGTTGGAT
AAAGCCGGCTTCATCAAAAGGCAGCTTGTTGAGACACGCCAGATCACCAA
GCACGTGGCCCAAATTCTCGATTCACGCATGAACACCAAGTACGATGAAA
ATGACAAACTGATTCGAGAGGTGAAAGTTATTACTCTGAAGTCTAAGCTG
GTCTCAGATTTCAGAAAGGACTTTCAGTTTTATAAGGTGAGAGAGATCAA
CAATTACCACCATGCGCATGATGCCTACCTGAATGCAGTGGTAGGCACTG
CACTTATCAAAAAATATCCCAAGCTTGAATCTGAATTTGTTTACGGAGAC
TATAAAGTGTACGATGTTAGGAAAATGATCGCAAAGTCTGAGCAGGAAAT
AGGCAAGGCCACCGCTAAGTACTTCTTTTACAGCAATATTATGAATTTTT
TCAAGACCGAGATTACACTGGCCAATGGAGAGATTCGGAAGCGACCACTT
ATCGAAACAAACGGAGAAACAGGAGAAATCGTGTGGGACAAGGGTAGGGA
TTTCGCGACAGTCCGGAAGGTCCTGTCCATGCCGCAGGTGAACATCGTTA
AAAAGACCGAAGTACAGACCGGAGGCTTCTCCAAGGAAAGTATCCTCCCG
AAAAGGAACAGCGACAAGCTGATCGCACGCAAAAAAGATTGGGACCCCAA
GAAATACGGCGGATTCGATTCTCCTACAGTCGCTTACAGTGTACTGGTTG
TGGCCAAAGTGGAGAAAGGGAAGTCTAAAAAACTCAAAAGCGTCAAGGAA
CTGCTGGGCATCACAATCATGGAGCGATCAAGCTTCGAAAAAAACCCCAT
CGACTTTCTCGAGGCGAAAGGATATAAAGAGGTCAAAAAAGACCTCATCA
TTAAGCTTCCCAAGTACTCTCTCTTTGAGCTTGAAAACGGCCGGAAACGA
ATGCTCGCTAGTGCGGGCGAGCTGCAGAAAGGTAACGAGCTGGCACTGCC
CTCTAAATACGTTAATTTCTTGTATCTGGCCAGCCACTATGAAAAGCTCA
AAGGGTCTCCCGAAGATAATGAGCAGAAGCAGCTGTTCGTGGAACAACAC
AAACACTACCTTGATGAGATCATCGAGCAAATAAGCGAATTCTCCAAAAG
AGTGATCCTCGCCGACGCTAACCTCGATAAGGTGCTTTCTGCTTACAATA
AGCACAGGGATAAGCCCATCAGGGAGCAGGCAGAAAACATTATCCACTTG
TTTACTCTGACCAACTTGGGCGCGCCTGCAGCCTTCAAGTACTTCGACAC
CACCATAGACAGAAAGCGGTACACCTCTACAAAGGAGGTCCTGGACGCCA
CACTGATTCATCAGTCAATTACGGGGCTCTATGAAACAAGAATCGACCTC
TCTCAGCTCGGTGGAGACAGCAGGGCTGACCCCAAGAAGAAGAGGAAGGT
GGAGGCCAGCGGTTCCGGACGGGCTGACGCATTGGACGATTTTGATCTGG
ATATGCTGGGAAGTGACGCCCTCGATGATTTTGACCTTGACATGCTTGGT
TCGGATGCCCTTGATGACTTTGACCTCGACATGCTCGGCAGTGACGCCCT
TGATGATTTCGACCTGGACATGCTGATTAACTCT

Nucleotide sequence of optimised AAV- Tet-On-dCAS9
VP64 vector (SEQ ID NO: 36) cctgcaggcagctgcgcgctcg
ctcgctcactgaggccgcccgggcaaagcccgggcgtcgggcgacctttg
gtcgcccggcctcagtgagcgagcgagcgcgcagagagggagtggccaac
tccatcactaggggttcctgcggcctCTAGACCAGTTTGGTTAGATCTCG
AGTTTACCACTCCCTATCAGTGATAGAGAAAAGTGAAAGTCGAGTTTACC
ACTCCCTATCAGTGATAGAGAAAAGTGAAAGTCGAGTTTACCACTCCCTA
TCAGTGATAGAGAAAAGTGAAAGTCGAGTTTACCACTCCCTATCAGTGAT
AGAGAAAAGTGAAAGTCGAGTTTACCACTCCCTATCAGTGATAGAGAAAA
GTGAAAGTCGAGTTTACCACTCCCTATCAGTGATAGAGAAAAGTGAAAGT
CGAGTTTACCACTCCCTATCAGTGATAGAGAAAAGTGAAAGTCGAGCTCG
GTACCCACCGGTGTCGACTCTAGAgccaccATGCCCAAGAAGAAGAGGAA
GGTGGGAAGGGGGATGGACAAGAAGTACTCCATTGGGCTCGCTATCGGCA
CAAACAGCGTCGGCTGGGCCGTCATTACGGACGAGTACAAGGTGCCGAGC
AAAAAATTCAAAGTTCTGGGCAATACCGATCGCCACAGCATAAAGAAGAA
CCTCATTGGCGCCCTCCTGTTCGACTCCGGGGAGACGGCCGAAGCCACGC
GGCTCAAAAGAACAGCACGGCGCAGATATACCCGCAGAAAGAATCGGATC
TGCTACCTGCAGGAGATCTTTAGTAATGAGATGGCTAAGGTGGATGACTC
TTTCTTCCATAGGCTGGAGGAGTCCTTTTTGGTGGAGGAGGATAAAAAGC
ACGAGCGCCACCCAATCTTTGGCAATATCGTGGACGAGGTGGCGTACCAT
GAAAAGTACCCAACCATATATCATCTGAGGAAGAAGCTTGTAGACAGTAC
TGATAAGGCTGACTTGCGGTTGATCTATCTCGCGCTGGCGCATATGATCA
AATTTCGGGGACACTTCCTCATCGAGGGGGACCTGAACCCAGACAACAGC
GATGTCGACAAACTCTTTATCCAACTGGTTCAGACTTACAATCAGCTTTT
CGAAGAGAACCCGATCAACGCATCCGGAGTTGACGCCAAAGCAATCCTGA
GCGCTAGGCTGTCCAAATCCCGGCGGCTCGAAAACCTCATCGCACAGCTC
CCTGGGGAGAAGAAGAACGGCCTGTTTGGTAATCTTATCGCCCTGTCACT
CGGGCTGACCCCCAACTTTAAATCTAACTTCGACCTGGCCGAAGATGCCA
AGCTTCAACTGAGCAAAGACACCTACGATGATGATCTCGACAATCTGCTG
GCCCAGATCGGCGACCAGTACGCAGACCTTTTTTTGGCGGCAAAGAACCT
GTCAGACGCCATTCTGCTGAGTGATATTCTGCGAGTGAACACGGAGATCA
CCAAAGCTCCGCTGAGCGCTAGTATGATCAAGCGCTATGATGAGCACCAC
CAAGACTTGACTTTGCTGAAGGCCCTTGTCAGACAGCAACTGCCTGAGAA
GTACAAGGAAATTTTCTTCGATCAGTCTAAAAATGGCTACGCCGGATACA
TTGACGGCGGAGCAAGCCAGGAGGAATTTTACAAATTTATTAAGCCCATC
TTGGAAAAAATGGACGGCACCGAGGAGCTGCTGGTAAAGCTTAACAGAGA
AGATCTGTTGCGCAAACAGCGCACTTTCGACAATGGAAGCATCCCCCACC
AGATTCACCTGGGCGAACTGCACGCTATCCTCAGGCGGCAAGAGGATTTC
TACCCCTTTTTGAAAGATAACAGGGAAAAGATTGAGAAAATCCTCACATT
TCGGATACCCTACTATGTAGGCCCCCTCGCCCGGGGAAATTCCAGATTCG
CGTGGATGACTCGCAAATCAGAAGAGACCATCACTCCCTGGAACTTCGAG
GAAGTCGTGGATAAGGGGGCCTCTGCCCAGTCCTTCATCGAAAGGATGAC
TAACTTTGATAAAAATCTGCCTAACGAAAAGGTGCTTCCTAAACACTCTC
TGCTGTACGAGTACTTCACAGTTTATAACGAGCTCACCAAGGTCAAATAC
GTCACAGAAGGGATGAGAAAGCCAGCATTCCTGTCTGGAGAGCAGAAGAA
AGCTATCGTGGACCTCCTCTTCAAGACGAACCGGAAAGTTACCGTGAAAC
AGCTCAAAGAAGACTATTTCAAAAAGATTGAATGTTTCGACTCTGTTGAA
ATCAGCGGAGTGGAGGATCGCTTCAACGCATCCCTGGGAACGTATCACGA
TCTCCTGAAAATCATTAAAGACAAGGACTTCCTGGACAATGAGGAGAACG
AGGACATTCTTGAGGACATTGTCCTCACCCTTACGTTGTTTGAAGATAGG
GAGATGATTGAAGAACGCTTGAAAACTTACGCTCATCTCTTCGACGACAA
AGTCATGAAACAGCTCAAGAGGCGCCGATATACAGGATGGGGGCGGCTGT
CAAGAAAACTGATCAATGGGATCCGAGACAAGCAGAGTGGAAAGACAATC
CTGGATTTTCTTAAGTCCGATGGATTTGCCAACCGGAACTTCATGCAGTT
GATCCATGATGACTCTCTCACCTTTAAGGAGGACATCCAGAAAGCACAAG
TTTCTGGCCAGGGGGACAGTCTTCACGAGCACATCGCTAATCTTGCAGGT
AGCCCAGCTATCAAAAAGGGAATACTGCAGACCGTTAAGGTCGTGGATGA
ACTCGTCAAAGTAATGGGAAGGCATAAGCCCGAGAATATCGTTATCGAGA
TGGCCCGAGAGAACCAAACTACCCAGAAGGGACAGAAGAACAGTAGGGAA
AGGATGAAGAGGATTGAAGAGGGTATAAAAGAACTGGGGTCCCAAATCCT
TAAGGAACACCCAGTTGAAAACACCCAGCTTCAGAATGAGAAGCTCTACC
TGTACTACCTGCAGAACGGCAGGGACATGTACGTGGATCAGGAACTGGAC
ATCAATCGGCTCTCCGACTACGACGTGGCTGCTATCGTGCCCCAGTCTTT
TCTCAAAGATGATTCTATTGATAATAAAGTGTTGACAAGATCCGATAAAg
cTAGAGGGAAGAGTGATAACGTCCCCTCAGAAGAAGTTGTCAAGAAAATG
AAAAATTATTGGCGGCAGCTGCTGAACGCCAAACTGATCACACAACGGAA
GTTCGATAATCTGACTAAGGCTGAACGAGGTGGCCTGTCTGAGTTGGATA
AAGCCGGCTTCATCAAAAGGCAGCTTGTTGAGACACGCCAGATCACCAAG
CACGTGGCCCAAATTCTCGATTCACGCATGAACACCAAGTACGATGAAAA
TGACAAACTGATTCGAGAGGTGAAAGTTATTACTCTGAAGTCTAAGCTGG
TCTCAGATTTCAGAAAGGACTTTCAGTTTTATAAGGTGAGAGAGATCAAC
AATTACCACCATGCGCATGATGCCTACCTGAATGCAGTGGTAGGCACTGC
ACTTATCAAAAAATATCCCAAGCTTGAATCTGAATTTGTTTACGGAGACT
ATAAAGTGTACGATGTTAGGAAAATGATCGCAAAGTCTGAGCAGGAAATA
GGCAAGGCCACCGCTAAGTACTTCTTTTACAGCAATATTATGAATTTTTT
CAAGACCGAGATTACACTGGCCAATGGAGAGATTCGGAAGCGACCACTTA
TCGAAACAAACGGAGAAACAGGAGAAATCGTGTGGGACAAGGGTAGGGAT
TTCGCGACAGTCCGGAAGGTCCTGTCCATGCCGCAGGTGAACATCGTTAA
AAAGACCGAAGTACAGACCGGAGGCTTCTCCAAGGAAAGTATCCTCCCGA
AAAGGAACAGCGACAAGCTGATCGCACGCAAAAAAGATTGGGACCCCAAG
AAATACGGCGGATTCGATTCTCCTACAGTCGCTTACAGTGTACTGGTTGT
GGCCAAAGTGGAGAAAGGGAAGTCTAAAAAACTCAAAAGCGTCAAGGAAC
TGCTGGGCATCACAATCATGGAGCGATCAAGCTTCGAAAAAAACCCCATC
GACTTTCTCGAGGCGAAAGGATATAAAGAGGTCAAAAAAGACCTCATCAT
TAAGCTTCCCAAGTACTCTCTCTTTGAGCTTGAAAACGGCCGGAAACGAA
TGCTCGCTAGTGCGGGCGAGCTGCAGAAAGGTAACGAGCTGGCACTGCCC
TCTAAATACGTTAATTTCTTGTATCTGGCCAGCCACTATGAAAAGCTCAA
AGGGTCTCCCGAAGATAATGAGCAGAAGCAGCTGTTCGTGGAACAACACA
AACACTACCTTGATGAGATCATCGAGCAAATAAGCGAATTCTCCAAAAGA
GTGATCCTCGCCGACGCTAACCTCGATAAGGTGCTTTCTGCTTACAATAA
GCACAGGGATAAGCCCATCAGGGAGCAGGCAGAAAACATTATCCACTTGT
TTACTCTGACCAACTTGGGCGCGCCTGCAGCCTTCAAGTACTTCGACACC
ACCATAGACAGAAAGCGGTACACCTCTACAAAGGAGGTCCTGGACGCCAC
ACTGATTCATCAGTCAATTACGGGGCTCTATGAAACAAGAATCGACCTCT
CTCAGCTCGGTGGAGACAGCAGGGCTGACCCCAAGAAGAAGAGGAAGGTG
GAGGCCAGCGGTTCCGGACGGGCTGACGCATTGGACGATTTTGATCTGGA
TATGCTGGGAAGTGACGCCCTCGATGATTTTGACCTTGACATGCTTGGTT
CGGATGCCCTTGATGACTTTGACCTCGACATGCTCGGCAGTGACGCCCTT
GATGATTTCGACCTGGACATGCTGATTAACTCTAGATAAGaattcAATAA
AAGATCTTTATTTTCATTAGATCTGTGTGTTGGTTTTTTGTGTgcggccg
caggaacccctagtgatggagttggccactccctctctgcgcgctcgctc
gctcactgaggccgggcgaccaaaggtcgcccgacgcccgggctttgccc
gggcggcctcagtgagcgagcgagcgcgcagctgcctgcaggggcgcctg
atgcggtattttctccttacgcatctgtgcggtatttcacaccgcatacg
tcaaagcaaccatagtacgcgccctgtagcggcgcattaagcgcggcggg
tgtggtggttacgcgcagcgtgaccgctacacttgccagcgccctagcgc
ccgctctttcgctttcttcccttcctttctcgccacgttcgccggctttc
cccgtcaagctctaaatcgggggctccctttagggttccgatttagtgct
ttacggcacctcgaccccaaaaaacttgatttgggtgatggttcacgtag
tgggccatcgccctgatagacggtttttcgccctttgacgttggagtcca
cgttctttaatagtggactcttgttccaaactggaacaacactcaaccct
atctcgggctattcttttgatttataagggattttgccgatttcggccta
ttggttaaaaaatgagctgatttaacaaaaatttaacgcgaattttaaca
aaatattaacgtttacaattttatggtgcactctcagtacaatctgctct
gatgccgcatagttaagccagccccgacacccgccaacacccgctgacgc
gccctgacgggcttgtctgctcccggcatccgcttacagacaagctgtga
ccgtctccgggagctgcatgtgtcagaggttttcaccgtcatcaccgaaa
cgcgcgagacgaaagggcctcgtgatacgcctatttttataggttaatgt
catgataataatggtttcttagacgtcaggtggcacttttcggggaaatg
tgcgcggaacccctatttgtttatttttctaaatacattcaaatatgtat
ccgctcatgagacaataaccctgataaatgcttcaataatattgaaaaag
gaagagtatgagtattcaacatttccgtgtcgcccttattcccttttttg
cggcattttgccttcctgtttttgctcacccagaaacgctggtgaaagta
aaagatgctgaagatcagttgggtgcacgagtgggttacatcgaactgga
tctcaacagcggtaagatccttgagagttttcgccccgaagaacgttttc
caatgatgagcacttttaaagttctgctatgtggcgcggtattatcccgt
attgacgccgggcaagagcaactcggtcgccgcatacactattctcagaa
tgacttggttgagtactcaccagtcacagaaaagcatcttacggatggca
tgacagtaagagaattatgcagtgctgccataaccatgagtgataacact
gcggccaacttacttctgacaacgatcggaggaccgaaggagctaaccgc
ttttttgcacaacatgggggatcatgtaactcgccttgatcgttgggaac
cggagctgaatgaagccataccaaacgacgagcgtgacaccacgatgcct
gtagcaatggcaacaacgttgcgcaaactattaactggcgaactacttac
tctagcttcccggcaacaattaatagactggatggaggcggataaagttg
caggaccacttctgcgctcggcccttccggctggctggtttattgctgat
aaatctggagccggtgagcgtggaagccgcggtatcattgcagcactggg
gccagatggtaagccctcccgtatcgtagttatctacacgacggggagtc
aggcaactatggatgaacgaaatagacagatcgctgagataggtgcctca
ctgattaagcattggtaactgtcagaccaagtttactcatatatacttta
gattgatttaaaacttcatttttaatttaaaaggatctaggtgaagatcc
tttttgataatctcatgaccaaaatcccttaacgtgagttttcgttccac
tgagcgtcagaccccgtagaaaagatcaaaggatcttcttgagatccttt
ttttctgcgcgtaatctgctgcttgcaaacaaaaaaaccaccgctaccag
cggtggtttgtttgccggatcaagagctaccaactctttttccgaaggta
actggcttcagcagagcgcagataccaaatactgtccttctagtgtagcc
gtagttaggccaccacttcaagaactctgtagcaccgcctacatacctcg
ctctgctaatcctgttaccagtggctgctgccagtggcgataagtcgtgt
cttaccgggttggactcaagacgatagttaccggataaggcgcagcggtc
gggctgaacggggggttcgtgcacacagcccagcttggagcgaacgacct
acaccgaactgagatacctacagcgtgagctatgagaaagcgccacgctt
cccgaagggagaaaggcggacaggtatccggtaagcggcagggtcggaac
aggagagcgcacgagggagcttccagggggaaacgcctggtatctttata
gtcctgtcgggtttcgccacctctgacttgagcgtcgatttttgtgatgc
tcgtcaggggggcggagcctatggaaaaacgccagcaacgcggccttttt
acggttcctggccttttgctggccttttgctcacatgt

Nucleotide sequence of sgRNA KCNA1 (SEQ ID NO: 37)
AGTCAATGATCACATCCTCC

Nucleotide sequence of sgRNA LacZ (control) (SEQ I
D NO: 38) TGCGAATACGCCCACGCGAT

Nucleotide sequence of optimised AAV- sgRNA KCNA1-
cFos-rTTA-EGFP vector (SEQ ID NO: 39) ctgcgcgctcgc
tcgctcactgaggccgcccgggcaaagcccgggcgtcgggcgacctttgg
tcgcccggcctcagtgagcgagcgagcgcgcagagagggagtggccaact
ccatcactaggggttcctgcggccgcacgcgtTTAACGAGGGCCTATTTC
CCATGATTCCTTCATATTTGCATATACGATACAAGGCTGTTAGAGAGATA
ATTAGAATTAATTTGACTGTAAACACAAAGATATTAGTACAAAATACGTG
ACGTAGAAAGTAATAATTTCTTGGGTAGTTTGCAGTTTTAAAATTATGTT
TTAAAATGGACTATCATATGCTTACCGTAACTTGAAAGTATTTCGATTTC
TTGGCTTTATATATCTTGTGGAAAGGACGAAACACCGagtcaatgatcac
atcctccGTTTTAGAGCTAGAAATAGCAAGTTAAAATAAGGCTAGTCCGT
TATCAACTTGAAAAAGTGGCACCGAGTCGGTGCTTTTTTGTTAACATCGA
TtCTCGAGTTCGCTATTACGCCAGTTTTATTGCGGCCGCAGCTTTCCTTT
AGGAACAGAGGCTTCGAGCCTTTAAGGCTGCGTACTTGCTTCTCCTAATA
CCAGAGACTCAAAAAAAAAAAAAAAGTTCCAGATTGCTGGACAATGACCC
GGGTCTCATCCCTTGACCCTGGGAACCGGGTCCACATTGAATCAGGTGCG
AATGTTCGCTCGCCTTCTCTGCCTTTCCCGCCTCCCCTCCCCCGGCCGCG
GCCCCGGTTCCCCCCCTGCGCTGCACCCTCAGAGTTGGCTGCAGCCGGCG
AGCTGTTCCCGTCAATCCCTCCCTCCTTTACACAGGATGTCCATATTAGG
ACATCTGCGTCAGCAGGTTTCCACGGCCGGTCCCTGTTGTTCTGGGGGGG
GGACCATCTCCGAAATCCTACACGCGGAAGGTCTAGGAGACCCCCTAAGA
TCCCAAATGTGAACACTCATAGGTGAAAGATGTATGCCAAGACGGGGGTT
GAAAGCCTGGGGCGTAGAGTTGACGACAGAGCGCCCGCAGAGGGCCTTGG
GGCGCGCTTCCCCCCCCTTCCAGTTCCGCCCAGTGACGTAGGAAGTCCAT
CCATTCACAGCGCTTCTATAAAGGCGCCAGCTGAGGCGCCTACTACTCCA
ACCGCGACTGCAGCGAGCAACTGAGAAGACTGGATAGAGCCGGCGGTTCC
GCGAACGAGCAGTGACCGCGCTCCCACCCAGCTCTGCTCTGCAGCTCCCA
CCAGTGTCTGGCCGCATCGATTCTAGAATTCGCTGTCTGCGAGGGCCAGC
TGTTGGGGTGAGTACTCCCTCTCAAAAGCGGGCATGACTTCTGCGCTAAG
ATTGTCAGTTTCCAAAAACGAGGAGGATTTGATATTCACCTGGCCCGCGG
TGATGCCTTTGAGGGTGGCCGCGTCCATCTGGTCAGAAAAGACAATCTTT
TTGTTGTCAAGCTTGAGGTGTGGCAGGCTTGAGATCTGGCCATACACTTG
AGTGACAATGACATCCACTTTGCCTTTCTCTCCACAGGTGTCCACTCCCA
GGTCCAACTGCAGCCCAAGCGGAGGATCCATGTCTAGACTGGACAAGAGC
AAAGTCATAAACGGCGCTCTGGAATTACTCAATGGAGTCGGTATCGAAGG
CCTGACGACAAGGAAACTCGCTCAAAAGCTGGGAGTTGAGCAGCCTACCC
TGTACTGGCACGTGAAGAACAAGCGGGCCCTGCTCGATGCCCTGCCAATC
GAGATGCTGGACAGGCATCATACCCACTTCTGCCCCCTGGAAGGCGAGTC
ATGGCAAGACTTTCTGCGGAACAACGCCAAGTCATTCCGCTGTGCTCTCC
TCTCACATCGCGACGGGGCTAAAGTGCATCTCGGCACCCGCCCAACAGAG
AAACAGTACGAAACCCTGGAAAATCAGCTCGCGTTCCTGTGTCAGCAAGG
CTTCTCCCTGGAGAACGCACTGTACGCTCTGTCCGCCGTGGGCCACTTTA
CACTGGGCTGCGTATTGGAGGAACAGGAGCATCAAGTAGCAAAAGAGGAA
AGAGAGACACCTACCACCGATTCTATGCCCCCACTTCTGAGACAAGCAAT
TGAGCTGTTCGACCGGCAGGGAGCCGAACCTGCCTTCCTTTTCGGCCTGG
AACTAATCATATGTGGCCTGGAGAAACAGCTAAAGTGCGAAAGCGGCGGG
CCGGCCGACGCCCTTGACGATTTTGACTTAGACATGCTCCCAGCCGATGC
CCTGACGACTTTGACCTTGATATGCTGCCTGCTGACGCTCTTGACGATTT
TGACCTTGACATGCTCCCCGGGTTCGAAGCtGAgGGTCGGGGCTCTCTGC
TCACATGTGGCGACGTCGAGGAGAATCCCGGACCGGCCCCgGGTGTACAA
atggtgagcaagggcgaggagctgttcaccggggtggtgcccatcctggt
cgagctggacggcgacgtaaacggccacaagttcagcgtgtccggcgagg
gcgagggcgatgccacctacggcaagctgaccctgaagttcatctgcacc
accggcaagctgcccgtgccctggcccaccctcgtgaccaccctgaccta
cggcgtgcagtgcttcagccgctaccccgaccacatgaagcagcacgact
tcttcaagtccgccatgcccgaaggctacgtccaggagcgcaccatcttc
ttcaaggacgacggcaactacaagacccgcgccgaggtgaagttcgaggg
cgacaccctggtgaaccgcatcgagctgaagggcatcgacttcaaggagg
acggcaacatcctggggcacaagctggagtacaactacaacagccacaac
gtctatatcatggccgacaagcagaagaacggcatcaaggtgaacttcaa
gatccgccacaacatcgaggacggcagcgtgcagctcgccgaccactacc
agcagaacacccccatcggcgacggccccgtgctgctgcccgacaaccac
tacctgagcacccagtccgccctgagcaaagaccccaacgagaagcgcga
tcacatggtcctgctggagttcgtgaccgccgccgggatcactctcggca
tggacgagctgtacaagtaaACCGGTGCTAGCtaaTctagagTCGACAAT
CAACCTCATcgataccgagcgctgctcgagagatctacgggtggcatccc
tgtgacccctccccagtgcctctcctggccctggaagttgccactccagt
gcccaccagccttgtcctaataaaattaagttgcatcattttgtctgact
aggtgtccttctataatattatggggtggaggggggtggtatggagcaag
gggcaagttgggaagacaacctgtagggcctgcggggtctattgggaacc
aagctggagtgcagtggcacaatcttggctcactgcaatctccgcctcct
gggttcaagcgattctcctgcctcagcctcccgagttgttgggattccag
gcatgcatgaccaggctcagctaatttttgtttttttggtagagacgggg
tttcaccatattggccaggctggtctccaactcctaatctcaggtgatct
acccaccttggcctcccaaattgctgggattacaggcgtgaaccactgct
cccttccctgtccttctgattttgtaggtaaccacgtgcggaccgagcgg
ccgcaggaacccctagtgatggagttggccactccctctctgcgcgctcg
ctcgctcactgaggccgggcgaccaaaggtcgcccgacgcccgggctttg
cccgggcggcctcagtgagcgagcgagcgcgcagctgcctgcaggggcgc
ctgatgcggtattttctccttacgcatctgtgcggtatttcacaccgcat
acgtcaaagcaaccatagtacgcgccctgtagcggcgcattaagcgcggc
gggtgtggtggttacgcgcagcgtgaccgctacacttgccagcgccctag
cgcccgctcctttcgctttcttcccttcctttctcgccacgttcgccggc
tttccccgtcaagctctaaatcgggggctccctttagggttccgatttag
tgctttacggcacctcgaccccaaaaaacttgatttgggtgatggttcac
gtagtgggccatcgccctgatagacggtttttcgccctttgacgttggag
tccacgttctttaatagtggactcttgttccaaactggaacaacactcaa
ccctatctcgggctattcttttgatttataagggattttgccgatttcgg
cctattggttaaaaaatgagctgatttaacaaaaatttaacgcgaatttt
aacaaaatattaacgtttacaattttatggtgcactctcagtacaatctg
ctctgatgccgcatagttaagccagccccgacacccgccaacacccgctg
acgcgccctgacgggcttgtctgctcccggcatccgcttacagacaagct
gtgaccgtctccgggagctgcatgtgtcagaggttttcaccgtcatcacc
gaaacgcgcgagacgaaagggcctcgtgatacgcctatttttataggtta
atgtcatgataataatggtttcttagacgtcaggtggcacttttcgggga
aatgtgcgcggaacccctatttgtttatttttctaaatacattcaaatat
gtatccgctcatgagacaataaccctgataaatgcttcaataatattgaa
aaaggaagagtatgagtattcaacatttccgtgtcgcccttattcccttt
tttgcggcattttgccttcctgtttttgctcacccagaaacgctggtgaa
agtaaaagatgctgaagatcagttgggtgcacgagtgggttacatcgaac
tggatctcaacagcggtaagatccttgagagttttcgccccgaagaacgt
tttccaatgatgagcacttttaaagttctgctatgtggcgcggtattatc
ccgtattgacgccgggcaagagcaactcggtcgccgcatacactattctc
agaatgacttggttgagtactcaccagtcacagaaaagcatcttacggat
ggcatgacagtaagagaattatgcagtgctgccataaccatgagtgataa
cactgcggccaacttacttctgacaacgatcggaggaccgaaggagctaa
ccgcttttttgcacaacatgggggatcatgtaactcgccttgatcgttgg
gaaccggagctgaatgaagccataccaaacgacgagcgtgacaccacgat
gcctgtagcaatggcaacaacgttgcgcaaactattaactggcgaactac
ttactctagcttcccggcaacaattaatagactggatggaggcggataaa
gttgcaggaccacttctgcgctcggcccttccggctggctggtttattgc
tgataaatctggagccggtgagcgtgggtctcgcggtatcattgcagcac
tggggccagatggtaagccctcccgtatcgtagttatctacacgacgggg
agtcaggcaactatggatgaacgaaatagacagatcgctgagataggtgc
ctcactgattaagcattggtaactgtcagaccaagtttactcatatatac
tttagattgatttaaaacttcatttttaatttaaaaggatctaggtgaag
atcctttttgataatctcatgaccaaaatcccttaacgtgagttttcgtt
ccactgagcgtcagaccccgtagaaaagatcaaaggatcttcttgagatc
ctttttttctgcgcgtaatctgctgcttgcaaacaaaaaaaccaccgcta
ccagcggtggtttgtttgccggatcaagagctaccaactctttttccgaa
ggtaactggcttcagcagagcgcagataccaaatactgtccttctagtgt
agccgtagttaggccaccacttcaagaactctgtagcaccgcctacatac
ctcgctctgctaatcctgttaccagtggctgctgccagtggcgataagtc
gtgtcttaccgggttggactcaagacgatagttaccggataaggcgcagc
ggtcgggctgaacggggggttcgtgcacacagcccagcttggagcgaacg
acctacaccgaactgagatacctacagcgtgagctatgagaaagcgccac
gcttcccgaagggagaaaggcggacaggtatccggtaagcggcagggtcg
gaacaggagagcgcacgagggagcttccagggggaaacgcctggtatctt
tatagtcctgtcgggtttcgccacctctgacttgagcgtcgatttttgtg
atgctcgtcaggggggcggagcctatggaaaaacgccagcaacgcggcct
ttttacggttcctggccttttgctggccttttgctca

Nucleotide sequence of optimised AAV- sgRNA LacZ-c
Fos-rTTA-EGFP vector (SEQ ID NO: 40) cctgcaggcagct
gcgcgctcgctcgctcactgaggccgcccgggcaaagcccgggcgtcggg
cgacctttggtcgcccggcctcagtgagcgagcgagcgcgcagagaggga
gtggccaactccatcactaggggttcctgcggccgcacgcgtTTAACGAG
GGCCTATTTCCCATGATTCCTTCATATTTGCATATACGATACAAGGCTGT
TAGAGAGATAATTAGAATTAATTTGACTGTAAACACAAAGATATTAGTAC
AAAATACGTGACGTAGAAAGTAATAATTTCTTGGGTAGTTTGCAGTTTTA
AAATTATGTTTTAAAATGGACTATCATATGCTTACCGTAACTTGAAAGTA
TTTCGATTTCTTGGCTTTATATATCTTGTGGAAAGGACGAAACACCGTGC
GAATACGCCCACGCGATGTTTTAGAGCTAGAAATAGCAAGTTAAAATAAG
GCTAGTCCGTTATCAACTTGAAAAAGTGGCACCGAGTCGGTGCTTTTTTG
TTAACATCGATtTCCCACGGGGTCTCGAGTTCGCTATTACGCCAGTTTTA
TTGCGGCCGCAGCTTTCCTTTAGGAACAGAGGCTTCGAGCCTTTAAGGCT
GCGTACTTGCTTCTCCTAATACCAGAGACTCAAAAAAAAAAAAAAAGTTC
CAGATTGCTGGACAATGACCCGGGTCTCATCCCTTGACCCTGGGAACCGG
GTCCACATTGAATCAGGTGCGAATGTTCGCTCGCCTTCTCTGCCTTTCCC
GCCTCCCCTCCCCCGGCCGCGGCCCCGGTTCCCCCCCTGCGCTGCACCCT
CAGAGTTGGCTGCAGCCGGCGAGCTGTTCCCGTCAATCCCTCCCTCCTTT
ACACAGGATGTCCATATTAGGACATCTGCGTCAGCAGGTTTCCACGGCCG
GTCCCTGTTGTTCTGGGGGGGGGACCATCTCCGAAATCCTACACGCGGAA
GGTCTAGGAGACCCCCTAAGATCCCAAATGTGAACACTCATAGGTGAAAG
ATGTATGCCAAGACGGGGGTTGAAAGCCTGGGGCGTAGAGTTGACGACAG
AGCGCCCGCAGAGGGCCTTGGGGCGCGCTTCCCCCCCCTTCCAGTTCCGC
CCAGTGACGTAGGAAGTCCATCCATTCACAGCGCTTCTATAAAGGCGCCA
GCTGAGGCGCCTACTACTCCAACCGCGACTGCAGCGAGCAACTGAGAAGA
CTGGATAGAGCCGGCGGTTCCGCGAACGAGCAGTGACCGCGCTCCCACCC
AGCTCTGCTCTGCAGCTCCCACCAGTGTCTGGCCGCATCGATTCTAGAAT
TCGCTGTCTGCGAGGGCCAGCTGTTGGGGTGAGTACTCCCTCTCAAAAGC
GGGCATGACTTCTGCGCTAAGATTGTCAGTTTCCAAAAACGAGGAGGATT
TGATATTCACCTGGCCCGCGGTGATGCCTTTGAGGGTGGCCGCGTCCATC
TGGTCAGAAAAGACAATCTTTTTGTTGTCAAGCTTGAGGTGTGGCAGGCT
TGAGATCTGGCCATACACTTGAGTGACAATGACATCCACTTTGCCTTTCT
CTCCACAGGTGTCCACTCCCAGGTCCAACTGCAGCCCAAGCGGAGGATCC
ATGTCTAGACTGGACAAGAGCAAAGTCATAAACGGCGCTCTGGAATTACT
CAATGGAGTCGGTATCGAAGGCCTGACGACAAGGAAACTCGCTCAAAAGC
TGGGAGTTGAGCAGCCTACCCTGTACTGGCACGTGAAGAACAAGCGGGCC
CTGCTCGATGCCCTGCCAATCGAGATGCTGGACAGGCATCATACCCACTT
CTGCCCCCTGGAAGGCGAGTCATGGCAAGACTTTCTGCGGAACAACGCCA
AGTCATTCCGCTGTGCTCTCCTCTCACATCGCGACGGGGCTAAAGTGCAT
CTCGGCACCCGCCCAACAGAGAAACAGTACGAAACCCTGGAAAATCAGCT
CGCGTTCCTGTGTCAGCAAGGCTTCTCCCTGGAGAACGCACTGTACGCTC
TGTCCGCCGTGGGCCACTTTACACTGGGCTGCGTATTGGAGGAACAGGAG
CATCAAGTAGCAAAAGAGGAAAGAGAGACACCTACCACCGATTCTATGCC
CCCACTTCTGAGACAAGCAATTGAGCTGTTCGACCGGCAGGGAGCCGAAC
CTGCCTTCCTTTTCGGCCTGGAACTAATCATATGTGGCCTGGAGAAACAG
CTAAAGTGCGAAAGCGGCGGGCCGGCCGACGCCCTTGACGATTTTGACTT
AGACATGCTCCCAGCCGATGCCCTTGACGACTTTGACCTTGATATGCTGC
CTGCTGACGCTCTTGACGATTTTGACCTTGACATGCTCCCCGGGTTCGAA
GCtGAgGGTCGGGGCTCTCTGCTCACATGTGGCGACGTCGAGGAGAATCC
CGGACCGGCCCCgGGTGTACAAatggtgagcaagggcgaggagctgttca
ccggggtggtgcccatcctggtcgagctggacggcgacgtaaacggccac
aagttcagcgtgtccggcgagggcgagggcgatgccacctacggcaagct
gaccctgaagttcatctgcaccaccggcaagctgcccgtgccctggccca
ccctcgtgaccaccctgacctacggcgtgcagtgcttcagccgctacccc
gaccacatgaagcagcacgacttcttcaagtccgccatgcccgaaggcta
cgtccaggagcgcaccatcttcttcaaggacgacggcaactacaagaccc
gcgccgaggtgaagttcgagggcgacaccctggtgaaccgcatcgagctg
aagggcatcgacttcaaggaggacggcaacatcctggggcacaagctgga
gtacaactacaacagccacaacgtctatatcatggccgacaagcagaaga
acggcatcaaggtgaacttcaagatccgccacaacatcgaggacggcagc
gtgcagctcgccgaccactaccagcagaacacccccatcggcgacggccc
cgtgctgctgcccgacaaccactacctgagcacccagtccgccctgagca
aagaccccaacgagaagcgcgatcacatggtcctgctggagttcgtgacc
gccgccgggatcactctcggcatggacgagctgtacaagtaaACCGGTGC
TAGCtaaTctagagTCGACAATCAACCTCATcgataccgagcgctgctcg
agagatctacgggtggcatccctgtgacccctccccagtgcctctcctgg
ccctggaagttgccactccagtgcccaccagccttgtcctaataaaatta
agttgcatcattttgtctgactaggtgtccttctataatattatggggtg
gaggggggtggtatggagcaaggggcaagttgggaagacaacctgtaggg
cctgcggggtctattgggaaccaagctggagtgcagtggcacaatcttgg
ctcactgcaatctccgcctcctgggttcaagcgattctcctgcctcagcc
tcccgagttgttgggattccaggcatgcatgaccaggctcagctaatttt
tgtttttttggtagagacggggtttcaccatattggccaggctggtctcc
aactcctaatctcaggtgatctacccaccttggcctcccaaattgctggg
attacaggcgtgaaccactgctcccttccctgtccttctgattttgtagg
taaccacgtgcggaccgagcggccgcaggaacccctagtgatggagttgg
ccactccctctctgcgcgctcgctcgctcactgaggccgggcgaccaaag
gtcgcccgacgcccgggctttgcccgggcggcctcagtgagcgagcgagc
gcgcagctgcctgcaggggcgcctgatgcggtattttctccttacgcatc
tgtgcggtatttcacaccgcatacgtcaaagcaaccatagtacgcgccct
gtagcggcgcattaagcgcggcgggtgtggtggttacgcgcagcgtgacc
gctacacttgccagcgccctagcgcccgctcctttcgctttcttcccttc
ctttctcgccacgttcgccggctttccccgtcaagctctaaatcgggggc
tccctttagggttccgatttagtgctttacggcacctcgaccccaaaaaa
cttgatttgggtgatggttcacgtagtgggccatcgccctgatagacggt
ttttcgccctttgacgttggagtccacgttctttaatagtggactcttgt
tccaaactggaacaacactcaaccctatctcgggctattcttttgattta
taagggattttgccgatttcggcctattggttaaaaaatgagctgattta
acaaaaatttaacgcgaattttaacaaaatattaacgtttacaattttat
ggtgcactctcagtacaatctgctctgatgccgcatagttaagccagccc
cgacacccgccaacacccgctgacgcgccctgacgggcttgtctgctccc
ggcatccgcttacagacaagctgtgaccgtctccgggagctgcatgtgtc
agaggttttcaccgtcatcaccgaaacgcgcgagacgaaagggcctcgtg
atacgcctatttttataggttaatgtcatgataataatggtttcttagac
gtcaggtggcacttttcggggaaatgtgcgcggaacccctatttgtttat
ttttctaaatacattcaaatatgtatccgctcatgagacaataaccctga
taaatgcttcaataatattgaaaaaggaagagtatgagtattcaacattt
ccgtgtcgcccttattcccttttttgcggcattttgccttcctgtttttg
ctcacccagaaacgctggtgaaagtaaaagatgctgaagatcagttgggt
gcacgagtgggttacatcgaactggatctcaacagcggtaagatccttga
gagttttcgccccgaagaacgttttccaatgatgagcacttttaaagttc
tgctatgtggcgcggtattatcccgtattgacgccgggcaagagcaactc
ggtcgccgcatacactattctcagaatgacttggttgagtactcaccagt
cacagaaaagcatcttacggatggcatgacagtaagagaattatgcagtg
ctgccataaccatgagtgataacactgcggccaacttacttctgacaacg
atcggaggaccgaaggagctaaccgcttttttgcacaacatgggggatca
tgtaactcgccttgatcgttgggaaccggagctgaatgaagccataccaa
acgacgagcgtgacaccacgatgcctgtagcaatggcaacaacgttgcgc
aaactattaactggcgaactacttactctagcttcccggcaacaattaat
agactggatggaggcggataaagttgcaggaccacttctgcgctcggccc
ttccggctggctggtttattgctgataaatctggagccggtgagcgtggg
tctcgcggtatcattgcagcactggggccagatggtaagccctcccgtat
cgtagttatctacacgacggggagtcaggcaactatggatgaacgaaata
gacagatcgctgagataggtgcctcactgattaagcattggtaactgtca
gaccaagtttactcatatatactttagattgatttaaaacttcattttta
atttaaaaggatctaggtgaagatcctttttgataatctcatgaccaaaa
tcccttaacgtgagttttcgttccactgagcgtcagaccccgtagaaaag
atcaaaggatcttcttgagatcctttttttctgcgcgtaatctgctgctt
gcaaacaaaaaaaccaccgctaccagcggtggtttgtttgccggatcaag
agctaccaactctttttccgaaggtaactggcttcagcagagcgcagata
ccaaatactgtccttctagtgtagccgtagttaggccaccacttcaagaa
ctctgtagcaccgcctacatacctcgctctgctaatcctgttaccagtgg
ctgctgccagtggcgataagtcgtgtcttaccgggttggactcaagacga
tagttaccggataaggcgcagcggtcgggctgaacggggggttcgtgcac
acagcccagcttggagcgaacgacctacaccgaactgagatacctacagc
gtgagctatgagaaagcgccacgcttcccgaagggagaaaggcggacagg
tatccggtaagcggcagggtcggaacaggagagcgcacgagggagcttcc
agggggaaacgcctggtatctttatagtcctgtcgggtttcgccacctct
gacttgagcgtcgatttttgtgatgctcgtcaggggggcggagcctatgg
aaaaacgccagcaacgcggcctttttacggttcctggccttttgctggcc
ttttgctcacatgt

1. An expression vector for use in a method of treatment of a neurological disorder associated with neuronal hyperexcitability in a subject, the vector comprising:
(a) (i) a polynucleotide sequence (“gene”) encoding a polypeptide (“gene product”) which ameliorates said disorder when expressed in the subject’s neural cells, wherein the gene is operably linked to
(ii) a neuronal activity-dependent promoter suitable to drive expression of the gene product in the subject’s neural cells; or
(b) (i) an intermediate polynucleotide sequence (“intermediate gene”) encoding an intermediate polypeptide (“intermediate gene product”) which alters expression of a further polynucleotide sequence (“further gene”), the further gene encoding a further polypeptide (“further gene product”) which ameliorates said disorder when expressed in the subject’s neural cells, wherein the intermediate gene is operably linked to:
(ii) a neuronal activity-dependent promoter suitable to drive expression of the intermediate gene product in the subject’s neural cells.

2. The expression vector for use of claim 1, wherein the level of expression of the gene product or intermediate gene product or further gene product increases when the neuron becomes more excited and decreases when the neuron becomes less excited.

3. The expression vector for use according to any one of the above claims, wherein the promoter is a pyramidal neuronal activity-dependent promoter.

4. The expression vector for use according to any one of the above claims, wherein the promoter is an immediate early gene (IEG) promoter.

5. The expression vector for use according to any one of the above claims, wherein the promoter is c-Fos.

6. The expression vector for use according to any one of the above claims, wherein the promoter has a nucleotide sequence comprising or consisting of the nucleotide sequence shown in SEQ ID NO: 3 or a nucleotide sequence having at least 80% identity to the nucleotide sequence shown in SEQ ID NO: 3.

7. The expression vector for use according to any one of claims 1-4, wherein the promoter is Arc, or wherein the promoter comprises a nucleotide sequences that comprises part of the Arc nucleotide sequence.

8. The expression vector for use according to any one of claims 1-4, wherein the promoter is mArc (“minimal Arc”).

9. The expression vector for use according to claim 7 or claim 8, wherein the promoter has a nucleotide sequence comprising or consisting of the nucleotide sequence shown in SEQ ID NO: 15 or a nucleotide sequence having at least 80% identity to the nucleotide sequence shown in SEQ ID NO: 15.

10. The expression vector for use according to any one of claims 1-4, wherein the promoter is ESARE (“enhanced synaptic activity-responsive element”).

11. The expression vector for use according to claim 9, wherein the promoter has a nucleotide sequence comprising or consisting of the nucleotide sequence shown in SEQ ID NO: 16 or a nucleotide sequence having at least 80% identity to the nucleotide sequence shown in SEQ ID NO: 16.

12. The expression vector for use according to any one of claims 1-4, wherein the promoter is NRAM (“Npas4-specific Robust Activity Marker”).

13. The expression vector for use according to claim 11, wherein the promoter has a nucleotide sequence comprising or consisting of the nucleotide sequence shown in SEQ ID NO: 17 or a nucleotide sequence having at least 80% identity to the nucleotide sequence shown in SEQ ID NO: 17.

14. The expression vector for use according to any one of claims 1-4, wherein the promoter is Egr1.

15. The expression vector for use according to claim 13, wherein the promoter has a nucleotide sequence comprising or consisting of the nucleotide sequence shown in SEQ ID NO: 18 or a nucleotide sequence having at least 80% identity to the nucleotide sequence shown in SEQ ID NO: 18.

16. The expression vector for use according to any one of the above claims, wherein the gene or further gene is an ion channel gene, and the gene product or further gene product is an ion channel.

17. The expression vector for use according to any one of the above claims, wherein the gene or further gene is a potassium ion channel gene, and the gene product or further gene product is a potassium ion channel.

18. The expression vector for use according to any one of the above claims, wherein the gene or further gene is a KCNA1 gene, and the gene product or further gene product is a Kv1.1 potassium channel.

19. The expression vector for use according to any one of the above claims, wherein the gene or further gene is an engineered KCNA1 gene, and the gene product or further gene product is an edited Kv1.1 potassium channel.

20. The expression vector for use according to claim 19, wherein the engineered KCNA1 gene has a nucleotide sequence having at least 90% sequence identity to the nucleotide sequence shown in SEQ ID NO: 1, and
wherein the edited Kv1.1 potassium channel has an amino acid sequence having at least 90% sequence identity to the amino acid sequence shown in SEQ ID NO: 2 and comprises a valine amino acid residue at a position corresponding to amino acid residue 400 shown in SEQ ID NO: 2.

21. The expression vector for use according to any one of claims 1-16, wherein the gene or further gene is a KNCJ2 gene, and the gene product or further gene product is a Kir2.1 potassium channel.

22. The expression vector for use according to claim 21, wherein the KNCJ2 gene has a nucleotide sequence having at least 90% sequence identity to the nucleotide sequence shown in SEQ ID NO: 13, and
wherein the Kir2.1 potassium channel has an amino acid sequence having at least 90% sequence identity to the amino acid sequence shown in SEQ ID NO: 14.

23. The expression vector for use according to any one of the above claims, wherein the further gene is an endogenous gene and the further gene product is an endogenous gene product.

24. The expression vector for use according to claim 23, wherein the endogenous gene is KCNA1 or KCNJ2.

25. The expression vector for use according to any one of the above claims, wherein the intermediate gene is endonuclease deficient cas (“dcas”), such as dcas9, spCas9 or saCas9.

26. The expression vector for use according to claim 25, further comprising:
(a) RNA polymerase III, optionally wherein the polymerase III is U6; and
(b) an sgRNA (“single guide RNA”) that targets the further gene.

27. The expression vector for use according to any one of the above claims, wherein the intermediate gene product increases expression of the further gene via an intermediate expression system, optionally an intermediate inducible expression system.

28. The expression vector for use according to claim 27, wherein the intermediate inducible expression system is a Tet-On expression system.

29. An expression vector system for use in a method of treatment of a neurological disorder associated with neuron hyperexcitability in a subject, comprising:
(a) a first nucleotide sequence comprising a neuronal activity-dependent promoter suitable to drive expression of reverse tetracycline-controlled transactivator (“rtTA”) in the subject’s neural cells; and
(b) a second nucleotide sequence comprising a Tet-On promoter suitable to drive expression of an intermediate gene or further gene according to any one of the above claims,
wherein either the first nucleotide, second nucleotide, or expression system optionally further comprises:
(c) an RNA polymerase, optionally wherein the RNA polymerase is RNA polymerase II or RNA polymerase III, further optionally wherein the RNA polymerase III is U6; and
(d) an sgRNA (“single guide RNA”) that targets the further gene. and/or
(e) a tetracycline, preferably doxycycline.

30. An expression vector system for use according to claim 29, comprising:
(a) a first nucleotide sequence comprising or consisting of the nucleotide sequence shown in SEQ ID NO: 37 or 39, or a nucleotide sequence having at least 80% identity to the nucleotide sequence shown in SEQ ID NO: 37 or 39; and
(b) a second nucleotide sequence comprising or consisting of the nucleotide sequence shown in SEQ ID NO: 35 or 36, or a nucleotide sequence having at least 80% identity to the nucleotide sequence shown in SEQ ID NO: 35 or 36.

31. The expression vector or vector system for use according to any one of the above claims,wherein the vector or vector system can cause a reduction in the spike frequency of a neuron of the subject by more than 5%, or more than 10%, or more than 20%, or more than 30%, or more than 40%, or more than 50%, or more than 60%, or more than 70%, or more than 80%, or more than 90%, or more than 91%, or more than 92%, or more than 93%, or more than 94%, or more than 95%, or more than 96%, or more than 97%, or more than 98%, or more than 99%, or 100%.

32. The expression vector or vector system for use according to claim 31, wherein the vector or vector system can cause a reduction in the spike frequency of a neuron of the subject by more than 75%.

33. The expression vector or vector system for use according to claim 32 or claim 31, wherein the reduction in the spike frequency of the neuron is measured using multi-electrode arrays on or after 21 DIV (days in vitro).

34. The expression vector or vector system for use according to any one of claims 31-33, wherein the reduction in the spike frequency of the neuron is measured relative to a vector comprising SEQ ID NO: 6.

35. The expression vector or vector system for use according to any one of claims 31-34, wherein the neuron is a primary cortical neuron.

36. The expression vector or vector system for use according to any one of the above claims, wherein the vector or vector system can cause fewer than 10 action potentials per second, or fewer than 5 action potentials per second, or fewer than 4 action potentials per second, or fewer than 3 action potentials per second, or fewer than 2 action potentials per second, or no action potentials per second, in a neuron.

37. The expression vector or vector system for use according to any one of the above claims, wherein the vector or vector system can cause a resting membrane potential in a neuron of less than -50 mV, or less than -60 mV, or less than -70 mV, or less than -80 mV, or less than -90 mV, or less than -100 mV.

38. The expression vector or vector system for use according to any of the above claims, wherein the vector or vector system can increase the threshold for action potentials in a neuron to more than 50 pA, or more than 75 pA, or more than 100 pA, or more than 150 pA, or more than 200 pA, or more than 250 pA, or more than 300 pA, or more than 350 pA, or more than 400 pA, or more than 450 pA, or more than 500 pA, or more than 550 pA, or more than 600 pA, or more than 700 pA, or more than 800 pA, or more than 900 pA, or more than 1000 pA, wherein the threshold is the sum of current threshold and holding current.

39. The expression vector or vector system for use according to any one of claims 36-38, wherein the number of action potentials, resting membrane potential, or threshold for action potentials is measured in an acute hippocampal slice from a subject.

40. The expression vector or vector system for use according to any one of claims 36-39, wherein the number of action potentials, resting membrane potential, or threshold for action potentials is measured using acute hippocampal slice electrophysiology and/or patch clamp electrophysiology.

41. The expression vector or vector system for use according to any one of claims 36-40, wherein the neuron is capable of driving a seizure and/or when the neuron generates sustained firing and/or when the neuron becomes over-depolarised.

42. The expression vector or vector system for use according to any one of the above claims, wherein the vector or vector system can cause a greater anti-epileptic effect in a neuron driving a second seizure in a subject, than the anti-epileptic effect in the neuron driving the first seizure in the subject, wherein the second seizure is subsequent to the first seizure.

43. The expression vector or vector system for use according to claim 42, wherein anti-epileptic effect is measured using any of the methods described in claims 23-33.

44. The expression vector or vector system for use according to any one of the above claims, wherein the vector or vector system can prevent a second seizure in a subject, wherein the second seizure is subsequent to a first seizure in the subject.

45. The expression vector or vector system for use of any of the above claims, wherein the method of treatment is close-loop therapy.

46. The expression vector or vector system for use according to any one of the above claims, wherein the neurological disorder is a seizure disorder.

47. The expression vector or vector system for use according to claim 46, wherein the seizure disorder is epilepsy, optionally neocortical epilepsy, temporal lobe epilepsy or refractory epilepsy.

48. The expression vector or vector system for use according to any one of claims 1-45, wherein the neurological disorder is Parkinson’s disease, chronic pain, sudden unexpected death in epilepsy (SUDEP), migraine, cluster headache, trigeminal neuralgia, post-herpetic neuralgia, paroxysmal movement disorders, uni-or bipolar affective disorders, anxiety, or phobias.

49. The expression vector or vector system for use according to any one of the above claims, wherein the vector or vector system is a viral vector or vector system.

50. The expression vector or vector system for use according to claim 49, wherein the viral vector or vector system is a recombinant adeno-associated virus (AAV) vector or vector system, or a lentiviral vector or vector system, optionally wherein the lentiviral vector or vector system is a non-integrating lentiviral vector or vector system.

51. The expression vector or vector system for use according to claim 49, wherein the vector or vector system comprises a nucleotide sequence having at least 95% identity to the nucleotide sequence of SEQ ID NO: 8, SEQ ID NO: 9 or SEQ ID NO: 10.

52. The expression vector or vector system for use according to claim 49, wherein the vector or vector system comprises a nucleotide sequence having at least 95% identity to any one of SEQ ID NOs: 20-34.

53. An expression vector comprising:
(a) an engineered KCNA1 gene having a nucleotide sequence having at least 90% sequence identity to the nucleotide sequence shown in SEQ ID NO: 1, encoding an edited Kv1.1 potassium channel having an amino acid sequence having at least 90% sequence identity to the amino acid sequence shown in SEQ ID NO: 2 and comprises a valine amino acid residue at a position corresponding to amino acid residue 400 shown in SEQ ID NO: 2; and
(b) an activity-dependent promoter having a nucleotide sequence comprising or consisting of the nucleotide sequence shown in SEQ ID NO: 3 or a nucleotide sequence having at least 80% identity to the nucleotide sequence shown in SEQ ID NO: 3,
wherein the gene is operably linked to the promoter.

54. An expression vector system comprising:
(a) (i) an engineered KCNA1 gene having a nucleotide sequence having at least 90% sequence identity to the nucleotide sequence shown in SEQ ID NO: 1, encoding an edited Kv1.1 potassium channel having an amino acid sequence having at least 90% sequence identity to the amino acid sequence shown in SEQ ID NO: 2 and comprises a valine amino acid residue at a position corresponding to amino acid residue 400 shown in SEQ ID NO: 2; or
(ii) a KCNJ2 gene having a nucleotide sequence having at least 90% sequence identity to the nucleotide sequence shown in SEQ ID NO: 13, encoding a Kir2.1 potassium channel having an amino acid sequence having at least 90% sequence identity to the amino acid sequence shown in SEQ ID NO: 14; and
(b) an activity-dependent promoter having a nucleotide sequence comprising or consisting of the nucleotide sequence shown in any one of SEQ ID NOs: 3 and 13-18, or a nucleotide sequence having at least 80% identity to the nucleotide sequence shown in any one of SEQ ID NOs: 3 and 13-18, wherein
(c) the KCNA1 or KCNJ 2 gene is operably linked to the promoter; or
(d) the KCNA1 or KCNJ 2 gene’s expression can be altered by an intermediate gene as defined in any one of the above claims, wherein the intermediate gene is operably linked to the activity-dependent promoter.

55. An in vitro method of making viral particles comprising:
transducing mammalian cells with a vector or vector system according to any one of the above claims and expressing viral packaging and envelope proteins necessary for particle formation in the cells; and
culturing the transduced cells in a culture medium, such that the cells produce viral particles that are released into the medium.

56. An in vitro method of claim 55, wherein the method comprises transducing the mammalian cells with one or more viral packaging and envelope expression vectors that encode the viral packaging and envelope proteins necessary for particle formation.

57. An in vitro method of claim 55 or claim 56, wherein the one or more packaging proteins includes a non-functional integrase enzyme such that the vector or vector system is unable to incorporate its viral genome into the genome of the cell.

58. An in vitro method of any one of claims 55-57, further comprising separating the viral particles from the culture medium and optionally concentrating the viral particles.

59. A viral particle produced by the method of any one of claims 55-58, the viral particle optionally comprising an RNA molecule or DNA molecule transcribed from the expression vector or vector system of any of the above claims.

60. A viral particle comprising a single stranded RNA molecule or DNA molecule encoding a gene, and/or intermediate gene, and/or further gene as described in any one of the above claims,
wherein the gene, and/or intermediate gene, and/or further gene encodes a gene product, and/or intermediate gene product, and/or further gene product as defined in any one of the above claims,
wherein the promoter is optionally as defined in any one of the above claims, and
wherein the viral particle is optionally an AAV.

61. A kit comprising an expression vector or vector system of any one of the above claims and one or more viral packaging and envelope expression vectors that encode viral packaging and envelope proteins necessary for particle formation when expressed in a cell.

62. A kit of claim 61, wherein the viral packaging expression vector is an integrase-deficient viral packaging expression vector.

63. A viral particle of claim 59 or claim 60 for use in a method of treatment, wherein the method of treatment is defined in any one of claims 45-48.

64. A method of treatment of a neurological disorder as defined in any one of claims 1 and 45-48, comprising administering to an individual with the neurological disorder the expression vector or vector system as defined in any one of the above claims, or the viral particle of claim 59 or 60.

65. A method of confirming the presence of a gene product and/or intermediate gene product, and/or further gene product as defined in any one of the above claims, the method comprising:
transducing a cell with an expression vector of any one of the above claims or administering a viral particle of claim 59 or 60 to a cell under conditions that permit expression of the gene product and/or intermediate gene product, and/or further gene product; and
detecting the presence of the gene product and/or intermediate gene product, and/or further gene product in the cell using a hybridisation assay.

66. An in vitro or ex vivo method of confirming the presence of a gene product and/or intermediate gene product, and/or further gene product as defined in any one of the above claims that has been obtained from a subject administered with a viral particle of claim 59 or 60, the method comprising:
detecting the presence of the gene product and/or intermediate gene product, and/or further gene product in the cell using a hybridisation assay.

67. A method of claim 65 or claim 66, wherein the hybridisation assay is an in situ hybridisation assay using a labelled RNA probe, optionally wherein the labelled RNA probe is fluorescently labelled.

68. A cell comprising the expression vector or vector system of any one of the above claims.
